(19) United States
(12) Patent Application Publication
(10) Pub. No.: US 2009/0325298 A1
(43) Pub. Date: Dec. 31, 2009

(54) Pro-Apoptotic Bacteria and Compositions for Delivery and Expression of Antigens

(76) Inventor: Douglas S. Kernodle, Brentwood, TN (US)

Correspondence Address: Ballard Spahr LLP, SUITE 1000, 999 PEACHTREE STREET, ATLANTA, GA, 30309-3915, US

(21) Appl. No.: 12/085,085

(22) PCT Filed: Nov. 15, 2006
(86) PCT No.: PCT/US06/44429

§ 371 Date: Mar. 27, 2009

Related U.S. Application Data
(60) Provisional application No. 60/737,525, filed on Nov. 15, 2005.

(51) Int. Cl. C12N 15/87; C12N 1/21

(52) U.S. Cl. 435/471; 435/440; 435/252.3; 435/252.34

(57) ABSTRACT

Whole-cell vaccines and methods for enhancing the immunogenicity of cellular microorganisms for use in producing protective immune responses in vertebrate hosts subsequently exposed to pathogenic bacteria or for use as vectors to express exogenous antigens and induce responses against other infectious agents or cancer cells. The present invention involves an additional method of enhancing antigen presentation by intracellular bacteria in a manner that improves vaccine efficacy. After identifying an enzyme that has an anti-apoptotic effect upon host cells infected by an intracellular microbe, the activity of the enzyme produced by the intracellular microbe is reduced by expressing a mutant copy of the enzyme, thereby modifying the microbe so that it increases immunogenicity.

### CROSS-REFERENCE TO RELATED APPLICATIONS

This application claims the benefit of Provisional Patent Application No. 60/737,525 filed Nov. 15, 2005, which application is incorporated herein by reference.

This invention was made with government support under NIH Grant AI 51561 and some of the work involved the use of research facilities in Department of Veteran's Affairs Medical Centers. The U.S. Government may have certain rights in this invention.

### BACKGROUND OF THE INVENTION

1. Field of the Invention

The present invention relates to the field of vaccination including the induction of strong immune responses and the prevention and treatment of infectious diseases and cancer. Specifically, the present invention relates to methods for enhancing the immunogenicity of a bacterium by expressing dominant-negative mutants of superoxide dismutase, glutamine synthase, and other anti-apoptotic enzymes. It further relates to methods for producing a safe and effective vaccine and methods for enhancing an effective immune response in host animals subsequently exposed to infection by bacterial pathogens, for example, Mycobacterium tuberculosis. The immunogenic vaccines constructed by using these methods can also be vectors for expressing exogenous antigens and used to induce an immune response against unrelated infectious agents and cancer.

2. Background

Adaptive immune responses involving B- and T-lymphocytes are an important component of how the immune system protects the host from infection and cancer. There is specialization in the adaptive immune response with different cells and factors conferring protection against different challenges. For example, humoral immune responses are mediated by B-cells that mature into plasma cells. These cells can produce neutralizing antibodies that inactivate microbial toxins (e.g., diphtheria toxin, pertussis toxin). Antibodies are soluble and can exert their effect over long distances. In contrast, T-cells mediate cellular immune responses that generally require direct or close cell-to-cell contact.

There are two broad categories of T-cells that mediate the effector functions of the adaptive immune response. These two types of T-cells are distinguishable by surface antigens and function [Seder, R. A. et al, 2000]. T-cells exhibiting a CD4 surface antigen include “helper cells.” Some helper cells produce IFN-gamma that activates macrophages to produce more reactive oxygen species and thereby enhances their microbicidal functions. Other CD4+ T-cells produce IL-2 and other interleukins that promote the proliferation of memory T-cell populations into effector T-cells during a subsequent challenge with an infectious agent. CD8+ cells exert their protective effect in several ways including cytotoxic T-lymphocyte (CTL) activity resulting in lysis of infected cells, by killing intracellular bacilli via the release of the antimicrobial peptide granulysin, and by IFN-gamma production [Cho, S. et al, 2000; Serbina, N. V. et al, 1999; Serbina, N. V. et al, 2000; Silva, C. L. et al, 1999].

Classic immunology teaches that CD4+ lymphocytes and CD8+ lymphocytes are primed for an immune response using different antigen presentation pathways [Seder, R. A. et al, 2000]. In inducing responses involving CD4+ T-cells, exogenous foreign antigens are taken up or recovered from ingested microbes within the phagosome of antigen presenting cells. There the antigens are degraded into fragments by cathepsins and bound on the surface of these cells to MHC Class II molecules for presentation to the CD4+ T-cells. This process is called the “exogenous” pathway of antigen presentation, as it deals with antigens that were originally outside of the cell and ingested by the cell. MHC Class II molecules are restricted primarily to some few types of leukocytes known as “antigen-presenting cells”, which includes macrophages and dendritic cells.

CD8+ T-cell activation is achieved via a different mechanism that involves MHC Class I molecules, which are found on essentially all nucleated cells. Proteins produced by the cell or introduced into the cytoplasm of the nucleated cell are degraded to peptides and presented on the cell surface in the context of MHC Class I molecules to CD8+ T-cells. MHC Class I antigen presentation is generally referred to as the “endogenous” pathway that deals with antigens coming from the cytoplasm, typically antigens from viruses that infect cells.

The current application discloses methods for reducing the activity of an anti-apoptotic microbial enzyme. Also disclosed are modified bacteria made in accordance with the disclosed methods that have enhanced immunogenicity.

### SUMMARY OF THE INVENTION

The present invention involves a method of modifying a bacterium to enhance antigen presentation in a manner that improves vaccine efficacy. Modifying an intracellular organism to express a pro-apoptotic phenotype is provided.

Also, as the induction of strong CD8+ T-cell responses has generally been difficult to achieve with current vaccination strategies, the present modified microbes provide a very effective way to access this arm of the immune system. The microbe can be further altered by adding exogenous DNA encoding immunodominant antigens from other pathogenic microbes including viruses, bacteria, protozoa, and fungi or with DNA encoding cancer antigens, and then used to vaccinate a host animal. Therefore, the present attenuated bacterium can be used as a vaccine delivery vehicle to present antigens for processing by MHC Class I and MHC Class II pathways. And because of strong co-stimulatory signals induced by microbial components in the vaccine vector that interact with T cell-like receptors on the host cell, this directs the host immune system to react against the exogenous antigen rather than develop immune tolerance. Furthermore, the simultaneous presentation of antigens by MHC Class I and MHC Class II pathways by dendritic cells facilitates the development of CD4 “help” for CD8 cytotoxic T-lymphocyte (CTL) responses, thereby overcoming limitations of antigen presentation by current vectors that have been designed to access either exogenous (e.g., many bacterial vectors, phagosome-associated) or endogenous (e.g., many viral vectors, cytoplasm and proteasome-associated) pathways of antigen presentation.

### BRIEF DESCRIPTION OF THE DRAWINGS

FIG. 1 shows figures of the iron co-factored superoxide dismutase of M. tuberculosis/BCG (SodA). (A) SodA monomer showing positions of deleted amino acids in the present SodA mutants. Other deletions, additions, and/or substitutions can be used to produce additional dominant-negative SodA mutants. (B) shows SodA tetramer with each rectangle indicating the position of two active site iron ions. The arrows identify active-site iron and E54 positions for the same monomer. The figure was downloaded from the National Center for Biotechnology Information (NCBI) web server (http://www.ncbi.nlm.nih.gov/entrez/query.fcgi?db=Structure&itool=toolbar) and modified to illustrate features.

FIG. 2 provides a map (A) and features (B) of mycobacterial chromosomal integration vector pMP399, and a map (C) and features (D) of plasmid vector pMP349 that expresses mutant SodA ΔH28ΔH76 in BCG. The name for the gene encoding iron co-factored superoxide dismutase in M. tuberculosis/BCG is sodA. It is expressed behind an inducible aceA(icl) promoter. The E. coli origin of replication (oriE) allows the plasmid to replicate in E. coli. The apramycin resistance gene (aacC41) and vectors pMP399 and pMP349 was developed by Consaul and Pavelka [Consaul, S. A. et al, 2004]. The apramycin resistance gene can be replaced by a different antibiotic resistance gene or the vector can contain a biosynthetic gene that complements amino acid auxotrophy in the bacterial strain, thereby allowing growth on media lacking the essential factor (e.g., the amino acid) to be used as a selectable marker for identification of successful recombinants.

FIG. 3 shows SOD activity in supernatants and lysates of BCG that expresses mutant SodA (ΔH28ΔH76) compared to SOD activity of the parent BCG strain. (A) and (B) show results from two separate experiments. The assay is performed using serial 2-fold dilutions of supernatant and lysate and monitoring the amount of reduced cytochrome C at a fixed time point. A unit of SOD activity inhibits cytochrome C reduction by 50% (of the maximal measured inhibition). The dilution that inhibits cytochrome C reduction by 50% (IC50 value) for each preparation is indicated by arrows. SodA is secreted by BCG and thus the SOD activity of BCG supernatant is greater than the SOD activity of BCG lysate.

FIG. 4 shows SOD activity in supernatants and lysates of BCG that expresses mutant SodA (ΔE54) compared to SOD activity of the parent BCG strain.

FIG. 5 shows comparative vaccine efficacy of BCG versus SD-BCG-AS-SOD. The SD-BCG (SodA-diminished BCG) strains used in these experiments were constructed using antisense techniques (see WO 02/062298 entitled “Pro-apoptotic bacterial vaccines to enhance cellular immune responses,” incorporated herein by reference for its teaching of antisense reduction in SOD activity), and exhibit about 1% of the SOD activity of the parent BCG strains. C57Bl/6 mice were vaccinated IV with BCG or SD-BCG-AS-SOD, rested for 7 months, and then challenged by aerosol with 30 cfu of an acriflavin-R mutant of the virulent Erdman strain of M. tuberculosis. At 14 wk post-challenge, unvaccinated and BCG-vaccinated mice displayed focal areas of densely cellular parenchymal lung inflammation (representative section shown in A, x2 and x10). In contrast, SD-BCG-vaccinated mice had less densely cellular areas of lung involvement (B, x2 and x10). Higher power views of B (C) show foamy cells with nuclear fragments suggestive of ingested apoptotic debris in an alveolus (left panel) and multinucleated giant cells (right panel). At the time of final harvest at six months post-challenge, Erdman cfu counts were lower in recipients of SD-BCG compared to recipients of BCG (D). The line within the box plot represents the median, the edges of the box indicate 25th and 75th percentiles, and the whiskers represent 10th and 90th percentiles. The difference between groups was statistically significant (P=0.04, two-sample t-test). Also at this time, the final mean weights of mice in each group were 28.3 and 31.0 gms, BCG [8 survivors from original 12 mice, 4 euthanized from skin problems] and SD-BCG [10 survivors] respectively, P=0.04, two-sample t-test. Thus, reducing SodA production by BCG enhanced its efficacy as a vaccine.

FIG. 6 shows that vaccination with SD-BCG-AS-SOD alters recall T-cell responses in the lungs of mice post-aerosol challenge with virulent M. tuberculosis. Mice were vaccinated with 2×10⁶ cfu subQ with either BCG, SD-BCG-AS-SOD, or phosphate-buffered saline (unvaccinated), rested for 100 days, and then challenged with 300 cfu of Erdman by aerosol. Values represent the number of cells expressing the indicated surface antigens (left column) recovered from the right lung of mice at 4, 10, and 18 days post-challenge. Both lungs were harvested from control mice. Each value represents the mean of 4 mice, except that 3 mice were used for the control values. The BCG-vaccinated group includes mice that received either BCG or C-BCG. Recipients of SD-BCG exhibited greater numbers of CD44+/CD45RB^{high} cells by day 4 post-infection. These cells were larger than other T-cell populations by forward scatter and may represent T-cells undergoing clonal expansion. By day 18, larger numbers of terminally-differentiated CD4+ effector T-cells (CD44+/CD45RB^{neg}) were observed in recipients of SD-BCG than BCG. *P=0.02; ¶ P<0.05, BCG versus SD-BCG, two-sample t-test.

FIG. 7 shows accelerated formation of Ghon lesions in mice vaccinated with SD-BCG-AS-SOD after aerosol challenge with 300 cfu of an acriflavin-R mutant of the virulent Erdman strain of M. tuberculosis. Low (×2) and mid (×20) power photomicrographs of left lungs at day 18 post-challenge are shown. Between day 10 and day 18 post-challenge, SD-BCG-vaccinated developed numerous small focal aggregates of cells in the lung parenchyma (right panels). Such changes between day 10 and day 18 were less apparent in BCG-vaccinated mice and not observed in unvaccinated mice. The small focal cell collections in SD-BCG mice differed in appearance from the expanding areas of granulomatous inflammation in BCG-vaccinated mice, showing more large mononuclear cells with pale cytoplasm and early foamy changes, often containing nuclear fragments suggestive of apoptotic cell debris.

FIG. 8 shows the map (A) and features (B) of the vector that was used to inactivate sigH on the chromosome of BCG and construct SIG-BCG (BCGΔsigH).

FIG. 9 shows lung cfu counts at 6 months post aerosol challenge. Mice were rested for 100 days following subQ vaccination with BCG or BCGΔsigH and then challenged with 300 cfu of an acriflavin-R mutant of the virulent Erdman strain of M. tuberculosis. The line within the box plot represents the median, the edges of the box indicate 25th and 75th percentiles, and the whiskers represent 10^{th} and 90^{th} percentiles. The difference between groups was statistically significant (P=0.019, two-sample T-test.).

FIG. 10 shows photomicrographs of lung sections of mice vaccinated with placebo (saline), BCG, or BCGΔsigH at 6 months post-challenge with 300 cfu of an acriflavin-R mutant of the virulent Erdman strain of M. tuberculosis. Lungs from two mice in each group were inflated with 10% buffered formalin and paraffin-embedded. Three low-power photomicrographs covering about 80% of the lung tissue sections shown on the microscope slide are displayed and show less diseased lungs in the mice vaccinated with BCGΔsigH. Boxes indicates regions shown under higher-power magnification in FIG. 11.

FIG. 11 shows the formation and evolution of Ghon lesions (arrows) at 22 days, 2 mo., and 6 mo post-aerosol challenge of mice with 300 cfu of an acriflavin-R mutant of the virulent Erdman strain of M. tuberculosis. Mice were vaccinated with placebo (saline), BCG, or BCGΔsigH subcutaneously and rested for 100 days before aerosol challenge. Ghon lesions develop earlier in BCGΔsigH-vaccinated mice and evolve with less granulomatous inflammation, thereby resulting in minimal lung damage. In contrast, areas of dense parenchymal infiltration by lymphocytes and macrophages develop in the lungs of unvaccinated and BCG-vaccinated mice. The 6-month photomicrographs correspond to the boxed regions in FIG. 10.

FIG. 12 illustrates sequential steps in immune activation and shows how microbial anti-oxidants can interfere with the activation of the immune response in its early stages. Reducing the activity of microbial anti-oxidants favors apoptosis and other immune functions during vaccination. This leads to strong memory T-cell responses and enhanced protection.

FIG. 13 shows a strategy for combining gene deletions and dominant-negative mutations in multiple genes to yield progressively more potent pro-apoptotic BCG strains to use as vaccines against tuberculosis and as vectors for expressing exogenous antigens. The pro-apoptotic vaccine strains are constructed using a “generation” approach where the 1^{st} generation involves modification of BCG to include a single gene inactivation or dominant-negative mutant enzyme expression, the 2^{nd} generation combines two modifications, the 3^{rd} generation combines three modifications, and the 4^{th} generation combines four modifications.

FIG. 14 shows SOD activity in supernatants and lysates of SIG-BCG and SAD-SIG-BCG. SIG-BCG (also referred to as “sigH-deleted BCG”, or “BCGΔsigH”) is designated BCGdSigH in this figure. SAD-SIG-BCG (also referred to as “BCGHΔsigH [mut sodA]” is designated BCGdSigH H28H76 (panels A and B) or BCGdSigH E54 (panel C), depending upon which dominant-negative mutant was tested. “supe” is an abbreviation for supernatant. The assay is performed using serial 2-fold dilutions of supernatant and lysate and monitoring the amount of reduced cytochrome C at a fixed time point. A unit of SOD activity inhibits cytochrome C reduction by 50% (of the maximal measured inhibition). The dilution that inhibits cytochrome C reduction by 50% (IC50 value) for each preparation is indicated by arrows.

FIG. 15 shows Southern hybridization results that verify the construction of DD-BCG (“double-deletion BCG”), as referred to as “BCGΔsigHΔsecA2.” Chromosomal DNA from four isolates was digested with DraIII, applied to lanes 1-4, and then hybridized with gene probes. The gene probes were directed against secA2, sigH, and hygR (the gene encoding a hygromycin resistance cassette used in the insertional inactivation of sigH). The hygromycin-resistance gene (hygR) had an internal restriction site predicted to yield 2.92 and 1.67 kb fragments when a double-crossover event between the vector and chromosome had eliminated sigH and thus provided additional assurance of success (beyond the absence of a sigHband). The sequence of events in the construction of DD-BCG included the following steps: Starting with the BCG Tice strain (Lane 1) the secA2 gene in BCG Tice was inactivated by using methods previously used to inactivate secA2 in a virulent M. tuberculosis strain [Braunstein, M. et al, 2002; Braunstein, M. et al, 2003, incorporated herein by reference for its teaching of methods to inactivate secA2], thereby producing BCGΔsecA2 (Lane 2). The allelic inactivation vector shown in FIG. 8 was used to inactivate sigH in BCG to yield BCGΔsigH (Lane 3) and also to delete sigH in BCGΔsecA2, thereby yielding BCGΔsigHΔsecA2 (Lane 4, DD-BCG).

FIG. 16 shows SOD activity in lysates of sigH-secA2-deleted BCG (BCGΔsigHΔsecA2, also referred to as double-deletion BCG [“DD-BCG”]) and DD-BCG strains that express mutant SodA (ΔE54) or mutant SodA (ΔH28ΔH76), which are also referred to as 3D-BCG-mutSodA(ΔE54), and 3D-BCG-mutSodA(ΔH28ΔH76). These examples of 3D-BCG strains involve the pMP399-derived vectors and have a mut sodA inserted into the chromosome (of DD-BCG). Panel (A) shows results for supernatants and lysates. Supernatants exhibit less SOD activity than lysates because of the inactivation of secA2, which encodes the secretion channel for SodA and catalase. Panels B-D show SOD activity results from three separate experiments involving lysates prepared on different days using independent cultures of each isolate. The assay is performed using serial 2-fold dilutions of supernatant and lysate and monitoring the amount of reduced cytochrome C at a fixed time point. A unit of SOD activity inhibits cytochrome C reduction by 50% (of the maximal measured inhibition). The dilution where that inhibits cytochrome C reduction by 50% (IC50 value) for each preparation is indicated by arrows.

FIG. 17 shows SDS-PAGE and Western hybridization of lysates of DD-BCG (lane 3), 3D-BCG-mutSodA(ΔE54) (lane 4), and 3D-BCG-mutSodA(ΔH28ΔH76) (lane 5). These examples of 3D-BCG strains have a mut sodA inserted into the chromosome of DD-BCG. The Western hybridization gel shows comparable amounts of SodA in lysates of DD-BCG and two 3D-BCG constructs. Undiluted lysates for PAGE and Western were prepared as described in the methods for the examples (below). BSA=bovine serum albumin, a prominent component in broth media. The E. coli SOD (lane 2) does not react with the antibody against M. tuberculosis SodA. The undiluted lysates applied to these gels are the same as the lysates used in the SOD activity assays shown in FIG. 16D. Thus, although the SOD activity is markedly reduced by expressing of the mutant soda genes, the amount of SodA protein as shown on SDS-PAGE and Western appear comparable. These data are consistent with a “dominant-negative” effect rendered by expression of the mutant SodA.

FIG. 18 shows a figure of the glnA1 hexameric ring comprised of six monomers. The figure was downloaded from the NCBI web server (http://www.ncbi.nlm.nih.gov/entrez/query.fcgi?db=Structure&itool=toolbar) and modified to illustrate features. GlnA1 monomers form dodecamers comprising two hexameric rings. The squares indicate the position of the active-sites, which are located between adjacent monomers and comprised of manganese ions and catalytic loops from the adjacent monomers. The deleted amino acids in the mutant glnA1 include an aspartic acid at amino acid 54 and glutamic acid at amino acid 335 (GlnA1ΔD54ΔE335), which are in the active-site and correspond to D50 and G327 of the Salmonella glutamine synthase.

FIG. 19 provides a map (A) and features (B) of the plasmid vector pHV203-mut glnA1 ΔD54ΔE335 that expresses the dominant-negative mutant glnA1 in BCG.

FIG. 20 provides a map (A) and features (B) of plasmid vector pMP349, and a map (C) and features (D) of the mycobacterial chromosomal integration vector pMP399 that express mutant SodA ΔH28ΔH76 and mutant glnA1 ΔD54ΔE335 in BCG.

FIG. 21 shows an example of exogenous antigen expression by pro-apoptotic BCG. SDS-PAGE (upper panel) and Western hybridization (lower panel) with an anti-BLS antibody verify expression of recombinant Brucella lumazine synthase (rBLS) by DD-BCG, which is seen as an 18-kDa band in lane 5 under inducing conditions. rBLS was cloned behind an aceA (icl) promoter. BSA=bovine serum albumin, which was present in broth cultures, other bands in lanes 4-6 represent proteins of DD-BCG or rBLS. Lanes 5 and 6 represent DD-BCGrBLS grown under conditions that induce (+, addition of acetate) and suppress (−, addition of succinate) the aceA (icl) promoter and thus the production of rBLS.

FIG. 22 shows the map (A) and features (B) of the vector used to inactivate thioredoxin (trxC) and thioredoxin reductase (trxB2) on the chromosome of BCG.

FIG. 23 shows the map (A) and features (B) of the vector to replace the wild-type alleles for thioredoxin (trxC) and thioredoxin reductase (trxB2) on the chromosome of BCG with mutant alleles in which six amino acids of each enzyme that correspond to the active sites have been eliminated.

FIG. 24 shows the map (A) and features (B) of the vector used to inactivate sigE on the chromosome of BCG.

FIG. 25 shows reduced glutamine synthetase activity in modified BCG strains that express the ΔD54ΔE335 dominant-negative mutant of glnA1 described in Example 8. Panel (A) shows SDS-PAGE (upper) and Western hybridization blot (lower) of lysates (L) of BCG, 3D-BCG, and 4D-BCG as well as partially-purified lysates following ammonium sulfate (AS) precipitation. 4D-BCG was constructed by electroporating the plasmid pHV203-mutGlnA1ΔD54ΔE335 (Table 1) into 3D-BCG. The GlnA1 monomer migrates between the 50- and 37-kDa markers and shows comparable amounts of GlnA1 produced by BCG, 3D-BCG, and 4D-BCG. Panel (B) shows the glutamine synthase activity in the AS-treated lysates of 3D-BCG and 4D-BCG, representing the same AS preparations shown in (A). The reaction was followed spectrophotometrically by monitoring absorbance over time. 3D-BCG AS lysate: ∘, undiluted; □, 2-fold dilution; Δ, 4-fold dilution; ⋄, 8-fold dilution. 4D-BCG AS lysate: , undiluted; ▪, 2-fold dilution. Despite comparable amounts of GlnA1 protein as shown in (A), enzyme activity was barely detected in 4D-BCG with the undiluted 4D-BCG prep exhibiting activity comparable to an 8-fold dilution of the 3D-BCG prep. This demonstrates that expression of the ΔD54ΔE335 monomer exerts a dominant-negative effect upon enzyme activity. Panel (C) shows a repeat enzyme activity assay involving two culture preparations of the pHV203-mutGlnA1ΔD54ΔE335 version of 4D-BCG. In addition, the pMP399 version of 4D-BCG was constructed by electroporating the chromosomal integration vector pMP399-mutSodAΔH28ΔH76,mutGlnA1ΔD54ΔE335 (Table 1) into DD-BCG. The pMP399 version of 4D-BCG does not achieve quite as potent a reduction of glutamine synthetase activity as does the pHV203 version, probably related to a copy number effect from expressing the D54ΔE335 GlnA1 mutant from the chromosome (i.e., single copy) versus a multicopy plasmid, respectively.

FIG. 26 shows the production of IFN-γ and IL-2 by CD4+ T-cells following vaccination with BCG and paBCG vaccines. (A) The percent of CD4+ T-cells from the spleens of C57Bl/6 mice that produce INF-γ and IL-2 were plotted against days after IV vaccination with BCG, DD-BCG, 3D-BCG, and 4D-BCG. Each data point in each panel represents a single mouse and displays the % of CD4+ splenocytes that produce INF-γ or IL-2 after overnight restimulation on BCG-infected macrophages minus the % cells producing INF-γ or IL-2 after restimulation on uninfected macrophages. The shaded area shows the mean value±2 standard deviations for splenocytes from PBS-vaccinated mice analyzed in a similar fashion, indicating very low background with the IFN-γ assays and relatively higher background with IL-2. (B) Summary of the % INF-γ+ and % IL-2+ CD4+ T-cells from BCG- versus paBCG-vaccinated mice, using only the subset of mice that had an IFN-γ value of ≧0.5%. This eliminated results from mice harvested before the onset of the primary T-cell response, as well as results from recipients of the more advanced 3D- and 4D-BCG vaccines in which cytokine production quickly declined to almost baseline values following primary proliferation (panel A) but then was rapidly recalled during reinfection (see FIG. 27). The dot-plots show median, 25-75 percentile (box), and 10-90 percentile 20 (whiskers) values. Whereas BCG typically induced more IFN-γ production, the IL-2 values were significantly higher in mice vaccinated with the paBCG vaccines, P=0.0024.

FIG. 27 shows T-cell responses to vaccination with BCG, DD-BCG, and 3D-BCG at day 25 and day 31 post-vaccination. BCG-specific cytokine production by splenocytes from mice vaccinated 25 days and 31 earlier. The vaccine dose was 5×10⁵ cfu administered intravenously. Splenocytes were incubated overnight on IFN-γ-treated uninfected bone marrow-derived macrophages (BMDMs) or IFN-γ-treated BCG-infected BMDMs. T-cells were then evaluated by flow cytometry for production of INF-gamma and IL-2 by intracellular cytokine staining techniques. The percent of IFN-γ-producing and IL-2-producing CD4+ and CD8+ T-cells is shown within the boxed areas. Background cytokine production was determined from the unstimulated values (uninfected macrophages). Note: In contrast to the data shown in FIG. 26A, the % values shown here represent % of the total CD4 population without subtracting the baseline value (uninfected BMDM) from the BCG-infected BMDM value after restimulation. Raw data from this plot were converted for incorporation into FIG. 26A. For example, the data points at 0.73% (0.86-0.13) and 1.47% (1.52-0.05) for IFN-y production at days 25 and 31, respectively, and −0.03% (0.15-0.18) and 0.18% (0.28-0.10) for IL-2, respectively, come from this experiment.

FIG. 28 shows secondary (recall) T-cell responses in BCG-vaccinated mice and 3DBCG-vaccinated mice at 5 days post-intratracheal challenge with 4×10⁷ cfu of BCG. Mice were vaccinated subQ with 5×10⁵ cfu of the vaccine strain three months earlier and from 4-8 weeks post-vaccination were treated with INH and rifampin to eliminate the vaccine strain. Antigen-specific production of IFN-γ was 1.35% (1.58-0.23) and 0.85% (2.09-1.24%) in two BCG-vaccinated mice versus 7.88% (8.09-0.21) and 3.85% (4.09-0.024) in two 3DBCG-vaccinated mice. Antigen-specific co-production of IFN-γ and IL-2 was 0.29% (0.29-0.0) and 0.10% (0.15-0.03) in the BCG mice versus 2.01% (2.02-0.01) and 1.09% (1.15-0.06) in 3DBCG mice.

### DETAILED DESCRIPTION OF THE INVENTION

It must be noted that, as used in the specification and the appended claims, the singular forms “a,” “an,” and “the” include plural referents unless the context clearly dictates otherwise. Thus, for example, reference to “an enzyme” includes multiple copies of the enzyme and can also include more than one particular species of enzyme.

A method of modifying a microbe to enhance the immunogenicity of the microbe is provided, comprising reducing the activity of an anti-apoptotic enzyme produced by the microbe by overexpressing a dominant-negative mutant enzyme and/or inactivation of a regulatory gene that controls the production of anti-apoptotic enzymes, whereby the bacterium has enhanced immunogenicity in a subject. The dominant-negative mutant of SodA or glutamine synthase is a mutant enzyme that when expressed by the bacterium reduces the total SOD or glutamine synthase activity of the bacterium. The modified bacteria can also contain a mutation in a regulatory gene that reduces its activity or inactivates it. As used herein, a mutation that causes reduced activity (an activity reducing mutation) encompasses an inactivating mutation. Thus, also provided is an intracellular microbe, modified to reduce the activity of an anti-apoptotic enzyme of the microbe.

The invention also provides a method of modifying an attenuated microbe to enhance the immunogenicity of the attenuated microbe, comprising reducing the activity of an anti-apoptotic enzyme produced by the attenuated microbe by overexpressing a dominant-negative mutant enzyme and/or inactivation of a regulatory gene that controls the production of anti-apoptotic enzymes, whereby the attenuated bacterium has enhanced immunogenicity in a subject. Thus, also provided is an attenuated intracellular microbe, further modified to reduce the activity of an anti-apoptotic enzyme of the microbe.

As noted above, the microbe can be any microbe described herein. The microbe can be an intracellular pathogen or an obligate intracellular pathogen. The microbe attenuated by the present methods can be a bacterium, protozoan, virus, or fungus. When the microbe is a bacterium, the bacterium can be, but is not limited to, for example, a Mycobacterium species. Examples of species of Mycobacterium include, but are not limited to, M. tuberculosis, M. bovis, M. bovis strain BCG including BCG substrains, M. avium, M. intracellulare, M. africanum, M. kansasii, M. marinum, M. ulcerans and M. paratuberculosis. It can also be a Nocardia species, including Nocardia asteroides or Nocardia farcinica. The construction of SOD-diminished mutants of these species can achieve both attenuation and confer the pro-apoptotic quality that enhances the development of strong cellular immune responses in a manner analogous to the present SOD-diminished BCG vaccine, as secretion of iron-manganese SOD is a common and distinctive attribute of many of the pathogenic species of mycobacteria (Raynaud et al., 1998) and Nocardia. Accordingly, SOD-diminished vaccines of these other mycobacterial species and Nocardia are expected to also be highly effective vaccine strains. Examples of other obligate and facultative intracellular bacterial species contemplated within the present invention include, but are not limited to, Legionella pneumophila, other Legionella species, Salmonella typhi, other Salmonella species, Shigella species, Listeria monocytogenes, Staphylococcus aureus, Staphylococcus epidermidis, Bacteroides fragilis, other Bacteroides species, Chlamydia pneumoniae, Chlamydia trachomatis, Chlamydia psittaci, Coxiella burnetii, other Rickettsial species, and Ehrlichia species.

Moreover, bacteria that cause diseases in livestock, animals and pets can be the targets of the methods of the present invention. Examples of veterinary bacterial pathogens include, but are not limited to, Brucella abortus and other Brucella species, Yersinia pestis, Pasteurella haemolytica, Pasteurella multocida and other Pasteurella species, Actinobacillus pleuropneumomia, Cowdria ruminantium, Mycobacterium avium subspecies paratuberculosis, and Listeria ivanovii.

Other intracellular microbes such as protozoa and fungi that exert an anti-apoptotic effect upon their host cell are likely to become both attenuated and pro-apototic, and therefore useful as vaccine strains, when the activity of a microbial enzyme that primarily mediates the anti-apoptotic effect is reduced. Thus, the invention provides a method of modifying a protozoan to enhance the immunogenicity of the protozoan, comprising reducing the activity of an anti-apoptotic enzyme produced by the protozoan, whereby the protozoan has enhanced immunogenicity in a subject and a method of modifying a fungus to enhance the immunogenicity of the fungus, comprising reducing the activity of an anti-apoptotic enzyme produced by the fungus, whereby the fungus has enhanced immunogenicity in a subject. Examples of protozoan and fungal species contemplated within the present invention include, but are not limited to, Plasmodium falciparum, other Plasmodium species, Toxoplasma gondii, Pneumocystis carinii, Trypanosoma cruzi, other trypanosomal species, Leishmania donovani, other Leishmania species, Theileria annulata, other Theileria species, Eimeria tenella, other Eimeria species, Histoplasma capsulatuin, Cryptococcus neoformans, Blastomyces dermatitidis, Coccidioides immitis, Paracoccidioides brasiliensis, Penicillium marneffei, and Candida species. Methods have been described for creating recombinant and attenuated mutants of protozoa and yeast species, and are known to a person of skill in the art. For example, transfection techniques and vectors for insertional mutagenesis and the expression of heterologous antigens have been described in Toxoplasma gondii (Chiang et al., 1999; Charest et al., 2000). As an iron-cofactored SOD of Toxoplasma gondii has been described (Odberg-Ferragut et al., 2000), such vectors and methods can be used to reduce its production, or that of another anti-apoptotic enzyme, by using allelic inactivation, antisense techniques, targeted incremental attenuation or a dominant/negative approach. Similarly, Trypanosoma and Leishmania species are susceptible to transformation and chromosomal integration of DNA (Brooks et al., 2000; Dumas et al., 1997), thereby enabling similar manipulations. Methods for performing genetic manipulations in fungal pathogens have also become recently available (Retallack et al., 1999; Woods, Heinecke, and Goldman, 1998; Varma and Kwon-Chung, 2000; Enloe, Diamond, and Mitchell, 2000; Wilson et al., 2000). A protozoan made in accordance with the method of the invention is provided, as is a fungus made in accordance with the method of invention.

Thus, a specific embodiment of the invention provides a live vaccine against tuberculosis, derived by diminishing the activity of iron-manganese superoxide dismutase (SOD) in a strain of M. tuberculosis or BCG by overexpressing a dominant-negative mutant SOD enzyme.

The invention provides a method of making a microbial vaccine, comprising reducing the activity of an anti-apoptotic enzyme produced by the microbe, wherein the reduction in the activity of the anti-apoptotic enzyme attenuates the microbe, whereby a microbial vaccine is produced.

The invention provides a method of making a microbial vaccine, comprising reducing in an attenuated microbe the activity of an anti-apoptotic enzyme produced by the microbe, whereby a microbial vaccine is produced.

The present invention provides a composition comprising a microbe comprising an enzyme modified by the methods of the present invention. The composition can further comprise a pharmaceutically acceptable carrier or a suitable adjuvant. Such a composition can be used as a vaccine.

The modified bacterium can include a dominant-negative mutant selected from the group consisting of a) SodA in which a deletion, insertion, and/or substitution of nucleotides in the naturally occurring nucleic acid encodes a molecule that reduces the SOD activity of the organism; and b) glutamine synthase in which a deletion, insertion, and/or substitution of nucleotides in the naturally occurring nucleic acid encodes a molecule that reduces the glutamine synthase activity of the organism. In one embodiment, the modified bacterium can be BCG. Thus, a BCG modified to express reduced SOD activity is provided.

The modified bacterium can comprise a further pro-apoptotic modification. The further pro-apoptotic modification can comprise one or more modification selected from the group consisting of inactivation of SigH, inactivation of sigE, inactivation of SecA2, inactivation of thioredoxin, inactivation of thioredoxin reductase and inactivation of glutaredoxin. Thus, a BCG modified to express reduced SOD activity and reduced or inactive SigH is provided. A BCG modified to express reduced SOD activity, reduced-activity or inactive SigH and reduced-activity or inactive sigE is provided. A BCG modified to express reduced SOD activity, reduced-activity or inactive SigH, reduced-activity or inactive sige is provided and reduced-activity or inactive SecA2 is also provided.

Specific examples of modified bacteria are described in the examples and Table 1. For example, the modified bacterium can comprise a mutant SodA having deletions of histidine at position 28 and histidine at position 76, a mutant SodA having a deletion of histidine at position 28 or a histidine at position 76, a mutant SodA having a deletion of glutamic acid at position 54, a mutant SodA having a deletion of glutamic acid at position 54 and the replacement of histidine with arginine at position 28. In further examples, the modified bacterium can comprise modifications selected from the group consisting of a mutant of SodA and an activity reducing mutation of sigh; a mutant of SodA and an activity reducing mutation of secA2; a mutant of SodA, an activity reducing mutation of sigh and an activity reducing mutation of secA2; and a mutant of SodA, a dominant-negative mutant of glnA1, an activity reducing mutation of sigH and an activity reducing mutation of secA2.

As further examples of the modified bacterium, the bacterium can comprise a mutation of glnA1 selected from the group consisting of deletions of aspartic acid at amino acid 54 and glutamic acid at amino acid 335; and a deletion of aspartic acid at amino acid 54 or a glutamic acid at amino acid 335. The bacterium with reduced glnA1 activity can further comprise an activity reducing mutation of secA2. The bacterium with reduced glnA1 activity can further comprise a dominant-negative mutant of SodA. In the bacterium with reduced glnA1 activity and a dominant-negative mutant of SodA, the mutant SodA can comprise deletions of histidine at position 28 and histidine at position 76. The bacterium with reduced glnA1 activity can further comprise an activity reducing mutation of sigH and an activity reducing mutation of secA2. The bacterium with reduced glnA1 activity can further comprise a dominant-negative mutant of SodA and an activity reducing mutation of sigh. In the bacterium with reduced glnA1 activity and a dominant-negative mutant of SodA, the dominant-negative mutant is a mutant SodA having a deletion of glutamic acid at position 54. In the bacterium with reduced glnA1 activity and a dominant-negative mutant of SodA, the dominant-negative mutant is a mutant SodA having deletions of histidine at position 28 and histidine at position 76. In the bacterium with reduced glnA1 activity activity and a dominant-negative mutant of SodA, the bacterium can further comprise a dominant-negative mutant of SodA and an activity reducing mutation of secA2. Methods of making the bacteria described in the description, in Table 1, the examples and figures are provided.

The modified bacterium of the invention can comprises an activity reducing mutation of sigH. The modified bacterium can comprise an activity reducing mutation of sigH and an activity reducing mutation of secA2.

The present invention additionally provides a method of producing an immune response in a subject by administering to the subject any of the compositions of this invention, including a composition comprising a pharmaceutically acceptable carrier and a microbe comprising an enzyme necessary for in vivo viability that has been modified according to the methods taught herein. The composition can further comprise a suitable adjuvant, as set forth herein. The subject can be a mammal and is preferably a human.

The present invention provides a method of preventing an infectious disease in a subject, comprising administering to the subject an effective amount of a composition of the present invention. In addition to preventing bacterial diseases, for example, tuberculosis, it is contemplated that the present invention can prevent infectious diseases of fungal, viral and protozoal etiology. The subject can be a mammal and preferably human.

It is contemplated that the above-described compositions of this invention can be administered to a subject or to a cell of a subject to impart a therapeutic benefit or immunity to prevent infection. Thus, the present invention further provides a method of producing an immune response in an immune cell of a subject, comprising contacting the cell with a composition of the present invention, comprising a microbe in which an enzyme necessary for in vivo viability has been modified by any of the methods taught herein. The cell can be in vivo or ex vivo and can be, but is not limited to, an MHC I-expressing antigen presenting cell, such as a dendritic cell, a macrophage or a monocyte. As used throughout, by a “subject” is meant an individual. Thus, the “subject” can include domesticated animals, such as cats, dogs, etc., livestock (e. g., cattle, horses, pigs, sheep, goats, etc.), laboratory animals (e. g., mouse, rabbit, rat, guinea pig, etc.) and birds. Preferably, the subject is a mammal such as a primate, and, more preferably, a human.

The invention, therefore, provides a method of enhancing the immunogenicity of an attenuated bacterium, comprising reducing the activity of an anti-apoptotic enzyme produced by the bacterium, whereby the bacterium has enhanced immunogenicity in a subject. The bacterium modified by reducing the activity of an anti-apoptotic enzyme can be selected from the group consisting of M. tuberculosis, M. bovis, M. avium, M. intracellulare, M. africanum, M. kansasii, M. marinum, M. ulcerans, M. paratuberculosis, Legionella pneumophila, other Legionella species, Salmonella typhi, other Salmonella species, Shigella species, Listeria monocytogenes, Nocardia asteroides, Listeria ivanovii, Brucella abortus, other Brucella species, and Cowdria ruminantium. For example, live-attenuated strains of Salmonella can be further modified using this invention to enhance their immunogenicity and increase their usefulness as vaccines against Salmonella infection and to enhance their ability to induce protective cellular immune responses to heterologous antigens, including antigens from other infectious organisms and cancer antigens.

Provided is a method for facilitating antigen presentation via construction of pro-apoptotic vaccines made by reducing the production of microbial anti-apoptotic enzymes including SOD, thioredoxin, thioredoxin reductase, glutamine synthetase, and other redox related enzymes such as glutathione reductase (glutaredoxin), other thioredoxin-like proteins, other thioredoxin reductase-like proteins, other glutaredoxin-like proteins, other thiol reductases, and other protein disulphide oxidoreductases. Many of these enzymes are highly conserved in all cellular life forms and many overlap or are identical to the enzymes that detoxify reactive oxygen intermediates due to the central role of reactive oxygen species (ROS) as a trigger for apoptosis. The premise of making pro-apoptotic vaccines relates to the capability of the enzyme from the intracellular pathogen to block apoptosis when the pathogen is within the host cell, as is the case with virulent strains of M. tuberculosis [Balcewicz-Sablinska, M. K. et al, 1998; Keane, J. et al, 2000]. For example, SodA produced by M. tuberculosis detoxifies superoxide (O₂⁻), which is an oxidant with pro-apoptotic biological effects that is produced by the phagocyte oxidase (NADPH oxidase) of immune cells. Accordingly, by reducing the activity of SodA and other microbial enzymes that inactivate the oxidants produced by host immune cells, one can simultaneously attenuate the microbe and enhance the presentation of its antigens, as dendritic and other immune cells process the apoptotic phagocytes (e.g., neutrophils, monocytes and/or macrophages) containing microbial antigens.

Some anti-apoptotic microbial enzymes can be eliminated without adversely affecting the ability to cultivate the microbe as a vaccine strain, and for such enzymes, traditional molecular genetic techniques including allelic inactivation can be used to construct the modified microbe. However, some enzymes are absolutely essential for the viability of the microbe, such that they cannot be eliminated entirely. For these enzymes, techniques of genetic manipulation by which mutants with a partial rather than complete reduction in the activity of the anti-apoptotic enzyme are constructed. Anti-sense RNA overexpression [Coleman, J. et al, 1984] is described in WO 02/062298 as one such strategy for constructing mutant strains with partial phenotypes, and its utility as a tool to screen and identify which essential enzymes can be reduced to render a pro-apoptotic phenotype was also emphasized.

The current invention outlines two additional strategies for achieving a partial reduction in the activity of anti-apoptotic microbial enzymes. The first strategy involves the overexpression of dominant-negative mutants of the enzyme. The second strategy involves allelic inactivation of a regulatory gene that governs the expression of the anti-apoptotic enzyme. Both strategies represent additional methods for stably modifying a microbe to render a partial phenotype, whereby the microbe retains or increases immunogenicity but loses or reduces pathogenicity in a subject, comprising reducing but not eliminating an activity of an enzyme produced by the microbe, whereby reducing the activity of the enzyme attenuates the microbe or further attenuates the microbe.

Dominant-negative enzyme mutants can comprise either mutations that yield a modified enzyme with partial enzyme activity or mutations that yield an inert enzyme completely devoid of enzyme activity. As the effect of co-expressing the mutant enzyme in a cell that also expresses the wild-type enzyme is typically a reduction rather than complete elimination of the whole-cell enzymatic activity, this strategy can be directed against genes that are essential for the viability of the microbe.

The strategy of reducing the activity of anti-apoptotic enzymes by using dominant-negative techniques can be employed in wild-type bacterial strains as a means to make the strain partially- or fully-attenuated while increasing its immunogenicity. It can also be applied to strains that are already attenuated and/or current vaccine strains, for example, to enhance the immunogenicity of Bacillus Calmette-Guerin (BCG), the current vaccine for tuberculosis.

Examples of the constructs provided herein and examples of contructs used to make the present constructs are provided in Table 1.

The compositions of the present invention can be administered in vivo to a subject in need thereof by commonly employed methods for administering compositions in such a way to bring the composition in contact with the population of cells. The compositions of the present invention can be administered orally, parenterally, intramuscularly, transdermally, percutaneously, subcutaneously, extracorporeally, topically or the like, although oral or parenteral administration are typically preferred. It can also be delivered by introduction into the circulation or into body cavities, by ingestion, or by inhalation. The vaccine strain is injected or otherwise delivered to the animal with a pharmaceutically acceptable liquid carrier, that is aqueous or partly aqueous, comprising pyrogen-free water, saline, or buffered solution. For example, an M. tuberculosis vaccine would most likely be administered similar to methods used with US BCG Tice strain, percutaneously using a sterile multipuncture disk.

Parenteral administration of the compositions of the present invention, if used, is generally characterized by injection. Injectables can be prepared in conventional forms, either as liquid solutions or suspensions, solid forms suitable for solution of suspension in liquid prior to injection, or as emulsions. As used herein, “parenteral administration” includes intradermal, subcutaneous, intramuscular, intraperitoneal, intravenous, intra-articular and intratracheal routes.

The dosage of the composition varies depending on the weight, age, sex, and method of administration. In one embodiment, the dosage of the compound is from 0.5×10² colony-forming units to 5×10⁸ colony-forming units of the viable live-attenuated microbial strain. More preferably, the compound is administered in vivo in an amount of about 1×10⁶ colony-forming units to 5×10⁷ colony-forming units of the viable live-attenuated microbial strain. The dosage can also be adjusted by the individual physician as called for based on the particular circumstances.

The compositions can be administered conventionally as vaccines containing the active composition as a predetermined quantity of active material calculated to produce the desired therapeutic or immunologic effect in association with the required pharmaceutically acceptable carrier or diluent (i. e., carrier or vehicle). By “pharmaceutically acceptable” is meant a material that is not biologically or otherwise undesirable, i. e., the material can be administered to an individual along with the selected composition without causing any undesirable biological effects or interacting in a deleterious manner with any of the other components of the pharmaceutical composition in which it is contained.

Although the examples provided below involve modifications of BCG, the current vaccine against tuberculosis, the invention teaches how vaccines of other intracellular pathogens can be developed by expressing dominant-negative mutants of anti-apoptotic bacterial enzymes.

Expression of Dominant-Negative Mutants of Microbian Anti-Apoptotic Enzymes

The primary utility of a dominant-negative approach over allelic inactivation for reducing the activity of an anti-apoptotic microbial enzyme is when the gene appears to be essential for survival of the microbe in vitro despite attempts to enrich the media in which the microorganism is cultivated. In these circumstances, allelic inactivation would interfere with cultivation of the mutant bacterium and make it unsuitable as a vaccine strain, and a method for rendering a partial phenotype with reduced activity of the essential enzyme that still enables the microbe to grow is favored. Antisense techniques and targeted incremental attenuation have been previously described in WO 02/062298 and can be used to reduce the activity of an essential microbial enzyme. The expression of dominant-negative enzyme mutants represents an alternative strategy that shares many of the methods described for practicing targeted incremental attenuation but differs in some important aspects.

Step 1. Identification of Anti-Apoptotic Microbial Enzymes

Detailed methods for identifying essential and anti-apoptotic microbial enzymes have been described in WO 02/062298. To verify that reducing the activity of the microbial enzyme renders a pro-apoptotic effect, host cell apoptosis can be monitored using either in vitro cell culture techniques (e.g., infected macrophages) or the recovery of cells or tissue of infected animals in vivo. There are a large number of techniques used to monitor apoptosis including flow cytometry, TUNEL stains, and DNA fragmentation assays that are well-known to those skilled in the art [Otsuki, Y. et al, 2003; Steensma, D. P. et al, 2003].

There are two important differences related to the selection of anti-apoptotic enzymes for practicing a dominant-negative strategy as compared to targeted incremental attenuation. First, for the dominant-negative approach it is best to select enzymes with known multimeric structure, whereas this is not important for practicing targeted incremental attenuation. This is because in the former the mechanism of reduced enzyme activity is believed to be mediated by interference by mutant enzyme monomers with either the formation of the enzymatically-active multimer or an alteration in tertiary configuration that adversely affects enzyme activity. A body of published literature demonstrates that several bacterial enzymes that inactivate host-derived oxidants and thus are likely to have anti-apoptotic effects are multimers in their biologically active form:
The iron co-factored superoxide dismutase of M. tuberculosis/bovis/BCG is tetrameric [Cooper, J. B. et al, 1995]
In general, thioredoxin appears to be biologically active as a monomer, however there may be exceptions and dimer formation described in some bacterial species [Rehse, P. H. et al, 2005]
Thioredoxin reductase forms homodimers in mammalian species [Zhong, L. et al, 2000] and some microorganisms including Plasmodium species [Wang, P. F. et al, 1999]
Glutamine synthase is dodecameric [Eisenberg, D. et al, 2000; Gill, H. S. et al, 1999]
Bacterial glutaredoxin (glutathione reductase) is monomeric in reduced form but dimeric in the oxidized form [Kelley, J. J., III et al, 1997]

Thus, with each of these enzymes one can reduce enzymatic activity by using a dominant-negative approach as taught in the current invention. Reducing SodA activity by using anti-sense techniques as described in WO 02/062298 results in stronger host immune responses and greater vaccine-induced protection against infection [Kernodle, D. et al, 2005; Kernodle, D. S. et al, 2001]. Reducing SodA activity by a dominant-negative strategy has a similar effect.

Second, although practice of the dominant-negative strategy and targeted incremental attenuation are not limited to essential microbial genes, that is the primary reason for preferring targeted incremental attenuation over simple allelic inactivation when the gene is essential. In contrast, there are some potential advantages of employing a dominant-negative strategy over allelic inactivation in some microorganisms even for non-essential genes. First, there are considerations of time and the ease of genetic modifications that are especially true for species in which it is difficult to achieve homologous recombination necessary for allelic inactivation, but for which overexpression of a gene can be accomplished on plasmids or other vectors. Another reason for selecting overexpression of a dominant-negative enzyme mutant over allelic inactivation is if the enzyme is or might be an important immunogen. In this situation, it may be important to allow the vaccine strain to continue to produce the enzyme as it may be a target against which an immune response can be directed. Thus, when the host subsequently becomes infected with the pathogen causing a disease that the vaccine is intended to prevent, the host has a more complete repertoire of immune responses to direct against the pathogen. This “antigen repertoire” consideration is unimportant under circumstances when the pro-apoptotic live-attenuated vaccine strain is used solely as a vector for expressing exogenous antigens, and the desired immune response is against the exogenous antigen. This will be discussed in more detail in the context of differences in the nature of a pro-apoptotic BCG vaccine to be used to vaccinate against tuberculosis versus a pro-apoptotic BCG vaccine to be used as a vector to vaccinate against an exogenous antigen.

Among the mycobacterial enzymes with known or suspected anti-apoptotic effects listed above, SodA and GlnA1 (glutamine synthase) appear to absolutely essential for bacterial growth [Dussurget, O. et al, 2001; Tullius, M. V. et al, 2003]. Thus, they are not good candidates for allelic inactivation for the purpose of making a vaccine but can be manipulated to achieve a partial reduction in enzyme activity achieved either through antisense techniques, targeted incremental attenuation, or a dominant-negative approach. As both SodA and GlnA1 have been implicated in immune evasion by M. tuberculosis [Edwards, K. M. et al, 2001; Miller, B. H. et al, 2000] and are also produced by BCG, they are favored targets for enhancing the immunogenicity of BCG. Examples below show that the SodA-diminished phenotype in BCG is also associated with enhanced vaccine efficacy.

Step 2. Generating Mutants of Anti-Apoptotic Enzymes

The methods for generating mutants of anti-apoptotic enzymes for practicing the dominant-negative strategy include those described in WO 02/062298 but also involve an important difference. In the targeted incremental attenuation strategy, the mutant enzyme is the sole source of enzyme activity. These mutants can exhibit enzymatic activity that is only, for example, 95%, 90%, 85%, 80%, 75%, 70%, 65%, 60%, 55%, 50%, 45%, 40%, 35%, 30%, 25%, 20%, 15%, 10%, 5%, 2%, etc. of the activity of the parent, natural enzyme. A series of mutant enzymes can be produced that have activities that fall within this range of reduction in activity. Thus, for essential enzymes where the practice of targeted incremental attenuation has its greatest utility, the mutant enzyme is expected to have some activity.

In contrast, in the dominant-negative strategy, the mutant enzyme can be completely inert, exhibiting 0% activity. This is because the dominant-negative strategy is based on interference between expressed mutant enzyme monomers and the wild-type enzyme monomers encoded by the parent gene. This interference leads to a reduction in total enzyme activity.

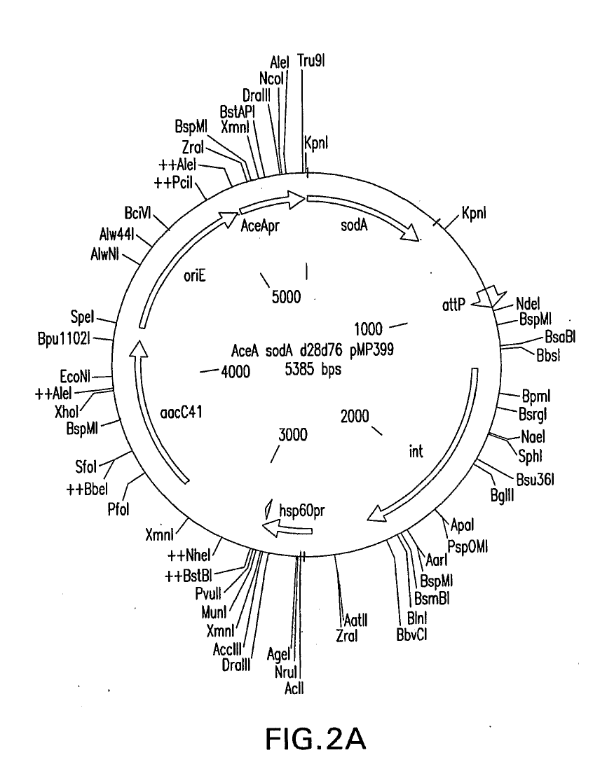

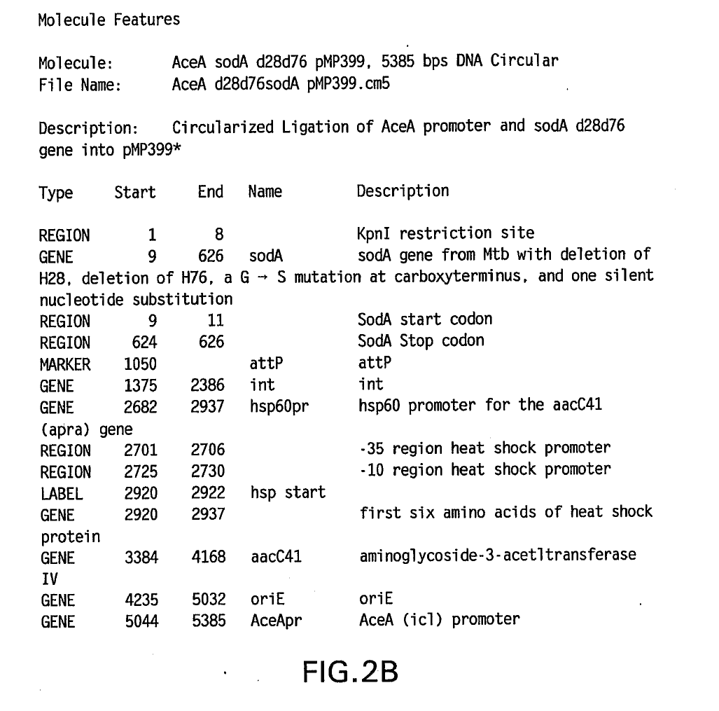

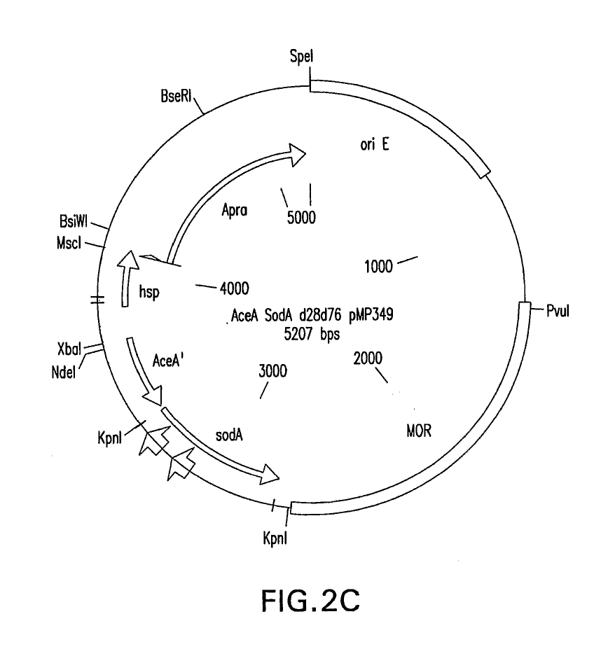

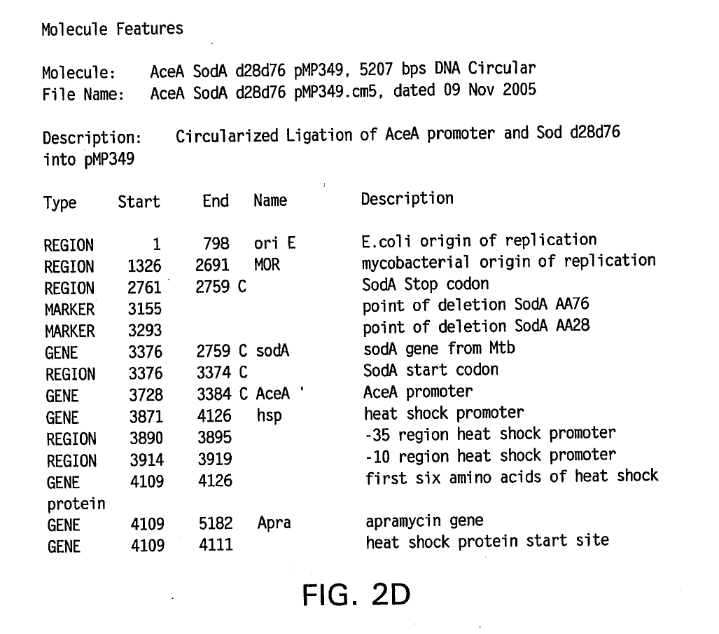

This difference has implications for the design of enzyme mutants to practice the dominant-negative strategy versus targeted incremental attenuation. Most notably, mutant enzymes used in the dominant-negative strategy are potentially easier to design as one strategy is simply to disable the active site of the enzyme. As noted in WO 02/062298, Xray crystallographic data are available for many of the bacterial enzymes that inactivate host oxidants, including identification of active site residues. Thus, information is available to help guide the construction of enzyme mutants in which active site residues are eliminated or replaced. This strategy was employed in the construction of a ΔH28ΔH76 mutant of SodA, in which two of the histidines that chelate the active site iron of SodA have been removed (FIG. 2, Example 1).

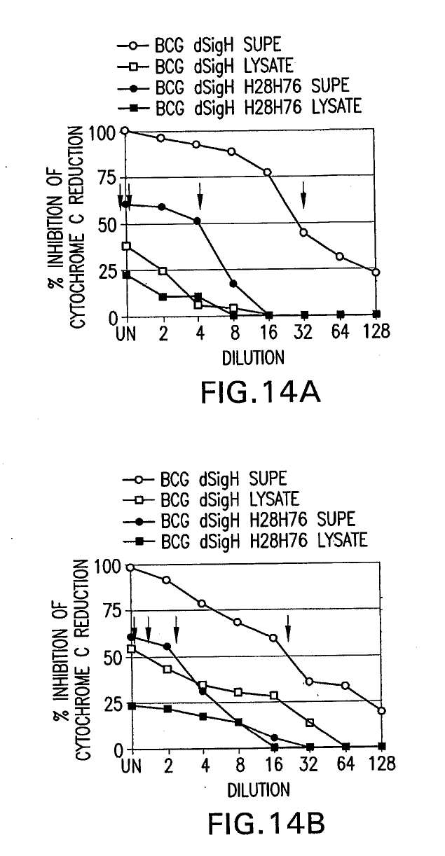

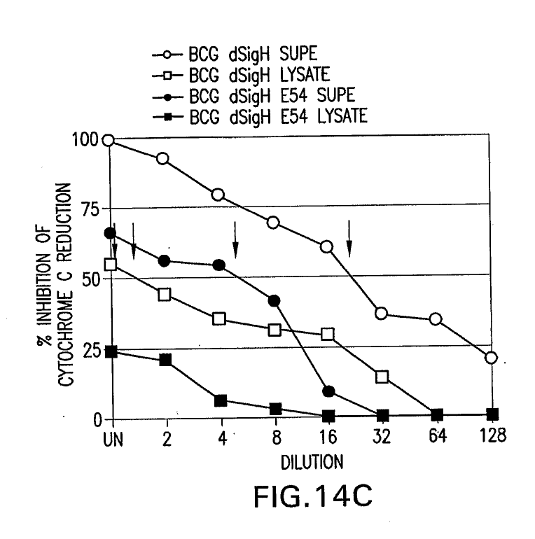

Also, in multimeric enzymes, for example glutamine synthase which has a dodecameric structure, the active site frequently lies between monomers and is formed by components of more than one monomer. This enables mutant enzymes to be designed in which the monomer has amino acid deletions, insertions, or substitutions that affect more than one active site. This strategy was employed in the construction of a ΔD54ΔE335 mutant of glnA1, which encodes the primary glutamine synthase of M. tuberculosis and BCG (FIG. 14).

However, some of the mutant enzymes constructed to practice targeted incremental attenuation can also be used to practice the dominant-negative strategy. For example, sodA mutant alleles on pLou1-mut-SodA (Table 1) were being placed into BCG to construct BCG(pLou1-mut SodA) (Table1) using techniques for targeted incremental attenuated described in WO 02/062298 when the recombinant BCG strains were noted to have reduced SOD activity (Example 1).

The genes encoding mutant enzymes with reduced enzymatic activity can have single or multiple nucleotide differences compared to the wild-type gene leading to single or multiple amino acid deletions, insertions, and/or substitutions. Nucleotide differences can be introduced using the wild-type gene as a substrate and using a variety of techniques to achieve site-directed mutagenesis known to those skilled in the art including PCR-based methods [Ho, S. N. et al, 1989]. Alternatively, the gene containing desired mutations can be synthesized de novo.

Step 3: Expression of the Mutant Enzyme by the Microbe

Next, the gene encoding the mutant enzyme is incorporated into a vector that either integrates into the chromosome of the bacterium or can be stably maintained as a plasmid within the bacterium. Methods for expressing DNA in BCG and other mycobacteria have been available since 1987 [Jacobs, W. R., Jr. et al, 1987], are well-known to those skilled in the art, and include techniques taught by Bloom et al (U.S. Pat. No. 5,504,005, Recombinant mycobacterial vaccine; U.S. Pat. No. 5,854,055 and U.S. Pat. No. 6,372,478, Recombinant mycobacteria), which are hereby incorporated by reference in their entirety for their teaching regarding methods for expressing DNA).

A variety of phage-based and plasmid vectors and genetic tools enabling genes to be incorporated within the bacterium on the chromosome or plasmids are available and will be described in more detail below in the context of their specific use.

Step 4: Identifying Mutant Bacteria to Use as a Vaccine or as a Host Strain to Express a Heterologous Antigen

Methods for identifying mutant bacteria to use as a vaccine are described in detail in WO 02/062298 and primarily involve observing a response in an animal model that correlates with enhanced vaccine-induced protection, for example, enhanced immune responses.

Another method for evaluating mutant bacterial strains for their function as a vaccine strain or as a vector for delivering exogenous antigens involves assays to determine the degree of reduction in enzyme activity in vitro. Reduction in the activity of an enzyme that normally renders an anti-apoptotic effect upon the host should result in increased host cell apoptosis when that bacterium is used to vaccinate a host animal, and would be predicted to be a more immunogenic vaccine than the parent bacterium. Thus, measuring enzyme activity in lysates and/or supernatants of parent bacterium and the mutant bacterium can be used to indicate whether dominant-negative expression of a specific mutant enzyme has produced the desired reduction in total enzyme activity. If total enzyme activity is reduced by the dominant-negative strategy and prior observations link enhanced vaccine efficacy to reduced enzyme activity achieved by another technique, for example antisense techniques, then it is expected that the bacterium with the dominant-negative enzyme reduction will similarly be a more efficacious vaccine strain.

Elimination of Sigma Factors and Other Regulatory Genes that Govern the Production of Microbial Anti-Apoptotic Enzymes

Step 1. Identification of Regulatory Genes of Anti-Apoptotic Microbial Enzymes

The production of some microbial anti-apoptotic enzymes is under the control of regulatory genes including sigma factors that govern the transcription of multiple genes via an effect upon promoter regions. Thus, allelic inactivation of such genes represents an additional way to reduce the production of anti-apoptotic microbial enzymes, with the potential for a pleiotropic effect in which the activity of several anti-apoptotic enzymes is reduced by a single genetic manipulation.

Regulatory genes can be identified by their effect upon the expression of other microbial factors, including anti-apoptotic enzymes. The screening of transposon and other random mutagenesis libraries for mutants that result in enhanced apoptosis of infected cells not only yields mutants with direct defects in anti-apoptotic enzymes but can also identify mutations in regulatory genes that influence the production of key anti-apoptotic microbial enzymes. There is strong homology amongst regulatory factors from different species and some investigators have identified novel sigma factors based on homology to known sigma factors by DNA or amino acid sequence.

The allelic inactivation of the gene encoding sigma factor H (sigH) of M. tuberculosis has been described [Kaushal, D. et al, 2002; Manganelli, R. et al, 2002; Raman, S. et al, 2001, incorporated herein by reference for their teaching of methods to inactivate sigH]. Inactivation of sigH was accompanied by an effect upon several mycobacterial enzymes including thioredoxin, thioredoxin reductase, and a glutaredoxin homolog. A sigH deletion was introduced into the chromosome of BCG, as described below. The enhanced efficacy of BCGΔsigH as a vaccine is described below.

Another modification expected to enhance BCG vaccine efficacy is the inactivation of sigE. This can be done alone or in addition to sigh inactivation. sigE inactivation also plays a role in the resistance of M. tuberculosis to oxidative stress and methods for inactivating sigE have been described in M. tuberculosis [Manganelli, R. et al, 2001; Manganelli, R. et al, 2004b; Manganelli, R. et al, 2004a, incorporated herein by reference for their teaching of methods to inactivate sigE].

Step 2. Inactivation of Regulatory Genes of Anti-Apoptotic Microbial Enzymes

The inactivation of regulatory and sigma factor genes can be performed using allelic inactivation techniques involving suicide plasmid vectors [Berthet, F. X. et al, 1998; Hinds, J. et al, 1999; Jackson, M. et al, 1999; Kaushal, D. et al, 2002; Parish, T. et al, 2000; Pavelka, M. S., Jr. et al, 1999; Pelicic, V. et al, 1997] or mycobacteriophage-derived genetic tools that are capable of replicating as a plasmid in E. coli and lysogenizing a mycobacterial host [Bardarov, S. et al, 1997; Braunstein, M. et al, 2002] [also Bardarov et al, U.S. Pat. No. 6,271,034]. These methods and tools are well-known to those skilled in the art.

Specific methods for inactivating sigH and sige in M. tuberculosis have already been described by several groups of investigators as noted above. The methods employed herein in allelic inactivation of sigH in BCG are shown below.

These examples show the enhancement of immunogenicity of bacteria by inactivating regulatory genes, which results in the reduced activity of anti-apoptotic microbial enzymes.

Using Pro-Apoptotic BCG Strains to Express Exogenous Antigens

Pro-apoptotic BCG and other pro-apoptotic bacterial vaccines constructed using the dominant-negative mutant enzyme strategy, either alone or in combination with pro-apoptotic modifications of a bacterium rendered either by inactivation of a sigma factor gene, antisense techniques, or targeted incremental attenuation can be used to express exogenous antigens. The foreign DNA can be DNA from other infectious agents, for example, DNA encoding Brucella lumazine synthase (BLS), which is an immunodominant T-cell antigen from Brucella abortus [Velikovsky, C. A. et al, 2002]. The construction of DD-BCGrBLS is described below. The foreign DNA can be DNA encoding antigens of human immunodeficiency virus (HIV), measles virus, other viruses, bacteria, fungi, or protozoan species. The foreign DNA can be a cancer antigen.

To express foreign DNA in pro-apoptotic BCG, the gene of interest is incorporated into a vector that either integrates into the chromosome of the bacterium or can be stably maintained as a plasmid within the bacterium. Methods for expressing foreign DNA in BCG and other mycobacteria have been available since 1987 [Jacobs, W. R., Jr. et al, 1987], are well-known to those skilled in the art, and include techniques taught by Bloom et al (U.S. Pat. No. 5,504,005, Recombinant mycobacterial vaccine; U.S. Pat. No. 5,854,055 and U.S. Pat. No. 6,372,478, Recombinant mycobacteria), which are hereby incorporated by reference in their entirety).

A variety of phage-based and plasmid vectors and genetic tools enabling genes to be incorporated within the bacterium on the chromosome or plasmids are available and will be described in more detail below in the context of their specific use.

By expressing the foreign antigen in pro-apoptotic bacterial vaccines that facilitate entry into apoptosis-associated cross priming pathways of antigen presentation, the foreign antigen is introduced into this antigen presentation pathway. Furthermore, it is presented in the context of very strong co-stimulatory signals from the bacterial host that influence antigen presentation by the dendritic cells in a manner that promotes protective responses rather than the induction of tolerance. Thus, this practice enables the development of very strong adaptive T-cell responses including both CD4 and CD8 T-cells and CD4 help for CD8 T-cell responses, which has been difficult to achieve using vectors designed to access either exogenous or endogenous pathways of antigen presentation.

Examples of the microbes made by overexpression of mutant SOD include, but are not limited to the following: a mutant M. tuberculosis or BCG in which glutamic acid is deleted at position 54 of superoxide dismutase; a mutant M. tuberculosis or BCG in which glutamic acid is deleted at position 54 and histidine at position 28 is replaced by arginine of superoxide dismutase; a mutant M. tuberculosis or BCG in which histidine is deleted at position 28 of superoxide dismutase; a mutant M. tuberculosis or BCG in which histidine is deleted at position 76 of superoxide dismutase; a mutant M. tuberculosis or BCG is which histidines are deleted at position 28 and at position 76 of superoxide dismutase, a mutant M. tuberculosis or BCG in which histidines are deleted at position 28 and at position 76 of superoxide dismutase and there is a glycine to serine substitution at the carboxyterminus. Examples of the microbes made by overexpression of glutamine synthetase (glnA1) include, but are not limited to the following: a mutant M. tuberculosis or BCG in which aspartic acid is deleted at position 54 of glutamine synthase; a mutant M. tuberculosis or BCG in which glutamic acid is deleted at position 335 of glutamine synthase; a mutant M. tuberculosis or BCG in which aspartic acid is deleted at position 54 and glutamic acid is deleted at position 335 of glutamine synthase.

The present invention further provides the attenuated microbes of the invention, further expressing a heterologous antigen. The pro-apoptotic, attenuated bacteria of the present invention are optionally capable of expressing one or more heterologous antigens. As a specific example, heterologous antigens are expressed in SOD-diminished BCG bacterium of the invention. Live-attenuated vaccines have the potential to serve as vectors for the expression of heterologous antigens from other pathogenic species (Dougan et al, U.S. Pat. No. 5,980,907; Bloom et al, U.S. Pat. No. 5,504,005). Thus, the microbes of the present invention having a reduction in the expression or activity of an anti-apoptotic or essential enzyme can further be modified to express an antigen from a different microbe. Such antigens can be from viral, bacterial, protozoal or fungal microorganisms. The recombinant pro-apoptotic microorganisms then form the basis of a bi- or multivalent vaccine. In this manner, multiple pathogens can be targeted by a single vaccine strain. The invention provides a method of making a multivalent vaccine comprising transforming the pro-apoptotic microbe of the invention with a nucleic acid encoding a heterologous antigen. For example, antigens of measles virus containing immunodominant CD4+ and CD8+ epitopes can be expressed in SOD-diminished BCG, with expression achieved by stably integrating DNA encoding the measles antigen of interest into genomic DNA of the pro-apoptotic BCG of the invention using techniques taught by Bloom et al (U.S. Pat. No. 5,504,005, which is hereby incorporated by reference in its entirety). Alternatively, the gene encoding the antigen can be expressed on a plasmid vector, for example, behind the promoter of the 65 kDa heat-shock protein of pHV203 or behind an aceA(icl) promoter on any chromosomal-integration or plasmid vector using standard techniques for expressing recombinant antigens that are well-known to those skilled in the art. The antigen does not have to consist of the entire antigen but can represent peptides of a protein or glycoprotein.

A recombinant pro-apoptotic BCG vaccine expressing measles antigens can replace regular BCG as a vaccine for administration at birth in developing countries with a high incidence of infant mortality from measles. The recombinant vaccine stimulates cellular immune responses to measles antigens that would protect the infant in the first few year of life when mortality from measles is the greatest. Recombinant pro-apoptotic BCG expressing measles antigens have advantages over the current live-attenuated measles vaccines, as the presence of maternal antibodies interferes with vaccination before 6 months of age, leaving the infant susceptible to measles during a period of life when they are at high risk of dying from measles. Instead, recombinant pro-apoptotic BCG expressing measles antigens will not be inactivated by maternal antibodies, and can induce protective cellular immune responses at an earlier point in life. Heterologous measles virus antigens contemplated by this invention include, but are not limited to, H glycoprotein (hemagglutinin), F glycoprotein, and M protein.

Other heterologous antigens of infectious pathogens contemplated by this invention include, but are not limited to, antigens of malaria sporozoites, antigens of malaria merozoites, human immunodeficiency virus antigens, and leishmania antigens. Heterologous malaria antigens contemplated by this invention include, but are not limited to, circumsporozoite antigen, TRAP antigen, liver-stage antigens (LSA 1, LSA3), blood stage molecules (MSP 1, MSP2, MSP3), PfEMP1 antigen, SP166, EBA 175, AMA1, Pfs25, and Pfs45-48. Heterologous human immunodeficiency virus type 1 (HIV-1) antigens contemplated by this invention include, but are not limited to, proteins and glycoproteins encoded by env, gag, and pol including gp120, gp41, p24, p17, p7, pr6tease, integrase, and reverse transcriptase as well as accessory gene products such as that, rev, vif, vpr, spu, and nef. Heterologous HIV antigens include antigens from different HIV Clades. Heterologous HIV antigens also include cytotoxic T-lymphocyte (CTL) escape epitopes that are not found in native wild-type virus but which have been shown to emerge under the selective pressure of the immune system. In this manner, it vaccination can preemptively prevent mutations that enable the virus to escape from immune containment and which represents a major driving force of HIV sequence diversity. Heterologous Leishmania antigens include antigens from any Leishmania species, including but not limited to, L. donovani, L., infantum, L. chagasi, L. amazonensis, L. tropica, and L. major. Heterologous Leishmania antigens contemplated by this invention include, but are not limited to, gp63, p36(LACK), the 36-kDa nucleoside hydrolase and other components of the Fucose-Mannose-ligand (FML) antigen, glucose regulated protein 78, acidic ribosomal P0 protein, kinetoplastid membrane protein-11, cysteine proteinases type I and II, Trp-Asp (WD) protein, P4 nuclease, papLe22, TSA, LmST11 and LeIF.

Other heterologous antigens of infectious protozoan pathogens contemplated by this invention include, but are not limited to, antigens of Trypanosoma species, Schistosoma species, and Toxoplasma gondii. Heterologous Trypanosoma antigens include antigens from any Trypanosoma species including Trypanosoma cruzi and Trypanosoma brucei. Heterologous Trypanosoma antigens contemplated by this invention include, but are not limited to, paraflagellar rod proteins (PFR), microtubule-associate protein (MAP p15), trans-sialidase family (ts) genes ASP-1, ASP-2, and TSA-1, the 75-77-kDa parasite antigen and variable surface glycoproteins. Heterologous Schistosoma antigens include antigens from any Schistosoma species including, but not limited to, S. mansoni, S. japonicum, S. haematobium, S. mekongi, and S. intercalatum. Heterologous Schistosoma antigens contemplated by this invention include, but are not limited to, cytosolic superoxide dismutase, integral membrane protein Sm23, the large subunit of calpain (Sm-p80), triose-phosphate isomerase, filamin, paramyosin, ECL, SM14, IRV5, and Sm37-GAPDH. Heterologous Toxoplasma antigens contemplated by this invention include, but are not limited to, GRA1, GRA3, GRA4, SAG1, SAG2, SRS1, ROP2, MIC3, HSP70, HSP30, P30, and the secreted 23-kilodalton major antigen.

Other heterologous antigens of infectious viral pathogens contemplated by this invention include, but are not limited to, antigens of Influenza Virus, Hepatitis C Virus (HCV) and Flaviviruses including Yellow Fever Virus, Dengue Virus, and Japanese Encephalitis Virus. Heterologous Influenza virus antigens contemplated by this invention include, but are not limited to, the hemagglutinin (HA), neuraminidase (NA), and M protein, including different antigenic subtypes of HA and NA. Heterologous HCV antigens contemplated by this invention include, but are not limited to, the 21-kDa core (C) protein, envelope glycoproteins E1 and E2, and non-structural proteins NS2, NS3, NS4, and NS5. Heterologous HCV antigens include antigens from the different genotypes of HCV. Heterologous Flavivirus antigens contemplated by this invention include capsid (C) protein, envelope (E) protein, membrane (M) protein, and non-structural (NS) proteins.

Other heterologous antigens of infectious viral pathogens contemplated by this invention include, but are not limited to, structural and non-structural proteins and glycoproteins of the Herpes Virus Family including Herpes Simplex Viruses (HSV) I and 2, Cytomegalovirus (CMV), Varicella-Zoster Virus (VZV), and Epstein-Barr Virus (EBV). Heterologous herpes antigens contemplated by this invention include, but are not limited to, structural proteins and glycoproteins in the spikes, envelope, tegument, nucleocapsid, and core. Also contemplated are non-structural proteins including thymidine kinases, DNA polymerases, ribonucleotide reductases, and exonucleases.

Other heterologous antigens of infectious viral pathogens contemplated by this invention include, but are not limited to, structural and non-structural proteins and glycoproteins of Rotavirus, Parainfluenza Virus, Human Metapneumovirus, Mumps Virus, Respiratory Syncytial Virus, Rabies Virus, Alphaviruses, Hepatitis B Virus, Parvoviruses, Papillomaviruses, Variola, Hemorrhagic Fever Viruses including Marburg and Ebola, Hantaviruses, Poliovirus, Hepatitis A Virus, and Coronavirus including the agent of SARS (severe acute respiratory syndrome).

Other heterologous antigens of infectious pathogens contemplated by this invention include, but are not limited to, antigens of Chlamydia species and Mycoplasma species, including C. pneumoniae, C. psittici, C. trachomatis, M. pneumonia, and M. hyopneumoniae. Heterologous Chlamydia antigens contemplated by this invention include, but are not limited to, major outer membrane protein (MOMP), outer membrane protein A (OmpA), outer membrane protein 2 (Omp2), and pgp3. Heterologous Mycoplasma antigens contemplated by this invention include, but are not limited to, heat shock protein P42.

Other heterologous antigens of infectious pathogens contemplated by this invention include, but are not limited to, antigens of Rickettsial species including Coxiella burnetti, Rickettsia proiwazekii, Rickettsia tsutsugamushi, and the Spotted Fever Group. Heterologous Rickettsial antigens contemplated by this invention include, but are not limited to, ompA, ompB, virB gene family, cap, tlyA, tlyC, the 56-kD outer membrane protein of Orientia tsutsugamushi, and the 47 kDa recombinant protein.

Other heterologous antigens of infectious pathogens contemplated by this invention include, but are not limited to, proteins and glycoproteins of bacterial pathogens including M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Treponema pallidum, other Treponema species, Leptospira species, Borrelia species, Yersinia enterolitica, and other Yersinia species.

Also, the microbes of the present invention can further be modified to express cancer antigens for use as immunotherapy against malignant neoplasms. Heterologous cancer antigens contemplated by this invention include, but are not limited to, tyrosinase, cancer-testes antigens (MAGE-1, -2, -3, -12), G-250, p53, Her-2/neu, HSP105, prostatic acid phosphatase (PAP), E6 and E7 oncoproteins of HPV16, 707 alanine proline (707-AP) (Takahashi T, et al. Clin Cancer Res. August 1997; 3(8):1363-70); alpha (α)-fetoprotein (AFP) (Accession No. CAA79592 (amino acid), Accession No. Z19532 (nucleic acid)); adenocarcinoma antigen recognized by T cells 4 (ART-4) (Accession No. BAA86961 (amino acid), Accession No. AB026125 (nucleic acid)); B antigen (BAGE) (Accession No. NP_{—}001178 (amino acid), Accession No. NM_{—}001187 (nucleic acid)); b-catenin/mutated (Robbins P F, et al. A mutated beta-catenin gene encodes a melanoma-specific antigen recognized by tumor infiltrating lymphocytes. J Exp Med. Mar. 1, 1996; 183(3): 1185-92.); breakpoint cluster region-Abelson (Bcr-abl) (Accession No. CAA10377 (amino acid), Accession No. AJ131467 (nucleic acid)); CTL-recognized antigen on melanoma (CAMEL) (Accession No. CAA10197 (amino acid), Accession No. AJ012835 (nucleic acid)); carcinoembryonic antigen peptide-1 (CAP-1) (Tsang K Y, Phenotypic stability of a cytotoxic T-cell line directed against an immunodominant epitope of human carcinoembryonic antigen. Clin Cancer Res. December 1997; 3(12 Pt 1):2439-49); caspase-8 (CASP-8) (Accession No. NP_{—}001219 (amino acid), Accession No. NM_{—}001228 (nucleic acid)); cell-divisioncycle 27 mutated (CD27m); cycline-dependent kinase 4 mutated (CDK4/m); carcinoembryonic antigen (CEA) (Accession No. AAB59513 (amino acid), Accession No. M17303 (nucleic acid); cancer/testis (antigen) (CT); cyclophilin B (Cyp-B) (Accession No. P23284 (amino acid)); differentiation antigen melanoma (DAM) (the epitopes of DAM-6 and DAM-10 are equivalent, but the gene sequences are different) (DAM-6/MAGE-B2—Accession No. NP_{—}002355 (amino acid), Accession No. NM_{—}002364 (nucleic acid)) (DAM-10/MAGE-B1—Accession No. NP_{—}002354 (amino acid), Accession No. NM_{—}002363 (nucleic acid)); elongation factor 2 mutated (ELF2m); E-26 transforming specific (Ets) variant gene 6/acute myeloid leukemia 1 gene ETS (ETV6-AML1); glycoprotein 250 (G250); G antigen (GAGE) (Accession No. AAA82744 (amino acid));N-acetylglucosaminyltransferase V (GnT-V); glycoprotein 100 kD (Gp100); helicose antigen (HAGE); human epidermal receptor-2/neurological (HER2/neu) (Accession No. AAA58637 (amino acid) and M11730 (nucleic acid); arginine (R) to isoleucine (I) exchange at residue 170 of the α-helix of the a2-domain in the HLA-A2 gene (HLA-A*0201-R170I); human papilloma virus E7 (HPV-E7); heat shock protein 70-2 mutated (HSP70-2M); human signet ring tumor-2 (HST-2); human telomerase reverse transcriptase (hTERT or hTRT); intestinal carboxyl esterase (iCE); KIAA0205; L antigen (LAGE); low density lipid receptor/GDP-L-fucose (LDLR/FUT): b-D-galactosidase 2-a-L-fucosyltransferase; melanoma antigen (MAGE). melanoma antigen recognized by T cells-1/Melanoma antigen A (MART-1/Melan-A)(Accession No. Q16655 (amino acid) and BC014423 (nucleic acid); melanocortin 1 receptor; Myosin/m; mucin 1 (MUC1) (Acession No. CAA56734 (amino acid) X80761 (nucleic acid)); melanoma ubiquitous mutated 1, 2, 3 (MUM-1, -2, -3); NA cDNA clone of patient M88 (NA88-A); New York-esophageous 1 (NY-ESO-1); protein 15 (P15); protein of 190 KD bcr-abl; promyelocytic leukaemia/retinoic acid receptor a (Pml/RARa). preferentially expressed antigen of melanoma (PRAME) (Accession No. AAC51160 (amino acid) and U65011 (nucleic acid)); prostate-specific antigen (PSA)(Accession No. AAA58802 (amino acid) and X07730 (nucleic acid)); prostate-specific membrane antigen ((PSM)(Accession No. AAA60209 (amino acid) and AF007544 (nucleic acid)); renal antigen (RAGE)(Accesssion No. AAH53536 (amino acid) and NM_{—}014226 (nucleic acid)); renal ubiquitous 1 or 2 (RU1 or RU2) (RU1 Accession No. AAF19794 (amino acid) and AF168132 (nucleic acid) or RU2 Accession No. AAF23610 (amino acid) AF181721 (nucleic acid)); sarcoma antigen (SAGE)(Accession No. NP_{—}005424 (amino acid) and NM_{—}018666 (nucleic acid)); squamous antigen recognized by T cells 1 or 3 (SART-1 or SART-3)(SART-1 Accession No. BAA24056 (amino acid) and NM_{—}005146 (nucleic acid) or SART-3 Accession No. BAA78384 (amino acid) AB020880 (nucleic acid)); translocation Ets-family leukemia/acute myeloid leukemia 1 (TEL/AML1); triosephosphate isomerase mutated (TPI/m); tyrosinase related protein 1 (TRP-1) (Accession No. NP_{—}000541 (amino acid) and NM_{—}000550 (nucleic acid)); tyrosinase related protein 2 (TRP-2)(Accession No. CAA04137 (amino acid) and AJ000503 (nucleic acid)); TRP-2/intron 2; and Wilms' tumor gene (WT1)(Accession No. CAC39220 (amino acid) and BC032861 (nucleic acid)), which are incorporated herein by reference.

The microbes of the disclosed methods and compositions can be constructed using the disclosed generational approach to bacterial modification. The list below shows additional combinations of the preferred modifications for introducing into BCG the pro-apoptotic phenotype associated with enhanced immunogenicity.

1^{st} Generation:
a. SAD-BCG (also referred to as: “SD-BCG [mut sodA]”)
b. SIG-BCG (also referred to as: “BCGΔsigH”)
c. SEC-BCG (also referred to as: “BCGΔsecA2”)
d. GLAD-BCG (also referred to as: “GSD-BCG [mut glnA1])

2^{nd} Generation:
a. SAD-SIG-BCG (also referred to as: “BCGΔsigH [mut sodA]”)
b. SAD-SEC-BCG (also referred to as: “BCGΔsecA2 [mut sodA]”)
c. DD-BCG (also referred to as: “BCGΔsigHΔsecA2”, “double-deletion BCG”)
d. GLAD-SIG-BCG (also referred to as: “BCGΔsigH [mut glnA1]”)
e. GLAD-SEC-BCG (also referred to as: “BCGΔsecA2 [mut glnA1]”)
f. GLAD-SAD-BCG (also referred to as: “BCG [mut sodA, mut glnA1])

3^{rd} Generation:
a. 3D-BCG (also referred to as: “BCGΔsigHΔsecA2 [mut sodA]”, “3^{rd}-generation BCG”). There are multiple contemplated 3D-BCG strains based on the nature of the dominant-negative mutant SodA that is expressed to reduce total SOD activity. The dominant-negative mutant sodA gene can be inserted into the chromosome of DD-BCG or expressed on a plasmid.
b. GLAD-DD-BCG (also referred to as: “BCGΔsigHΔsecA2 [mut ginA1]”)
c. GLAD-SAD-SIG-BCG (also referred to as: “BCGΔsigH [mut sodA, mut glnA1]”)
d. GLAD-SAD-SEC-BCG (also referred to as: “BCGΔsecA2 [mut sodA, mut glnA1]”)

4^{th} Generation:

4D-BCG (also referred to as: “BCGΔsigHΔsecA2 [mut soda, mut glnA1]”, “4^{th}-generation BCG”. There are 4 major types of 4D-BCG. All involve the addition of dominant-negative sodA and glnA1 mutants to DD-BCG, but vary in where the genes are inserted.
Form 1—the mutant sodA and glnA1 alleles are inserted into the chromosome
Form 2—the mutant sodA and glnA1 alleles are expressed on a plasmid
Form 3—the mutant sodA allele is inserted into the chromosome and the mutant glnA1 allele is expressed on a plasmid
Form 4—the mutant sodA allele is expressed on a plasmid and the mutant glnA1 allele is inserted into the chromosome

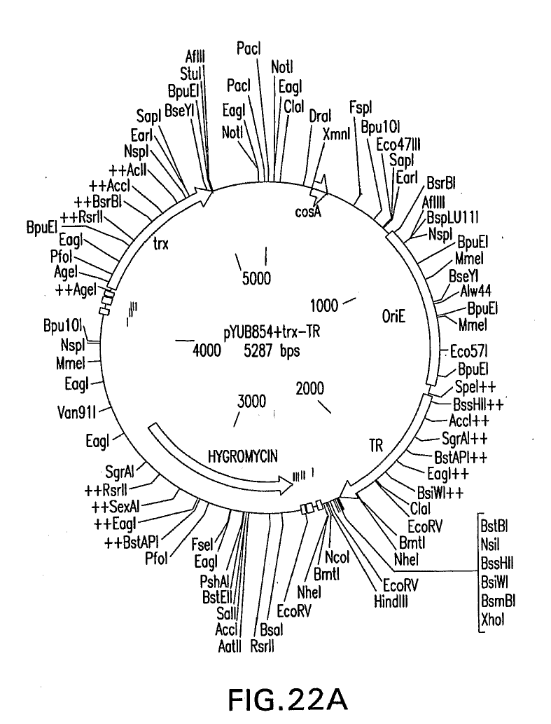

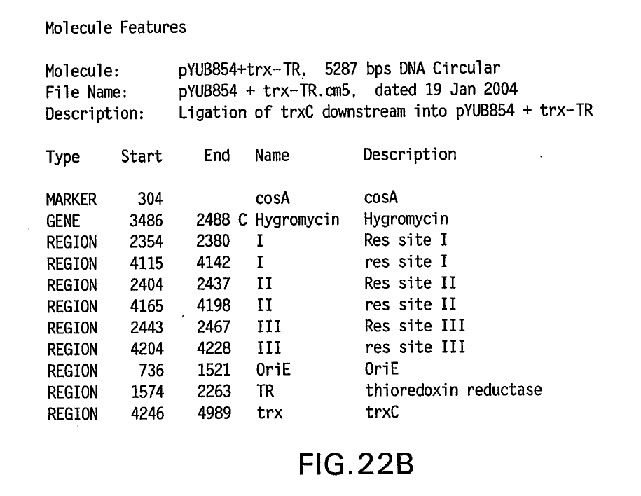

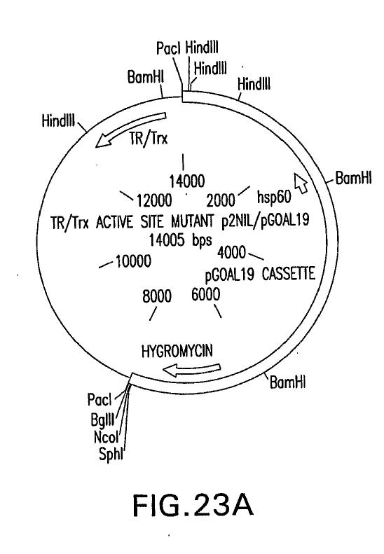

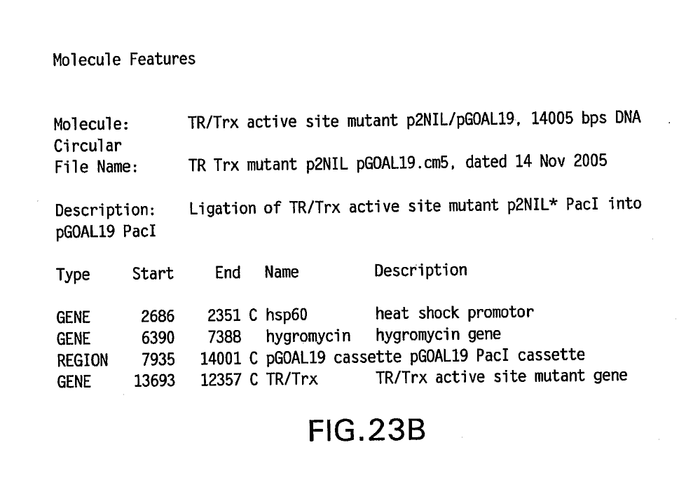

As inactivation of sigH affects the expression of multiple bacterial factors, some of which are important targets of the immune response, there are advantages to substituting the inactivation of sigH with the inactivation (or dominant-negative mutant enzyme expression) of one or more of the antioxidants whose expression is controlled by sigH. These include thioredoxin, thioredoxin reductase, a glutaredoxin homolog, and biosynthetic enzymes involved in the production of mycothiol [Kaushal, D. et al, 2002; Manganelli, R. et al, 2002; Raman, S. et al, 2001], a small molecular weight reducing agent similar to mammalian gluthathione. This manipulation can have advantages over inactivating sigH when the pro-apoptotic BCG strain will be used to vaccinate a host against tuberculosis, as the benefit of having the host respond to the sigH-controlled factors as immune targets may outweigh the benefit of having a vaccine strain that is less able to inhibit apoptosis. In contrast, the sigH-inactivated vaccines described herein are ideal vectors to use in expressing exogenous antigens, as the presence of a complete or near-complete antigen repertoire of BCG is not important when the modified BCG strain is used primarily to induce an immune response against an exogenous antigen, e. g, for immunizing against other infectious agents or cancer antigens. To further teach how to practice the substitution of inactivating sigH-regulated anti-apoptotic genes instead of inactivating sigH, mutant alleles designed to inactivate thioredoxin and thioredoxin reductase are shown in FIG. 22 and FIG. 23. This approach is applicable to M. bovis strains other than BCG.

The paBCG vaccines disclosed herein are more immunogenic than the parent BCG vaccine strain. Furthermore, each vaccine generation exhibits progressive increases in immunogenicity. Compared to BCG they exhibit the following traits:

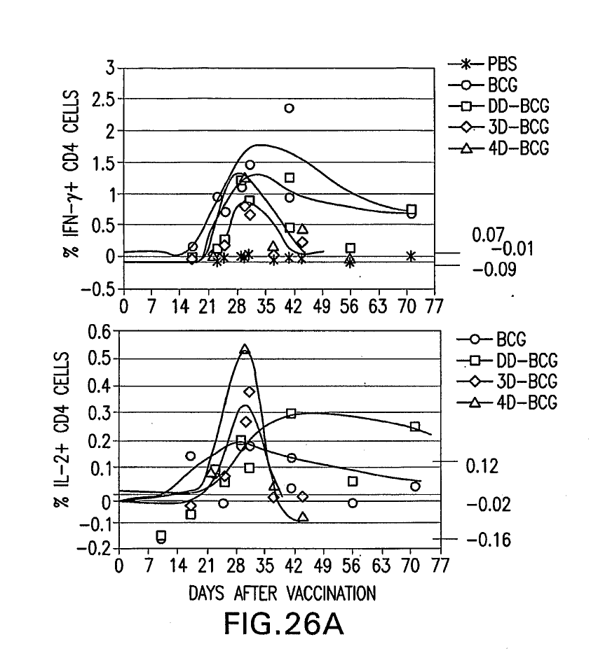

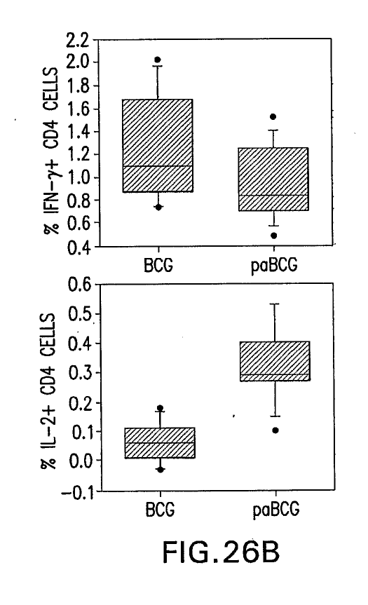

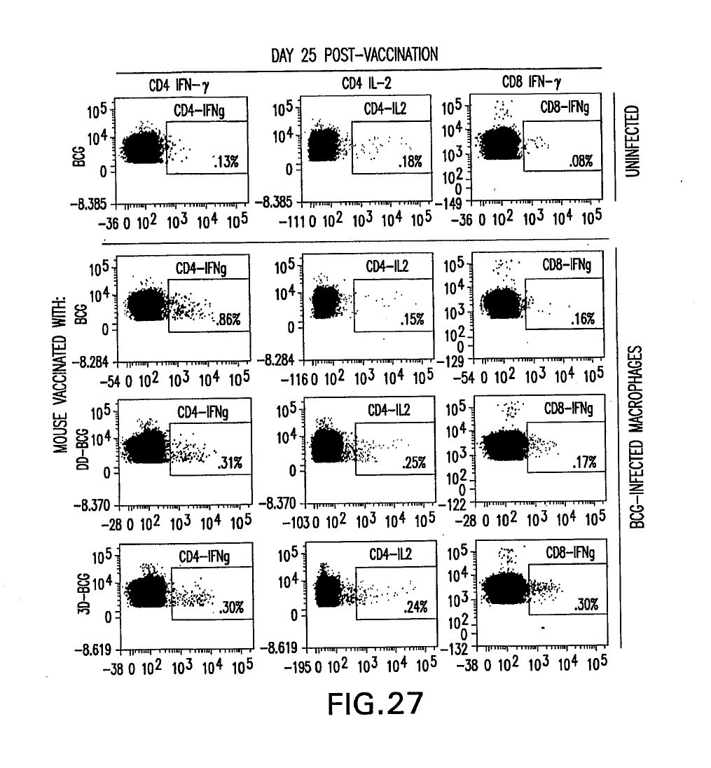

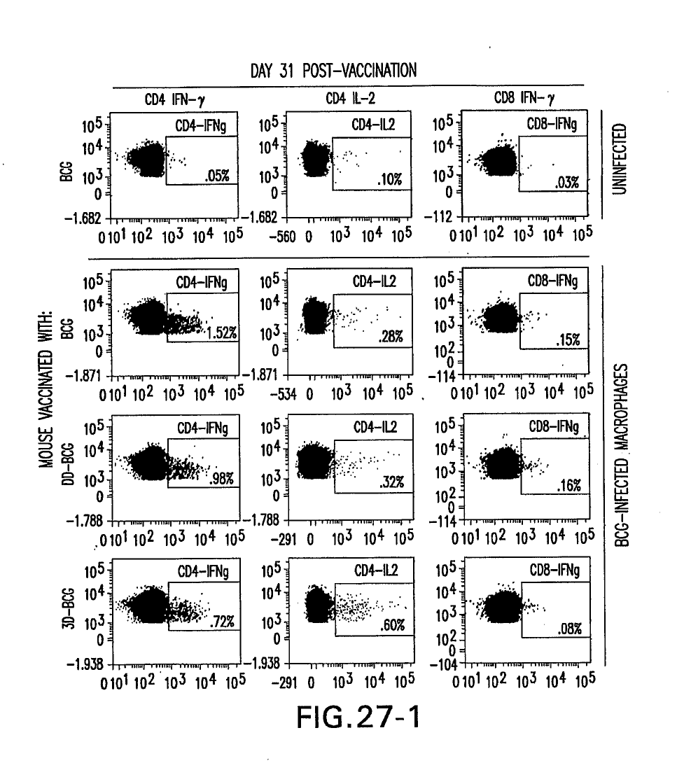

1. They induce a qualitatively and quantitatively different pattern of CD4+ T-cell responses during primary vaccination with higher peak IL-2 production and less prolonged IFN-γ release (Example 13, FIGS. 26 and 27). Both of these differences can be important in generating memory T-cells. First, IL-2 enhances the survival of antigen-specific T-cells, and is required for the generation of robust secondary responses. Second, although IFN-γ is a commonly measured effector function of effector T-cells that activates MΦs, it promotes T-cell apoptosis during the contraction phase of primary proliferation.

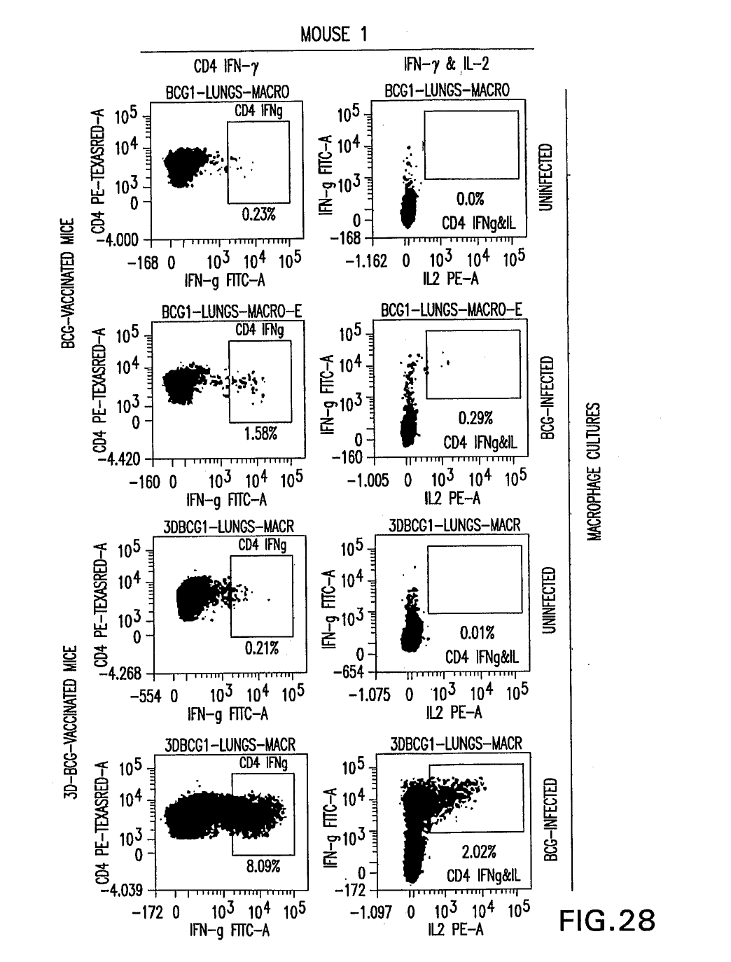

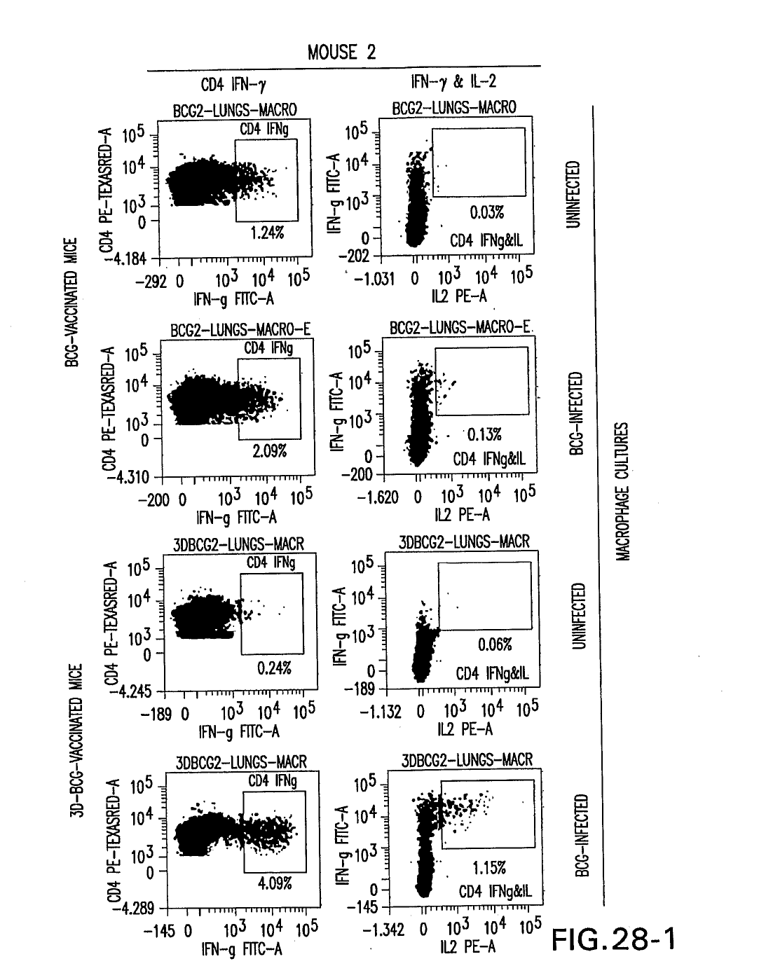

2. They induce more rapid recall T-cell responses to a second exposure. Strong T-cell responses are detected within 5 days post-challenge in mice previously subQ-vaccinated with 3DBCG (Example 14, FIG. 28). This compares favorably to recall responses in BCG-vaccinated mice which peak at day 11-14.

In summary, the results show that the modified BCG induces a better immune response to vaccination.

The present invention is more particularly described in the following examples which are intended as illustrative only since numerous modifications and variations therein will be apparent to those skilled in the art.

The methods, bacterial isolates, plasmids, and other tools for performing genetic manipulations described in WO 02/062298 are hereby incorporated by reference in their entirety for the teaching of these compositions and methods.

### Examples

General Methods.

Bacterial isolates, plasmids, chemicals, and culture media: Bacterial isolates and plasmids used are shown in Table 1. E. coli strain TOP 10 was used as the host for cloning PCR products and E. coli strain DH5α was used as the host for other molecular genetic manipulations unless otherwise indicated. E. coli strains were grown in LB media (Gibco/BRL, Gaithersburg, Md.). BCG Tice was grown in Middlebrook 7H9 liquid media (Difco Laboratories, Detroit, Mich.) supplemented with 0.2% glycerol, 10% Middlebrook OADC enrichment (Becton Dickinson & Co., Cockeysville, Md.), and 0.05% Tween80. Alternatively, it was grown on Middlebrook 7H10 agar (Difco) supplemented with glycerol and OADC. Kanamycin at a concentration of 50 μg/ml or 25 μg/ml, apramycin at a concentration of 50 μg/ml, or hygromycin at a concentration of 100 μg/ml or 50 μg/ml was used in E. coli DH5α or BCG to select for transformants containing plasmids or chromosomal integration vectors.

Gene mutagenesis. The genes for iron co-factored superoxide dismutase (sodA) and glutamine synthase (glnA1) were PCR-amplified from chromosomal DNA of M. tuberculosis strain H37RV and cloned in plasmids that replicate in E. coli. DNA sequence data stored in the TubercuList web server (http://genolist.pasteur.fr/TubercuList/site), also stored in GeneBank, was used to guide the construction of DNA primers. Site-directed mutagenesis using the PCR-based primer overlap extension methods [Ho, S. N. et al, 1989] or other methods are employed to eliminate, substitute, or add nucleotides. This produces mutant genes that encode mutant enzymes with deletions, substitutions, or additions of amino acids. Gene sequence is confirmed by DNA sequencing. Alternatively, gene synthesis techniques can be used to produce the genes with the desired sequence.

Expression of mutant enzyme genes in BCG. Genes encoding mutant enzymes were ligated into one or more of the following vectors: pMH94, pHV202, pMP349, and pMP399. Other vectors can also be used to practice this invention. Expression of mutant SodA in the chromosomal integration-proficient vector pLou1 was achieved using the cloned wild-type sodA promoter as part of an alternative strategy for practicing targeted incremental attenuation as described in WO 02/062298. This alternative strategy involved first inserting the mutant sodA allele encoding an enzyme exhibiting diminished SOD activity into the attB phage integration site on the mycobacterial chromosome. The transformants of pMH94-mut sodA grew slower than the parent BCG strain. The slow growth of these strains was similar to the slow-growth phenotype observed in M. tuberculosis and BCG strains in which antisense overexpression techniques had been used to reduce SOD activity. Realizing that this represented a dominant-negative effect of expressing the mutant SodA, the mutant SodA was then expressed in pMP349 and pMP399. In these constructs, the sodA promoter was eliminated and the mutant SodA open reading frame was placed behind a 350+ base pair region that includes the promoter for aceA (also called icl) [Graham, J. E. et al, 1999; McKinney, J. D. et al, 2000; McKinney, J. D. et al, 2000]. A kpn1 restriction site was used in ligation and the complete sequence of promoter-Kpn1 site-mutant SodA reading frame is shown in Example 1. The aceA promoter is macrophage-inducible and expression can also be regulated in vitro, a feature that offers potential advantages if the gene being expressed interferes with bacterial growth. Results involving mutant SodA expressed in pMP399 are shown in the examples and figures. Expression of mutant glnA1 in pMP349 and pMP399 was performed using the cloned glnA1 promoter.

The vectors were electroporated into BCG Tice using standard methods [Hondalus, M. K. et al, 2000] except that when the A₆₀₀ of the mycobacterial cultures reached 0.6, they were incubated in 37° C. and 5% CO₂ with 1.5% glycine and 50 ug/ml m-fluoro-DL-phenylalanine (MFP) for 48 hrs to enhance electroporation efficiency. The inycobacteria were washed twice and resuspended in ice-cold 10% glycerol. The Gene Pulser apparatus with the Pulse Controller accessory (Bio-Rad Laboratories, Hercules, Calif.) was used for all electroporations at 25 F and 2.5 kV with the pulse controller set at 1000 ohms. After electroporation, 1 ml of Middlebrook 7H9 media was added to the samples, and the transformants were allowed to incubate in 37 C and 5% CO₂ for 24 hrs. Transformants were plated on Middlebrook 7H10 agar containing either kanamycin, apramycin, or hygromycin as needed. Successful transformation was confirmed by PCR of DNA unique to the vector construct.

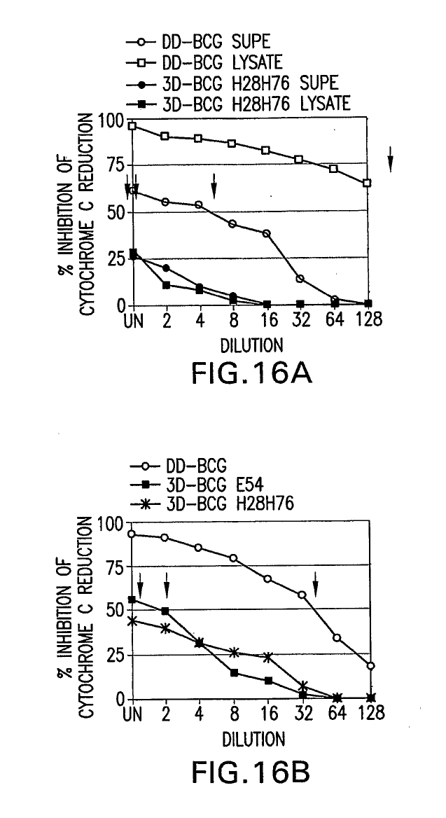

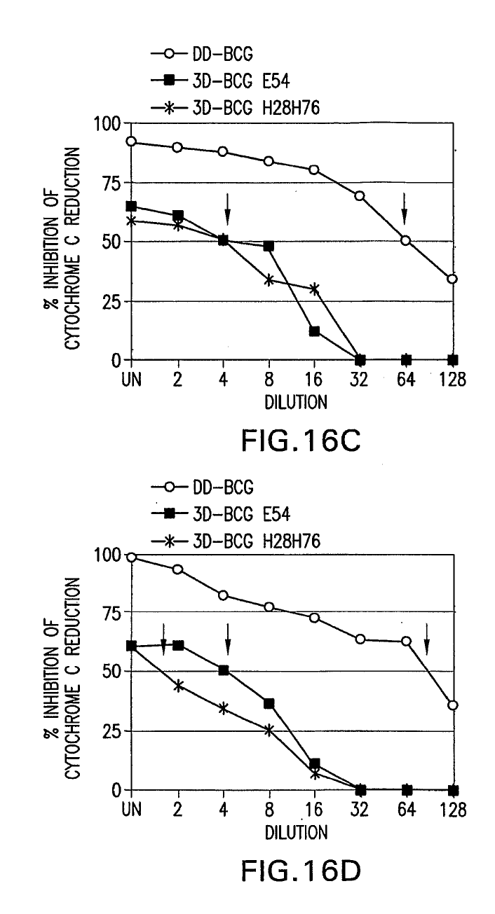

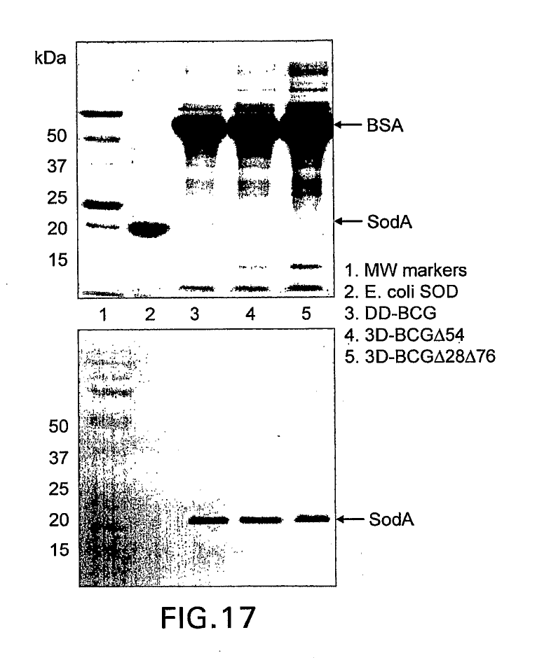

Assays of enzyme amount and activity. The dominant-negative mutant enzyme strategy involves the expression of mutant enzyme monomers in the bacterium that interact with the bacterium's own chromosomally-encoded wild-type enzyme monomers in a manner that reduces the total activity of the enzyme produced by the bacterium. Thus, to obtain information that confirms success in the dominant-negative strategy, a non-enzymatic assay to measure enzyme quantity (e.g., Western hybridization) as well as an assay of enzyme activity were performed. The result is that compared to the parent BCG strain, the mutant BCG strains demonstrated comparable or elevated enzyme quantity (FIG. 17) but diminished enzyme activity (FIG. 16).

To prepare supernatants and lysates for enzyme activity assays, a fresh culture of each BCG strain was prepared by resuspending a washed cell pellet in 25 ml of 7H9 broth containing OADC to achieve an A600 value of 0.5. Broth was grown without shaking for 72 hours. The broth culture was centrifuged and supernatant separated from the cell pellet. Concentrated supernatants for enzyme activity determinations were prepared by concentrating the 25 ml supernatant to 1.0 ml using a centrifuge-based separation device with a 10,000 kDA membrane. Lysates for testing enzyme activity were prepared by resuspending the cell pellet in 1 ml of phosphate buffered saline and lysing with a microbead-beater apparatus. Lysates from different strains were adjusted to a standard A280 value for comparison.

Western hybridization was used to quantity the amount of SOD. Samples consisting of undiluted cell lysates as prepared above were adjusted to a standard A280 values, applied to and electrophoresed on a 12% PAGE gel, and transferred to Hybond ECL nitrocellulose membranes (Amersham, Arlington Heights, Ill.). Membranes were hybridized with rabbit polyclonal antisera raised against PAGE-purified recombinant SodA expressed in E. coli as previously described [Lakey, D. L. et al, 2000]. The recombinant SodA used to generate antibodies was purified by nickel affinity column chromatography. The nitrocellulose membranes were incubated first with antisera at the dilutions noted above followed by incubation with a 1:1000 dilution of horseradish peroxidase-conjugated goat anti-rabbit antibodies (Boehringer Mannheim, Indianapolis, Ind.). The immunoblots were developed with ECL Western blot detection reagents (Amersham Pharmacia, Arlington Heights, Ill.).

SOD activity was measured spectrophotometrically by its ability to interfere with the reduction of cytochrome C by superoxide using a commercial kit utilizing xanthine oxidase-generated superoxide and based on the methods of McCord and Fridovich [McCord, J. M. et al, 1969; Beyer, W. F., Jr. et al, 1987]. One SOD unit was defined as the amount of SodA that inhibited cytochrome C reduction by 50% (IC50 value).

Glutamine synthase activity was measured spectrophotometrically by using the methods of Woolfolk et al [Woolfolk, C. A. et al, 1966].

In vivo Challenge-Protection Studies.

To prepare vaccine strain inocula for injection into C57BL/6 mice, BCG Tice and the pro-apoptotic BCG vaccine strains were grown in modified Middlebrook 7H10 broth (7H10 agar formulation with malachite green and agar deleted) containing 10% OADC (Difco). The suspensions were diluted to achieve a 100 Klett unit reading (approximately 5×10⁷ cfu/ml) on a Klett-Summerson Colorimeter (Klett Manufacturing, Brooklyn, N.Y.). Aliquots of the inocula were serially diluted and directly plated to 7H10 agar containing 10% OADC for backcounts to determine the precise inoculum size.

Female C57BL/6 mice aged 5-6 weeks were purchased from Jackson Laboratories, Bar Harbor, Me. Infected and uninfected control mice were maintained in a pathogen-free Biosafety Level-3 facility at the Syracuse VA Medical Center. Animal experiments were approved by the Syracuse VAMC Subcommittee on Animal Studies and performed in an AALAC-approved facility.

Unless otherwise stated, the experimental design for vaccination-challenge experiments involved subcutaneous inoculation of 5×10⁶ cfu of the vaccine strain, rest for 100 days, and then challenge with an aerosol inoculum of 300 cfu of strain Erdman or acrR-Erdman. Euthanasia was achieved by CO₂ inhalation. Spleens and right lungs were removed aseptically, tissues were placed in a sealed grinding assembly (IdeaWorks! Laboratory Products, Syracuse, N.Y.) attached to a Glas-Col Homogenizer (Terre Haute, Ind.) and homogenized. Viable cell counts were determined by titration on 7H10 agar plates containing 10% OADC.

Histopathologic evaluation: Left lungs were harvested from mice and fixed in 10% formalin (Accustain, Sigma). Lungs were paraffin-embedded, cut in 4-μm sections and stained with hematoxylin and eosin.

Flow cytometry and tissue stains. Cell populations were analyzed on a Becton-Dickinson FACScalibur flow cytometer with Mac Workstation. Data were collected in listmode and offline analyses were performed using PC platform Winlist software (Verity Software House, Topsham Me.). Antibodies for flow cytometry were purchased from BD Pharmingen (San Diego, Calif.). Samples were incubated with Rat anti-Mouse anti-CD16/CD32 clone 2.4G2 (Fc Block, BD Pharmingen) for 15 minutes to reduce background. A total of 10,000 gated events in each specimen were collected and analysis gates included a lymphocyte gate and non-lymphocyte gate based on cell size and granularity, with gate dimensions kept constant between experiments.

### Example 1

### Construction of SAD-BCG ΔH28ΔH76 [also Referred to as “BCG (mut sodA ΔH28ΔH76)”, or “SodA-Diminished BCG Expressing Dominant-Negative ΔH28ΔH76 Mutant SodA”] and Documentation of Reduced SOD Activity In vitro

To construct SAD-BCG ΔH28ΔH76, a ΔH28ΔH76 soda mutant in pCR2.1-TOPO was made by performing PCR-based site-directed mutagenesis on the wild-type sodA allele that had been PCR-amplified from chromosomal DNA from M. tuberculosis H37Rv. The open reading frame of the ΔH28ΔH76 mutant soda allele is shown below. Initiation and stop codons are bold, and --- shows the position of the two deleted CAC (histidine-encoding) codons corresponding to amino acid 28 and amino acid 76 of the enzyme.

SEQ ID NO: 1

| | |
|---|---|
| 1 | gtg gcc gaa tac acc ttg cca gac ctg gac |
| 31 | tgg gac tac gga gca ctg gaa ccg cac atc |
| 61 | tcg ggt cag atc aac gag ctt cac --- agc |
| 91 | aag cac cac gcc acc tac gta aag ggc gcc |
| 121 | aat gac gcc gtc gcc aaa ctc gaa gag gcg |
| 151 | cgc gcc aag gaa gat cac tca gcg atc ttg |
| 181 | ctg aac gaa aag aat cta gct ttc aac ctc |
| 211 | gcc ggc cac gtc aat --- acc atc tgg tgg |
| 241 | aag aac ctg tcg cct aac ggt ggt gac aag |
| 271 | ccc acc ggc gaa ctc gcc gca gcc atc gcc |
| 301 | gac gcg ttc ggt tcg ttc gac aag ttc cgt |
| 331 | gcg cag ttc cac gcg gcc gct acc acc gtg |
| 361 | cag ggg tcg ggc tgg gcg gca ctg ggc tgg |
| 391 | gac aca ctc ggc aac aag ctg ctg ata ttc |
| 421 | cag gtt tac gac cac cag acg aac ttc ccg |
| 451 | cta ggc att gtt ccg ctg ctg ctg ctc gac |
| 481 | atg tgg gaa cac gcc ttc tac ctg cag tac |
| 511 | aag aac gtc aaa gtc gac ttt gcc aag gcg |
| 541 | ttt tgg aac gtc gtg aac tgg gcc gat gtg |
| 571 | cag tca cgg tat gcg gcc gcg acc tcg cag |
| 601 | acc aag ggg ttg ata ttc ggc tga |

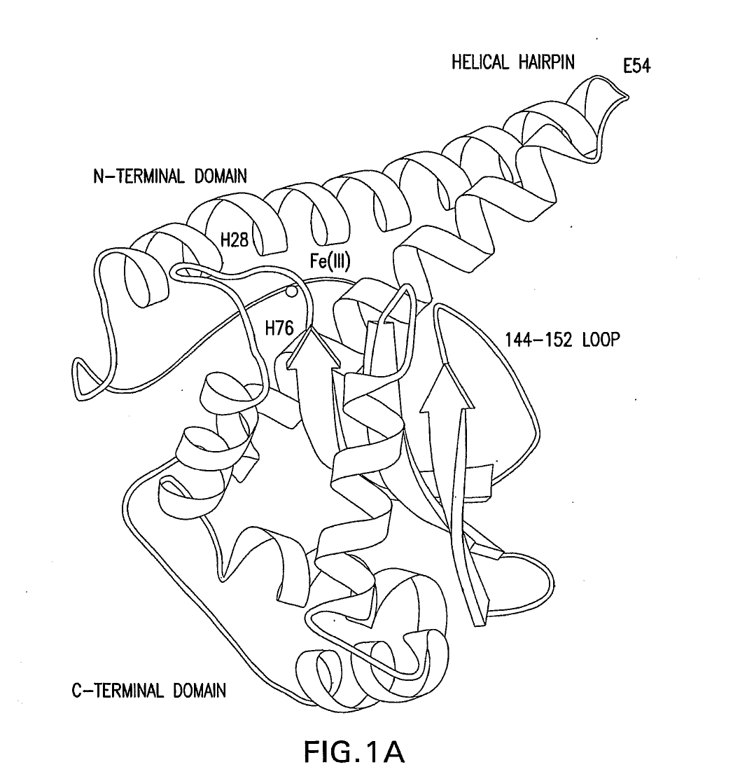

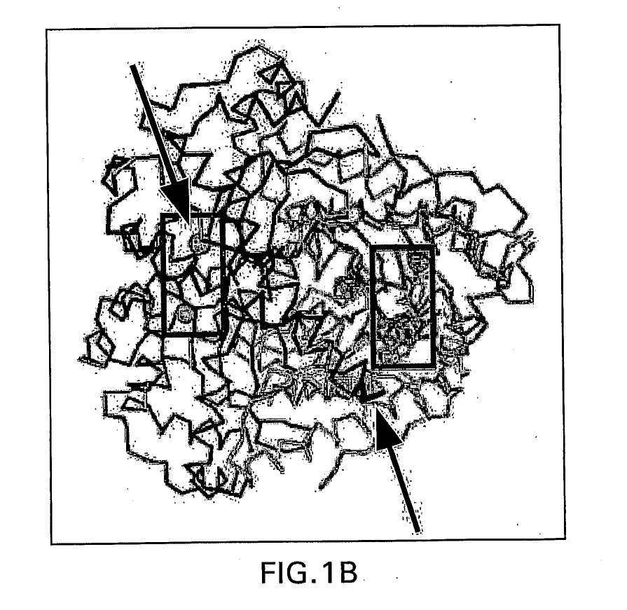

The positions of these amino acid deletions in the context of major alpha helices, beta-strands, and the active site Fe(III) of the SodA monomer are shown in FIG. 1.

A BLASTN query of this DNA sequence against the nucleotide sequence of the complete M. tuberculosis H37Rv sequence was performed using the BLAST server of the TubercuList World Wide Web site (http://genolist.pasteur.fr/TubercuList/), documenting the deletion of the two CAC (histidine) codons.
≧M. tuberculosis H37Rv|null M. tuberculis H37RV (4411532 bp)

Length=4411532
Score=1181 bits (596), Expect=0.0
Identities=618/624 (99%), Gaps 6/624 (0%)
Strand=Plus/Plus

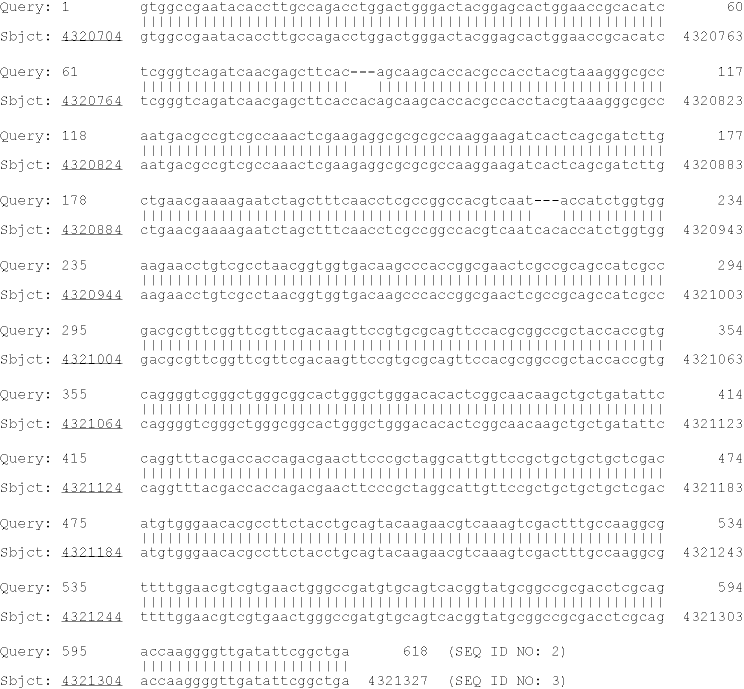

A TBLASTN query was also performed against translated nucleotide sequence data at the TubercuList BLAST site (http://genolist.pasteur.fr/TubercuList/), showing the positions of the deleted histidines.
M. tuberculosis H37Rv|null M. tuberculis H37RV (4411532 bp) Length=4411532
Score=418 bits (1075), Expect=e-118
Identities=205/207 (99%), Positives=205/207 (99%), Gaps=2/207 (0%)
Frame=+2

| | | | | |
|---|---|---|---|---|
| Query: | 1 | VAEYTLPDLDWDYGALEPHISGQINELH-SKHHATYVKGANDAVAKLEEARAKEDHSAIL | 59 | (SEQ ID NO: 4) |
| Sbjct: | 4320704 | VAEYTLPDLDWDYGALEPHISGQINELHHSKHHATYVKGANDAVAKLEEARAKEDHSAIL | 4320883 | (SEQ ID NO: 5) |
| Query: | 60 | LNEKNLAFNLAGHVN-TIWWKNLSPNGGDKPTGELAAAIADAFGSFDKFRAQFHAAATTV | 118 | |
| Sbjct: | 4320884 | LNEKNLAFNLAGHVNHTIWWKNLSPNGGDKPTGELAAAIADAFGSFDKFRAQFHAAATTV | 4321063 | |
| Query: | 119 | QGSGWAALGWDTLGNKLLIFQVYDHQTNFPLGIVPLLLLDMWEHAFYLQYKNVKVDFAKA | 178 | |
| Sbjct: | 4321064 | QGSGWAALGWDTLGNKLLIFQVYDHQTNFPLGIVPLLLLDMWEHAFYLQYKNVKVDFAKA | 4321243 | |
| Query: | 179 | FWNVVNWADVQSRYAAATSQTKGLIFG | 205 | |
| Sbjct: | 4321244 | FWNVVNWADVQSRYAAATSQTKGLIFG | 4321324 | |

BLASTN and TBLASTN queries were also performed against nucleotide sequence data in the M. bovis BLAST server of the Sanger Centre (http://www.sanger.ac.ul/cgibin/blast/submitblast/m_bovis). The Sanger Centre is sequencing Mycobacterium bovis BCG Pasteur and the preliminary M. bovis BCG assembly was used. The results (below) show that in addition to the two CAC codon deletions, in BCG there is an additional T-C nucleotide difference that yields a an I→T amino acid substitution at position 203.

BLASTN results:
>BCG79c08.s1k 19151 bp, 160 reads, 36.25 AT
[Full Sequence]
Length=19,151
Plus Strand HSPs:
Score=3021 (459.3 bits), Expect=6.4e-132, P=6.4e-132
Identities=617/624 (98%), Positives=617/624 (98%), Strand=Plus/Plus
[HSP Sequence]

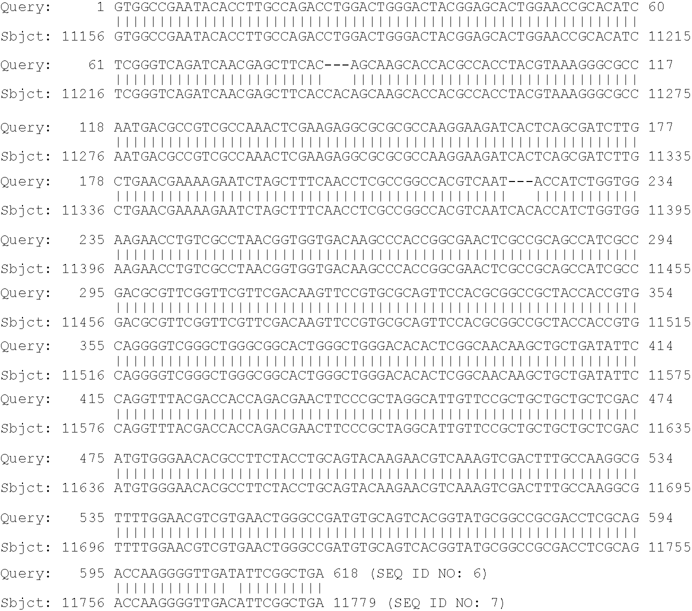

TBLASTN results:
>BCG79c08.s1k 19151 bp, 160 reads, 36.25 AT
[Full Sequence]
Length=19,151
Plus Strand HSPs:
Score=1076 (383.8 bits), Expect=3.6e-109, P=3.6e-109
Identities=204/207 (98%), Positives=204/207 (98%), Frame=+2

[HSP Sequence]

| | | | | |
|---|---|---|---|---|
| Query: | 1 | VAEYTLPDLDWDYGALEPHISGQINELH-SKHHATYVKGANDAVAKLEEARAKEDHSAIL | 59 | (SEQ ID NO: 8) |
| Sbjct: | 11156 | VAEYTLPDLDWDYGALEPHISGQINELHHSKHHATYVKGANDAVAKLEEARAKEDHSAIL | 11335 | (SEQ ID NO: 9) |
| Query: | 60 | LNEKNLAFNLAGHVN-TIWWKNLSPNGGDKPTGELAAAIADAFGSFDKFRAQFHAAATTV | 118 | |
| Sbjct: | 11336 | LNEKNLAFNLAGHVNHTIWWKNLSPNGGDKPTGELAAAIADAFGSFDKFRAQFHAAATTV | 11515 | |
| Query: | 119 | QGSGWAALGWDTLGNKLLIFQVYDHQTNFPLGIVPLLLLDMWEHAFYLQYKNVKVDFAKA | 178 | |
| Sbjct: | 11516 | QGSGWAALGWDTLGNKLLIFQVYDHQTNFPLGIVPLLLLDMWEHAFYLQYKNVKVDFAKA | 11695 | |
| Query: | 179 | FWNVVNWADVQSRYAAATSQTKGLIFG | 205 | |
| Sbjct: | 11696 | FWNVVNWADVQSRYAAATSQTKGLTFG | 11776 | |

Next, the mutant sodA allele was ligated into the chromosomal integration vector pMP399 and the plasmid vector pMP349 behind an aceA(icl) promoter to yield pMP399-mut SodA ΔH28ΔH76 and pMP349-mut SodA ΔH28ΔH76 (Table 1). The plasmid maps are shown in FIG. 2 and the complete nucleotide sequences of these constructs are included in the footnotes of Table 1. The sequence shown below highlights the nucleotide sequence of the aceA(icl) promoter through the mutant sodA open reading frame. Key features are: [a] the sequence encoding the aceA(icl)-associated promoter (base 5044 to base 5385, [b] the open reading frame for the sodA(ΔH28ΔH76) mutant (base 9-base 626), and [c] a Kpn1 restriction site (base 1-base 8) used to connect [a] and [b]:

| SEQ ID NO: 10 | |
|---|---|
| 5041 | ctgttac aacgctcaca tatgtggttg gcgacgagcc caaggcagtc gcctcgctgt |
| 5101 | tcaatctgtg accggatccg caggacgtcg atccgtgggt ttacctgcgg atttgtcgtt |
| 5161 | actggcgggt agcttctgaa acggttcagt ttttgggcga cttcgcaaaa tttgcaaaaa |
| 5221 | gtccgcaggc cgttgccgaa attcgcaagt gaaatgggtg gaccagcgtt gacacgctgt |
| 5281 | gccatggtcg agttagcaca ccagtgaagc tgcgccgttg acaccgcctg gacgacggta |
| 5341 | gggcgtcagc gttttcggca atgaaagacc gttaaggagt tgtct |
| 1 | ggtaccccgt ggccgaatac accttgccag acctggactg ggactacgga gcactggaac |
| 61 | cgcacatctc gggtcagatc aacgagcttc acagcaagca ccacgccacc tacgtaaagg |
| 121 | gcgccaatga cgccgtcgcc aaactcgaag aggcgcgcgc caaggaagat cactcagcga |
| 181 | tcttgctgaa cgaaaagaat ctagctttca acctcgccgg ccacgttaat accatctggt |
| 241 | ggaagaacct gtcgcctaac ggtggtgaca agcccaccgg cgaactcgcc gcagccatcg |
| 301 | ccgacgcgtt cggttcgttc gacaagttcc gtgcgcagtt ccacgcggcc gctaccaccg |
| 361 | tgcaggggtc gggctgggcg gcactgggct gggacacact cggcaacaag ctgctgatat |
| 421 | tccaggttta cgaccaccag acgaacttcc cgctaggcat tgttccgctg ctgctgctcg |
| 481 | acatgtggga acacgccttc tacctgcagt acaagaacgt caaagtcgac tttgccaagg |
| 541 | cgttttggaa cgtcgtgaac tgggccgatg tgcagtcacg gtatgcggcc gcgacctcgc |
| 601 | agaccaaggg gttgatattc agctga |

Next, pMP399-mut SodA ΔH28ΔH76 was electroporated into BCG Tice to produce SAD-BCG ΔH28ΔH76 (SodA-Diminished BCG, also called BCG (mut sodA ΔH28ΔH76). Transformants were selected on agar containing apramycin. PCR of chromosomal DNA using nucleotide sequences unique to the pMP399 vector was used to verify successful integration of the vector into the BCG chromosome.

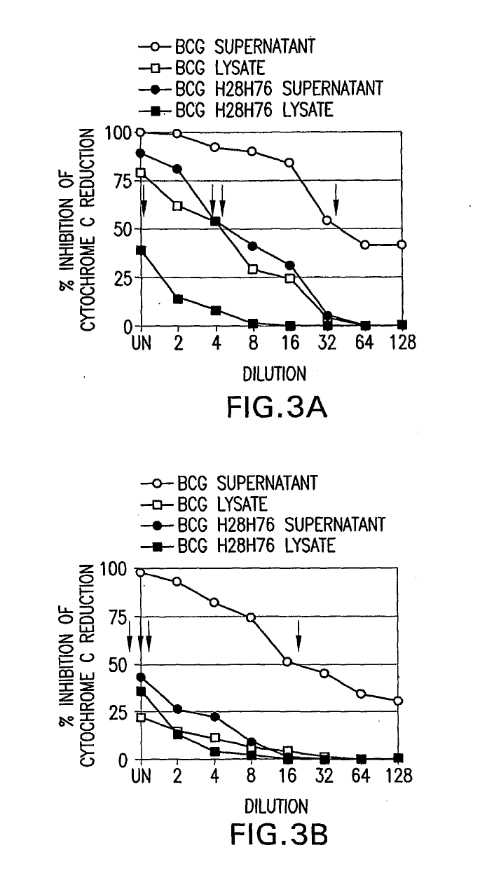

To determine the effect of expressing mutant ΔH28ΔH76 SodA upon the SOD activity of the whole bacterium, supernatants and lysates of BCG and SAD-BCG ΔH28ΔH76 were prepared as described above and compared for SOD activity by monitoring interference (by SOD) with reduction of cytoclrome C by xanthine oxidase-generated superoxide (O2−). Results are shown in FIG. 3 and demonstrate that most of the activity can be found in the supernatant, and that the dominant-negative strategy results in an approximately 8- to 16-fold reduction in SOD activity.

### Example 2

### Construction of SAD-BCG ΔE54 [Aka BCG (Mut SodA ΔE54), or SodA-Diminished BCG Expressing Dominant-Negative ΔE54 Mutant SodA] and Documentation of Reduced SOD Activity In vitro

An additional dominant-negative sodA mutant with a ΔE54 deletion was constructed using the techniques described. The position of this amino acid deletion in the context of major alpha helices, beta-strands, and the active site Fe(III) of the SodA monomer are shown in FIG. 1. DNA sequencing of the gene in pCR2.1-TOPO identified an additional nucleotide substitution that introduced a histidine→arginine substitution at position 28.

The mutant ΔE54 sodA allele was ligated into the chromosomal integration vector pMP399 and the plasmid vector pMP349 behind an aceA(icl) promoter to yield pMP399-mut SodA ΔE54 and pMP349-mut SodA ΔE54 (Table 1). The complete nucleotide sequences of these constructs are included in the footnotes of Table 1. pMP399-mut SodA ΔE54 was electroporated into BCG Tice to produce SAD-BCG ΔE54 (SodA-Diminished BCG, also called BCG (mut sodA ΔE54).

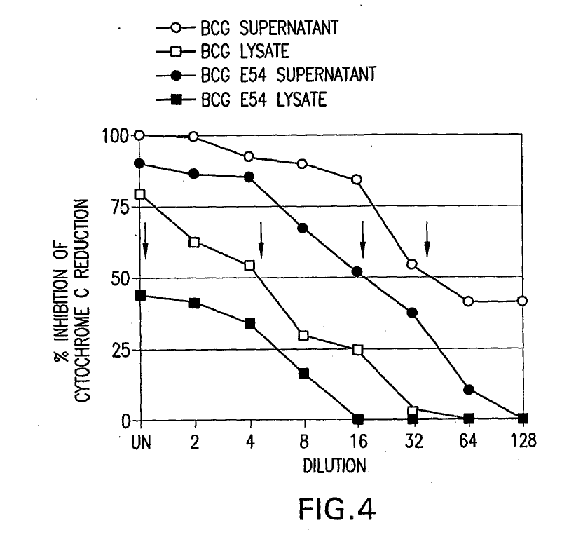

To determine the effect of expressing mutant ΔE54 SodA upon the SOD activity of the whole bacterium, supernatants and lysates of BCG and SAD-BCG ΔE54 were prepared as described above and compared for SOD activity. Results are shown in FIG. 4 and demonstrate a less marked reduction in total SOD activity than was observed with SAD-BCG ΔH28ΔH76.

### Example 3

### The Vaccine Efficacy of SD-BCG-AS-SOD—Implications Regarding the Usefulness of Dominant-Negative SodA-Diminished BCG Strains

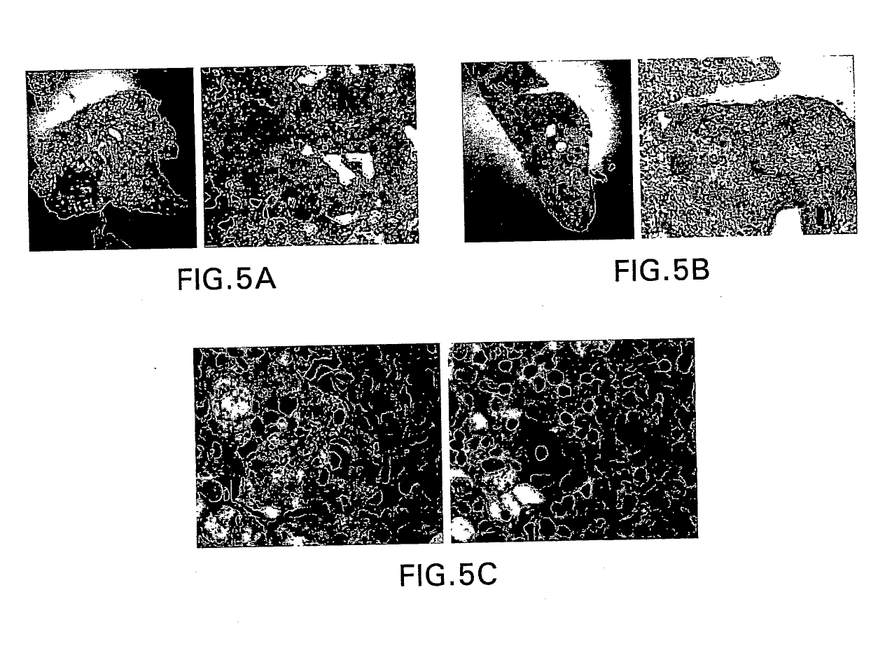

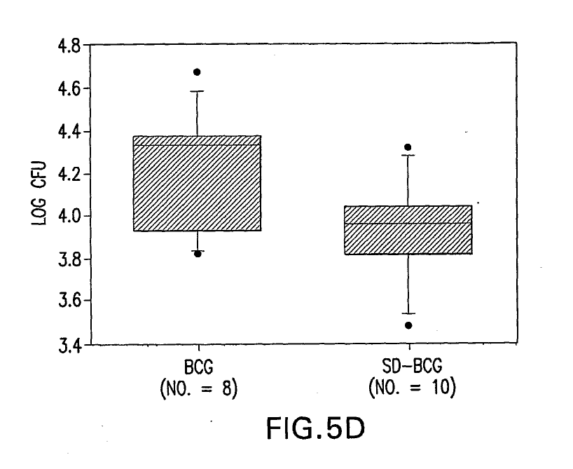

To quantify the amount of improvement in vaccine efficacy that occurs as a consequence of reducing SodA production by BCG, BCG and SD-BCG-AS-SOD (SodA-diminished BCG constructed by using antisense techniques as previously described in WO 02/062298) were compared. Experimental details and results are shown in FIG. 5 and indicate that C57Bl/6 mice vaccinated with SD-BCG-AS-SOD had lower lung cfu counts and less lung damage than mice vaccinated with BCG at six months following aerosol challenge with virulent M. tuberculosis.

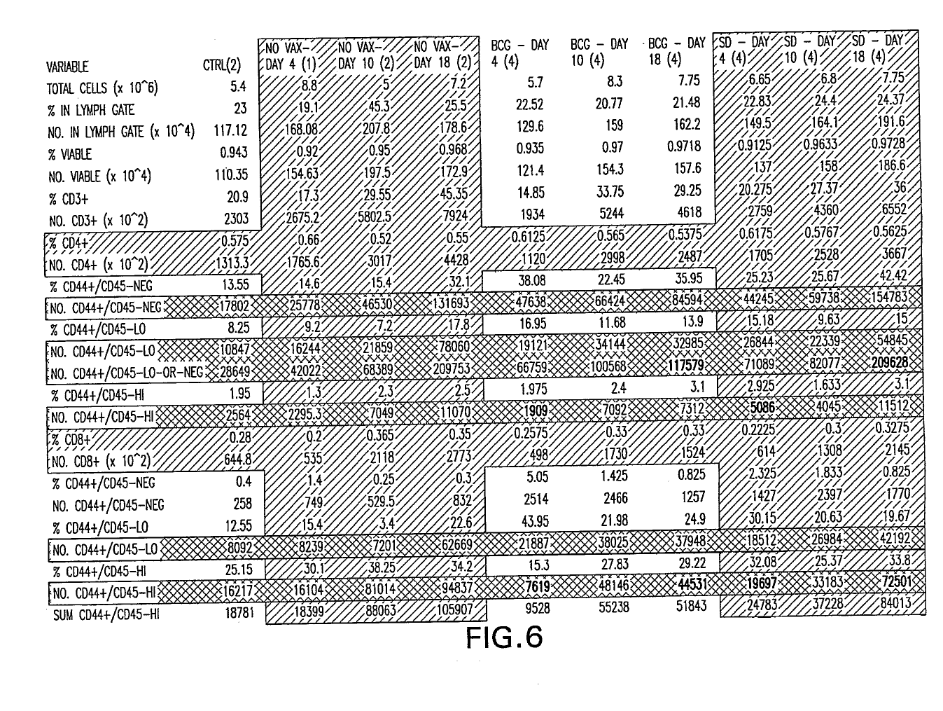

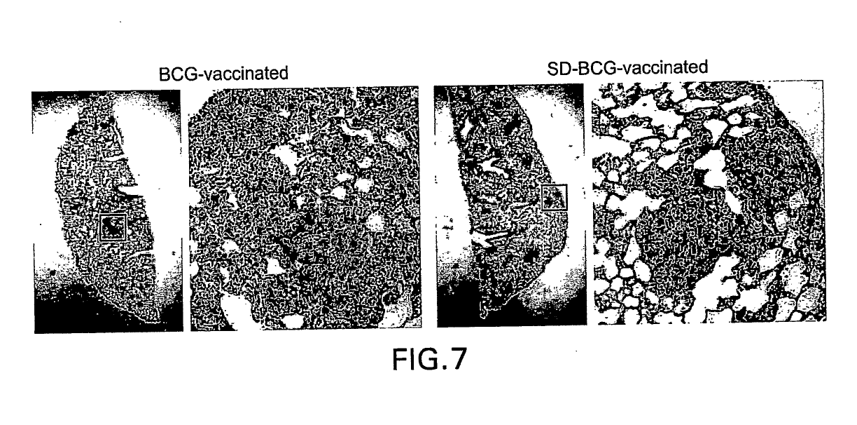

In a separate vaccination-challenge experiment, C57Bl/6 mice were vaccinated subcutaneously, rested for 100 days, and harvested for analysis of T-cell responses in the lung at 4, 10, and 18 days post-aerosol challenge with virulent M. tuberculosis. Compared to mice vaccinated with BCG, mice vaccinated with SD-BCG-AS-SOD exhibited greater numbers of CD4+ and CD8+ T-cells that were CD44+ /CD45RBhigh at 4 days post-challenge, and greater numbers of CD4+ T-cells that were CD44+ /CD45RBneg at 18 days (FIG. 6). These differences in T-cell responses were associated with a difference in the histopathologic appearance of the lungs early post-challenge including the more rapid development of Ghon lesions (FIG. 7).

Based on these results and results reported elsewhere herein, comparable enhancement of vaccine efficacy is expected with the SAD-BCG strains constructed by using dominant-negative mutant SodA expression as described above.

### Example 4

### Construction and Vaccine Evaluation of SIG-BCG (also Referred to as: BCGΔsigH)

The effect of diminishing other antioxidants produced by BCG upon vaccine efficacy was assessed. As discussed above, sigH is a sigma factor implicated in the bacterial response to oxidative stress and regulates the production of thioredoxin, thioredoxin reductase, and a glutaredoxin homolog.

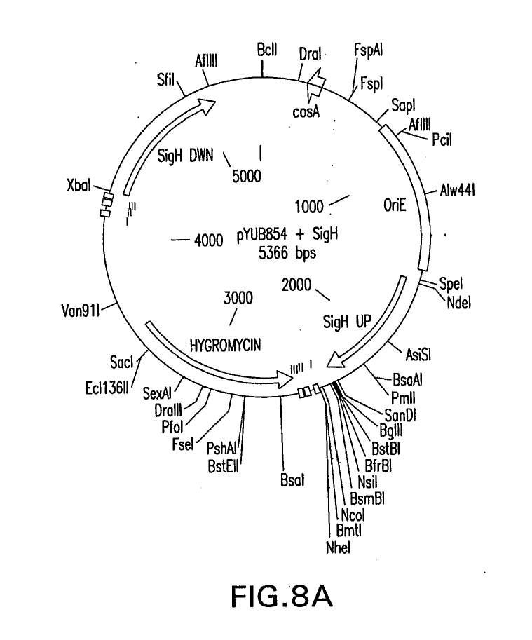

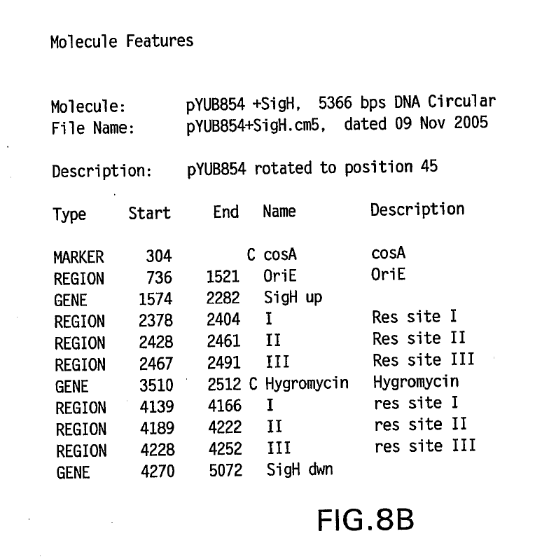

SigH on the chromosome of BCG Tice was inactivated by using the phasmid system of William Jacobs, Jr. from Albert Einstein College of Medicine, using published methods for applying this system to inactivate genes in mycobacteria [Braunstein, M. et al, 2002]. Upstream and downstream regions of sigH were cloned into pYUB854 to construct the allelic inactivation vector—the DNA sequence of pYUB854-sigH is shown in the footnotes of Table 1 and the map and features of this vector are shown in FIG. 8.

An alternative strategy for constructing SIG-BCG (BCGΔsigH) involves the use of suicide plasmid vectors as described and referenced above, the use of which are well-known among those skilled in the art.

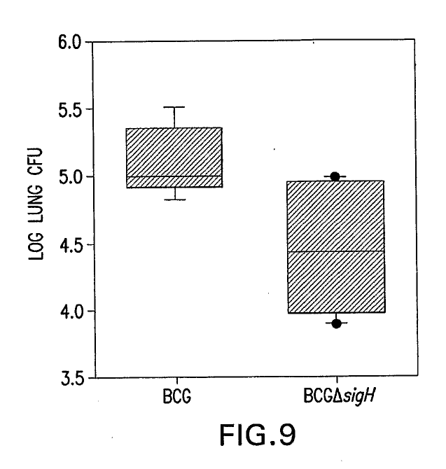

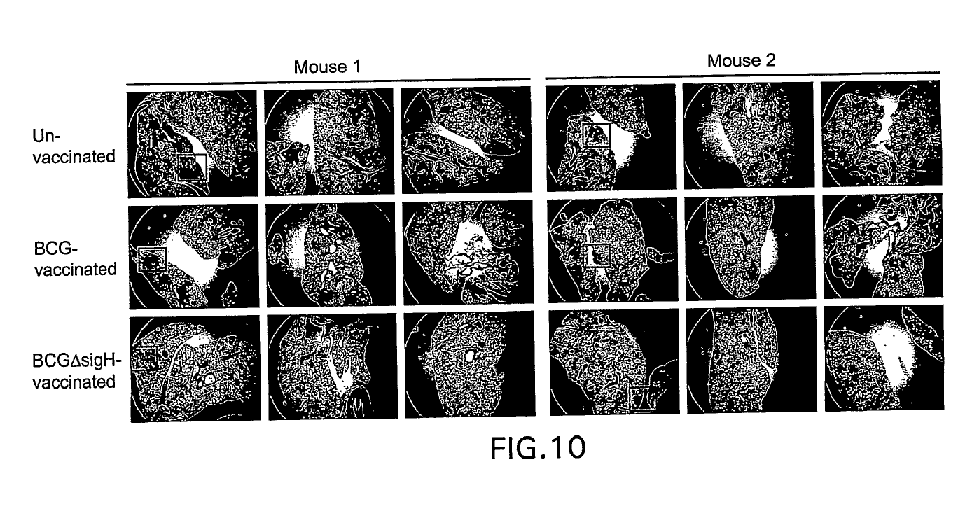

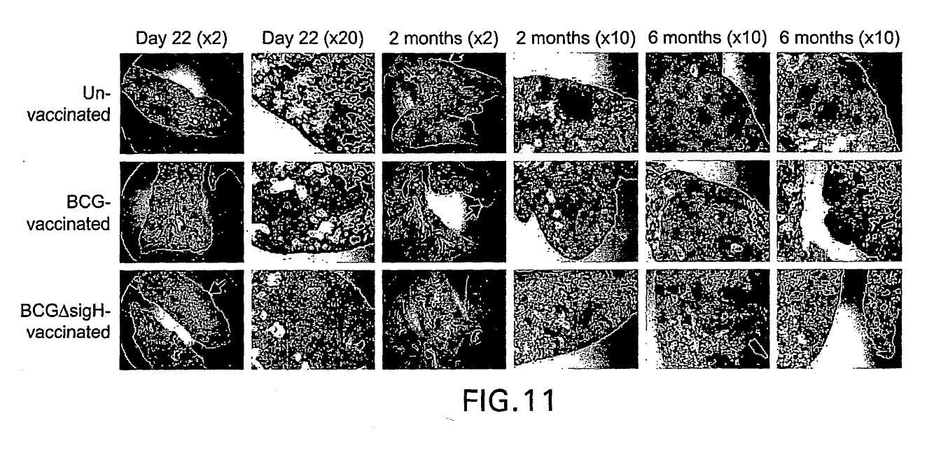

SIG-BCG was tested as a vaccine. C57Bl/6 mice were vaccinated subcutaneously with either BCG or SIG-BCG, rested for 100 days, and then challenged by aerosol with the AcrR-Erdman strain of virulent M. tuberculosis. At six months post-challenge, mice vaccinated with SIG-BCG had lower lung cfu counts of virulent M. tuberculosis (FIG. 9) and less lung damage (FIG. 10) than mice vaccinated with BCG. The histopathologic appearance over time of the lungs of SIG-BCG-vaccinated mice challenged with virulent M. tuberculosis showed similarities to results shown above for mice vaccinated with SD-BCG-AS-SOD (example 4)—most notable were the earlier development of Ghon lesions in mice vaccinated with SIG-BCG and their apparent resolution over time (FIG. 11) that corresponded with the lower lung cfu counts.

### Example 5

### Construction of SAD-SIG-BCG, a “Second-Generation Pro-Apoptotic BCG Vaccine”, and Documentation of Reduced SOD Activity In vitro

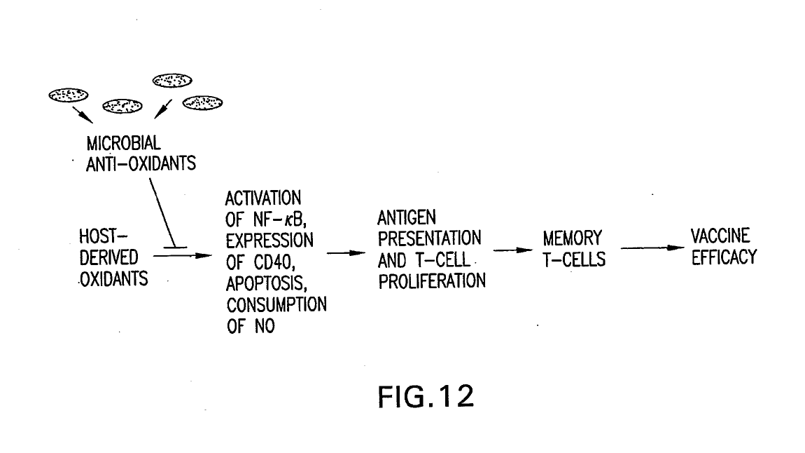

The increased vaccine efficacy of two different pro-apoptotic BCG vaccines (SD-BCG-AS-SOD and SIG-BCG) as exemplified in examples 3 and 4 shows that host-generated oxidants have important functions in the host immune response. Microbial anti-oxidants interfere with these important functions of oxidants (FIG. 12) and thereby disrupt the early signaling needed to develop a strong protective immune response.

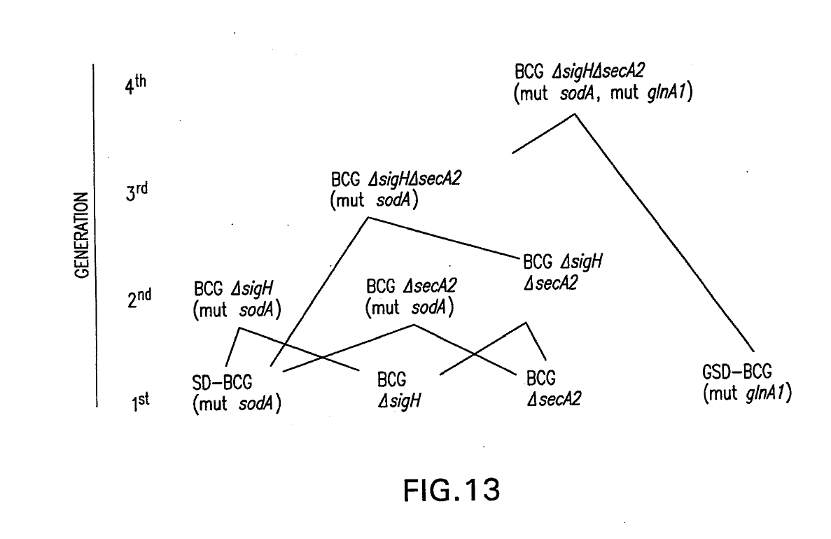

The observations of examples 3 and 4 involving two pro-apoptotic BCG vaccines, each with a single genetic modification, indicate that introducing two or more defects in antioxidant production by BCG yields a more potent vaccine. As discussed above, microorganisms produce a diverse array of anti-apoptotic enzymes, many of which are involved in inactivating host oxidants. FIG. 13 shows a strategy for combining genetic modifications in BCG (and M. tuberculosis) to introduce one, two, three, or four genetic manipulations that reduce antioxidant production, yielding respectively, 1st, 2nd, 3rd, and 4th generation pro-apoptotic vaccines.

To produce “2nd generation” pro-apoptotic BCG vaccines, dominant-negative mutant sodA expression vectors (pMP399-mut SodA ΔH28ΔH76; pMP349-mut SodA ΔH28ΔH76; pMP399-mut SodA ΔE54; and pMP349-mut SodA ΔE54) were electroporated into SIG-BCG to yield SAD-SIG-BCG. The results of SOD activity assays on lysates and supernatants of these strains are shown in FIG. 14 and demonstrate similar reductions in SOD activity to those shown with the 1st generation SAD-BCG vaccines. Overexpression of the dominant-negative ΔH28ΔH76 sodA mutant resulted in greater reduction in SOD activity (about 8-fold) than overexpression of the ΔE54 sodA mutant (about 4-fold).

### Example 6

### Construction of DD-BCG (also Referred to as: BCGΔsigHΔsecA2)

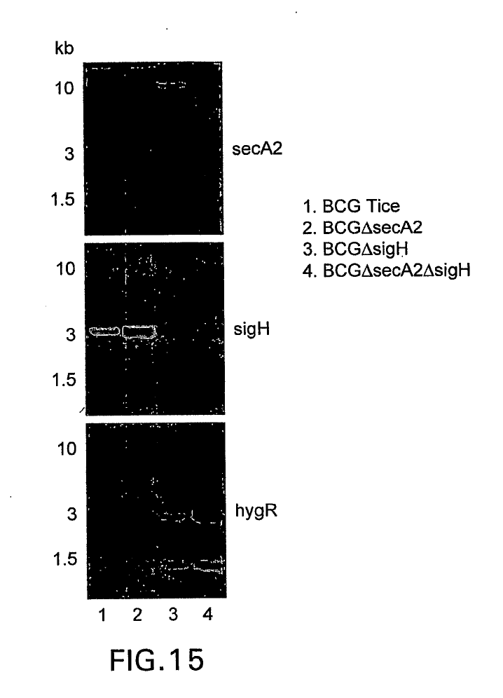

Another “2nd generation” pro-apoptotic BCG vaccine was produced by using the methods outlined in example 4 to inactivate sigH on the chromosome of SEC-BCG (also referred to as: “BCGΔsecA2”) to produce DD-BCG, which is an abbreviation of “double-deletion BCG”. FIG. 15 shows a Southern hybridization membrane that documents the successful construction of DD-BCG.

### Example 7

### Construction of 3D-BCG and Documentation of Reduced SOD Activity In vitro

To produce “3rd generation” pro-apoptotic BCG vaccines, dominant-negative mutant sodA expression vectors (pMP399-mut SodA ΔH28ΔH76; pMP349-mut SodA ΔH28ΔH76; pMP399-mut SodA ΔE54; and pMP349-mut SodA ΔE54) were electroporated into DD-BCG to yield 3D-BCG.

The results of SOD activity assays on lysates and supernatants of these strains are shown in FIG. 16. In contrast to results involving SAD-BCG and SAD-SIG-BCG in which the SOD activity was predominantly in the supernatant (FIGS. 3, 4, 14), the results in FIG. 16A show that the SOD activity in DD-BCG and 3D-BCG is predominantly in the cell lysates. This reversal occurs because the inactivation of secA2 in BCG disrupts the secretion channel for SodA, causing it to be withheld by the bacterium rather than secreted extracellularly.

This localization of SodA in the lysates of these strains facilitated the use of other techniques to quantify the amount of SodA. FIG. 17 shows SDS-PAGE and Western hybridization results comparing the amount of SodA as determined by direct observation of the 23-kDa SodA band on SDS-PAGE and after hybridization with rabbit polyclonal anti-SodA antibody (Western). These results indicate that despite the marked reduction in SOD activity exhibited by 3D-BCG isolates in which the ΔH28ΔH76 and ΔE54 SodA mutants have been overexpressed, there is a comparable amount of SodA protein. This indicates that the overexpression of ΔH28ΔH76 and ΔE54 SodA mutants induces a dominant-negative effect, interfering with the biological activity of SodA despite comparable amounts of total (wild-type plus mutant) SodA protein.

These results also indicate that there can be an advantage of practicing the dominant-negative mutant SodA strategy in combination with allelic inactivation of secA2. There appears to be a greater overall reduction in total SOD activity in strains with the secA2 deletion compared to strains without this deletion. For example, whereas SAD-BCG and SAD-SIG-BCG isolates with overexpressed dominant-negative ΔH28ΔH76 SodA mutant exhibited an 8- to 16-fold reduction in total SOD activity (FIGS. 3, 14), the reduction appeared to be 32-fold or greater when the ΔH28ΔH76 SodA mutant was added to DD-BCG (FIG. 16). Similarly, a greater reduction in SOD activity was achieved when the ΔE54 SodA mutant was put into DD-BCG (FIG. 16; 16-fold reduction) than in BCG or SIG-BCG (FIGS. 4, 14; 2- to 4-fold reduction).

### Example 8

### Addition of Dominant-Negative Glutamine Synthase to 3D-BCG to Yield 4D-BCG Vaccines

Glutamate and glutamine exert pro- and anti-apoptotic effects, respectively, upon mammalian cells. Glutamine synthase (also called “glutamine synthetase”) catalyzes the reaction between glutamate and ammonia to yield glutamine. M. tuberculosis and BCG have several alleles on their chromosome that encode glutamine synthase or homologs. One of these, glnA1, is produced in large amounts and secreted extracellularly.

To construct 4D-BCG, a dominant-negative glnA1 mutant in pCR2.1-TOPO was constructed by performing PCR-based site-directed mutagenesis on the wild-type glnA1 allele that had been PCR-amplified from chromosomal DNA from M. tuberculosis H37Rv. The open reading frame of the ΔD54ΔE335 mutant glnA1 allele is shown below. Initiation and stop codons are bold, and --- shows the position of the two deleted codons corresponding to amino acid 54 and amino acid 335 of the enzyme.

SEQ ID NO: 11

| | |
|---|---|
| 1 | gtg acg gaa aag acg ccc gac gac gtc ttc |
| 31 | aaa ctt gcc aag gac gag aag gtc gaa tat |
| 61 | gtc gac gtc cgg ttc tgt gac ctg cct ggc |
| 91 | atc atg cag cac ttc acg att ccg gct tcg |
| 121 | gcc ttt gac aag agc gtg ttt gac gac ggc |
| 151 | ttg gcc ttt --- ggc tcg tcg att cgc ggg |
| 181 | ttc cag tcg atc cac gaa tcc gac atg ttg |
| 211 | ctt ctt ccc gat ccc gag acg gcg cgc atc |
| 241 | gac ccg ttc cgc gcg gcc aag acg ctg aat |
| 271 | atc aac ttc ttt gtg cac gac ccg ttc acc |
| 301 | ctg gag ccg tac tcc cgc gac ccg cgc aac |
| 331 | atc gcc cgc aag gcc gag aac tac ctg atc |
| 361 | agc act ggc atc gcc gac acc gca tac ttc |
| 391 | ggc gcc gag gcc gag ttc tac att ttc gat |
| 421 | tcg gtg agc ttc gac tcg cgc gcc aac ggc |
| 451 | tcc ttc tac gag gtg gac gcc atc tcg ggg |
| 481 | tgg tgg aac acc ggc gcg gcg acc gag gcc |
| 511 | gac ggc agt ccc aac cgg ggc tac aag gtc |
| 541 | cgc cac aag ggc ggg tat ttc cca gtg gcc |
| 571 | ccc aac gac caa tac gtc gac ctg cgc gac |
| 601 | aag atg ctg acc aac ctg atc aac tcc ggc |
| 631 | ttc atc ctg gag aag ggc cac cac gag gtg |
| 661 | ggc agc ggc gga cag gcc gag atc aac tac |
| 691 | cag ttc aat tcg ctg ctg cac gcc gcc gac |
| 721 | gac atg cag ttg tac aag tac atc atc aag |
| 751 | aac acc gcc tgg cag aac ggc aaa acg gtc |
| 781 | acg ttc atg ccc aag ccg ctg ttc ggc gac |
| 811 | aac ggg tcc ggc atg cac tgt cat cag tcg |
| 841 | ctg tgg aag gac ggg gcc ccg ctg atg tac |
| 871 | gac gag acg ggt tat gcc ggt ctg tcg gac |
| 901 | acg gcc cgt cat tac atc ggc ggc ctg tta |
| 931 | cac cac gcg ccg tcg ctg ctg gcc ttc acc |
| 961 | aac ccg acg gtg aac tcc tac aag cgg ctg |
| 991 | gtt ccc ggt tac --- gcc ccg atc aac ctg |
| 1021 | gtc tat agc cag cgc aac cgg tcg gca tgc |
| 1051 | gtg cgc atc ccg atc acc ggc agc aac ccg |
| 1081 | aag gcc aag cgg ctg gag ttc cga agc ccc |
| 1111 | gac tcg tcg ggc aac ccg tat ctg gcg ttc |
| 1141 | tcg gcc atg ctg atg gca ggc ctg gac ggt |
| 1171 | atc aag aac aag atc gag ccg cag gcg ccc |
| 1201 | gtc gac aag gat ctc tac gag ctg ccg ccg |
| 1231 | gaa gag gcc gcg agt atc ccg cag act ccg |
| 1261 | acc cag ctg tca gat gtg atc gac cgt ctc |
| 1291 | gag gcc gac cac gaa tac ctc acc gaa gga |
| 1321 | ggg gtg ttc aca aac gac ctg atc gag acg |
| 1351 | tgg atc agt ttc aag cgc gaa aac gag atc |
| 1381 | gag ccg gtc aac atc cgg ccg cat ccc tac |
| 1411 | gaa ttc gcg ctg tac tac gac gtt taa |

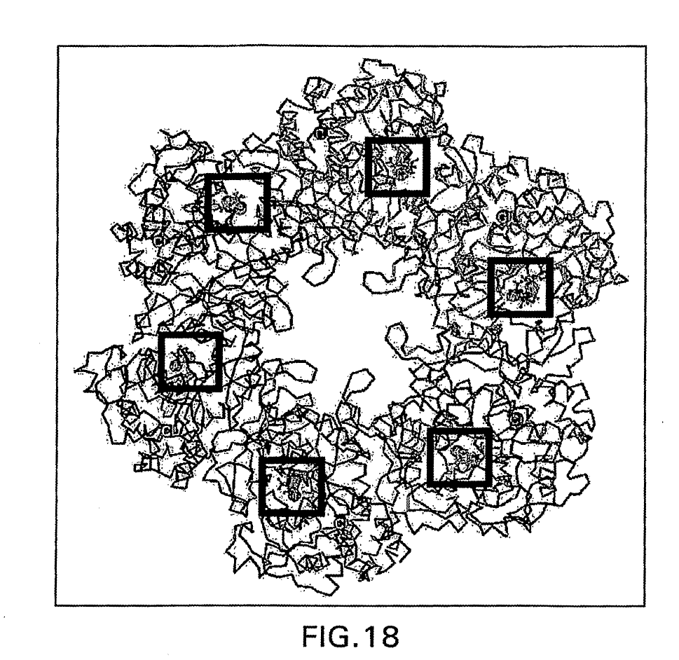

The positions of these amino acid deletions in the context of the active-site manganese ions of the hexameric glnA1 ring are shown in FIG. 18. As the D54 and E335 from adjacent monomers are involved in forming the active sites, which lie between monomers, introducing both deletions in a single monomer disrupts the active sites on each side of the monomer as it assembles into rings with wild-type monomers. Thus, it induces a dominant-negative effect.

A BLASTN query of this DNA sequence against the nucleotide sequence of the complete M. tuberculosis H37Rv sequence was performed using the BLAST server of the TubercuList World Wide Web site (http://genolist.pasteur.fr/TubercuList/), documenting the deletion of the two codons.

>M. tuberculosis H37Rv|null M. tuberculis H37RV (4411532 bp)

Length=4411532

Score=2793 bits (1409), Expect=0.0

Identities=1431/1437 (99%), Gaps=6/1437 (0%)

Strand=Plus/Plus

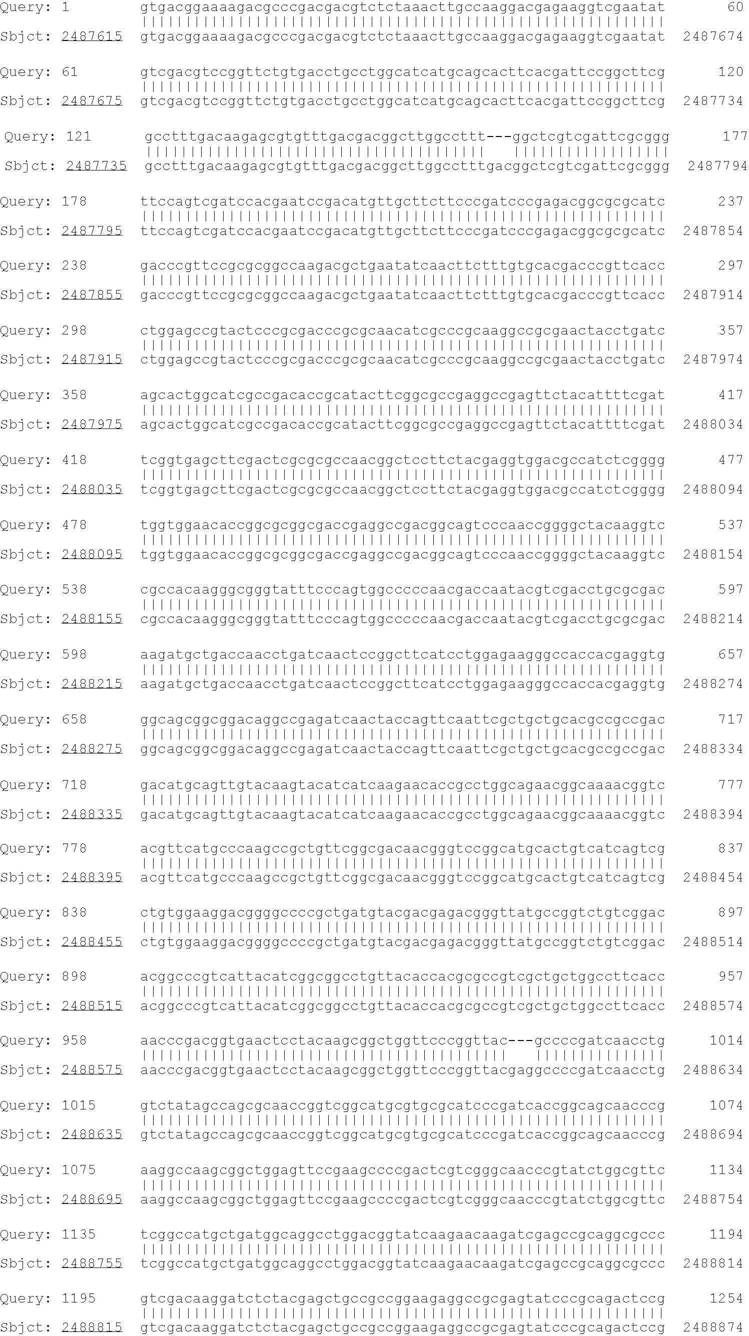

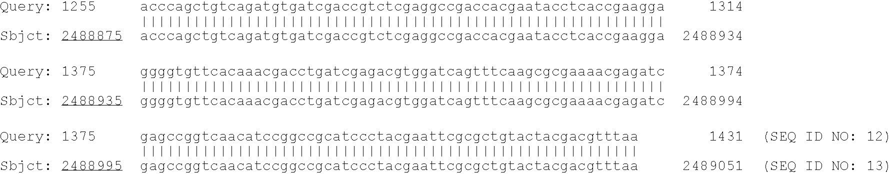

A TBLASTN query was also performed against translated nucleotide sequence data at the TubercuList BLAST site (http://genolist.pasteur.fr/TubercuList/), showing the positions of the deleted aspartic acid and glutamic acid.

>M. tuberculosis H37Rv|null M. tuberculis H37RV (4411532 bp)

Length=4411532

Score=973 bits (2515), Expect=0.0

Identities=476/478 (99%), Positives=476/478 (99%), Gaps=2/478 (0%)

Frame=+3

| | | | | |
|---|---|---|---|---|
| Query: | 1 | VTEKTPDDVFKLAKDEKVEYVDVRFCDLPGIMQHFTIPASAFDKSVFDDGLAF-GSSIRG | 59 | (SEQ ID NO: 14) |
| | | VTEKTPDDVFKLAKDEKVEYVDVRFCDLPGIMQHFTIPASAFDKSVFDDGLAF GSSIRG | | (SEQ ID NO: 14) |
| Sbjct: | 2487615 | VTEKTPDDVFKLAKDEKVEYVDVRFCDLPGIMQHFTIPASAFDKSVFDDGLAFDGSSIRG | 2487794 | (SEQ ID NO: 15) |
| Query: | 60 | FQSIHESDMLLLPDPETARIDPFRAAKTLNINFFVHDPFTLEPYSRDPRNIARKAENYLI | 119 | |
| | | FQSIHESDMLLLPDPETARIDPFRAAKTLNINFFVHDPFTLEPYSRDPRNIARKAENYLI | | |
| Sbjct: | 2487795 | FQSIHESDMLLLPDPETARIDPFRAAKTLNINFFVHDPFTLEPYSRDPRNIARKAENYLI | 2487974 | |
| Query: | 120 | STGIADTAYFGAEAEFYIFDSVSFDSRANGSFYEVDAISGWWNTGAATEADGSPNRGYKV | 179 | |
| | | STGIADTAYFGAEAEFYIFDSVSFDSRANGSFYEVDAISGWWNTGAATEADGSPNRGYKV | | |
| Sbjct: | 2487975 | STGIADTAYFGAEAEFYIFDSVSFDSRANGSFYEVDAISGWWNTGAATEADGSPNRGYKV | 2488154 | |
| Query: | 180 | RHKGGYFPVAPNDQYVDLRDKMLTNLINSGFILEKGHHEVGSGGQAEINYQFNSLLHAAD | 239 | |
| | | RHKGGYFPVAPNDQYVDLRDKMLTNLINSGFILEKGHHEVGSGGQAEINYQFNSLLHAAD | | |
| Sbjct: | 2488155 | RHKGGYFPVAPNDQYVDLRDKMLTNLINSGFILEKGHHEVGSGGQAEINYQFNSLLHAAD | 2488334 | |
| Query: | 240 | DMQLYKYIIKNTAWQNGKTVTFMPKPLFGDNGSGMHCHQSLWKDGAPLMYDETGYAGLSD | 299 | |
| | | DMQLYKYIIKNTAWQNGKTVTFMPKPLFGDNGSGMHCHQSLWKDGAPLMYDETGYAGLSD | | |
| Sbjct: | 2488335 | DMQLYKYIIKNTAWQNGKTVTFMPKPLFGDNGSGMHCHQSLWKDGAPLMYDETGYAGLSD | 2488514 | |
| Query: | 300 | TARHYIGGLLHHAPSLLAFTNPTVNSYKRLVPGY-APINLVYSQRNRSACVRIPITGSNP | 358 | |
| | | TARHYIGGLLHHAPSLLAFTNPTVNSYKRLVPGY APINLVYSQRNRSACVRIPITGSNP | | |
| Sbjct: | 2488515 | TARHYIGGLLHHAPSLLAFTNPTVNSYKRLVPGYEAPINLVYSQRNRSACVRIPITGSNP | 2488694 | |
| Query: | 359 | KAKRLEFRSPDSSGNPYLAFSAMLMAGLDGIKNKIEPQAPVDKDLYELPPEEAASIPQTP | 418 | |
| | | KAKRLEFRSPDSSGNPYLAFSAMLMAGLDGIKNKIEPQAPVDKDLYELPPEEAASIPQTP | | |
| Sbjct: | 2488695 | KAKRLEFRSPDSSGNPYLAFSAMLMAGLDGIKNKIEPQAPVDKDLYELPPEEAASIPQTP | 2488874 | |
| Query: | 419 | TQLSDVIDRLEADHEYLTEGGVFTNDLIETWISFKRENEIEPVNIRPHPYEFALYYDV | 476 | |
| | | TQLSDVIDRLEADHEYLTEGGVFTNDLIETWISFKRENEIEPVNIRPHPYEFALYYDV | | |
| Sbjct: | 2488875 | TQLSDVIDRLEADHEYLTEGGVFTNDLIETWISFKRENEIEPVNIRPHPYEFALYYDV | 2489048 | |

BLASTN and TBLASTN queries were also performed against nucleotide sequence data in the M. bovis BLAST server of the Sanger Centre (http://www.sanger.ac.uk/cgibin/blast/submitblast/m_bovis). The Sanger Centre is sequencing Mycobacterium bovis BCG Pasteur the preliminary M. bovis BCG assembly was used. The results show that the glnA1 nucleotide sequence in BCG Pasteur is identical to the glnA1 nucleotide sequence in M. tuberculosis H37Rv.

BLASTN results:

>BCG260c11.q1k 3891 bp, 23 reads, 35.42 AT

[Full Sequence]

Length=3891

Minus Strand HSPs:

Score=7095 (1070.6 bits), Expect=0., P=0.

Identities=1431/1437 (99%), Positives=1431/1437 (99%), Strand=Minus Plus

[HSP Sequence]

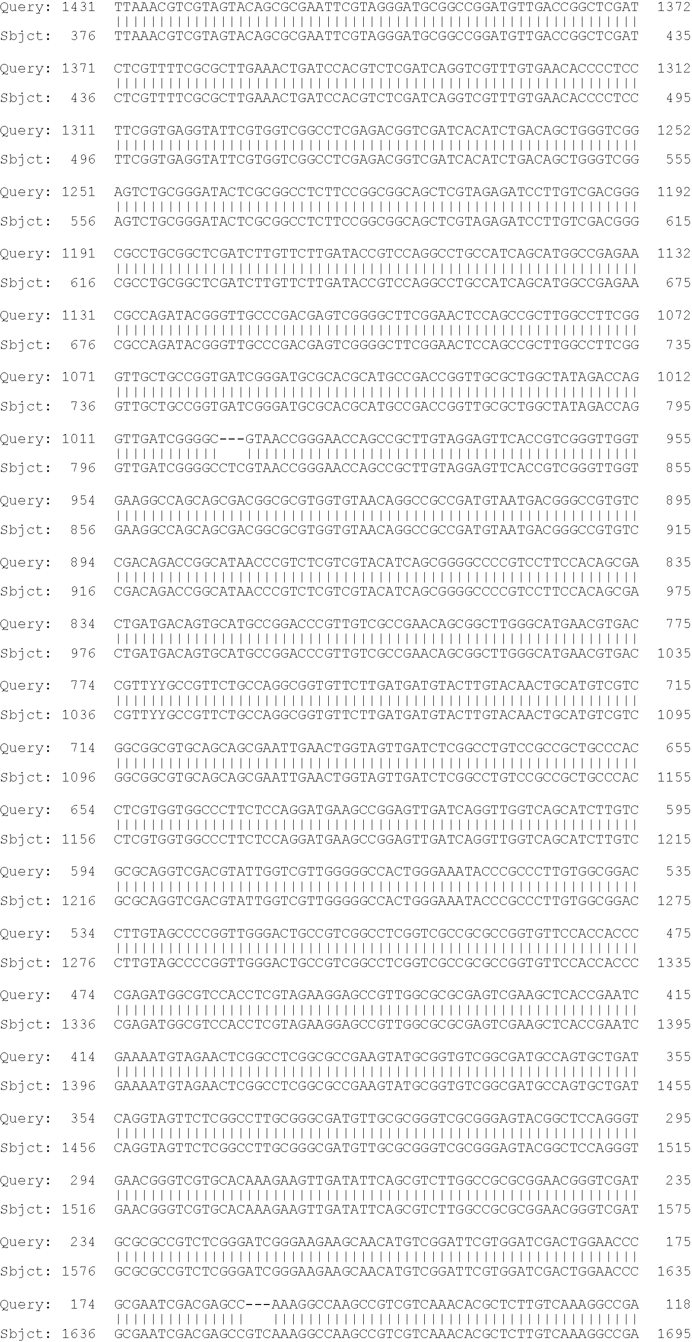

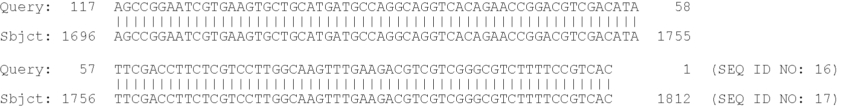

TBLASTN results:

>BCG260c11.q1k 3891 bp, 23 reads, 35.42 AT

[Full Sequence]

Length=3891

Minus Strand HSPs:

Score=2521 (892.5 bits), Expect=9.0e-264, P=9.0e-264

Identities=476/478 (99%), Positives=476/478 (99%), Frame=−1

[HSP Sequence]

| | | | | |
|---|---|---|---|---|
| Query: | 1 | VTEKTPDDVFKLAKDEKVEYVDVRFCDLPGIMQHFTIPASAFDKSVFDDGLAF-GSSIRG | 59 | (SEQ ID NO: 18) |
| | | VTEKTPDDVFKLAKDEKVEYVDVRFCDLPGIMQHFTIPASAFDKSVFDDGLAF GSSIRG | | (SEQ ID NO: 18) |
| Sbjct: | 1812 | VTEKTPDDVFKLAKDEKVEYVDVRFCDLPGIMQHFTIPASAFDKSVFDDGLAFDGSSIRG | 1633 | (SEQ ID NO: 19) |
| Query: | 60 | FQSIHESDMLLLPDPETARIDPFRAAKTLNINFFVHDPFTLEPYSRDPRNIARKAENYLI | 119 | |
| | | FQSIHESDMLLLPDPETARIDPFRAAKTLNINFFVHDPFTLEPYSRDPRNIARKAENYLI | | |
| Sbjct: | 1632 | FQSIHESDMLLLPDPETARIDPFRAAKTLNINFFVHDPFTLEPYSRDPRNIARKAENYLI | 1453 | |
| Query: | 120 | STGIADTAYFGAEAEFYIFDSVSFDSRANGSFYEVDAISGWWNTGAATEADGSPNRGYKV | 179 | |
| | | STGIADTAYFGAEAEFYIFDSVSFDSRANGSFYEVDAISGWWNTGAATEADGSPNRGYKV | | |
| Sbjct: | 1452 | STGIADTAYFGAEAEFYIFDSVSFDSRANGSFYEVDAISGWWNTGAATEADGSPNRGYKV | 1273 | |
| Query: | 180 | RHKGGYFPVAPNDQYVDLRDKMLTNLINSGFILEKGHHEVGSGGQAEINYQFNSLLHAAD | 239 | |
| | | RHKGGYFPVAPNDQYVDLRDKMLTNLINSGFILEKGHHEVGSGGQAEINYQFNSLLHAAD | | |
| Sbjct: | 1272 | RHKGGYFPVAPNDQYVDLRDKMLTNLINSGFILEKGHHEVGSGGQAEINYQFNSLLHAAD | 1093 | |
| Query: | 240 | DMQLYKYIIKNTAWQNGKTVTFMPKPLFGDNGSGMHCHQSLWKDGAPLMYDETGYAGLSD | 299 | |
| | | DMQLYKYIIKNTAWQNGKTVTFMPKPLFGDNGSGMHCHQSLWKDGAPLMYDETGYAGLSD | | |
| Sbjct: | 1092 | DMQLYKYIIKNTAWQNGKTVTFMPKPLFGDNGSGMHCHQSLWKDGAPLMYDETGYAGLSD | 913 | |
| Query: | 300 | TARHYIGGLLHHAPSLLAFTNPTVNSYKRLVPGY-APINLVYSQRNRSACVRIPITGSNP | 358 | |
| | | TARHYIGGLLHHAPSLLAFTNPTVNSYKRLVPGY APINLVYSQRNRSACVRIPITGSNP | | |
| Sbjct: | 912 | TARHYIGGLLHHAPSLLAFTNPTVNSYKRLVPGYEAPINLVYSQRNRSACVRIPITGSNP | 733 | |
| Query: | 359 | KAKRLEFRSPDSSGNPYLAFSAMLMAGLDGIKNKIEPQAPVDKDLYELPPEEAASIPQTP | 418 | |
| | | KAKRLEFRSPDSSGNPYLAFSAMLMAGLDGIKNKIEPQAPVDKDLYELPPEEAASIPQTP | | |
| Sbjct: | 732 | KAKRLEFRSPDSSGNPYLAFSAMLMAGLDGIKNKIEPQAPVDKDLYELPPEEAASIPQTP | 553 | |
| Query: | 419 | TQLSDVIDRLEADHEYLTEGGVFTNDLIETWISFKRENEIEPVNIRPHPYEFALYYDV | 476 | |
| | | TQLSDVIDRLEADHEYLTEGGVFTNDLIETWISFKRENEIEPVNIRPHPYEFALYYDV | | |
| Sbjct: | 552 | TQLSDVIDRLEADHEYLTEGGVFTNDLIETWISFKRENEIEPVNIRPHPYEFALYYDV | 379 | |

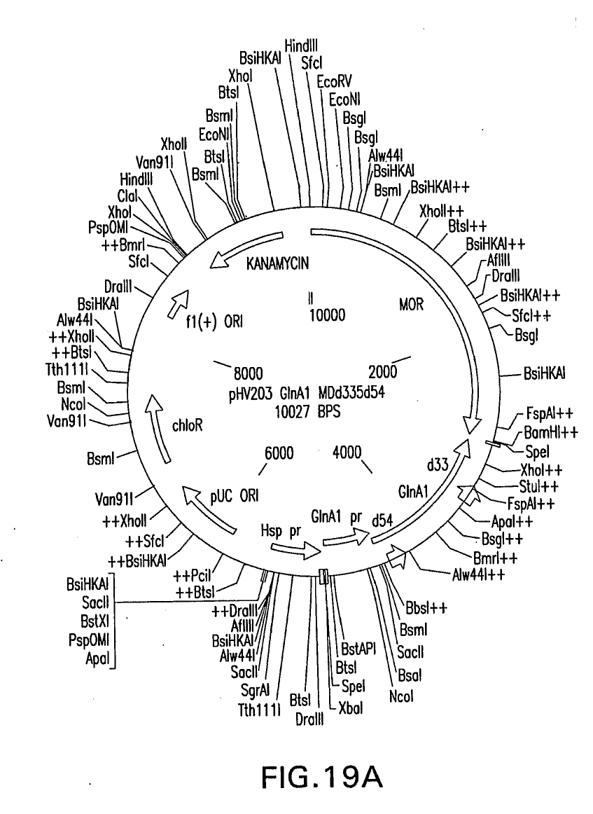

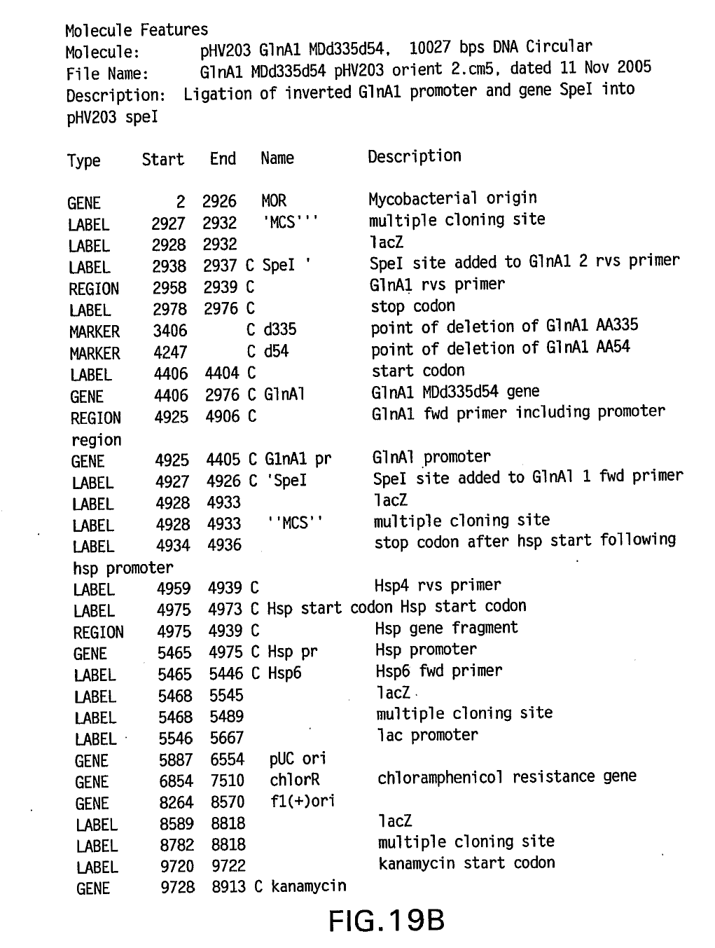

Next, the mutant glnA1 allele including its own promoter region was ligated into a speI site in pHV203 to yield pHV203-mut glnA1 ΔD54ΔE335 and also into the chromosomal integration vector pMP399 and the plasmid vector pMP349 promoter to yield pMP399-mut glnA1 ΔD54ΔE335 and pMP349-mut glnA1 ΔD54ΔE335 (Table 1). The pHV203-mut glnA1 ΔD54ΔE335 plasmid map is shown in FIG. 19 and the complete nucleotide sequences of each of these plasmids are included in the footnotes of Table 1. The pHV203-mut glnA1 ΔD54ΔE335 plasmid was electroporated into the 3D-BCG vaccines to yield 4D-BCG vaccines. These vectors can be introduced into BCG as well as 1st, 2nd, and 3rd generation pro-apoptotic BCG vaccines to yield, respectively, 1st, 2nd, 3rd, and 4th generation vaccines.

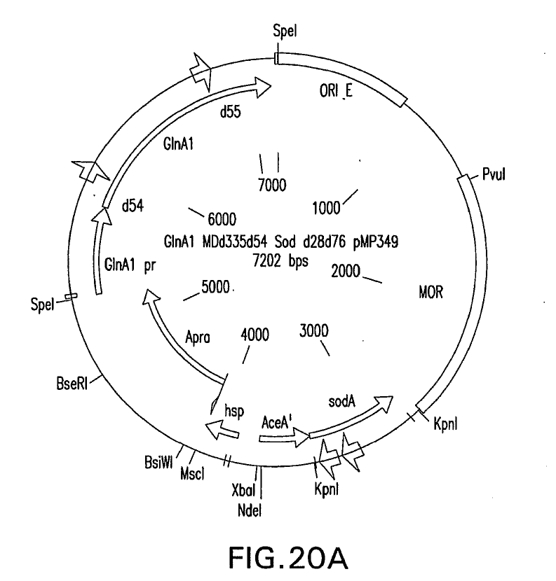

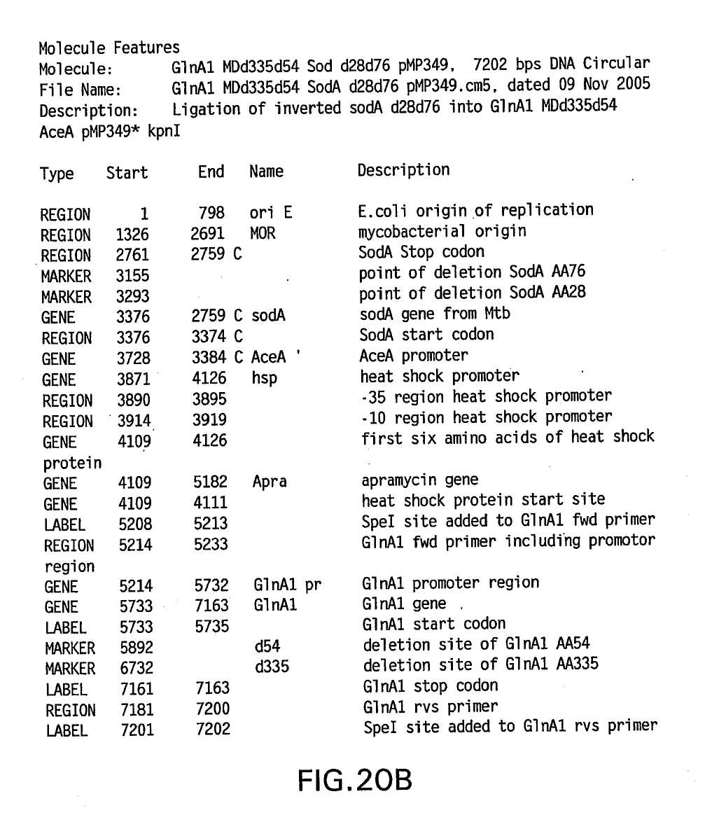

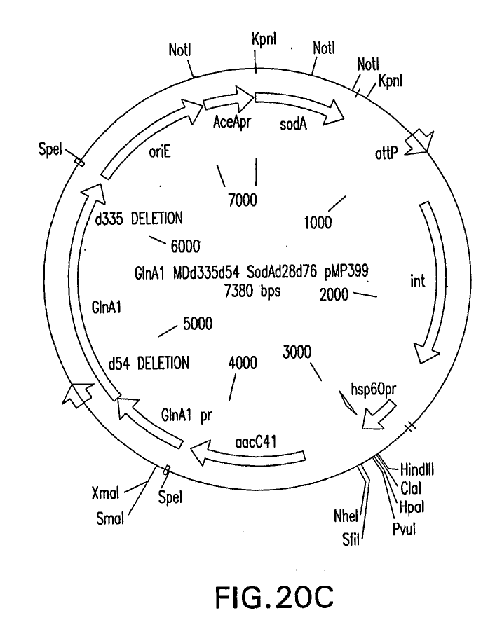

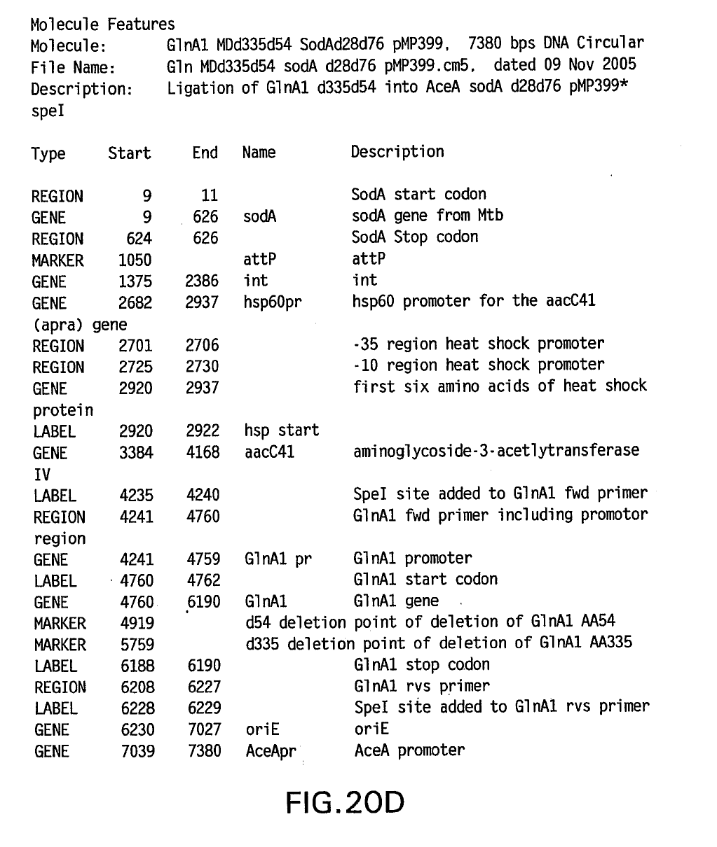

Additional plasmids and chromosomal-integration vectors were built that combined a mutant sodA allele and a mutant glnA1 allele on the same vector. These include pMP399-mut SodA ΔH28ΔH76 mut glnA1 ΔD54ΔE335 (FIG. 20), pMP399-mut SodA ΔE54 mut glnA1 ΔD54ΔE335, pMP349-mut SodA ΔH28ΔH76 mut glnA1 ΔD54ΔE335 (FIG. 20), and pMP349-mut SodA ΔE54 mut glnA1 ΔD54ΔE335 (Table 1). These vectors were introduced into BCG as well as 1st and 2nd generation pro-apoptotic BCG vaccines to yield, respectively, 2nd, 3rd, and 4th generation vaccines.

### Example 9

### Expression of an Exogenous Antigen by Pro-Apoptotic BCG

The pro-apoptotic BCG vaccines described above can be used to express exogenous antigens, including antigens from other infectious agents and cancer antigens.

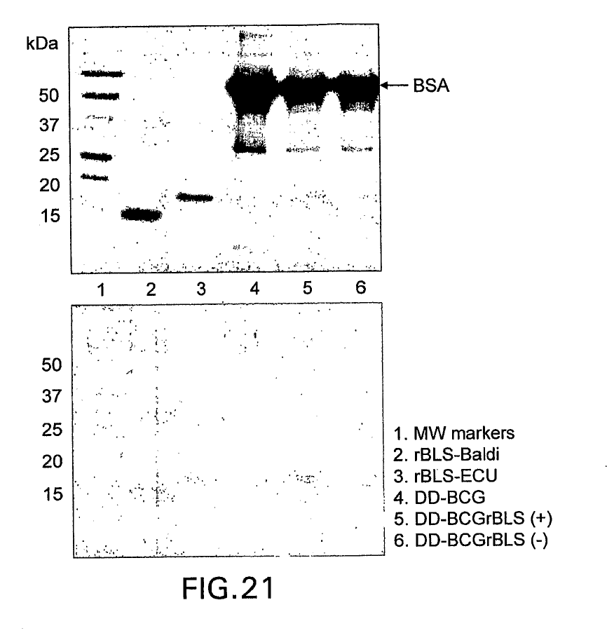

DD-BCGrBLS was constructed in which recombinant Brucella lumazine synthase, an immunodominant T-cell antigen of Brucella abortus [Velikovsky, C. A. et al, 2002], is expressed by DD-BCG. The bls gene was ligated behind an aceA(icl) promoter in pMP349 to produce pMP349-rBLS (Table 1). This plasmid was electroporated into DD-BCG to yield DD-BCGrBLS. The expression of rBLS by DD-BCGrBLS is shown in FIG. 21.

These results demonstrate that foreign antigens can be expressed in pro-apoptotic BCG. This capability allows the construction of a new generation of vaccines that induce strong T-cell responses by using pro-apoptotic intracellular bacteria as a vehicle for accessing apoptosis-associated cross priming pathways of antigen presentation. In this way, exogenous antigens can be delivered to dendritic cells to induce strong CD4 and CD8 T-cell responses. For example, the DD-BCGrBLS strain shown here or other pro-apoptotic intacellular bacterial vaccines expressing recombinant Brucella antigens can be used to immunize cattle or other mammalian hosts. This technology can be used to simultaneously protect cattle against bovine tuberculosis and brucellosis.

Due to differences in codon usage among different species, it may be helpful to optimize codons in foreign genes for expression in mycobacteria. This can be done routinely by either using site-directed mutagenesis to alter the gene or by constructing synthetic genes that follow the codon usage preferences of mycobacteria. Such alterations are well-known to those skilled in the art.

### Example 10

### An Alternative to sigH Deletion Comprising Allelic Inactivation of Thioredoxin, Thioredoxin Reductase, and Glutaredoxin

The inactivation of sigH affects the production of multiple microbial factors, some of which may be important targets for the host immune response. At present this is a hypothetical concern and the current data support the proposition that the low levels of sigH-regulated proteins expressed by a sigH deletion mutant are sufficient to induce strong T-cell responses against these proteins. However, as an alternative to sigH inactivation for pro-apoptotic BCG vaccines used to induce protection against tuberculosis, there may be an advantage to directly reducing the activity of key anti-apoptotic enzymes under the control of sigH to minimize effects upon the stress-associated proteome. Under circumstances where the pro-apoptotic BCG vaccine is used primarily to express exogenous antigens from other infectious agents or cancer antigens, the sigH deletion is preferred and provides a mechanism for reducing the production of multiple anti-apoptotic antioxidants.

Thioredoxin (trxC, also trx, MPT46) and thioredoxin reductase (trxB2, also trxr) are sigH-regulated genes that are a prominent part of the bacterial response to oxidative stress. They are located adjacent to each other on the M. tuberculosis/BCG chromosome (trxB2 at bases 4,404,728-4,402,735 and trxC at 4,402,732-4,403,082 in the H37Rv chromosome, per complete genome sequence at TubercuList web server). A phasmid-based vector (pYUB854-trx-trxr) to knock out both trxB2 and trxC simultaneously has been constructed, and the sequence data are provided in Table 1. The map and features of this vector are shown in FIG. 22.

An alternative strategy for constructing TRX-TRXR-BCG (BCGΔtrxCΔtrxB2) involves the use of suicide plasmid vectors as described and referenced above, the use of which are well-known among those skilled in the art. One potential advantage of the plasmid-based system is greater ease in achieving unmarked deletions in which the allele is replaced by an inactive mutant rather than interrupted with an antibiotic resistance determinant. The active sites of thioredoxin, thioredoxin reductase, and many other redox repair enzymes contain active cysteines that form a disulfide bridge when oxidized. The “thioredoxin active-site motif” is a sequence of C—X—X—C where C=cysteine and X=any amino acids. This signature makes it routine to identify the active site of redox-active enzymes. Then the gene can be mutagenized or synthesized to eliminate the active site.

The following amino acid sequences of thioredoxin and thioredoxin reductase show the CXXC motifs in bold, at residues 37-40 and 145-148, respectively:

```
>M. tuberculosis H37Rv|Rv3914|TrxC:
116 aa-THIOREDOXIN TRXC (TRX) (MPT46)
(SEQ ID NO: 20)
    1-MTDSEKSATI KVTDASFATD VLSSNKPVLV DFWATWCGPC KMVAPVLEEI ATERATDLTV
  61-AKLDVDTNPE TARNFQVVSI PTLILFKDGQ PVKRIVGAKG KAALLRELSD VVPNLN
>M. tuberculosis H37Rv|Rv3913|TrxB2:
335 aa-PROBABLE THIOREDOXIN REDUCTASE TRXB2 (TRXR) (TR)
(SEQ ID NO: 21)
    1-MTAPPVHDRA HHPVRDVIVI GSGPAGYTAA LYAARAQLAP LVFEGTSFGG ALMTTTDVEN
  61-YPGFRNGITG PELMDEMREQ ALRFGADLRM EDVESVSLHG PLKSVVTADG QTHRARAVIL
121-AMGAAARYLQ VPGEQELLGR GVSSCATCDG FFFRDQDIAV IGGGDSAMEE ATFLTRFARS
181-VTLVHRRDEF RASKIMLDRA RNNDKIRFLT NHTVVAVDGD TTVTGLRVRD TNTGAETTLP
241-VTGVFVAIGH EPRSGLVREA IDVDPDGYVL VQGRTTSTSL PGVFAAGDLV DRTYRQAVTA
301-AGSGCAAAID AERWLAEHAA TGEADSTDAL IGAQR
```

Using PCR-based gene mutagenesis techniques involving overlapping primers, genes encoding inactive mutants were constructed. The trxC allele encodes an inactive thioredoxin mutant that lacks the “WCGPCK” active-site and the trxB2 allele encodes an inactive thioredoxin reductase sequence that lacks the “SCATCD” active-site. These mutant alleles were incorporated into the p2NIL-pGOAL19 allelic inactivation vector system described by Parish and Stoker [Parish, T. et al, 2000] for introducing “unmarked” (i.e., the final construct lacks antibiotic resistance genes) to produce p2NIL/GOAL19-mut trxC-mut trxB2 (FIG. 23 and Table 1).

This strategy can also be applied to other sigH-regulated genes. For example, RV2466c is sigH-regulated, is a glutaredoxin homolog, and possesses a C—X—X—C motif:

```
>M. tuberculosis H37Rv|Rv2466c|Rv2466c:
207 aa-CONSERVED HYPOTHETICAL PROTEIN
(SEQ ID NO: 22)
    1-MLEKAPQKSV ADFWFDPLCP WCWITSRWIL EVAKVRDIEV NFHVMSLAIL NENRDDLPEQ
  61-YREGMARAWG PVRVAIAAEQ AHGAKVLDPL YTAMGNRIHN QGNHELDEVI TQSLADAGLP
121-AELAKAATSD AYDNALRKSH HAGMDAVGED VGTPTIHVNG VAFFGPVLSK IPRGEEAGKL
181-WDASVTFASY PHFFELKRTR TEPPQFD
```

### Example 11

### Deletion of Sigma Factor E (sigE) to Further Reduce the Production of Anti-Apoptotic Microbial Enzymes by BCG

As noted above, other sigma factors regulate the production of microbial factors important for the response to stress stimuli. Sigma factor E (sigE) has been shown to have an effect upon the production of SodA and glnA1 [Manganelli, R. et al, 2001]. Thus, inactivation of sigE introduces a defect in the production of microbial anti-apoptotic enzymes analogous to other defects described above, and thus can be used alone or combined with other mutations to make a pro-apoptotic BCG strain more potent.

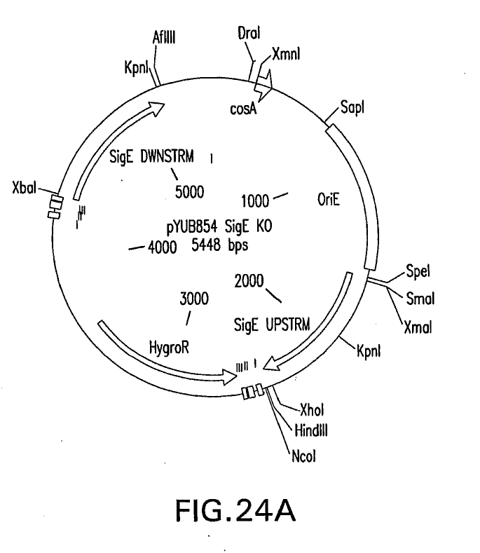

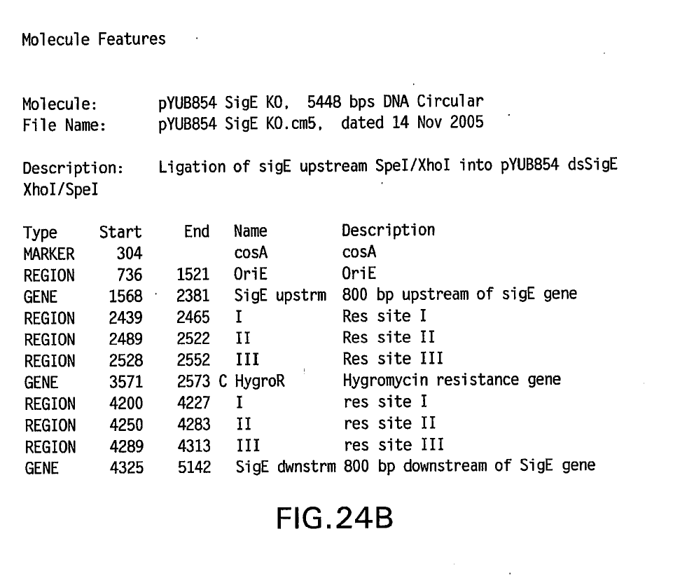

A phasmid-based vector (pYUB854-sigE) to inactivate sigE has been constructed, and the sequence data are provided in Table 1. The map and features of this vector are shown in FIG. 24.

### Example 12

### Documentation of Reduced Glutamine Synthase Activity by 4D-BCG In vitro

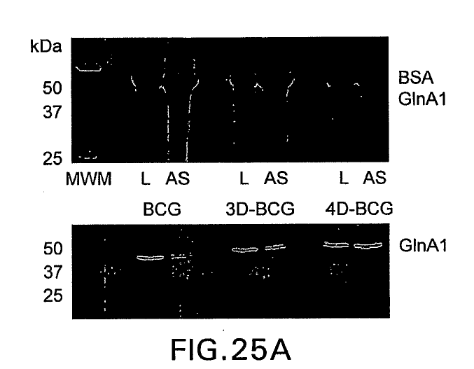

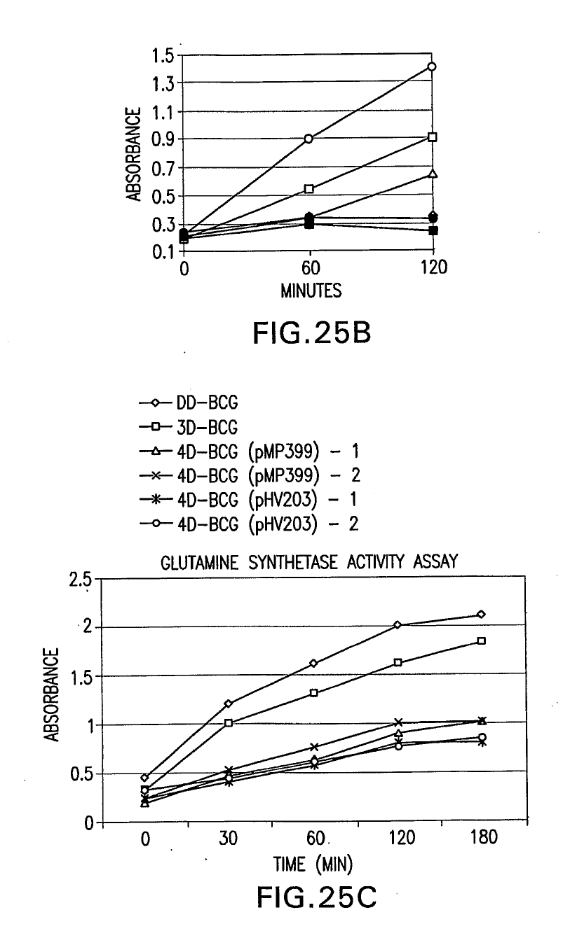

To determine the effect of expressing mutant ΔD55ΔE335 GlnA1 upon the glutamine synthetase activity of the whole bacterium, lysates of DD-BCG, 3D-BCG and two versions of 4D-BCG involving either plasmid or chromosomal expression of the mutant ΔD55ΔE335 GlnA1 were prepared and compared for glutamine synthetase activity. Activity assays were performed using the transfer reaction described by Woolfolk et al. by monitoring absorbance at 540 nm to detect the formation of gamma-glutamic acid hydroxamate. Results are shown in FIG. 25 and demonstrate that the dominant-negative strategy results in a 4- to 8-fold reduction in glutamine synthase activity.

### Example 13

### Splenocytes from Mice Vaccinated with DD-BCG, 3D-BCG, and 4D-BCG Exhibit Enhanced IL-2 Production Compared to Mice Vaccinated with the Parent BCG Strain

To evaluate immune responses to selected pro-apoptotic BCG (paBCG) vaccines and the parent BCG Tice vaccine strain, an IV vaccination model in C57Bl/6 mice was used, comprising administering approximately 5×10⁵ cfu of the vaccine strain as a single dose. Spleens are harvested and splenocytes are restimulated overnight on uninfected or BCG-infected bone marrow-derived macrophages (BMDMs) from these mice strains that have been stimulated with IFN-gamma to promote presentation of bacterial antigens. Thus, this is a very physiologic assay in which lymphocytes are harvested from vaccinated mice and then tested for their ability to make cytokines in response to an in vitro macrophage infection model that bears many similarities with in vivo infection. Intracellular cytokine staining (ICS) is performed with anti-CD3, anti-CD4, and anti-CD8 surface antibodies, and anti-IFN-gamma, anti-IL2 and anti-TNF-alpha intracellular antibodies. The specimens are then analyzed on a FACSaria sorter. BCG antigen-specific responses are determined by comparing IFN-γ, IL-2, and occasionally TNF-α production by splenocytes restimulated overnight on BCG-infected BMDMs versus cytokine production incubated overnight on uninfected BMDMs.

To determine immunologic responses, multiple experiments were performed comparing BCG, DD-BCG, 3D-BCG, and 4D-BCG (FIG. 26). After vaccination with BCG and DD-BCG sustained cytokine production was observed. About 0.7% of CD4 T-cells in the spleens of mice were able to produce IFN-γ in response to antigenic stimulation at day 70 post-vaccination. At 259 days post-vaccination, 0.30% and 0.27% of splenic CD4 cells still made IFN-γ in BCG and DD-BCG vaccinees, respectively (data not shown in Figure). These results correlate with prolonged survival of both BCG and DD-BCG in the spleens of C57Bl/6 mice, a strain well-known for its “BCG-susceptibility” related to a mutant Nramp1 locus [Govoni, G. et al, 1996].

Differences in the production of specific cytokines were also noted. BCG-vaccinated mice exhibited a predominant IFN-γ response and the IL-2 production in BCG-vaccinated mice was not reliably above the natural variability in the assay (i.e., the range of IL-2 values observed in mice vaccinated with phosphate-buffered saline [sham-vaccinated controls] as indicated by the shaded area). When IL-2 production was observed in BCG-vaccinated mice, it was at low levels and detected around the time of the peak of the primary T-cell response at 4 weeks. In contrast, mice vaccinated with DD-BCG had fewer IFN-γ-producing CD4 cells relative to BCG-vaccinated mice but more IL-2-producing cells. The % of CD4+ T-cells producing IL-2 roughly correlated with the “generation” of paBCG vaccine under evaluation, and the induction of IL-2+ CD4+ T-cell responses was greater for 4D-BCG>3D-BCG>DD-BCG>BCG (FIG. 26A, lower panel). These results show that the pro-apoptotic modifications have an additive effect and when combined produce progressive enhancements in IL-2 production during primary vaccination.

The ratio of IFN-γ-producing to IL-2-producing CD4 cells in the same spleen typically averaged about 10:1 and 3:1 for recipients of BCG and the paBCG vaccines, respectively (FIG. 26B, in which the IL-2+ background values from uninfected BMDMs have been subtracted). This observation, combined with some other differences shown below, show that there is a qualitative enhancement in immune response induced by the paBCG vaccines compared to the immune response induced BCG.

The differences in cytokine production are best illustrated by comparing results around the peak of the primary T-cell response. FIG. 27 shows results from day 25 and day 31 post-vaccination in an experiment that compared BCG, DD-BCG, and 3D-BCG. In addition to the differences in IFN-γ production by CD4 T-cells (BCG>>DD-BCG>3D-BCG) and differences in IL-2 production by CD4+ T-cells (3D-BCG>>DD-BCG>BCG), the results also show increased IFN-γ production by CD8+ T-cells in the 3D-BCG-vaccinated mouse on day 25 (0.30%). Although the percentages of CD4 and CD8 IFN-γ-producing cells were identical, this mouse had a higher number of circulating CD8 cells, so in absolute terms the number of CD8+ IFN-γ+ cells was higher than the number of CD4 IFN-γ+ cells on day 25. Differences in values associated with DD-BCG versus 3D-BCG again suggest each pro-apoptotic modification has an additive effect in enhancing the immunogenicity of BCG.

In summary, the pattern of T-cell effector cytokines induced by the paBCG vaccines during primary vaccination is different from the pattern of T-cell effector cytokines induced by BCG. As shown below in additional immunologic studies performed in the context of vaccination-challenge experiments, these differences during primary vaccination facilitate the development of memory responses that enable the vaccinated host to respond quickly to infection. The greater induction of IL-2 production by paBCG vaccine strains should promote T-cell growth, as the presence of IL-2 during the contraction phase of the primary T-cell response enhances the survival of antigen-specific T-cells [Blattman, J. N. et al, 2003].

### Example 14

### Enhanced Recall T-Cell Responses after Intratracheal Challenge of Mice Previously Vaccinated with 3D-BCG Compared to Mice Previously Vaccinated with BCG

The goal of vaccination is to generate a memory lymphocyte population in the immunized host that is directed against the infectious agent and can respond briskly to infection. To determine the kinetics and magnitude of recall T-cell responses, mice were subcutaneously vaccinated with 5×10⁵ cfu of BCG or 3D-BCG. Control mice were sham-vaccinated with phosphate-buffered saline (PBS). Thirty days following vaccination, mice were treated with antibiotics to eradicate any persisting vaccine bacilli. Although preliminary data indicate that the 3D-BCG and 4D-BCG vaccines are cleared as the adaptive immune response develops, BCG persists indefinitely in C57Bl/6 mice and in the spleen for at least five months after subQ vaccination [Olsen, A. W. et al, 2004]. Thus, to avoid interference by the persistence of BCG, the vaccine strains were eliminated by treating all mice with isoniazid and rifampin in the drinking water starting at one month post-vaccination. This was found to be effective in reducing the number of BCG in the spleen below the lower limits of detection. After a month of treatment and an additional four weeks of rest, the mice receive an intratracheal challenge of 4×10⁷ cfu of BCG (all groups of mice, regardless of the initial vaccine strain). Baseline (day 0) numbers of cytokine+ T-cells before challenge were low (not shown). Five days after challenge, the mice were euthanized and lungs were harvested to determine T-cell responses. The results are shown in FIG. 28 and show much stronger CD4+ T-cell responses in the mice vaccinated with 3D-BCG compared to the mice vaccinated with BCG. The 10-fold higher percent of IL-2+ CD4+ T-cells from mice vaccinated with 3D-BCG versus BCG recapitulates the greater IL-2 production seen during primary vaccination (FIGS. 26 and 27). Although the challenge dose used in this experiment is high/non-physiologic for TB infection, the design does allow us to assess the rapidity of secondary T-cell responses under conditions of a relatively high antigen load. Thus, the results support the vector function of paBCG for delivering antigens of infectious agents that may rise to high titer very soon after inoculation (e.g., viral pathogens, malaria).

In summary, the secondary T-cell responses observed after challenge of mice vaccinated with 3D-BCG are stronger than secondary T-cell responses observed in mice vaccinated with BCG. The results show that paBCG is better than BCG in inducing a population of memory T-cells that can respond rapidly to challenge during a secondary (recall) response. Combined with greater attenuation and its ability to induce greater protection against tuberculosis than the current BCG vaccine, the immunologic studies highlight the use of paBCG as a platform technology for delivering exogenous antigens against other important infectious diseases and to target cancer.

TABLE 1
Bacterial strains, tools for genetic manipulations, and genetic constructs

| | | |
|---|---|---|
| Strains and | | |
| genetic tools | Description | Reference or source |

Strains

| | | |
|---|---|---|
| H37Rv | Virulent M. tuberculosis reference strain, | ATCC 25618 |
| | source of template chromosomal DNA for | |
| | gene mutations | |
| Erdman | Virulent M. tuberculosis reference strain, | ATCC 35801 |
| | commonly used as challenge strain in | |
| | experiments to evaluate vaccine efficacy | |
| AcrR-Erdman | Acriflavin-resistant mutant of Erdman, | Sheldon Morris, FDA |
| | also used as challenge strain for vaccine | [Repique, C. J. et al, |
| | efficacy | 2002] |
| TOP 10 | Host strain for cloning PCR products, | Invitrogen Corp., |
| | used in combination with pCR2.1-TOPO | Carlsbad, California |
| DH5α | E. coli host strain for genetic | [Hanahan, D., 1983] |
| | manipulation, construction of mutant | |
| | enzyme expression vectors | |
| BCG Tice | Bacillus Calmette-Guerin, substrain Tice | Organon Teknika |
| | | Corp., Durham, NC |
| SD-BCG-AS-SOD | SodA-diminished BCG containing either | [Edwards, K. M. et al, |
| | pHV203-AS-SOD or pLUC10-AS-SOD | 2001] and WO |
| | to practice antisense strategy | 02/062298 |
| C-BCG | Control BCG with either pHV203 or | [Edwards, K. M. et al, |
| | pLUC10 plasmid containing 151-bp of | 2001] and WO |
| | SodA but not in antisense orientation | 02/062298 |
| BCG (pLou1-mut | BCG with pLou1 chromosomal | Work related to the |
| SodA) | integration vector expressing mutant | teachings of WO |
| | SodA - BCG(pLou1-mut SodA) strains | 02/062298, mutant |
| | containing the following mutant SodA | SodA enzymes |
| | genes were constructed: H76K, ΔG134, | described in Table 11 |
| | H145K, H164K, ΔV184 | |
| | 1^{st} generation pro-apoptotic BCG | |
| | vaccines | |
| SAD-BCGΔE54 | SodA-diminished BCG containing either | This invention |
| (aka SD-BCG | pMP349-mut SodA ΔE54 or pMP399- | |
| ΔE54) | mut SodA ΔE54 to practice dominant- | |
| | negative strategy | |
| SAD-BCG | SodA-diminished BCG containing either | This invention |
| ΔH28ΔH76 (aka | pMP349-mut SodA ΔH28ΔH76 or | |
| SD-BCG | pMP399-mut SodA ΔH28ΔH76 | |
| ΔH28ΔH76) | | |
| SIG-BCG (aka | BCG with allelic inactivation of sigH | This invention |
| BCGΔsigH) | | |
| SEC-BCG (aka | BCG with allelic inactivation of secA2 | Miriam Braunstein, |
| BCGΔsecA2) | | UNC, Chapel Hill |
| | | using methods |
| | | described in |
| | | [Braunstein, M. et al, |
| | | 2003; Braunstein, M. |
| | | et al, 2002] |
| GLAD-BCG | glnA1-diminished BCG containing either | This invention |
| | pMP349-mut glnA1 ΔD54ΔE335, | |
| | pHV203-mut glnA1 ΔD54ΔE335, or | |
| | pMP399-mut glnA1 ΔD54ΔE335 to | |
| | practice dominant-negative strategy | |
| | 2^{nd} generation pro-apoptotic BCG | |
| | vaccines | |
| SAD-SIG-BCG | BCGΔsigH that is also sodA-diminished | This invention |
| ΔE54 (aka | by containing either pMP349-mut SodA | |
| BCGΔsigH ΔE54) | ΔE54 or pMP399-mut SodA ΔE54 | |
| SAD-SIG-BCG | BCGΔsigH that is also sodA-diminished | This invention |
| ΔH28ΔH76 (aka | by containing either pMP349-mut SodA | |
| BCGΔsigH | ΔH28ΔH76 or pMP399-mut SodA | |
| ΔH28ΔH76) | ΔH28ΔH76 | |
| SAD-SEC-BCG | BCGΔsecA2 that is also sodA-diminished | This invention |
| ΔE54 (aka | by containing either pMP349-mut SodA | |
| BCGΔsecA2 ΔE54) | ΔE54 or pMP399-mut SodA ΔE54 | |
| SAD-SEC-BCG | BCGΔsecA2 that is also sodA-diminished | This invention |
| ΔH28ΔH76 (aka | by containing either pMP349-mut SodA | |
| BCGΔsecA2 | ΔH28ΔH76 or pMP399-mut SodA | |
| ΔH28ΔH76) | ΔH28ΔH76 | |
| DD-BCG (aka | BCGΔsigHΔsecA2, also referred to as | This invention |
| BCGΔsigHΔsecA2) | “double-deletion” BCG | |
| GLAD-SIG-BCG | BCGΔsigH that is also glnA1-diminished | This invention |
| (aka BCGΔsigH | by containing either pMP349-mut glnA1 | |
| mut glnA1) | ΔD54ΔE335 or pMP399-mut glnA1 | |
| | ΔD54ΔE335 | |
| GLAD-SEC-BCG | BCGΔsecA2 that is also glnA1- | This invention |
| (aka BCGΔsecA2 | diminished by containing either pMP349- | |
| mut glnA1) | mut glnA1 ΔD54ΔE335 or pMP399-mut | |
| | glnA1 ΔD54ΔE335 | |
| GLAD-SAD-BCG | glnA1- and SodA-diminished BCG due to | This invention |
| ΔE54 | overexpression of mut glnA1 | |
| | ΔD54ΔE335 PLUS mut SodA ΔE54 | |
| GLAD-SAD-BCG | glnA1- and SodA-diminished BCG due to | This invention |
| ΔH28ΔH76 | overexpression of mut ginA1 | |
| | ΔD54ΔE335 PLUS mut SodA | |
| | ΔH28ΔH76 | |
| | 3^{rd} generation pro-apoptotic BCG | |
| | vaccines | |
| 3D-BCG ΔE54 | DD-BCG that overexpresses mut SodA | This invention |
| | ΔE54 | |
| 3D-BCG | DD-BCG that overexpresses mut SodA | This invention |
| ΔH28ΔH76 | ΔH28ΔH76 | |
| GLAD-DD-BCG | DD-BCG that overexpresses mut glnA1 | This invention |
| | ΔD54ΔE335 | |
| GLAD-SAD-SIG- | BCGΔsigH that overexpresses mut glnA1 | This invention |
| BCG ΔE54 | ΔD54ΔE335 PLUS mut SodA ΔE54 | |
| GLAD-SAD-SIG- | BCGΔsigH that overexpresses mut glnA1 | This invention |
| BCG ΔH28ΔH76 | ΔD54ΔE335 PLUS mut SodA | |
| | ΔH28ΔH76 | |
| GLAD-SAD-SEC- | BCGΔsecA2 that overexpresses mut | This invention |
| BCG ΔE54 | glnA1 ΔD54ΔE335 PLUS mut SodA | |
| | ΔE54 | |
| GLAD-SAD-SEC- | BCGΔsecA2 that overexpresses mut | This invention |
| BCG ΔH28ΔH76 | glnA1 ΔD54ΔE335 PLUS mut SodA | |
| | ΔH28ΔH76 | |
| | 4^{th} generation pro-apoptotic BCG | |
| | vaccines | |
| 4D-BCG ΔE54 | DD-BCG that overexpresses mut glnA1 | This invention |
| | ΔD54ΔE335 PLUS mut SodA ΔE54 | |
| 4D-BCG | DD-BCG that overexpresses mut glnA1 | This invention |
| ΔH28ΔH76 | ΔD54ΔE335 PLUS mut SodA | |
| | ΔH28ΔH76 | |
| | Pro-apoptotic BCG expressing | |
| | exogenous antigen | |
| DD-BCGrBLS | DD-BCG expressing recombinant | This invention |
| | Brucella lumazine synthase, from | |
| | Brucella abortus | |

Plasmids

| | | |
|---|---|---|
| pCR2.1-TOPO | Plasmid for cloning PCR products | Invitrogen Corp., |
| | | Carlsbad, California |
| pBC SK+ | E. coli phagemid vector | Stratagene, La Jolla, |
| | | CA |
| pMP349 | E. coli - mycobacterial shuttle plasmid | Martin Pavelka |
| | containing aacC41 gene encoding | [Consaul, S. A. et al, |
| | apramycin resistance | 2004] |
| pMP349-mut SodA | pMP349 with ΔE54 mutant SodA gene | This invention - |
| ΔE54 | cloned behind aceA (icl) promoter - mut | sequence footnote A |
| | SodA also contains H28R substitution | |
| pMP349-mut SodA | pMP349 with ΔH28ΔH76 mutant SodA | This invention - |
| ΔH28ΔH76 | gene cloned behind aceA (icl) promoter - | sequence footnote B |
| | mut SodA also contains G→S substitution | |
| | at C-terminus | |
| pMP349-mut | pMP349 with ΔD54ΔE335 mutant glnA1 | This invention - |
| glnA1 ΔD54ΔE335 | gene with its own promoter | sequence footnote C |
| pMP349-mut SodA | pMP349 with ΔH28ΔH76 mutant SodA | This invention - |
| ΔH28ΔH76, mut | gene cloned behind aceA (icl) promoter | sequence footnote D |
| glnA1 ΔD54ΔE335 | and ΔD54ΔE335 mutant glnA1 gene with | |
| | its own promoter | |
| pHV203* | E. coli-mycobacterial shuttle plasmid with | [Edwards, K. M. et al, |
| | kanamycin resistance gene | 2001] and WO |
| | | 02/062298 |
| pHV203-AS-SOD | pHV203 containing a 151-bp fragment of | [Edwards, K. M. et al, |
| | sodA cloned in an antisense orientation | 2001] and WO |
| | behind promoter of 65 kDa heat-shock | 02/062298 |
| | protein | |
| pHV203-mut | pHV203 with ΔD54ΔE335 mutant glnA1 | This invention - |
| glnA1 ΔD54ΔE335 | gene with its own promoter | sequence footnote E |
| pLUC10 | E. coli-mycobacterial shuttle plasmid | Robert Cooksey, |
| | containing firefly luciferase gene | CDC, Atlanta, |
| | | Georgia [Cooksey, R. C. |
| | | et al, 1993] |
| pLUC10-AS-SOD | pLUC10 containing a 151-bp fragment of | [Edwards, K. M. et al, |
| | sodA cloned in an antisense orientation | 2001] and WO |
| | behind promoter of 65 kDa heat-shock | 02/062298 |
| | protein | |
| pY6002 | Plasmid containing aph gene from Tn903, | Richard Young, MIT |
| | conferring resistance to kanamycin | [Aldovini, A. et al, |
| | | 1993] |
| pBAK14 | E. coli-mycobacterial shuttle plasmid | Douglas Young, |
| | containing the origin of replication from | Hammersmith |
| | the M. fortuitum plasmid pAL5000 | Hospital, London |
| | | [Zhang, Y. et al, 1991] |
| p16R1 | E. coli-mycobacterial shuttle plasmid for | Douglas Young, |
| | expressing SodA in mycobacteria, with | Hammersmith |
| | hygromycin resistance gene | Hospital, London |
| pNBV-1 | E. coli-mycobacterial shuttle plasmid with | [Howard, N. S. et al, |
| | hygromycin resistance gene | 1995] |

Chromosomal integration vectors

| | | |
|---|---|---|
| pMH94 | E. coli-mycobacterial attB integration | [Lee, M. H. et al, |
| | vector | 1991] |
| pLou1 | E. coli-mycobacterial attB integration | Jim Graham, |
| | vector | University of |
| | | Louisville |
| pLou1-mut SodA | pLou1 containing mutant SodA, pLou1 | Work related to the |
| | containing the following mutant SodA | teachings of WO |
| | genes were constructed: pLou1-H76K, | 02/062298 |
| | pLou1-ΔG134, pLou1-H145K, pLou1- | |
| | H164K, pLou1-ΔV184 | |
| pMP399 | E. coli-mycobacterial attB integration | Martin Pavelka |
| | vector containing aacC41 gene encoding | [Consaul, S. A. et al, |
| | apramycin resistance | 2004] |
| pMP399-mut SodA | pMP399 with ΔE54 mutant SodA gene | This invention - |
| ΔE54 | cloned behind aceA (icl) promoter - mut | sequence footnote F |
| | SodA also contains H28R substitution | |
| pMP399-mut SodA | pMP399 with ΔH28ΔH76 mutant SodA | This invention - |
| ΔH28ΔH76 | gene cloned behind aceA (icl) promoter - | sequence footnote G |
| | mut SodA also contains G→S substitution | |
| | at C-terminus | |
| pMP399-mut | pMP399 with ΔD54ΔE335 mutant glnA1 | This invention - |
| glnA1 ΔD54ΔE335 | gene with its own promoter | sequence footnote H |
| pMP399-mut SodA | pMP399 with Δ54 mutant SodA gene | This invention - |
| ΔE54, mut glnA1 | cloned behind aceA (icl) promoter and | sequence footnote I |
| ΔD54ΔE335 | ΔD54ΔE335 mutant glnA1 gene with its | |
| | own promoter | |
| pMP399-mut SodA | pMP399 with ΔH28ΔH76 mutant SodA | This invention - |
| ΔH28ΔH76, mut | gene cloned behind aceA (icl) promoter | sequence footnote J |
| glnA1 ΔD54ΔE335 | and ΔD54ΔE335 mutant glnA1 gene with | |
| | its own promoter | |

Allelic inactivation tools for chromosomal genes

| | | |
|---|---|---|
| pYUB854, | phasmid chromosomal gene inactivation | William Jacobs, Jr., |
| pHAE87, | system for mycobacteria | Albert Einstein |
| pHAE159 | | College of Medicine |
| | | [Braunstein, M. et al, |
| | | 2002] |
| pYUB854-sigH | phasmid system vector for sigH | This invention - |
| | inactivation, used to construct | sequence footnote K |
| | BCGΔsigH | |
| pYUB854-trx-trxr | phasmid system vector for inactivation | This invention - |
| | of thioredoxin and thioredoxin | sequence footnote L |
| | reductase, used to construct | |
| | BCGΔtrxΔtrxr | |
| pYUB854-sigE | phasmid system vector for sigE | This invention - |
| | inactivation, used to construct | sequence footnote M |
| | BCGΔsigH | |
| p1NIL, p2NIL, | suicide plasmid system for use in allelic | [Parish, T. et al, |
| pGOAL17, | replacement in mycobacteria | 2000] |
| pGOAL19 | | |
| p2NIL/GOAL19- | suicide plasmid for introducing | This invention - |
| mut trxC-mut | unmarked active-site mutations into trxC | sequence footnote N |
| trxB2 | and trxB2 | |

Exogenous antigen expression vectors

| | | |
|---|---|---|
| pMP349-rBLS | pMP349 with recombinant Brucella | This invention - |
| | lumazine synthase behind aceA(icl) | sequence footnote O, |
| | promoter | bls allele provided by |
| | | Martin Roop, ECU |

*Note: the terms pHV202 and pHV203 are used interchangeably. pHV203 was derived from pHV202 by repairing a mutation in the promoter region of the 65 kDa heat-shock protein used to drive expression of antisense DNA, and the inclusion of a larger upstream region of DNA to enhance stability.

Sequence Footnotes:

(A) pMP349-mut SodA ΔE54 (SEQ ID NO: 23)

Full nucleotide sequence of plasmid vector pMP349-mut SodA ΔE54 used to express the mutant sodA in BCG to create SAD-BCGAE54 (plasmid-expressed). It can also be added to 1^{st}, 2^{nd}, and 3^{rd} generation mutants of pro-apoptotic BCG to render, respectively, 2^{nd}, 3^{rd}, and 4^{th} generation pro-apoptotic BCG vaccines.

| | |
|---|---|
| 1 | ctagttccac tgagcgtcag accccgtaga aaagatcaaa ggatcttctt gagatccttt |
| 61 | ttttctgcgc gtaatctgct gcttgcaaac aaaaaaacca ccgctaccag cggtggtttg |
| 121 | tttgccggat caagagctac caactctttt tccgaaggta actggcttca gcagagcgca |
| 181 | gataccaaat actgtccttc tagtgtagcc gtagttaggc caccacttca agaactctgt |
| 241 | agcaccgcct acatacctcg ctctgctaat cctgttacca gtggctgctg ccagtggcga |
| 301 | taagtcgtgt cttaccgggt tggactcaag acgatagtta ccggataagg cgcagcggtc |
| 361 | gggctgaacg gggggttcgt gcacacagcc cagcttggag cgaacgacct acaccgaact |
| 421 | gagataccta cagcgtgagc attgagaaag cgccacgctt cccgaaggga gaaaggcgga |
| 481 | caggtatccg gtaagcggca gggtcggaac aggagagcgc acgagggagc ttccaggggg |
| 541 | aaacgcctgg tatctttata gtcctgtcgg gtttcgccac ctctgacttg agcgtcgatt |
| 601 | tttgtgatgc tcgtcagggg ggcggagcct atggaaaaac gccagcaacg cggccttttt |
| 661 | acggttcctg gccttttgct ggccttttgc tcacatgttc tttcctgcgt tatcccctga |
| 721 | ttctgtggat aaccgtatta ccgcctttga gtgagctgat accgctcgcc gcagccgaac |
| 781 | gaccgagcgc aacgcgtgag cccaccagct ccgtaagttc gggtgctgtg tggctcgtac |
| 841 | ccgcgcattc aggcggcagg gggtctaacg ggtctaaggc ggcgtgtacg gccgccacag |
| 901 | cggctcttag cggcccggaa acgtcctcga aacgacgcat gtgttcctcc tggttggtac |
| 961 | aggtggttgg gggtgctcgg ctgtcgctgg tgtttcatca tcagggctcg acgggagagc |
| 1021 | gggggagtgt gcagttgtgg ggtggcccct cagcgaaata tctgacttgg agctcgtgtc |
| 1081 | ggaccataca ccggtgatta atcgtggttt attatcaagc gtgagccacg tcgccgacga |
| 1141 | atttgagcag ctctggctgc cgtactggtc cctggcaagc gacgatctgc tcgaggggat |
| 1201 | ctaccgccaa agccgcgcgt cggccctagg ccgccggtac atcgaggcga acccaacagc |
| 1261 | gctggcaaac ctgctggtcg tggacgtaga ccatccagac gcagcgctcc gagcgctcag |
| 1321 | cgcccggggg tcccatccgc tgcccaacgc gatcgtgggc aatcgcgcca acggccacgc |
| 1381 | acacgcagtg tgggcactca acgcccctgt tccacgcacc gaatacgcgc ggcgtaagcc |
| 1441 | gctcgcatac atggcggcgt gcgccgaagg ccttcggcgc gccgtcgatg gcgaccgcag |
| 1501 | ttactcaggc ctcatgacca aaaaccccgg ccacatcgcc tgggaaacgg aatggctcca |
| 1561 | ctcagatctc tacacactca gccacatcga ggccgagctc ggcgcgaaca tgccaccgcc |
| 1621 | gcgctggcgt cagcagacca cgtacaaagc ggctccgacg ccgctagggc ggaattgcgc |
| 1681 | actgttcgat tccgtcaggt tgtgggccta tcttcccgcc ctcatgcgga tctacctgcc |
| 1741 | gacccggaac gtggacggac tcggccgcgc gatctatgcc gagtgccacg cgcgaaacgc |
| 1801 | cgaatttccg tgcaacgacg tgtgtcccgg accgctaccg gacagcgagg tccgcgccat |
| 1861 | cgccaacagc atttggcgtt ggatcacaac caagtcgcgc atttgggcgg acgggatcgt |
| 1921 | ggtctacgag gccacactca gtgcgcgcca tgcggccatc tcgcggaagg gcgcagcagc |
| 1981 | gcgcacggcg gcgagcacag ttgcgcggcg cgcaaagtcc gcgtcagcca tggaggcatt |
| 2041 | gctatgagcg acggctacag cgacggctac agcgacggct acaactggca gccgactgtc |
| 2101 | cgcaaaaagc ggcgcgtgac cgccgccgaa ggcgctcgaa tcaccggact atccgaacgc |
| 2161 | cacgtcgtcc ggctcgtggc gcaggaacgc agcgagtggt tcgccgagca ggctgcacgc |
| 2221 | cgcgaacgca tccgcgccta tcacgacgac gagggccact cttggccgca aacggccaaa |
| 2281 | catttcgggc tgcatctgga caccgttaag cgactcggct atcgggcgag gaaagagcgt |
| 2341 | gcggcagaac aggaagcggc tcaaaaggcc cacaacgaag ccgacaatcc accgctgttc |
| 2401 | taacgcaatt ggggagcggg tgtcgcgggg gttccgtggg gggttccgtt gcaacgggtc |
| 2461 | ggacaggtaa aagtcctggt agacgctagt tttctggttt gggccatgcc tgtctcgttg |
| 2521 | cgtgtttcgt tgcgtccgtt ttgaatacca gccagacgag acggggttct acgaatcttg |
| 2581 | gtcgatacca agccatttcc gctgaatatc gtggagctca ccgccagaat cggtggttgt |
| 2641 | ggtgatgtac gtggcgaact ccgttgtagt gcttgtggtg gcatccgtgg cgcggccgcg |
| 2701 | gtaccccatg gtgatgcgcg actccggaat actgagcccg acgcttgcgg cagcggggtc |
| 2761 | agctgaatat caaccccttg gtctgcgagg tcgcggccgc ataccgtgac tgcacatcgg |
| 2821 | cccagttcac gacgttccaa aacgccttgg caaagtcgac tttgacgttc ttgtactgca |
| 2881 | ggtagaaggc gtgttcccac atgtcgagca gcagcagcgg aacaatgcct agcgggaagt |
| 2941 | tcgtctggtg gtcgtaaacc tggaatatca gcagcttgtt gccgagtgtg tcccagccca |
| 3001 | gtgccgccca gcccgacccc tgcacggtgg tagcggccgc gtggaactgc gcacggaact |
| 3061 | tgtcgaacga accgaacgcg tcggcgatgg ctgcggcgag ttcgccggtg ggcttgtcac |
| 3121 | caccgttagg cgacaggttc ttccaccaga tggtgtgatt aacgtggccg gcgaggttga |
| 3181 | aagctagatt cttttcgttc agcatgatcg ctgagtgatc cttggcgcgc gcctcttcga |
| 3241 | gtttggcgac ggcgtcattg gcgcccttta cgtaggtggc gtggtgcttg ctgtggcgaa |
| 3301 | gctcgttgat ctgacccgag atgtgcggtt ccagtgctcc gtagtcccag tccaggtctg |
| 3361 | gcaaggtgta ttcggccacg gggtacccca gacaactcct taacggtctt tcattgccga |
| 3421 | aaacgctgac gccctaccgt cgtccaggcg gtgtcaacgg cgcagcttca ctggtgtgct |
| 3481 | aactcgacca tggcacagcg tgtcaacgct ggtccaccca tttcacttgc gaatttcggc |
| 3541 | aacggcctgc ggactttttg caaattttgc gaagtcgccc aaaaactgaa ccgtttcaga |
| 3601 | agctacccgc cagtaacgac aaatccgcag gtaaacccac ggatcgacgt cctgcggatc |
| 3661 | cggtcacaga ttgaacagcg aggcgactgc cttgggctcg tcgccaacca catatgtgag |
| 3721 | cgttgtaaca tctagaggtg accacaacga cgcgcccgct ttgatcgggg acgtctgcgg |
| 3781 | ccgaccattt acgggtcttg ttgtcgttgg cggtcatggg ccgaacatac tcacccggat |
| 3841 | cggagggccg aggacaaggt cgaacgaggg gcatgacccg gtgcggggct tcttgcactc |
| 3901 | ggcataggcg agtgctaaga ataacgttgg cactcgcgac cggtgagtcg taggtcggga |
| 3961 | cggtgaggcc aggcccgtcg tcgcagcgag tggcagcgag gacaacttga gccgtccgtc |
| 4021 | gcgggcactg cgcccggcca gcgtaagtag cggggttgcc gtcacccggt gacccccggt |
| 4081 | ttcatccccg atccggagga atcacttcgc aatggccaag acaattgcgg atccagctgc |
| 4141 | agaattcctg cagctcacgg taactgatgc cgtatttgca gtaccagcgt acggcccaca |
| 4201 | gaatgatgtc acgctgaaaa tgccggcctt tgaatgggtt catgtgcagc tccatcagca |
| 4261 | aaaggggatg ataagtttat caccaccgac tatttgcaac agtgccgttg atcgtgctat |
| 4321 | gatcgactga tgtcatcagc ggtggagtgc aatgtcgtgc aatacgaatg gcgaaaagcc |
| 4381 | gagctcatcg gtcagcttct caaccttggg gttacccccg gcggtgtgct gctggtccac |
| 4441 | agctccttcc gtagcgtccg gcccctcgaa gatgggccca cttggactga tcgaggccct |
| 4501 | gcgtgctacg ctgggtccgg gagggacgct cgtcatgccc tcgtggtcag gtctggacga |
| 4561 | cgagccgttc gatcctgcca cgtcgcccgt tacaccggac cttggagttg tctctgacac |
| 4621 | attctggcgc ctgccaaatg taaagcgcag cgcccatcca tttgcctttg cggcagcggg |
| 4681 | gccacaggca gagcagatca tctctgatcc attgcccctg ccaccttact cgcctgcaag |
| 4741 | cccggtcgcc cgtgtccatg aactcgatgg gcaggtactt ctcctcggcg tgggacacga |
| 4801 | tgccaacacg acgctgcatc ttgccgagtt gatggcaaag gttccctatg gggtgccgag |
| 4861 | acactgcacc attcttcagg atggcaagtt ggtacgcgtc gattatctcg agaatgacca |
| 4921 | ctgctgtgag cgctttgcct tggcgggaca ggtggctcaa ggagaagagc cttcagaagg |
| 4981 | aaggtccagt cggtcatgcc tttgctcggt tgatccgctc ccgcgacatt gtggcgacag |
| 5041 | ccctgggtca actgggccga gatccgttga tcttcctgca tccgccagag ggcgggatgc |
| 5101 | gaagaatgcg atgccgctcg ccagtcgatt ggctgagctc atgagcggag aacgagatga |
| 5161 | cgttggaggg gcaaggtcgc gctgattgct ggggcaacac gggggatcca |

(B) pMP349-mut SodA ΔH28ΔH76 (SEQ ID NO: 24)

Full nucleotide sequence of plasmid vector pMP349-mut SodA ΔH28ΔH76 used to express the mutant soda in BCG to create SAD-BCGΔH28ΔH76 (plasmid-expressed). It can also be added to 1^{st}, 2^{nd}, and 3^{rd} generation mutants of pro-apoptotic BCG to render, respectively, 2^{nd}, 3^{rd}, and 4^{th} generation pro-apoptotic BCG vaccines.

| | |
|---|---|
| 1 | ctagttccac tgagcgtcag accccgtaga aaagatcaaa ggatcttctt |
| 51 | gagatccttt ttttctgcgc gtaatctgct gcttgcaaac aaaaaaacca |
| 101 | ccgctaccag cggtggtttg tttgccggat caagagctac caactctttt |
| 151 | tccgaaggta actggcttca gcagagcgca gataccaaat actgtccttc |
| 201 | tagtgtagcc gtagttaggc caccacttca agaactctgt agcaccgcct |
| 251 | acatacctcg ctctgctaat cctgttacca gtggctgctg ccagtggcga |
| 301 | taagtcgtgt cttaccgggt tggactcaag acgatagtta ccggataagg |
| 351 | cgcagcggtc gggctgaacg gggggttcgt gcacacagcc cagcttggag |
| 401 | cgaacgacct acaccgaact gagataccta cagcgtgagc attgagaaag |
| 451 | cgccacgctt cccgaaggga gaaaggcgga caggtatccg gtaagcggca |
| 501 | gggtcggaac aggagagcgc acgagggagc ttccaggggg aaacgcctgg |
| 551 | tatctttata gtcctgtcgg gtttcgccac ctctgacttg agcgtcgatt |
| 601 | tttgtgatgc tcgtcagggg ggcggagcct atggaaaaac gccagcaacg |
| 651 | cggccttttt acggttcctg gccttttgct ggccttttgc tcacatgttc |
| 701 | tttcctgcgt tatcccctga ttctgtggat aaccgtatta ccgcctttga |
| 751 | gtgagctgat accgctcgcc gcagccgaac gaccgagcgc aacgcgtgag |
| 801 | cccaccagct ccgtaagttc gggtgctgtg tggctcgtac ccgcgcattc |
| 851 | aggcggcagg gggtctaacg ggtctaaggc ggcgtgtacg gccgccacag |
| 901 | cggctcttag cggcccggaa acgtcctcga aacgacgcat gtgttcctcc |
| 951 | tggttggtac aggtggttgg gggtgctcgg ctgtcgctgg tgtttcatca |
| 1001 | tcagggctcg acgggagagc gggggagtgt gcagttgtgg ggtggcccct |
| 1051 | cagcgaaata tctgacttgg agctcgtgtc ggaccataca ccggtgatta |
| 1101 | atcgtggttt attatcaagc gtgagccacg tcgccgacga atttgagcag |
| 1151 | ctctggctgc cgtactggtc cctggcaagc gacgatctgc tcgaggggat |
| 1201 | ctaccgccaa agccgcgcgt cggccctagg ccgccggtac atcgaggcga |
| 1251 | acccaacagc gctggcaaac ctgctggtcg tggacgtaga ccatccagac |
| 1301 | gcagcgctcc gagcgctcag cgcccggggg tcccatccgc tgcccaacgc |
| 1351 | gatcgtgggc aatcgcgcca acggccacgc acacgcagtg tgggcactca |
| 1401 | acgcccctgt tccacgcacc gaatacgcgc ggcgtaagcc gctcgcatac |
| 1451 | atggcggcgt gcgccgaagg ccttcggcgc gccgtcgatg gcgaccgcag |
| 1501 | ttactcaggc ctcatgacca aaaaccccgg ccacatcgcc tgggaaacgg |
| 1551 | aatggctcca ctcagatctc tacacactca gccacatcga ggccgagctc |
| 1601 | ggcgcgaaca tgccaccgcc gcgctggcgt cagcagacca cgtacaaagc |
| 1651 | ggctccgacg ccgctagggc ggaattgcgc actgttcgat tccgtcaggt |
| 1701 | tgtgggccta tcttcccgcc ctcatgcgga tctacctgcc gacccggaac |
| 1751 | gtggacggac tcggccgcgc gatctatgcc gagtgccacg cgcgaaacgc |
| 1801 | cgaatttccg tgcaacgacg tgtgtcccgg accgctaccg gacagcgagg |
| 1851 | tccgcgccat cgccaacagc atttggcgtt ggatcacaac caagtcgcgc |
| 1901 | atttgggcgg acgggatcgt ggtctacgag gccacactca gtgcgcgcca |
| 1951 | tgcggccatc tcgcggaagg gcgcagcagc gcgcacggcg gcgagcacag |
| 2001 | ttgcgcggcg cgcaaagtcc gcgtcagcca tggaggcatt gctatgagcg |
| 2051 | acggctacag cgacggctac agcgacggct acaactggca gccgactgtc |
| 2101 | cgcaaaaagc ggcgcgtgac cgccgccgaa ggcgctcgaa tcaccggact |
| 2151 | atccgaacgc cacgtcgtcc ggctcgtggc gcaggaacgc agcgagtggt |
| 2201 | tcgccgagca ggctgcacgc cgcgaacgca tccgcgccta tcacgacgac |
| 2251 | gagggccact cttggccgca aacggccaaa catttcgggc tgcatctgga |
| 2301 | caccgttaag cgactcggct atcgggcgag gaaagagcgt gcggcagaac |
| 2351 | aggaagcggc tcaaaaggcc cacaacgaag ccgacaatcc accgctgttc |
| 2401 | taacgcaatt ggggagcggg tgtcgcgggg gttccgtggg gggttccgtt |
| 2451 | gcaacgggtc ggacaggtaa aagtcctggt agacgctagt tttctggttt |
| 2501 | gggccatgcc tgtctcgttg cgtgtttcgt tgcgtccgtt ttgaatacca |
| 2551 | gccagacgag acggggttct acgaatcttg gtcgatacca agccatttcc |
| 2601 | gctgaatatc gtggagctca ccgccagaat cggtggttgt ggtgatgtac |
| 2651 | gtggcgaact ccgttgtagt gcttgtggtg gcatccgtgg cgcggccgcg |
| 2701 | gtaccccatg gtgatgcgcg actccggaat actgagcccg acgcttgcgg |
| 2751 | cagcggggtc agctgaatat caaccccttg gtctgcgagg tcgcggccgc |
| 2801 | ataccgtgac tgcacatcgg cccagttcac gacgttccaa aacgccttgg |
| 2851 | caaagtcgac tttgacgttc ttgtactgca ggtagaaggc gtgttcccac |
| 2901 | atgtcgagca gcagcagcgg aacaatgcct agcgggaagt tcgtctggtg |
| 2951 | gtcgtaaacc tggaatatca gcagcttgtt gccgagtgtg tcccagccca |
| 3001 | gtgccgccca gcccgacccc tgcacggtgg tagcggccgc gtggaactgc |
| 3051 | gcacggaact tgtcgaacga accgaacgcg tcggcgatgg ctgcggcgag |
| 3101 | ttcgccggtg ggcttgtcac caccgttagg cgacaggttc ttccaccaga |
| 3151 | tggtattaac gtggccggcg aggttgaaag ctagattctt ttcgttcagc |
| 3201 | aagatcgctg agtgatcttc cttggcgcgc gcctcttcga gtttggcgac |
| 3251 | ggcgtcattg gcgcccttta cgtaggtggc gtggtgcttg ctgtgaagct |
| 3301 | cgttgatctg acccgagatg tgcggttcca gtgctccgta gtcccagtcc |
| 3351 | aggtctggca aggtgtattc ggccacgggg taccccagac aactccttaa |
| 3401 | cggtctttca ttgccgaaaa cgctgacgcc ctaccgtcgt ccaggcggtg |
| 3451 | tcaacggcgc agcttcactg gtgtgctaac tcgaccatgg cacagcgtgt |
| 3501 | caacgctggt ccacccattt cacttgcgaa tttcggcaac ggcctgcgga |
| 3551 | ctttttgcaa attttgcgaa gtcgcccaaa aactgaaccg tttcagaagc |
| 3601 | tacccgccag taacgacaaa tccgcaggta aacccacgga tcgacgtcct |
| 3651 | gcggatccgg tcacagattg aacagcgagg cgactgcctt gggctcgtcg |
| 3701 | ccaaccacat atgtgagcgt tgtaacatct agaggtgacc acaacgacgc |
| 3751 | gcccgctttg atcggggacg tctgcggccg accatttacg ggtcttgttg |
| 3801 | tcgttggcgg tcatgggccg aacatactca cccggatcgg agggccgagg |
| 3851 | acaaggtcga acgaggggca tgacccggtg cggggcttct tgcactcggc |
| 3901 | ataggcgagt gctaagaata acgttggcac tcgcgaccgg tgagtcgtag |
| 3951 | gtcgggacgg tgaggccagg cccgtcgtcg cagcgagtgg cagcgaggac |
| 4001 | aacttgagcc gtccgtcgcg ggcactgcgc ccggccagcg taagtagcgg |
| 4051 | ggttgccgtc acccggtgac ccccggtttc atccccgatc cggaggaatc |
| 4101 | acttcgcaat ggccaagaca attgcggatc cagctgcaga attcctgcag |
| 4151 | ctcacggtaa ctgatgccgt atttgcagta ccagcgtacg gcccacagaa |
| 4201 | tgatgtcacg ctgaaaatgc cggcctttga atgggttcat gtgcagctcc |
| 4251 | atcagcaaaa ggggatgata agtttatcac caccgactat ttgcaacagt |
| 4301 | gccgttgatc gtgctatgat cgactgatgt catcagcggt ggagtgcaat |
| 4351 | gtcgtgcaat acgaatggcg aaaagccgag ctcatcggtc agcttctcaa |
| 4401 | ccttggggtt acccccggcg gtgtgctgct ggtccacagc tccttccgta |
| 4451 | gcgtccggcc cctcgaagat gggcccactt ggactgatcg aggccctgcg |
| 4501 | tgctacgctg ggtccgggag ggacgctcgt catgccctcg tggtcaggtc |
| 4551 | tggacgacga gccgttcgat cctgccacgt cgcccgttac accggacctt |
| 4601 | ggagttgtct ctgacacatt ctggcgcctg ccaaatgtaa agcgcagcgc |
| 4651 | ccatccattt gcctttgcgg cagcggggcc acaggcagag cagatcatct |
| 4701 | ctgatccatt gcccctgcca ccttactcgc ctgcaagccc ggtcgcccgt |
| 4751 | gtccatgaac tcgatgggca ggtacttctc ctcggcgtgg gacacgatgc |
| 4801 | caacacgacg ctgcatcttg ccgagttgat ggcaaaggtt ccctatgggg |
| 4851 | tgccgagaca ctgcaccatt cttcaggatg gcaagttggt acgcgtcgat |
| 4901 | tatctcgaga atgaccactg ctgtgagcgc tttgccttgg cgggacaggt |
| 4951 | ggctcaagga gaagagcctt cagaaggaag gtccagtcgg tcatgccttt |
| 5001 | gctcggttga tccgctcccg cgacattgtg gcgacagccc tgggtcaact |
| 5051 | gggccgagat ccgttgatct tcctgcatcc gccagagggc gggatgcgaa |
| 5101 | gaatgcgatg ccgctcgcca gtcgattggc tgagctcatg agcggagaac |
| 5151 | gagatgacgt tggaggggca aggtcgcgct gattgctggg gcaacacggg |
| 5201 | ggatcca |

(C) PMP349-mut glnA1 ΔD54ΔE335 (SEQ ID NO:25)

Full nucleotide sequence of plasmid vector pMP349-mut glnA1 ΔD54ΔE335 used to express the mutant glnA1 in BCG to create GLAD-BCG (plasmid-expressed). It can also be added to 1^{st}, 2^{nd}, and 3^{rd} generation mutants of pro-apoptotic BCG to render, respectively, 2^{nd}, 3^{rd}, and 4^{th} generation pro-apoptotic BCG vaccines.

| | |
|---|---|
| 1 | ctagttccac tgagcgtcag accccgtaga aaagatcaaa ggatcttctt |
| 51 | gagatccttt ttttctgcgc gtaatctgct gcttgcaaac aaaaaaacca |
| 101 | ccgctaccag cggtggtttg tttgccggat caagagctac caactctttt |
| 151 | tccgaaggta actggcttca gcagagcgca gataccaaat actgtccttc |
| 201 | tagtgtagcc gtagttaggc caccacttca agaactctgt agcaccgcct |
| 251 | acatacctcg ctctgctaat cctgttacca gtggctgctg ccagtggcga |
| 301 | taagtcgtgt cttaccgggt tggactcaag acgatagtta ccggataagg |
| 351 | cgcagcggtc gggctgaacg gggggttcgt gcacacagcc cagcttggag |
| 401 | cgaacgacct acaccgaact gagataccta cagcgtgagc attgagaaag |
| 451 | cgccacgctt cccgaaggga gaaaggcgga caggtatccg gtaagcggca |
| 501 | gggtcggaac aggagagcgc acgagggagc ttccaggggg aaacgcctgg |
| 551 | tatctttata gtcctgtcgg gtttcgccac ctctgacttg agcgtcgatt |
| 601 | tttgtgatgc tcgtcagggg ggcggagcct atggaaaaac gccagcaacg |
| 651 | cggccttttt acggttcctg gccttttgct ggccttttgc tcacatgttc |
| 701 | tttcctgcgt tatcccctga ttctgtggat aaccgtatta ccgcctttga |
| 751 | gtgagctgat accgctcgcc gcagccgaac gaccgagcgc aacgcgtgag |
| 801 | cccaccagct ccgtaagttc gggtgctgtg tggctcgtac ccgcgcattc |
| 851 | aggcggcagg gggtctaacg ggtctaaggc ggcgtgtacg gccgccacag |
| 901 | cggctcttag cggcccggaa acgtcctcga aacgacgcat gtgttcctcc |
| 951 | tggttggtac aggtggttgg gggtgctcgg ctgtcgctgg tgtttcatca |
| 1001 | tcagggctcg acgggagagc gggggagtgt gcagttgtgg ggtggcccct |
| 1051 | cagcgaaata tctgacttgg agctcgtgtc ggaccataca ccggtgatta |
| 1101 | atcgtggttt attatcaagc gtgagccacg tcgccgacga atttgagcag |
| 1151 | ctctggctgc cgtactggtc cctggcaagc gacgatctgc tcgaggggat |
| 1201 | ctaccgccaa agccgcgcgt cggccctagg ccgccggtac atcgaggcga |
| 1251 | acccaacagc gctggcaaac ctgctggtcg tggacgtaga ccatccagac |
| 1301 | gcagcgctcc gagcgctcag cgcccggggg tcccatccgc tgcccaacgc |
| 1351 | gatcgtgggc aatcgcgcca acggccacgc acacgcagtg tgggcactca |
| 1401 | acgcccctgt tccacgcacc gaatacgcgc ggcgtaagcc gctcgcatac |
| 1451 | atggcggcgt gcgccgaagg ccttcggcgc gccgtcgatg gcgaccgcag |
| 1501 | ttactcaggc ctcatgacca aaaaccccgg ccacatcgcc tgggaaacgg |
| 1551 | aatggctcca ctcagatctc tacacactca gccacatcga ggccgagctc |
| 1601 | ggcgcgaaca tgccaccgcc gcgctggcgt cagcagacca cgtacaaagc |
| 1651 | ggctccgacg ccgctagggc ggaattgcgc actgttcgat tccgtcaggt |
| 1701 | tgtgggccta tcttcccgcc ctcatgcgga tctacctgcc gacccggaac |
| 1751 | gtggacggac tcggccgcgc gatctatgcc gagtgccacg cgcgaaacgc |
| 1801 | cgaatttccg tgcaacgacg tgtgtcccgg accgctaccg gacagcgagg |
| 1851 | tccgcgccat cgccaacagc atttggcgtt ggatcacaac caagtcgcgc |
| 1901 | atttgggcgg acgggatcgt ggtctacgag gccacactca gtgcgcgcca |
| 1951 | tgcggccatc tcgcggaagg gcgcagcagc gcgcacggcg gcgagcacag |
| 2001 | ttgcgcggcg cgcaaagtcc gcgtcagcca tggaggcatt gctatgagcg |
| 2051 | acggctacag cgacggctac agcgacggct acaactggca gccgactgtc |
| 2101 | cgcaaaaagc ggcgcgtgac cgccgccgaa ggcgctcgaa tcaccggact |
| 2151 | atccgaacgc cacgtcgtcc ggctcgtggc gcaggaacgc agcgagtggt |
| 2201 | tcgccgagca ggctgcacgc cgcgaacgca tccgcgccta tcacgacgac |
| 2251 | gagggccact cttggccgca aacggccaaa catttcgggc tgcatctgga |
| 2301 | caccgttaag cgactcggct atcgggcgag gaaagagcgt gcggcagaac |
| 2351 | aggaagcggc tcaaaaggcc cacaacgaag ccgacaatcc accgctgttc |
| 2401 | taacgcaatt ggggagcggg tgtcgcgggg gttccgtggg gggttccgtt |
| 2451 | gcaacgggtc ggacaggtaa aagtcctggt agacgctagt tttctggttt |
| 2501 | gggccatgcc tgtctcgttg cgtgtttcgt tgcgtccgtt ttgaatacca |
| 2551 | gccagacgag acggggttct acgaatcttg gtcgatacca agccatttcc |
| 2601 | gctgaatatc gtggagctca ccgccagaat cggtggttgt ggtgatgtac |
| 2651 | gtggcgaact ccgttgtagt gcttgtggtg gcatccgtgg cgcggccgcg |
| 2701 | gtaccccaga caactcctta acggtctttc attgccgaaa acgctgacgc |
| 2751 | cctaccgtcg tccaggcggt gtcaacggcg cagcttcact ggtgtgctaa |
| 2801 | ctcgaccatg gcacagcgtg tcaacgctgg tccacccatt tcacttgcga |
| 2851 | atttcggcaa cggcctgcgg actttttgca aattttgcga agtcgcccaa |
| 2901 | aaactgaacc gtttcagaag ctacccgcca gtaacgacaa atccgcaggt |
| 2951 | aaacccacgg atcgacgtcc tgcggatccg gtcacagatt gaacagcgag |
| 3001 | gcgactgcct tgggctcgtc gccaaccaca tatgtgagcg ttgtaacatc |
| 3051 | tagaggtgac cacaacgacg cgcccgcttt gatcggggac gtctgcggcc |
| 3101 | gaccatttac gggtcttgtt gtcgttggcg gtcatgggcc gaacatactc |
| 3151 | acccggatcg gagggccgag gacaaggtcg aacgaggggc atgacccggt |
| 3201 | gcggggcttc ttgcactcgg cataggcgag tgctaagaat aacgttggca |
| 3251 | ctcgcgaccg gtgagtcgta ggtcgggacg gtgaggccag gcccgtcgtc |
| 3301 | gcagcgagtg gcagcgagga caacttgagc cgtccgtcgc gggcactgcg |
| 3351 | cccggccagc gtaagtagcg gggttgccgt cacccggtga cccccggttt |
| 3401 | catccccgat ccggaggaat cacttcgcaa tggccaagac aattgcggat |
| 3451 | ccagctgcag aattcctgca gctcacggta actgatgccg tatttgcagt |
| 3501 | accagcgtac ggcccacaga atgatgtcac gctgaaaatg ccggcctttg |
| 3551 | aatgggttca tgtgcagctc catcagcaaa aggggatgat aagtttatca |
| 3601 | ccaccgacta tttgcaacag tgccgttgat cgtgctatga tcgactgatg |
| 3651 | tcatcagcgg tggagtgcaa tgtcgtgcaa tacgaatggc gaaaagccga |
| 3701 | gctcatcggt cagcttctca accttggggt tacccccggc ggtgtgctgc |
| 3751 | tggtccacag ctccttccgt agcgtccggc ccctcgaaga tgggcccact |
| 3801 | tggactgatc gaggccctgc gtgctacgct gggtccggga gggacgctcg |
| 3851 | tcatgccctc gtggtcaggt ctggacgacg agccgttcga tcctgccacg |
| 3901 | tcgcccgtta caccggacct tggagttgtc tctgacacat tctggcgcct |
| 3951 | gccaaatgta aagcgcagcg cccatccatt tgcctttgcg gcagcggggc |
| 4001 | cacaggcaga gcagatcatc tctgatccat tgcccctgcc accttactcg |
| 4051 | cctgcaagcc cggtcgcccg tgtccatgaa ctcgatgggc aggtacttct |
| 4101 | cctcggcgtg ggacacgatg ccaacacgac gctgcatctt gccgagttga |
| 4151 | tggcaaaggt tccctatggg gtgccgagac actgcaccat tcttcaggat |
| 4201 | ggcaagttgg tacgcgtcga ttatctcgag aatgaccact gctgtgagcg |
| 4251 | ctttgccttg gcgggacagg tggctcaagg agaagagcct tcagaaggaa |
| 4301 | ggtccagtcg gtcatgcctt tgctcggttg atccgctccc gcgacattgt |
| 4351 | ggcgacagcc ctgggtcaac tgggccgaga tccgttgatc ttcctgcatc |
| 4401 | cgccagaggg cgggatgcga agaatgcgat gccgctcgcc agtcgattgg |
| 4451 | ctgagctcat gagcggagaa cgagatgacg ttggaggggc aaggtcgcgc |
| 4501 | tgattgctgg ggcaacacgg gggatccact agtccaccac cagacggccg |
| 4551 | atccccaccg gccgccggcc acccactgcc accacgacca gacccagcat |
| 4601 | caactgcccg ggtgtgaatc cgaacaagcg gaccgccgcc accccgagca |
| 4651 | gcagccaaat caccaggaca accgtcgaca gcatcggggt cgaccaaaca |
| 4701 | ccgaattcca cgcccagcaa cgccagaccg taggcgatca gccagtcgat |
| 4751 | cagcagagcc gccagccggc gccccatcgg agccagcgaa cccggtccgg |
| 4801 | tgtccggcaa gcccagcgtc ttgccgggat agtcgggcgg cgatttcgcc |
| 4851 | gtcatcgggc agacccgata accaggttcc cgttcggcat gccaccggtt |
| 4901 | acgatcttgc cgaccatggc cccacaatag ggccggggag acccggcgtc |
| 4951 | agtggtgggc ggcacggtca gtaacgtctg cgcaacacgg ggttgactga |
| 5001 | cgggcaatat cggctccata gcgtcggccg cggatacagt aaaggagcat |
| 5051 | tctgtgacgg aaaagacgcc cgacgacgtc ttcaaacttg ccaaggacga |
| 5101 | gaaggtcgaa tatgtcgacg tccggttctg tgacctgcct ggcatcatgc |
| 5151 | agcacttcac gattccggct tcggcctttg acaagagcgt gtttgacgac |
| 5201 | ggcttggcct ttggctcgtc gattcgcggg ttccagtcga tccacgaatc |
| 5251 | cgacatgttg cttcttcccg atcccgagac ggcgcgcatc gacccgttcc |
| 5301 | gcgcggccaa gacgctgaat atcaacttct ttgtgcacga cccgttcacc |
| 5351 | ctggagccgt actcccgcga cccgcgcaac atcgcccgca aggccgagaa |
| 5401 | ctacctgatc agcactggca tcgccgacac cgcatacttc ggcgccgagg |
| 5451 | ccgagttcta cattttcgat tcggtgagct tcgactcgcg cgccaacggc |
| 5501 | tccttctacg aggtggacgc catctcgggg tggtggaaca ccggcgcggc |
| 5551 | gaccgaggcc gacggcagtc ccaaccgggg ctacaaggtc cgccacaagg |
| 5601 | gcgggtattt cccagtggcc cccaacgacc aatacgtcga cctgcgcgac |
| 5651 | aagatgctga ccaacctgat caactccggc ttcatcctgg agaagggcca |
| 5701 | ccacgaggtg ggcagcggcg gacaggccga gatcaactac cagttcaatt |
| 5751 | cgctgctgca cgccgccgac gacatgcagt tgtacaagta catcatcaag |
| 5801 | aacaccgcct ggcagaacgg caaaacggtc acgttcatgc ccaagccgct |
| 5851 | gttcggcgac aacgggtccg gcatgcactg tcatcagtcg ctgtggaagg |
| 5901 | acggggcccc gctgatgtac gacgagacgg gttatgccgg tctgtcggac |
| 5951 | acggcccgtc attacatcgg cggcctgtta caccacgcgc cgtcgctgct |
| 6001 | ggccttcacc aacccgacgg tgaactccta caagcggctg gttcccggtt |
| 6051 | acgccccgat caacctggtc tatagccagc gcaaccggtc ggcatgcgtg |
| 6101 | cgcatcccga tcaccggcag caacccgaag gccaagcggc tggagttccg |
| 6151 | aagccccgac tcgtcgggca acccgtatct ggcgttctcg gccatgctga |
| 6201 | tggcaggcct ggacggtatc aagaacaaga tcgagccgca ggcgcccgtc |
| 6251 | gacaaggatc tctacgagct gccgccggaa gaggccgcga gtatcccgca |
| 6301 | gactccgacc cagctgtcag atgtgatcga ccgtctcgag gccgaccacg |
| 6351 | aatacctcac cgaaggaggg gtgttcacaa acgacctgat cgagacgtgg |
| 6401 | atcagtttca agcgcgaaaa cgagatcgag ccggtcaaca tccggccgca |
| 6451 | tccctacgaa ttcgcgctgt actacgacgt ttaaggactc ttcgcagtcc |
| 6501 | gggtgtagag ggagcggcgt gga |

(D) pMP349-mut SodA ΔH28ΔH76, mut glnA1 ΔD54ΔE335 (SEQ ID NO:26)

Full nucleotide sequence of plasmid vector pMP349-mut SodA ΔH28ΔH76, mut glnA1 ΔD54ΔE335 used to simultaneously express the ΔH28ΔH76 mutant sodA and the ΔD54ΔE335 mutant glnA1 in BCG to create GLAD-SAD-BCG ΔH28ΔH76 (plasmid-expressed). It can also be added to 1^{st} and 2^{nd} generation mutants of pro-apoptotic BCG to render, respectively, 3^{rd} and 4^{th} generation pro-apoptotic BCG vaccines.

| | |
|---|---|
| 1 | ctagttccac tgagcgtcag accccgtaga aaagatcaaa ggatcttctt |
| 51 | gagatccttt ttttctgcgc gtaatctgct gcttgcaaac aaaaaaacca |
| 101 | ccgctaccag cggtggtttg tttgccggat caagagctac caactctttt |
| 151 | tccgaaggta actggcttca gcagagcgca gataccaaat actgtccttc |
| 201 | tagtgtagcc gtagttaggc caccacttca agaactctgt agcaccgcct |
| 251 | acatacctcg ctctgctaat cctgttacca gtggctgctg ccagtggcga |
| 301 | taagtcgtgt cttaccgggt tggactcaag acgatagtta ccggataagg |
| 351 | cgcagcggtc gggctgaacg gggggttcgt gcacacagcc cagcttggag |
| 401 | cgaacgacct acaccgaact gagataccta cagcgtgagc attgagaaag |
| 451 | cgccacgctt cccgaaggga gaaaggcgga caggtatccg gtaagcggca |
| 501 | gggtcggaac aggagagcgc acgagggagc ttccaggggg aaacgcctgg |
| 551 | tatctttata gtcctgtcgg gtttcgccac ctctgacttg agcgtcgatt |
| 601 | tttgtgatgc tcgtcagggg ggcggagcct atggaaaaac gccagcaacg |
| 651 | cggccttttt acggttcctg gccttttgct ggccttttgc tcacatgttc |
| 701 | tttcctgcgt tatcccctga ttctgtggat aaccgtatta ccgcctttga |
| 751 | gtgagctgat accgctcgcc gcagccgaac gaccgagcgc aacgcgtgag |
| 801 | cccaccagct ccgtaagttc gggtgctgtg tggctcgtac ccgcgcattc |
| 851 | aggcggcagg gggtctaacg ggtctaaggc ggcgtgtacg gccgccacag |
| 901 | cggctcttag cggcccggaa acgtcctcga aacgacgcat gtgttcctcc |
| 951 | tggttggtac aggtggttgg gggtgctcgg ctgtcgctgg tgtttcatca |
| 1001 | tcagggctcg acgggagagc gggggagtgt gcagttgtgg ggtggcccct |
| 1051 | cagcgaaata tctgacttgg agctcgtgtc ggaccataca ccggtgatta |
| 1101 | atcgtggttt attatcaagc gtgagccacg tcgccgacga atttgagcag |
| 1151 | ctctggctgc cgtactggtc cctggcaagc gacgatctgc tcgaggggat |
| 1201 | ctaccgccaa agccgcgcgt cggccctagg ccgccggtac atcgaggcga |
| 1251 | acccaacagc gctggcaaac ctgctggtcg tggacgtaga ccatccagac |
| 1301 | gcagcgctcc gagcgctcag cgcccggggg tcccatccgc tgcccaacgc |
| 1351 | gatcgtgggc aatcgcgcca acggccacgc acacgcagtg tgggcactca |
| 1401 | acgcccctgt tccacgcacc gaatacgcgc ggcgtaagcc gctcgcatac |
| 1451 | atggcggcgt gcgccgaagg ccttcggcgc gccgtcgatg gcgaccgcag |
| 1501 | ttactcaggc ctcatgacca aaaaccccgg ccacatcgcc tgggaaacgg |
| 1551 | aatggctcca ctcagatctc tacacactca gccacatcga ggccgagctc |
| 1601 | ggcgcgaaca tgccaccgcc gcgctggcgt cagcagacca cgtacaaagc |
| 1651 | ggctccgacg ccgctagggc ggaattgcgc actgttcgat tccgtcaggt |
| 1701 | tgtgggccta tcttcccgcc ctcatgcgga tctacctgcc gacccggaac |
| 1751 | gtggacggac tcggccgcgc gatctatgcc gagtgccacg cgcgaaacgc |
| 1801 | cgaatttccg tgcaacgacg tgtgtcccgg accgctaccg gacagcgagg |
| 1851 | tccgcgccat cgccaacagc atttggcgtt ggatcacaac caagtcgcgc |
| 1901 | atttgggcgg acgggatcgt ggtctacgag gccacactca gtgcgcgcca |
| 1951 | tgcggccatc tcgcggaagg gcgcagcagc gcgcacggcg gcgagcacag |
| 2001 | ttgcgcggcg cgcaaagtcc gcgtcagcca tggaggcatt gctatgagcg |
| 2051 | acggctacag cgacggctac agcgacggct acaactggca gccgactgtc |
| 2101 | cgcaaaaagc ggcgcgtgac cgccgccgaa ggcgctcgaa tcaccggact |
| 2151 | atccgaacgc cacgtcgtcc ggctcgtggc gcaggaacgc agcgagtggt |
| 2201 | tcgccgagca ggctgcacgc cgcgaacgca tccgcgccta tcacgacgac |
| 2251 | gagggccact cttggccgca aacggccaaa catttcgggc tgcatctgga |
| 2301 | caccgttaag cgactcggct atcgggcgag gaaagagcgt gcggcagaac |
| 2351 | aggaagcggc tcaaaaggcc cacaacgaag ccgacaatcc accgctgttc |
| 2401 | taacgcaatt ggggagcggg tgtcgcgggg gttccgtggg gggttccgtt |
| 2451 | gcaacgggtc ggacaggtaa aagtcctggt agacgctagt tttctggttt |
| 2501 | gggccatgcc tgtctcgttg cgtgtttcgt tgcgtccgtt ttgaatacca |
| 2551 | gccagacgag acggggttct acgaatcttg gtcgatacca agccatttcc |
| 2601 | gctgaatatc gtggagctca ccgccagaat cggtggttgt ggtgatgtac |
| 2651 | gtggcgaact ccgttgtagt gcttgtggtg gcatccgtgg cgcggccgcg |
| 2701 | gtaccccatg gtgatgcgcg actccggaat actgagcccg acgcttgcgg |
| 2751 | cagcggggtc agctgaatat caaccccttg gtctgcgagg tcgcggccgc |
| 2801 | ataccgtgac tgcacatcgg cccagttcac gacgttccaa aacgccttgg |
| 2851 | caaagtcgac tttgacgttc ttgtactgca ggtagaaggc gtgttcccac |
| 2901 | atgtcgagca gcagcagcgg aacaatgcct agcgggaagt tcgtctggtg |
| 2951 | gtcgtaaacc tggaatatca gcagcttgtt gccgagtgtg tcccagccca |
| 3001 | gtgccgccca gcccgacccc tgcacggtgg tagcggccgc gtggaactgc |
| 3051 | gcacggaact tgtcgaacga accgaacgcg tcggcgatgg ctgcggcgag |
| 3101 | ttcgccggtg ggcttgtcac caccgttagg cgacaggttc ttccaccaga |
| 3151 | tggtattaac gtggccggcg aggttgaaag ctagattctt ttcgttcagc |
| 3201 | aagatcgctg agtgatcttc cttggcgcgc gcctcttcga gtttggcgac |
| 3251 | ggcgtcattg gcgcccttta cgtaggtggc gtggtgcttg ctgtgaagct |
| 3301 | cgttgatctg acccgagatg tgcggttcca gtgctccgta gtcccagtcc |
| 3351 | aggtctggca aggtgtattc ggccacgggg taccccagac aactccttaa |
| 3401 | cggtctttca ttgccgaaaa cgctgacgcc ctaccgtcgt ccaggcggtg |
| 3451 | tcaacggcgc agcttcactg gtgtgctaac tcgaccatgg cacagcgtgt |
| 3501 | caacgctggt ccacccattt cacttgcgaa tttcggcaac ggcctgcgga |
| 3551 | ctttttgcaa attttgcgaa gtcgcccaaa aactgaaccg tttcagaagc |
| 3601 | tacccgccag taacgacaaa tccgcaggta aacccacgga tcgacgtcct |
| 3651 | gcggatccgg tcacagattg aacagcgagg cgactgcctt gggctcgtcg |
| 3701 | ccaaccacat atgtgagcgt tgtaacatct agaggtgacc acaacgacgc |
| 3751 | gcccgctttg atcggggacg tctgcggccg accatttacg ggtcttgttg |
| 3801 | tcgttggcgg tcatgggccg aacatactca cccggatcgg agggccgagg |
| 3851 | acaaggtcga acgaggggca tgacccggtg cggggcttct tgcactcggc |
| 3901 | ataggcgagt gctaagaata acgttggcac tcgcgaccgg tgagtcgtag |
| 3951 | gtcgggacgg tgaggccagg cccgtcgtcg cagcgagtgg cagcgaggac |
| 4001 | aacttgagcc gtccgtcgcg ggcactgcgc ccggccagcg taagtagcgg |
| 4051 | ggttgccgtc acccggtgac ccccggtttc atccccgatc cggaggaatc |
| 4101 | acttcgcaat ggccaagaca attgcggatc cagctgcaga attcctgcag |
| 4151 | ctcacggtaa ctgatgccgt atttgcagta ccagcgtacg gcccacagaa |
| 4201 | tgatgtcacg ctgaaaatgc cggcctttga atgggttcat gtgcagctcc |
| 4251 | atcagcaaaa ggggatgata agtttatcac caccgactat ttgcaacagt |
| 4301 | gccgttgatc gtgctatgat cgactgatgt catcagcggt ggagtgcaat |
| 4351 | gtcgtgcaat acgaatggcg aaaagccgag ctcatcggtc agcttctcaa |
| 4401 | ccttggggtt acccccggcg gtgtgctgct ggtccacagc tccttccgta |
| 4451 | gcgtccggcc cctcgaagat gggcccactt ggactgatcg aggccctgcg |
| 4501 | tgctacgctg ggtccgggag ggacgctcgt catgccctcg tggtcaggtc |
| 4551 | tggacgacga gccgttcgat cctgccacgt cgcccgttac accggacctt |
| 4601 | ggagttgtct ctgacacatt ctggcgcctg ccaaatgtaa agcgcagcgc |
| 4651 | ccatccattt gcctttgcgg cagcggggcc acaggcagag cagatcatct |
| 4701 | ctgatccatt gcccctgcca ccttactcgc ctgcaagccc ggtcgcccgt |
| 4751 | gtccatgaac tcgatgggca ggtacttctc ctcggcgtgg gacacgatgc |
| 4801 | caacacgacg ctgcatcttg ccgagttgat ggcaaaggtt ccctatgggg |
| 4851 | tgccgagaca ctgcaccatt cttcaggatg gcaagttggt acgcgtcgat |
| 4901 | tatctcgaga atgaccactg ctgtgagcgc tttgccttgg cgggacaggt |
| 4951 | ggctcaagga gaagagcctt cagaaggaag gtccagtcgg tcatgccttt |
| 5001 | gctcggttga tccgctcccg cgacattgtg gcgacagccc tgggtcaact |
| 5051 | gggccgagat ccgttgatct tcctgcatcc gccagagggc gggatgcgaa |
| 5101 | gaatgcgatg ccgctcgcca gtcgattggc tgagctcatg agcggagaac |
| 5151 | gagatgacgt tggaggggca aggtcgcgct gattgctggg gcaacacggg |
| 5201 | ggatccacta gtccaccacc agacggccga tccccaccgg ccgccggcca |
| 5251 | cccactgcca ccacgaccag acccagcatc aactgcccgg gtgtgaatcc |
| 5301 | gaacaagcgg accgccgcca ccccgagcag cagccaaatc accaggacaa |
| 5351 | ccgtcgacag catcggggtc gaccaaacac cgaattccac gcccagcaac |
| 5401 | gccagaccgt aggcgatcag ccagtcgatc agcagagccg ccagccggcg |
| 5451 | ccccatcgga gccagcgaac ccggtccggt gtccggcaag cccagcgtct |
| 5501 | tgccgggata gtcgggcggc gatttcgccg tcatcgggca gacccgataa |
| 5551 | ccaggttccc gttcggcatg ccaccggtta cgatcttgcc gaccatggcc |
| 5601 | ccacaatagg gccggggaga cccggcgtca gtggtgggcg gcacggtcag |
| 5651 | taacgtctgc gcaacacggg gttgactgac gggcaatatc ggctccatag |
| 5701 | cgtcggccgc ggatacagta aaggagcatt ctgtgacgga aaagacgccc |
| 5751 | gacgacgtct tcaaacttgc caaggacgag aaggtcgaat atgtcgacgt |
| 5801 | ccggttctgt gacctgcctg gcatcatgca gcacttcacg attccggctt |
| 5851 | cggcctttga caagagcgtg tttgacgacg gcttggcctt tggctcgtcg |
| 5901 | attcgcgggt tccagtcgat ccacgaatcc gacatgttgc ttcttcccga |
| 5951 | tcccgagacg gcgcgcatcg acccgttccg cgcggccaag acgctgaata |
| 6001 | tcaacttctt tgtgcacgac ccgttcaccc tggagccgta ctcccgcgac |
| 6051 | ccgcgcaaca tcgcccgcaa ggccgagaac tacctgatca gcactggcat |
| 6101 | cgccgacacc gcatacttcg gcgccgaggc cgagttctac attttcgatt |
| 6151 | cggtgagctt cgactcgcgc gccaacggct ccttctacga ggtggacgcc |
| 6201 | atctcggggt ggtggaacac cggcgcggcg accgaggccg acggcagtcc |
| 6251 | caaccggggc tacaaggtcc gccacaaggg cgggtatttc ccagtggccc |
| 6301 | ccaacgacca atacgtcgac ctgcgcgaca agatgctgac caacctgatc |
| 6351 | aactccggct tcatcctgga gaagggccac cacgaggtgg gcagcggcgg |
| 6401 | acaggccgag atcaactacc agttcaattc gctgctgcac gccgccgacg |
| 6451 | acatgcagtt gtacaagtac atcatcaaga acaccgcctg gcagaacggc |
| 6501 | aaaacggtca cgttcatgcc caagccgctg ttcggcgaca acgggtccgg |
| 6551 | catgcactgt catcagtcgc tgtggaagga cggggccccg ctgatgtacg |
| 6601 | acgagacggg ttatgccggt ctgtcggaca cggcccgtca ttacatcggc |
| 6651 | ggcctgttac accacgcgcc gtcgctgctg gccttcacca acccgacggt |
| 6701 | gaactcctac aagcggctgg ttcccggtta cgccccgatc aacctggtct |
| 6751 | atagccagcg caaccggtcg gcatgcgtgc gcatcccgat caccggcagc |
| 6801 | aacccgaagg ccaagcggct ggagttccga agccccgact cgtcgggcaa |
| 6851 | cccgtatctg gcgttctcgg ccatgctgat ggcaggcctg gacggtatca |
| 6901 | agaacaagat cgagccgcag gcgcccgtcg acaaggatct ctacgagctg |
| 6951 | ccgccggaag aggccgcgag tatcccgcag actccgaccc agctgtcaga |
| 7001 | tgtgatcgac cgtctcgagg ccgaccacga atacctcacc gaaggagggg |
| 7051 | tgttcacaaa cgacctgatc gagacgtgga tcagtttcaa gcgcgaaaac |
| 7101 | gagatcgagc cggtcaacat ccggccgcat ccctacgaat tcgcgctgta |
| 7151 | ctacgacgtt taaggactct tcgcagtccg ggtgtagagg gagcggcgtg |
| 7201 | ga |

(E) pHV203-mut glnA1 ΔD54ΔE335 (SEQ ID NO:27)

Full nucleotide sequence of plasmid vector pHV203-mut glnA1 ΔD54ΔE335 used to express the mutant glnA1 in BCG to create GLAD-BCGΔD54ΔE335 (plasmid-expressed). It can also be added to 1^{st}, 2^{nd}, and 3^{rd} generation mutants of pro-apoptotic BCG to render, respectively, 2^{nd}, 3^{rd}, and 4^{th} generation pro-apoptotic BCG vaccines.

| | |
|---|---|
| 1 | aagctttaat gcggtagttt atcacagtta aattgctaac gcagtcaggc accgtgtatg |
| 61 | aaatctaaca atgcgctcat cgtcatcctc ggcaccgtca ccctggatgc tgtaggcata |
| 121 | ggcttggtta tgccggtact gccgggcctc ttgcgggata tcgagccgag aacgttatcg |
| 181 | aagttggtca tgtgtaatcc cctcgtttga actttggatt aagcgtagat acacccttgg |
| 241 | acaagccagt tggattcgga gacaagcaaa ttcagcctta aaaagggcga ggccctgcgg |
| 301 | tggtggaaca ccgcagggcc tctaaccgct cgacgcgctg caccaaccag cccgcgaacg |
| 361 | gctggcagcc agcgtaaggc gcggctcatc gggcggcgtt cgccacgatg tcctgcactt |
| 421 | cgagccaagc ctcgaacacc tgctggtgtg cacgactcac ccggttgttg acaccgcgcg |
| 481 | cggccgtgcg ggctcggtgg ggcggctctg tcgcccttgc cagcgtgagt agcgcgtacc |
| 541 | tcacctcgcc caacaggtcg cacacagccg attcgtacgc cataaagcca ggtgagccca |
| 601 | ccagctccgt aagttcgggc gctgtgtggc tcgtacccgc gcattcaggc ggcagggggt |
| 661 | ctaacgggtc taaggcggcg tgtacgcggc cacagcggct ctcagcggcc cggaaacgtc |
| 721 | ctcgaaacga cgcatgtgtt cctcctggtt ggtacaggtg gttgggggtg ctcggctgtc |
| 781 | gcggttgttc caccaccagg gctcgacggg agagcggggg agtgtgcagt tgtggggtgg |
| 841 | cccctcagcg aaatatctga cttggagctc gtgtcggacc atacaccggt gattaatcgt |
| 901 | ggtctactac caagcgtgag ccacgtcgcc gacgaatttg agcagctctg gctgccgtac |
| 961 | tggccgctgg caagcgacga tctgctcgag gggatctacc gccaaagccg cgcgtcggcc |
| 1021 | ctaggccgcc ggtacatcga ggcgaaccca acagcgctgg caaacctgct ggtcgtggac |
| 1081 | gtagaccatc cagacgcagc gctccgagcg ctcagcgccc gggggtccca tccgctgccc |
| 1141 | aacgcgatcg tgggcaatcg cgccaacggc cacgcacacg cagtgtgggc actcaacgcc |
| 1201 | cctgttccac gcaccgaata cgcgcggcgt aagccgctcg catacatggc ggcgtgcgcc |
| 1261 | gaaggccttc ggcggccgtc gacggcgacc gcagttactc aggcctcatg accaaaaacc |
| 1321 | ccggccacat cgcctgggaa acggaatggc tccactcaga tctctacaca ctcagccaca |
| 1381 | tcgaggccga gctcggcgcg aacatgccac cgccgcgctg gcgtcagcag accacgtaca |
| 1441 | aagcggctcc gacgccgcta gggcggaatt gcgcactgtt cgattccgtc aggttgtggg |
| 1501 | cctatcgtcc cgccctcatg cggatctacc tgccgacccg gaacgtggac ggactcggcc |
| 1561 | gcgcgatcta tgccgagtgc cacgcgcgaa acgccgaatt cccgtgcaac gacgtgtgtc |
| 1621 | ccggaccgct accggacagc gaggtccgcg ccatcgccaa cagcatttgg cgttggatca |
| 1681 | caaccaagtc gcgcatttgg gcggacggga tcgtggtcta cgaggccaca ctcagtgcgc |
| 1741 | gccagtcggc catctcgcgg aagggcgcag cagcgcgcac ggcggcgagc acagttgcgc |
| 1801 | ggcgcgcaaa gtccgcgtca gccatggagg cattgctatg agcgacggct acagcgacgg |
| 1861 | ctacagcgac ggctacaacc ggcagccgac tgtccgcaaa aagccgtgac gcgccgaagg |
| 1921 | cgctcgaatc accggactat ccgaacgcca cgtcgtccgg ctcgtggcgc aggaacgcag |
| 1981 | cgagtggctc gccgagcagg ctgcacgcgc gcgaagcatc cgcgcctatc acgacgacga |
| 2041 | gggccactct tggccgcaaa cggccaaaca tttcgggctg catctggaca ccgttaagcg |
| 2101 | actcggctat cgggcgagga aagagcgtgc ggcagaacag gaagcggctc aaaaggccca |
| 2161 | caacgaagcc gacaatccac cgctgttcta acgcaattgg ggacgggtgt cgcgggggtt |
| 2221 | ccgtgggggg ttccgttgca acgggtcgga caggtaaaag tcctggtaga cgctagtttt |
| 2281 | ctggtttggg ccatgcctgt ctcgttgcgt gtttcgttgc gccgttttga ataccagcca |
| 2341 | gacgagacgg ggttctacga atcttggtcg ataccaagcc atttccgctg aatatcgggg |
| 2401 | agctcaccgc cagaatcggt ggttgtggtg atgtacgtgg cgaactccgt tgtagtgcct |
| 2461 | gtggtggcat ccgtggccac tctcgttgca cggttcgttg tgccgttaca ggccccgttg |
| 2521 | acagctcacc gaacgtagtt aaaacatgct ggtcaaacta ggtttaccaa cgatacgagt |
| 2581 | cagctcatct agggccagtt ctaggcgttg ttcgttgcgc ggttcgttgc gcatgtttcg |
| 2641 | tgtggttgct agatggctcc gcaaccacac gcttcgaggt tgagtgcttc cagcacgggc |
| 2701 | gcgatccaga agaacttcgt cgtgcgactg tcctcgttat cgtccattcc gacagcatcg |
| 2761 | ccagtcacta tggcgtgctg ctagcgctat atgcgttgat gcaatttcta tgcgcacccg |
| 2821 | ttctcggagc actgtccgac cgctttggcc gccgcccagt cctgctcgct tcgctacttg |
| 2881 | gagccactat cgactacgcg atcatggcga ccacacccgt cctgtggatc cactagtcca |
| 2941 | cgccgctccc tctacacccg gactgcgaag agtccttaaa cgtcgtagta cagcgcgaat |
| 3001 | tcgtagggat gcggccggat gttgaccggc tcgatctcgt tttcgcgctt gaaactgatc |
| 3061 | cacgtctcga tcaggtcgtt tgtgaacacc cctccttcgg tgaggtattc gtggtcggcc |
| 3121 | tcgagacggt cgatcacatc tgacagctgg gtcggagtct gcgggatact cgcggcctct |
| 3181 | tccggcggca gctcgtagag atccttgtcg acgggcgcct gcggctcgat cttgttcttg |
| 3241 | ataccgtcca ggcctgccat cagcatggcc gagaacgcca gatacgggtt gcccgacgag |
| 3301 | tcggggcttc ggaactccag ccgcttggcc ttcgggttgc tgccggtgat cgggatgcgc |
| 3361 | acgcatgccg accggttgcg ctggctatag accaggttga tcggggcgta accgggaacc |
| 3421 | agccgcttgt aggagttcac cgtcgggttg gtgaaggcca gcagcgacgg cgcgtggtgt |
| 3481 | aacaggccgc cgatgtaatg acgggccgtg tccgacagac cggcataacc cgtctcgtcg |
| 3541 | tacatcagcg gggccccgtc cttccacagc gactgatgac agtgcatgcc ggacccgttg |
| 3601 | tcgccgaaca gcggcttggg catgaacgtg accgttttgc cgttctgcca ggcggtgttc |
| 3661 | ttgatgatgt acttgtacaa ctgcatgtcg tcggcggcgt gcagcagcga attgaactgg |
| 3721 | tagttgatct cggcctgtcc gccgctgccc acctcgtggt ggcccttctc caggatgaag |
| 3781 | ccggagttga tcaggttggt cagcatcttg tcgcgcaggt cgacgtattg gtcgttgggg |
| 3841 | gccactggga aatacccgcc cttgtggcgg accttgtagc cccggttggg actgccgtcg |
| 3901 | gcctcggtcg ccgcgccggt gttccaccac cccgagatgg cgtccacctc gtagaaggag |
| 3961 | ccgttggcgc gcgagtcgaa gctcaccgaa tcgaaaatgt agaactcggc ctcggcgccg |
| 4021 | aagtatgcgg tgtcggcgat gccagtgctg atcaggtagt tctcggcctt gcgggcgatg |
| 4081 | ttgcgcgggt cgcgggagta cggctccagg gtgaacgggt cgtgcacaaa gaagttgata |
| 4141 | ttcagcgtct tggccgcgcg gaacgggtcg atgcgcgccg tctcgggatc gggaagaagc |
| 4201 | aacatgtcgg attcgtggat cgactggaac ccgcgaatcg acgagccaaa ggccaagccg |
| 4261 | tcgtcaaaca cgctcttgtc aaaggccgaa gccggaatcg tgaagtgctg catgatgcca |
| 4321 | ggcaggtcac agaaccggac gtcgacatat tcgaccttct cgtccttggc aagtttgaag |
| 4381 | acgtcgtcgg gcgtcttttc cgtcacagaa tgctccttta ctgtatccgc ggccgacgct |
| 4441 | atggagccga tattgcccgt cagtcaaccc cgtgttgcgc agacgttact gaccgtgccg |
| 4501 | cccaccactg acgccgggtc tccccggccc tattgtgggg ccatggtcgg caagatcgta |
| 4561 | accggtggca tgccgaacgg gaacctggtt atcgggtctg cccgatgacg gcgaaatcgc |
| 4621 | cgcccgacta tcccggcaag acgctgggct tgccggacac cggaccgggt tcgctggctc |
| 4681 | cgatggggcg ccggctggcg gctctgctga tcgactggct gatcgcctac ggtctggcgt |
| 4741 | tgctgggcgt ggaattcggt gtttggtcga ccccgatgct gtcgacggtt gtcctggtga |
| 4801 | tttggctgct gctcggggtg gcggcggtcc gcttgttcgg attcacaccc gggcagttga |
| 4861 | tgctgggtct ggtcgtggtg gcagtgggtg gccggcggcc ggtggggatc ggccgtctgg |
| 4921 | tggtggacta gttctagaga cgggcctctt cgtcgtacgc aattgtcttg gccattgcga |
| 4981 | agtgattcct ccggatcggg gatgaaacgg gggtcaccgg gtgacggcaa ccccgctact |
| 5041 | tacgctggcc gggcgcagtg cccgcgacgg acggctcaag ttgtcctcgc tgccactcgc |
| 5101 | tgcgacgacg ggcctggcct caccgtcccg acctagcact caccggtcgc gagtgccaac |
| 5161 | gttattctta gcactcgcct atgccgagtg caagaagccc cgcaccgggt catgcccctc |
| 5221 | gttcgaccgt gtcctcggcc ctccgatccg ggtgagtatg ttcggcccat gaccgccaac |
| 5281 | gacaacaaga cccgtaaatg gtcggccgca gacgtccccg atcaaagcgg gcgcgtcgtt |
| 5341 | gtggtcaccg gcgccaacac cggcatcggc taccacaccg ccgccgtgtt tgccgaccgc |
| 5401 | ggtgcacacg tagtgttggc cgtccgcaat ctcgagaagg gcaacgccgc ccgggcccgc |
| 5461 | atcatgcggc cgccaccgcg gtggagctcc agcttttgtt ccctttagtg agggttaatt |
| 5521 | gcgcgcttgg cgtaatcatg gtcatagctg tttcctgtgt gaaattgtta tccgctcaca |
| 5581 | attccacaca acatacgagc cggaagcata aagtgtaaag cctggggtgc ctaatgagtg |
| 5641 | agctaactca cattaattgc gttgcgctca ctgcccgctt tccagtcggg aaacctgtcg |
| 5701 | tgccagctgc attaatgaat cggccaacgc gcggggagag gcggtttgcg tattgggcgc |
| 5761 | tcttccgctt cctcgctcac tgactcgctg cgctcggtcg ttcggctgcg gcgagcggta |
| 5821 | tcagctcact caaaggcggt aatacggtta tccacagaat caggggataa cgcaggaaag |
| 5881 | aacatgtgag caaaaggcca gcaaaaggcc aggaaccgta aaaaggccgc gttgctggcg |
| 5941 | tttttccata ggctccgccc ccctgacgag catcacaaaa atcgacgctc aagtcagagg |
| 6001 | tggcgaaacc cgacaggact ataaagatac caggcgtttc cccctggaag ctccctcgtg |
| 6061 | cgctctcctg ttccgaccct gccgcttacc ggatacctgt ccgcctttct cccttcggga |
| 6121 | agcgtggcgc tttctcatag ctcacgctgt aggtatctca gttcggtgta ggtcgttcgc |
| 6181 | tccaagctgg gctgtgtgca cgaacccccc gttcagcccg accgctgcgc cttatccggt |
| 6241 | aactatcgtc ttgagtccaa cccggtaaga cacgacttat cgccactggc agcagccact |
| 6301 | ggtaacagga ttagcagagc gaggtatgta ggcggtgcta cagagttctt gaagtggtgg |
| 6361 | cctaactacg gctacactag aaggacagta tttggtatct gcgctctgct gaagccagtt |
| 6421 | accttcggaa aaagagttgg tagctcttga tccggcaaac aaaccaccgc tggtagcggt |
| 6481 | ggtttttttg tttgcaagca gcagattacg cgcagaaaaa aaggatctca agaagatcct |
| 6541 | ttgatctttt ctacggggtc tgacgctcag tggaacgaaa actcacgtta agggattttg |
| 6601 | gtcatgagat tatcaaaaag gatcttcacc tagatccttt tcgaccgaat aaatacctgt |
| 6661 | gacggaagat cacttcgcag aataaataaa tcctggtgtc cctgttgata ccgggaagcc |
| 6721 | ctgggccaac ttttggcgaa aatgagacgt tgatcggcac gtaagaggtt ccaactttca |
| 6781 | ccataatgaa ataagatcac taccgggcgt attttttgag ttgtcgagat tttcaggagc |
| 6841 | taaggaagct aaaatggaga aaaaaatcac tggatatacc accgttgata tatcccaatg |
| 6901 | gcatcgtaaa gaacattttg aggcatttca gtcagttgct caatgtacct ataaccagac |
| 6961 | cgttcagctg gatattacgg cctttttaaa gaccgtaaag aaaaataagc acaagtttta |
| 7021 | tccggccttt attcacattc ttgcccgcct gatgaatgct catccggaat tacgtatggc |
| 7081 | aatgaaagac ggtgagctgg tgatatggga tagtgttcac ccttgttaca ccgttttcca |
| 7141 | tgagcaaact gaaacgtttt catcgctctg gagtgaatac cacgacgatt tccggcagtt |
| 7201 | tctacacata tattcgcaag atgtggcgtg ttacggtgaa aacctggcct atttccctaa |
| 7261 | agggtttatt gagaatatgt ttttcgtctc agccaatccc tgggtgagtt tcaccagttt |
| 7321 | tgatttaaac gtggccaata tggacaactt cttcgccccc gttttcacca tgggcaaata |
| 7381 | ttatacgcaa ggcgacaagg tgctgatgcc gctggcgatt caggttcatc atgccgtttg |
| 7441 | tgatggcttc catgtcggca gaatgcttaa tgaattacaa cagtactgcg atgagtggca |
| 7501 | gggcggggcg taattttttt aaggcagtta ttggtgccct taaacgcctg gttgctacgc |
| 7561 | ctgaataagt gataataagc ggatgaatgg cagaaattcg aaagcaaatt cgacccggtc |
| 7621 | gtcggttcag ggcagggtcg ttaaatagcc gcttatgtct attgctggtt taccggttta |
| 7681 | ttgactaccg gaagcagtgt gaccgtgtgc ttctcaaatg cctgaggcca gtttgctcag |
| 7741 | gctctccccg tggaggtaat aattgacgat atgatccttt ttttctgatc aaaagtgctc |
| 7801 | atcattggaa aacgttcttc ggggcgaaaa ctctcaagga tcttaccgct gttgagatcc |
| 7861 | agttcgatgt aacccactcg tgcacccaac tgatcttcag catcttttac tttcaccagc |
| 7921 | gtttctgggt gagcaaaaac aggaaggcaa aatgccgcaa aaaagggaat aagggcgaca |
| 7981 | cggaaatgtt gaatactcat actcttcctt tttcaatatt attgaagcat ttatcaaggg |
| 8041 | ttattgtctc atgagcggat acatatttga atgtatttag aaaaataaac aaataggggt |
| 8101 | tccgcgcaca tttccccgaa aagtgccacc taaattgtaa gcgttaatat tttgttaaaa |
| 8161 | ttcgcgttaa atttttgtta aatcagctca ttttttaacc aataggccga aatcggcaaa |
| 8221 | atcccttata aatcaaaaga atagaccgag atagggttga gtgttgttcc agtttggaac |
| 8281 | aagagtccac tattaaagaa cgtggactcc aacgtcaaag ggcgaaaaac cgtctatcag |
| 8341 | ggcgatggcc cactacgtga accatcaccc taatcaagtt ttttggggtc gaggtgccgt |
| 8401 | aaagcactaa atcggaaccc taaagggagc ccccgattta gagcttgacg gggaaagccg |
| 8461 | gcgaacgtgg cgagaaagga agggaagaaa gcgaaaggag cgggcgctag ggcgctggca |
| 8521 | agtgtagcgg tcacgctgcg cgtaaccacc acacccgccg cgcttaatgc gccgctacag |
| 8581 | ggcgcgtccc attcgccatt caggctgcgc aactgttggg aagggcgatc ggtgcgggcc |
| 8641 | tcttcgctat tacgccagct ggcgaaaggg ggatgtgctg caaggcgatt aagttgggta |
| 8701 | acgccagggt tttcccagtc acgacgttgt aaaacgacgg ccagtgagcg cgcgtaatac |
| 8761 | gactcactat agggcgaatt gggtaccggg ccccccctcg aggtcgacgg tatcgataag |
| 8821 | cttagcaaaa gttcgattta ttcaacaaag ccgccgtccc gtcaagtcag cgtaatgctc |
| 8881 | tgccagtgtt acaaccaatt aaccaattct gattagaaaa actcatcgag catcaaatga |
| 8941 | aactgcaatt tattcatatc aggattatca ataccatatt tttgaaaaag ccgtttctgt |
| 9001 | aatgaaggag aaaactcacc gaggcagttc cataggatgg caagatcctg gtatcggtct |
| 9061 | gcgattccga ctcgtccaac atcaatacaa cctattaatt tcccctcgtc aaaaataagg |
| 9121 | ttatcaagtg agaaatcacc atgagtgacg actgaatccg gtgagaatgg caaaagatta |
| 9181 | tgcatttctt tccagacttg ttcaacaggc cagccattac gctcgtcatc aaaatcactc |
| 9241 | gcatcaacca aaccgttatt cattcgtgat tgcgcctgag cgagacgaaa tacgcgatcg |
| 9301 | ctgttaaaag gacaattaca aacaggaatc gaatgcaacc ggcgcaggaa cactgccagc |
| 9361 | gcatcaacaa tattttcacc tgaatcagga tattcttcta atacctggaa tgctgttttc |
| 9421 | ccggggatcg cagtggtgag taaccatgca tcatcaggag tacggataaa atgcttgatg |
| 9481 | gtcggaagag gcataaattc cgtcagccag tttagtctga ccatctcatc tgtaacatca |
| 9541 | ttggcaacgc tacctttgcc atgtttcaga aacaactctg gcgcatgggg cttcccatac |
| 9601 | aagcgataga ttgtcgcacc tgattgcccg acattatcgc gagcccattt atacccatat |
| 9661 | aaatcagcat ccatgttgga atttaatcgc ggcctcgagc aagacgtttc ccgttgaata |
| 9721 | tggctcataa caccccttgt attactgttt atgtaagcag acagttttat tgttcatgat |
| 9781 | gatatatttt tatcttgtgc aatgtaacat cagagatttt gagacacaac gtcgctttgt |
| 9841 | tggctagctc acacaaccgg tcgtgacttt tagggctccg agagaagctc ctcgatgtcg |
| 9901 | tctggccacg accagaggag ttcaccctcg gcggtgaggt tggtgtgctc gttcacccgg |
| 9961 | atcaggagat cgtcatcctc gatgcctcgg gggacgtacc tgaacccgcc gccggccata |
| 10021 | ccttcgt |

(F) pMP399-mut SodA ΔE54 (SEQ ID NO:28)

Full nucleotide sequence of chromosomal integration vector pMV399-mut SodA ΔE54 used to express the mutant sodA in BCG to create SAD-BCGΔE54 (chromosome-expressed). It can also be added to 1^{st}, and 2^{nd}, and 3^{rd} generation mutants of pro-apoptotic BCG to construct, respectively, 2^{nd}, 3^{rd}, and 4^{th} generation pro-apoptotic BCG vaccines.

| | |
|---|---|
| 1 | ggtaccccgt ggccgaatac accttgccag acctggactg ggactacgga |
| 51 | gcactggaac cgcacatctc gggtcagatc aacgagcttc gccacagcaa |
| 101 | gcaccacgcc acctacgtaa agggcgccaa tgacgccgtc gccaaactcg |
| 151 | aagaggcgcg cgccaaggat cactcagcga tcatgctgaa cgaaaagaat |
| 201 | ctagctttca acctcgccgg ccacgttaat cacaccatct ggtggaagaa |
| 251 | cctgtcgcct aacggtggtg acaagcccac cggcgaactc gccgcagcca |
| 301 | tcgccgacgc gttcggttcg ttcgacaagt tccgtgcgca gttccacgcg |
| 351 | gccgctacca ccgtgcaggg gtcgggctgg gcggcactgg gctgggacac |
| 401 | actcggcaac aagctgctga tattccaggt ttacgaccac cagacgaact |
| 451 | tcccgctagg cattgttccg ctgctgctgc tcgacatgtg ggaacacgcc |
| 501 | ttctacctgc agtacaagaa cgtcaaagtc gactttgcca aggcgttttg |
| 551 | gaacgtcgtg aactgggccg atgtgcagtc acggtatgcg gccgcgacct |
| 601 | cgcagaccaa ggggttgata ttcagctgac cccgctgccg caagcgtcgg |
| 651 | gctcagtatt ccggagtcgc gcatcaccat ggggtacctc tagagtcgac |
| 701 | caccaagggc accatctctg cttgggccac cccgttggcc gcagccagct |
| 751 | cgctgagagc cgtgaacgac agggcgaacg ccagcccgcc gacggcgagg |
| 801 | gttccgaccg ctgcaactcc cggtgcaacc ttgtcccggt ctattctctt |
| 851 | cactgcacca gctccaatct ggtgtgaatg cccctcgtct gttcgcgcag |
| 901 | gcggggggct ctattcgttt gtcagcatcg aaagtagcca gatcagggat |
| 951 | gcgttgcaac cgcgtatgcc caggtcagaa gagtcgcaca agagttgcag |
| 1001 | acccctggaa agaaaaatgg ccagagggcg aaaacaccct ctgaccagcg |
| 1051 | gagcgggcga cgggaatcga acccgcgtag ctagtttgga agaatgggtg |
| 1101 | tctgccgacc acatatgggc cggtcaagat aggtttttac cccctctcgg |
| 1151 | ctgcatcctc taagtggaaa gaaattgcag gtcgtagaag cgcgttgaag |
| 1201 | cctgagagtt gcacaggagt tgcaacccgg tagccttgtt cacgacgaga |
| 1251 | ggagacctag ttggcacgtc gcggatgggg atcgctgaag actcagcgca |
| 1301 | gcgggaggat ccaagcctca tacgtcaacc cgcaggacgg tgtgaggtac |
| 1351 | tacgcgctgc agacctacga caacaagatg gacgccgaag cctggctcgc |
| 1401 | gggcgagaag cggctcatcg agatggagac ctggacccct ccacaggacc |
| 1451 | gggcgaagaa ggcagccgcc agcgccatca cgctggagga gtacacccgg |
| 1501 | aagtggctcg tggagcgcga cctcgcagac ggcaccaggg atctgtacag |
| 1551 | cgggcacgcg gagcgccgca tctacccggt gctaggtgaa gtggcggtca |
| 1601 | cagagatgac gccagctctg gtgcgtgcgt ggtgggccgg gatgggtagg |
| 1651 | aagcacccga ctgcccgccg gcatgcctac aacgtcctcc gggcggtgat |
| 1701 | gaacacagcg gtcgaggaca agctgatcgc agagaacccg tgccggatcg |
| 1751 | agcagaaggc agccgatgag cgcgacgtag aggcgctgac gcctgaggag |
| 1801 | ctggacatcg tcgccgctga gatcttcgag cactaccgga tcgcggcata |
| 1851 | catcctggcg tggacgagcc tccggttcgg agagctgatc gagcttcgcc |
| 1901 | gcaaggacat cgtggacgac ggcatgacga tgaagctccg ggtgcgccgt |
| 1951 | ggcgcttccc gcgtggggaa caagatcgtc gttggcaacg ccaagaccgt |
| 2001 | ccggtcgaag cgtcctgtga cggttccgcc tcacgtcgcg gagatgatcc |
| 2051 | gagcgcacat gaaggaccgt acgaagatga acaagggccc cgaggcattc |
| 2101 | ctggtgacca cgacgcaggg caaccggctg tcgaagtccg cgttcaccaa |
| 2151 | gtcgctgaag cgtggctacg ccaagatcgg tcggccggaa ctccgcatcc |
| 2201 | acgacctccg cgctgtcggc gctacgttcg ccgctcaggc aggtgcgacg |
| 2251 | accaaggagc tgatggcccg tctcggtcac acgactccta ggatggcgat |
| 2301 | gaagtaccag atggcgtctg aggcccgcga cgaggctatc gctgaggcga |
| 2351 | tgtccaagct ggccaagacc tcctgaaacg caaaaagccc ccctcccaag |
| 2401 | gacactgagt cctaaagagg ggggtttctt gtcagtacgc gaagaaccac |
| 2451 | gcctggccgc gagcgccagc accgccgctc tgtgcggaga cctgggcacc |
| 2501 | agccccgccg ccgccaggag cattgccgtt cccgccagaa atctagaggt |
| 2551 | gaccacaacg acgcgcccgc tttgatcggg gacgtctgcg gccgaccatt |
| 2601 | tacgggtctt gttgtcgttg gcggtcatgg gccgaacata ctcacccgga |
| 2651 | tcggagggcc gaggacaagg tcgaacgagg ggcatgaccc ggtgcggggc |
| 2701 | ttcttgcact cggcataggc gagtgctaag aataacgttg gcactcgcga |
| 2751 | ccggtgagtc gtaggtcggg acggtgaggc caggcccgtc gtcgcagcga |
| 2801 | gtggcagcga ggacaacttg agccgtccgt cgcgggcact gcgcccggcc |
| 2851 | agcgtaagta gcggggttgc cgtcacccgg tgacccccgg tttcatcccc |
| 2901 | gatccggagg aatcacttcg caatggccaa gacaattgcg gatccagctg |
| 2951 | cagaattcga agcttatcga tgtcgacgta gttaactagc gtacgatcga |
| 3001 | ctgccaggca tcaaataaaa cgaaaggctc agtcgaaaga ctgggccttt |
| 3051 | cgttttatct gttgtttgtc cggccatcat ggccgcggtg atcagctagc |
| 3101 | caacaaagcg acgttgtgtc tcaaaatctc tgatgttaca ttgcacaaga |
| 3151 | taaaaatata tcatcatgat cgaattcctg cagctcacgg taactgatgc |
| 3201 | cgtatttgca gtaccagcgt acggcccaca gaatgatgtc acgctgaaaa |
| 3251 | tgccggcctt tgaatgggtt catgtgcagc tccatcagca aaaggggatg |
| 3301 | ataagtttat caccaccgac tatttgcaac agtgccgttg atcgtgctat |
| 3351 | gatcgactga tgtcatcagc ggtggagtgc aatgtcgtgc aatacgaatg |
| 3401 | gcgaaaagcc gagctcatcg gtcagcttct caaccttggg gttacccccg |
| 3451 | gcggtgtgct gctggtccac agctccttcc gtagcgtccg gcccctcgaa |
| 3501 | gatgggccac ttggactgat cgaggccctg cgtgctacgc tgggtccggg |
| 3551 | agggacgctc gtcatgccct cgtggtcagg tctggacgac gagccgttcg |
| 3601 | atcctgccac gtcgcccgtt acaccggacc ttggagttgt ctctgacaca |
| 3651 | ttctggcgcc tgccaaatgt aaagcgcagc gcccatccat ttgcctttgc |
| 3701 | ggcagcgggg ccacaggcag agcagatcat ctctgatcca ttgcccctgc |
| 3751 | caccttactc gcctgcaagc ccggtcgccc gtgtccatga actcgatggg |
| 3801 | caggtacttc tcctcggcgt gggacacgat gccaacacga cgctgcatct |
| 3851 | tgccgagttg atggcaaagg ttccctatgg ggtgccgaga cactgcacca |
| 3901 | ttcttcagga tggcaagttg gtacgcgtcg attatctcga gaatgaccac |
| 3951 | tgctgtgagc gctttgcctt ggcggacagg tggctcaagg agaagagcct |
| 4001 | tcagaaggaa ggtccagtcg gtcatgcctt tgctcggttg atccgctccc |
| 4051 | gcgacattgt ggcgacagcc ctgggtcaac tgggccgaga tccgttgatc |
| 4101 | ttcctgcatc cgccagaggc gggatgcgaa gaatgcgatg ccgctcgcca |
| 4151 | gtcgattggc tgagctcatg agcggagaac gagatgacgt tggaggggca |
| 4201 | aggtcgcgct gattgctggg gcaacacggg ggatccacta gttccactga |
| 4251 | gcgtcagacc ccgtagaaaa gatcaaagga tcttcttgag atcctttttt |
| 4301 | tctgcgcgta atctgctgct tgcaaacaaa aaaaccaccg ctaccagcgg |
| 4351 | tggtttgttt gccggatcaa gagctaccaa ctctttttcc gaaggtaact |
| 4401 | ggcttcagca gagcgcagat accaaatact gtccttctag tgtagccgta |
| 4451 | gttaggccac cacttcaaga actctgtagc accgcctaca tacctcgctc |
| 4501 | tgctaatcct gttaccagtg gctgctgcca gtggcgataa gtcgtgtctt |
| 4551 | accgggttgg actcaagacg atagttaccg gataaggcgc agcggtcggg |
| 4601 | ctgaacgggg ggttcgtgca cacagcccag cttggagcga acgacctaca |
| 4651 | ccgaactgag atacctacag cgtgagcatt gagaaagcgc cacgcttccc |
| 4701 | gaagggagaa aggcggacag gtatccggta agcggcaggg tcggaacagg |
| 4751 | agagcgcacg agggagcttc cagggggaaa cgcctggtat ctttatagtc |
| 4801 | ctgtcgggtt tcgccacctc tgacttgagc gtcgattttt gtgatgctcg |
| 4851 | tcaggggggc ggagcctatg gaaaaacgcc agcaacgcgg cctttttacg |
| 4901 | gttcctggcc ttttgctggc cttttgctca catgttcttt cctgcgttat |
| 4951 | cccctgattc tgtggataac cgtattaccg cctttgagtg agctgatacc |
| 5001 | gctcgccgca gccgaacgac cgagcgcaac gcgtgcggcc gctaaactgt |
| 5051 | tacaacgctc acatatgtgg ttggcgacga gcccaaggca gtcgcctcgc |
| 5101 | tgttcaatct gtgaccggat ccgcaggacg tcgatccgtg ggtttacctg |
| 5151 | cggatttgtc gttactggcg ggtagcttct gaaacggttc agtttttggg |
| 5201 | cgacttcgca aaatttgcaa aaagtccgca ggccgttgcc gaaattcgca |
| 5251 | agtgaaatgg gtggaccagc gttgacacgc tgtgccatgg tcgagttagc |
| 5301 | acaccagtga agctgcgccg ttgacaccgc ctggacgacg gtagggcgtc |
| 5351 | agcgttttcg gcaatgaaag accgttaagg agttgtct |

(G) pMP399-mut SodA ΔH28ΔH76 (SEQ ID NO:29)

Full nucleotide sequence of chromosomal integration vector pMP399-mut SodA ΔH28ΔH76 used to express the mutant sodA in BCG to create SAD-BCGΔH28ΔH76 (chromosome-expressed). It can also be added to 1^{st}, and 2^{nd}, and 3^{rd} generation mutants of pro-apoptotic BCG to render, respectively, 2^{nd}, 3^{rd}, and 4^{th} generation pro-apoptotic BCG vaccines.

| | |
|---|---|
| 1 | ggtaccccgt ggccgaatac accttgccag acctggactg ggactacgga |
| 51 | gcactggaac cgcacatctc gggtcagatc aacgagcttc acagcaagca |
| 101 | ccacgccacc tacgtaaagg gcgccaatga cgccgtcgcc aaactcgaag |
| 151 | aggcgcgcgc caaggaagat cactcagcga tcttgctgaa cgaaaagaat |
| 201 | ctagctttca acctcgccgg ccacgttaat accatctggt ggaagaacct |
| 251 | gtcgcctaac ggtggtgaca agcccaccgg cgaactcgcc gcagccatcg |
| 301 | ccgacgcgtt cggttcgttc gacaagttcc gtgcgcagtt ccacgcggcc |
| 351 | gctaccaccg tgcaggggtc gggctgggcg gcactgggct gggacacact |
| 401 | cggcaacaag ctgctgatat tccaggttta cgaccaccag acgaacttcc |
| 451 | cgctaggcat tgttccgctg ctgctgctcg acatgtggga acacgccttc |
| 501 | tacctgcagt acaagaacgt caaagtcgac tttgccaagg cgttttggaa |
| 551 | cgtcgtgaac tgggccgatg tgcagtcacg gtatgcggcc gcgacctcgc |
| 601 | agaccaaggg gttgatattc agctgacccc gctgccgcaa gcgtcgggct |
| 651 | cagtattccg gagtcgcgca tcaccatggg gtacctctag agtcgaccac |
| 701 | caagggcacc atctctgctt gggccacccc gttggccgca gccagctcgc |
| 751 | tgagagccgt gaacgacagg gcgaacgcca gcccgccgac ggcgagggtt |
| 801 | ccgaccgctg caactcccgg tgcaaccttg tcccggtcta ttctcttcac |
| 851 | tgcaccagct ccaatctggt gtgaatgccc ctcgtctgtt cgcgcaggcg |
| 901 | gggggctcta ttcgtttgtc agcatcgaaa gtagccagat cagggatgcg |
| 951 | ttgcaaccgc gtatgcccag gtcagaagag tcgcacaaga gttgcagacc |
| 1001 | cctggaaaga aaaatggcca gagggcgaaa acaccctctg accagcggag |
| 1051 | cgggcgacgg gaatcgaacc cgcgtagcta gtttggaaga atgggtgtct |
| 1101 | gccgaccaca tatgggccgg tcaagatagg tttttacccc ctctcggctg |
| 1151 | catcctctaa gtggaaagaa attgcaggtc gtagaagcgc gttgaagcct |
| 1201 | gagagttgca caggagttgc aacccggtag ccttgttcac gacgagagga |
| 1251 | gacctagttg gcacgtcgcg gatggggatc gctgaagact cagcgcagcg |
| 1301 | ggaggatcca agcctcatac gtcaacccgc aggacggtgt gaggtactac |
| 1351 | gcgctgcaga cctacgacaa caagatggac gccgaagcct ggctcgcggg |
| 1401 | cgagaagcgg ctcatcgaga tggagacctg gacccctcca caggaccggg |
| 1451 | cgaagaaggc agccgccagc gccatcacgc tggaggagta cacccggaag |
| 1501 | tggctcgtgg agcgcgacct cgcagacggc accagggatc tgtacagcgg |
| 1551 | gcacgcggag cgccgcatct acccggtgct aggtgaagtg gcggtcacag |
| 1601 | agatgacgcc agctctggtg cgtgcgtggt gggccgggat gggtaggaag |
| 1651 | cacccgactg cccgccggca tgcctacaac gtcctccggg cggtgatgaa |
| 1701 | cacagcggtc gaggacaagc tgatcgcaga gaacccgtgc cggatcgagc |
| 1751 | agaaggcagc cgatgagcgc gacgtagagg cgctgacgcc tgaggagctg |
| 1801 | gacatcgtcg ccgctgagat cttcgagcac taccggatcg cggcatacat |
| 1851 | cctggcgtgg acgagcctcc ggttcggaga gctgatcgag cttcgccgca |
| 1901 | aggacatcgt ggacgacggc atgacgatga agctccgggt gcgccgtggc |
| 1951 | gcttcccgcg tggggaacaa gatcgtcgtt ggcaacgcca agaccgtccg |
| 2001 | gtcgaagcgt cctgtgacgg ttccgcctca cgtcgcggag atgatccgag |
| 2051 | cgcacatgaa ggaccgtacg aagatgaaca agggccccga ggcattcctg |
| 2101 | gtgaccacga cgcagggcaa ccggctgtcg aagtccgcgt tcaccaagtc |
| 2151 | gctgaagcgt ggctacgcca agatcggtcg gccggaactc cgcatccacg |
| 2201 | acctccgcgc tgtcggcgct acgttcgccg ctcaggcagg tgcgacgacc |
| 2251 | aaggagctga tggcccgtct cggtcacacg actcctagga tggcgatgaa |
| 2301 | gtaccagatg gcgtctgagg cccgcgacga ggctatcgct gaggcgatgt |
| 2351 | ccaagctggc caagacctcc tgaaacgcaa aaagcccccc tcccaaggac |
| 2401 | actgagtcct aaagaggggg gtttcttgtc agtacgcgaa gaaccacgcc |
| 2451 | tggccgcgag cgccagcacc gccgctctgt gcggagacct gggcaccagc |
| 2501 | cccgccgccg ccaggagcat tgccgttccc gccagaaatc tagaggtgac |
| 2551 | cacaacgacg cgcccgcttt gatcggggac gtctgcggcc gaccatttac |
| 2601 | gggtcttgtt gtcgttggcg gtcatgggcc gaacatactc acccggatcg |
| 2651 | gagggccgag gacaaggtcg aacgaggggc atgacccggt gcggggcttc |
| 2701 | ttgcactcgg cataggcgag tgctaagaat aacgttggca ctcgcgaccg |
| 2751 | gtgagtcgta ggtcgggacg gtgaggccag gcccgtcgtc gcagcgagtg |
| 2801 | gcagcgagga caacttgagc cgtccgtcgc gggcactgcg cccggccagc |
| 2851 | gtaagtagcg gggttgccgt cacccggtga cccccggttt catccccgat |
| 2901 | ccggaggaat cacttcgcaa tggccaagac aattgcggat ccagctgcag |
| 2951 | aattcgaagc ttatcgatgt cgacgtagtt aactagcgta cgatcgactg |
| 3001 | ccaggcatca aataaaacga aaggctcagt cgaaagactg ggcctttcgt |
| 3051 | tttatctgtt gtttgtccgg ccatcatggc cgcggtgatc agctagccaa |
| 3101 | caaagcgacg ttgtgtctca aaatctctga tgttacattg cacaagataa |
| 3151 | aaatatatca tcatgatcga attcctgcag ctcacggtaa ctgatgccgt |
| 3201 | atttgcagta ccagcgtacg gcccacagaa tgatgtcacg ctgaaaatgc |
| 3251 | cggcctttga atgggttcat gtgcagctcc atcagcaaaa ggggatgata |
| 3301 | agtttatcac caccgactat ttgcaacagt gccgttgatc gtgctatgat |
| 3351 | cgactgatgt catcagcggt ggagtgcaat gtcgtgcaat acgaatggcg |
| 3401 | aaaagccgag ctcatcggtc agcttctcaa ccttggggtt acccccggcg |
| 3451 | gtgtgctgct ggtccacagc tccttccgta gcgtccggcc cctcgaagat |
| 3501 | gggccacttg gactgatcga ggccctgcgt gctacgctgg gtccgggagg |
| 3551 | gacgctcgtc atgccctcgt ggtcaggtct ggacgacgag ccgttcgatc |
| 3601 | ctgccacgtc gcccgttaca ccggaccttg gagttgtctc tgacacattc |
| 3651 | tggcgcctgc caaatgtaaa gcgcagcgcc catccatttg cctttgcggc |
| 3701 | agcggggcca caggcagagc agatcatctc tgatccattg cccctgccac |
| 3751 | cttactcgcc tgcaagcccg gtcgcccgtg tccatgaact cgatgggcag |
| 3801 | gtacttctcc tcggcgtggg acacgatgcc aacacgacgc tgcatcttgc |
| 3851 | cgagttgatg gcaaaggttc cctatggggt gccgagacac tgcaccattc |
| 3901 | ttcaggatgg caagttggta cgcgtcgatt atctcgagaa tgaccactgc |
| 3951 | tgtgagcgct ttgccttggc ggacaggtgg ctcaaggaga agagccttca |
| 4001 | gaaggaaggt ccagtcggtc atgcctttgc tcggttgatc cgctcccgcg |
| 4051 | acattgtggc gacagccctg ggtcaactgg gccgagatcc gttgatcttc |
| 4101 | ctgcatccgc cagaggcggg atgcgaagaa tgcgatgccg ctcgccagtc |
| 4151 | gattggctga gctcatgagc ggagaacgag atgacgttgg aggggcaagg |
| 4201 | tcgcgctgat tgctggggca acacggggga tccactagtt ccactgagcg |
| 4251 | tcagaccccg tagaaaagat caaaggatct tcttgagatc ctttttttct |
| 4301 | gcgcgtaatc tgctgcttgc aaacaaaaaa accaccgcta ccagcggtgg |
| 4351 | tttgtttgcc ggatcaagag ctaccaactc tttttccgaa ggtaactggc |
| 4401 | ttcagcagag cgcagatacc aaatactgtc cttctagtgt agccgtagtt |
| 4451 | aggccaccac ttcaagaact ctgtagcacc gcctacatac ctcgctctgc |
| 4501 | taatcctgtt accagtggct gctgccagtg gcgataagtc gtgtcttacc |
| 4551 | gggttggact caagacgata gttaccggat aaggcgcagc ggtcgggctg |
| 4601 | aacggggggt tcgtgcacac agcccagctt ggagcgaacg acctacaccg |
| 4651 | aactgagata cctacagcgt gagcattgag aaagcgccac gcttcccgaa |
| 4701 | gggagaaagg cggacaggta tccggtaagc ggcagggtcg gaacaggaga |
| 4751 | gcgcacgagg gagcttccag ggggaaacgc ctggtatctt tatagtcctg |
| 4801 | tcgggtttcg ccacctctga cttgagcgtc gatttttgtg atgctcgtca |
| 4851 | ggggggcgga gcctatggaa aaacgccagc aacgcggcct ttttacggtt |
| 4901 | cctggccttt tgctggcctt ttgctcacat gttctttcct gcgttatccc |
| 4951 | ctgattctgt ggataaccgt attaccgcct ttgagtgagc tgataccgct |
| 5001 | cgccgcagcc gaacgaccga gcgcaacgcg tgcggccgct aaactgttac |
| 5051 | aacgctcaca tatgtggttg gcgacgagcc caaggcagtc gcctcgctgt |
| 5101 | tcaatctgtg accggatccg caggacgtcg atccgtgggt ttacctgcgg |
| 5151 | atttgtcgtt actggcgggt agcttctgaa acggttcagt ttttgggcga |
| 5201 | cttcgcaaaa tttgcaaaaa gtccgcaggc cgttgccgaa attcgcaagt |
| 5251 | gaaatgggtg gaccagcgtt gacacgctgt gccatggtcg agttagcaca |
| 5301 | ccagtgaagc tgcgccgttg acaccgcctg gacgacggta gggcgtcagc |
| 5351 | gttttcggca atgaaagacc gttaaggagt tgtct |

(H) pMP399-mut glnA1 ΔD54ΔE335 (SEQ ID NO:30)

Full nucleotide sequence of chromosomal integration vector pMP399-mut glnA1 ΔD54ΔE335 used to express the mutant glnA1 in BCG to create GLAD-BCG (chromosome-expressed). It can also be added to 1^{st}, and 2^{nd}, and 3^{rd} generation mutants of pro-apoptotic BCG to render, respectively, 2^{nd}, 3^{rd}, and 4^{th} generation pro-apoptotic BCG vaccines.

| | |
|---|---|
| 1 | gctagccaac aaagcgacgt tgtgtctcaa aatctctgat gttacattgc acaagataaa |
| 61 | aatatatcat catgatcgaa ttcctgcagc tcacggtaac tgatgccgta tttgcagtac |
| 121 | cagcgtacgg cccacagaat gatgtcacgc tgaaaatgcc ggcctttgaa tgggttcatg |
| 181 | tgcagctcca tcagcaaaag gggatgataa gtttatcacc accgactatt tgcaacagtg |
| 241 | ccgttgatcg tgctatgatc gactgatgtc atcagcggtg gagtgcaatg tcgtgcaata |
| 301 | cgaatggcga aaagccgagc tcatcggtca gcttctcaac cttggggtta cccccggcgg |
| 361 | tgtgctgctg gtccacagct ccttccgtag cgtccggccc ctcgaagatg ggccacttgg |
| 421 | actgatcgag gccctgcgtg ctacgctggg tccgggaggg acgctcgtca tgccctcgtg |
| 481 | gtcaggtctg gacgacgagc cgttcgatcc tgccacgtcg cccgttacac cggaccttgg |
| 541 | agttgtctct gacacattct ggcgcctgcc aaatgtaaag cgcagcgccc atccatttgc |
| 601 | ctttgcggca gcggggccac aggcagagca gatcatctct gatccattgc ccctgccacc |
| 661 | ttactcgcct gcaagcccgg tcgcccgtgt ccatgaactc gatgggcagg tacttctcct |
| 721 | cggcgtggga cacgatgcca acacgacgct gcatcttgcc gagttgatgg caaaggttcc |
| 781 | ctatggggtg ccgagacact gcaccattct tcaggatggc aagttggtac gcgtcgatta |
| 841 | tctcgagaat gaccactgct gtgagcgctt tgccttggcg gacaggtggc tcaaggagaa |
| 901 | gagccttcag aaggaaggtc cagtcggtca tgcctttgct cggttgatcc gctcccgcga |
| 961 | cattgtggcg acagccctgg gtcaactggg ccgagatccg ttgatcttcc tgcatccgcc |
| 1021 | agaggcggga tgcgaagaat gcgatgccgc tcgccagtcg attggctgag ctcatgagcg |
| 1081 | gagaacgaga tgacgttgga ggggcaaggt cgcgctgatt gctggggcaa cacgggggat |
| 1141 | ccactagtcc accaccagac ggccgatccc caccggccgc cggccaccca ctgccaccac |
| 1201 | gaccagaccc agcatcaact gcccgggtgt gaatccgaac aagcggaccg ccgccacccc |
| 1261 | gagcagcagc caaatcacca ggacaaccgt cgacagcatc ggggtcgacc aaacaccgaa |
| 1321 | ttccacgccc agcaacgcca gaccgtaggc gatcagccag tcgatcagca gagccgccag |
| 1381 | ccggcgcccc atcggagcca gcgaacccgg tccggtgtcc ggcaagccca gcgtcttgcc |
| 1441 | gggatagtcg ggcggcgatt tcgccgtcat cgggcagacc cgataaccag gttcccgttc |
| 1501 | ggcatgccac cggttacgat cttgccgacc atggccccac aatagggccg gggagacccg |
| 1561 | gcgtcagtgg tgggcggcac ggtcagtaac gtctgcgcaa cacggggttg actgacgggc |
| 1621 | aatatcggct ccatagcgtc ggccgcggat acagtaaagg agcattctgt gacggaaaag |
| 1681 | acgcccgacg acgtcttcaa acttgccaag gacgagaagg tcgaatatgt cgacgtccgg |
| 1741 | ttctgtgacc tgcctggcat catgcagcac ttcacgattc cggcttcggc ctttgacaag |
| 1801 | agcgtgtttg acgacggctt ggcctttggc tcgtcgattc gcgggttcca gtcgatccac |
| 1861 | gaatccgaca tgttgcttct tcccgatccc gagacggcgc gcatcgaccc gttccgcgcg |
| 1921 | gccaagacgc tgaatatcaa cttctttgtg cacgacccgt tcaccctgga gccgtactcc |
| 1981 | cgcgacccgc gcaacatcgc ccgcaaggcc gagaactacc tgatcagcac tggcatcgcc |
| 2041 | gacaccgcat acttcggcgc cgaggccgag ttctacattt tcgattcggt gagcttcgac |
| 2101 | tcgcgcgcca acggctcctt ctacgaggtg gacgccatct cggggtggtg gaacaccggc |
| 2161 | gcggcgaccg aggccgacgg cagtcccaac cggggctaca aggtccgcca caagggcggg |
| 2221 | tatttcccag tggcccccaa cgaccaatac gtcgacctgc gcgacaagat gctgaccaac |
| 2281 | ctgatcaact ccggcttcat cctggagaag ggccaccacg aggtgggcag cggcggacag |
| 2341 | gccgagatca actaccagtt caattcgctg ctgcacgccg ccgacgacat gcagttgtac |
| 2401 | aagtacatca tcaagaacac cgcctggcag aacggcaaaa cggtcacgtt catgcccaag |
| 2461 | ccgctgttcg gcgacaacgg gtccggcatg cactgtcatc agtcgctgtg gaaggacggg |
| 2521 | gccccgctga tgtacgacga gacgggttat gccggtctgt cggacacggc ccgtcattac |
| 2581 | atcggcggcc tgttacacca cgcgccgtcg ctgctggcct tcaccaaccc gacggtgaac |
| 2641 | tcctacaagc ggctggttcc cggttacgcc ccgatcaacc tggtctatag ccagcgcaac |
| 2701 | cggtcggcat gcgtgcgcat cccgatcacc ggcagcaacc cgaaggccaa gcggctggag |
| 2761 | ttccgaagcc ccgactcgtc gggcaacccg tatctggcgt tctcggccat gctgatggca |
| 2821 | ggcctggacg gtatcaagaa caagatcgag ccgcaggcgc ccgtcgacaa ggatctctac |
| 2881 | gagctgccgc cggaagaggc cgcgagtatc ccgcagactc cgacccagct gtcagatgtg |
| 2941 | atcgaccgtc tcgaggccga ccacgaatac ctcaccgaag gaggggtgtt cacaaacgac |
| 3001 | ctgatcgaga cgtggatcag tttcaagcgc gaaaacgaga tcgagccggt caacatccgg |
| 3061 | ccgcatccct acgaattcgc gctgtactac gacgtttaag gactcttcgc agtccgggtg |
| 3121 | tagagggagc ggcgtggact agttccactg agcgtcagac cccgtagaaa agatcaaagg |
| 3181 | atcttcttga gatccttttt ttctgcgcgt aatctgctgc ttgcaaacaa aaaaaccacc |
| 3241 | gctaccagcg gtggtttgtt tgccggatca agagctacca actctttttc cgaaggtaac |
| 3301 | tggcttcagc agagcgcaga taccaaatac tgtccttcta gtgtagccgt agttaggcca |
| 3361 | ccacttcaag aactctgtag caccgcctac atacctcgct ctgctaatcc tgttaccagt |
| 3421 | ggctgctgcc agtggcgata agtcgtgtct taccgggttg gactcaagac gatagttacc |
| 3481 | ggataaggcg cagcggtcgg gctgaacggg gggttcgtgc acacagccca gcttggagcg |
| 3541 | aacgacctac accgaactga gatacctaca gcgtgagcat tgagaaagcg ccacgcttcc |
| 3601 | cgaagggaga aaggcggaca ggtatccggt aagcggcagg gtcggaacag gagagcgcac |
| 3661 | gagggagctt ccagggggaa acgcctggta tctttatagt cctgtcgggt ttcgccacct |
| 3721 | ctgacttgag cgtcgatttt tgtgatgctc gtcagggggg cggagcctat ggaaaaacgc |
| 3781 | cagcaacgcg gcctttttac ggttcctggc cttttgctgg ccttttgctc acatgttctt |
| 3841 | tcctgcgtta tcccctgatt ctgtggataa ccgtattacc gcctttgagt gagctgatac |
| 3901 | cgctcgccgc agccgaacga ccgagcgcaa cgcgtgcggc cgcggtaccc ggggatcctc |
| 3961 | tagagtcgac caccaagggc accatctctg cttgggccac cccgttggcc gcagccagct |
| 4021 | cgctgagagc cgtgaacgac agggcgaacg ccagcccgcc gacggcgagg gttccgaccg |
| 4081 | ctgcaactcc cggtgcaacc ttgtcccggt ctattctctt cactgcacca gctccaatct |
| 4141 | ggtgtgaatg cccctcgtct gttcgcgcag gcggggggct ctattcgttt gtcagcatcg |
| 4201 | aaagtagcca gatcagggat gcgttgcaac cgcgtatgcc caggtcagaa gagtcgcaca |
| 4261 | agagttgcag acccctggaa agaaaaatgg ccagagggcg aaaacaccct ctgaccagcg |
| 4321 | gagcgggcga cgggaatcga acccgcgtag ctagtttgga agaatgggtg tctgccgacc |
| 4381 | acatatgggc cggtcaagat aggtttttac cccctctcgg ctgcatcctc taagtggaaa |
| 4441 | gaaattgcag gtcgtagaag cgcgttgaag cctgagagtt gcacaggagt tgcaacccgg |
| 4501 | tagccttgtt cacgacgaga ggagacctag ttggcacgtc gcggatgggg atcgctgaag |
| 4561 | actcagcgca gcgggaggat ccaagcctca tacgtcaacc cgcaggacgg tgtgaggtac |
| 4621 | tacgcgctgc agacctacga caacaagatg gacgccgaag cctggctcgc gggcgagaag |
| 4681 | cggctcatcg agatggagac ctggacccct ccacaggacc gggcgaagaa ggcagccgcc |
| 4741 | agcgccatca cgctggagga gtacacccgg aagtggctcg tggagcgcga cctcgcagac |
| 4801 | ggcaccaggg atctgtacag cgggcacgcg gagcgccgca tctacccggt gctaggtgaa |
| 4861 | gtggcggtca cagagatgac gccagctctg gtgcgtgcgt ggtgggccgg gatgggtagg |
| 4921 | aagcacccga ctgcccgccg gcatgcctac aacgtcctcc gggcggtgat gaacacagcg |
| 4981 | gtcgaggaca agctgatcgc agagaacccg tgccggatcg agcagaaggc agccgatgag |
| 5041 | cgcgacgtag aggcgctgac gcctgaggag ctggacatcg tcgccgctga gatcttcgag |
| 5101 | cactaccgga tcgcggcata catcctggcg tggacgagcc tccggttcgg agagctgatc |
| 5161 | gagcttcgcc gcaaggacat cgtggacgac ggcatgacga tgaagctccg ggtgcgccgt |
| 5221 | ggcgcttccc gcgtggggaa caagatcgtc gttggcaacg ccaagaccgt ccggtcgaag |
| 5281 | cgtcctgtga cggttccgcc tcacgtcgcg gagatgatcc gagcgcacat gaaggaccgt |
| 5341 | acgaagatga acaagggccc cgaggcattc ctggtgacca cgacgcaggg caaccggctg |
| 5401 | tcgaagtccg cgttcaccaa gtcgctgaag cgtggctacg ccaagatcgg tcggccggaa |
| 5461 | ctccgcatcc acgacctccg cgctgtcggc gctacgttcg ccgctcaggc aggtgcgacg |
| 5521 | accaaggagc tgatggcccg tctcggtcac acgactccta ggatggcgat gaagtaccag |
| 5581 | atggcgtctg aggcccgcga cgaggctatc gctgaggcga tgtccaagct ggccaagacc |
| 5641 | tcctgaaacg caaaaagccc ccctcccaag gacactgagt cctaaagagg ggggtttctt |
| 5701 | gtcagtacgc gaagaaccac gcctggccgc gagcgccagc accgccgctc tgtgcggaga |
| 5761 | cctgggcacc agccccgccg ccgccaggag cattgccgtt cccgccagaa atctagaggt |
| 5821 | gaccacaacg acgcgcccgc tttgatcggg gacgtctgcg gccgaccatt tacgggtctt |
| 5881 | gttgtcgttg gcggtcatgg gccgaacata ctcacccgga tcggagggcc gaggacaagg |
| 5941 | tcgaacgagg ggcatgaccc ggtgcggggc ttcttgcact cggcataggc gagtgctaag |
| 6001 | aataacgttg gcactcgcga ccggtgagtc gtaggtcggg acggtgaggc caggcccgtc |
| 6061 | gtcgcagcga gtggcagcga ggacaacttg agccgtccgt cgcgggcact gcgcccggcc |
| 6121 | agcgtaagta gcggggttgc cgtcacccgg tgacccccgg tttcatcccc gatccggagg |
| 6181 | aatcacttcg caatggccaa gacaattgcg gatccagctg cagaattcga agcttatcga |
| 6241 | tgtcgacgta gttaactagc gtacgatcga ctgccaggca tcaaataaaa cgaaaggctc |
| 6301 | agtcgaaaga ctgggccttt cgttttatct gttgtttgtc cggccatcat ggccgcggtg |
| 6361 | atca |

(I) pMP399-mut SodA ΔE54, mut glnA1 ΔD54ΔE335 (SEQ ID NO:31)

Full nucleotide sequence of plasmid vector pMP399-mut SodA ΔE54, mut glnA1 ΔD54ΔE335 used to simultaneously express the ΔE54 mutant sodA and the ΔD54ΔE335 mutant glnA1 in BCG to create GLAD-SAD-BCG ΔE54 (chromosome-expressed). It can also be added to 1^{st} and 2^{nd} generation mutants of pro-apoptotic BCG to render, respectively, 3^{rd} and 4^{th} generation pro-apoptotic BCG vaccines.

| | |
|---|---|
| 1 | ggtaccccgt ggccgaatac accttgccag acctggactg ggactacgga gcactggaac |
| 61 | cgcacatctc gggtcagatc aacgagcttc gccacagcaa gcaccacgcc acctacgtaa |
| 121 | agggcgccaa tgacgccgtc gccaaactcg aagaggcgcg cgccaaggat cactcagcga |
| 181 | tcatgctgaa cgaaaagaat ctagctttca acctcgccgg ccacgttaat cacaccatct |
| 241 | ggtggaagaa cctgtcgcct aacggtggtg acaagcccac cggcgaactc gccgcagcca |
| 301 | tcgccgacgc gttcggttcg ttcgacaagt tccgtgcgca gttccacgcg gccgctacca |
| 361 | ccgtgcaggg gtcgggctgg gcggcactgg gctgggacac actcggcaac aagctgctga |
| 421 | tattccaggt ttacgaccac cagacgaact tcccgctagg cattgttccg ctgctgctgc |
| 481 | tcgacatgtg ggaacacgcc ttctacctgc agtacaagaa cgtcaaagtc gactttgcca |
| 541 | aggcgttttg gaacgtcgtg aactgggccg atgtgcagtc acggtatgcg gccgcgacct |
| 601 | cgcagaccaa ggggttgata ttcagctgac cccgctgccg caagcgtcgg gctcagtatt |
| 661 | ccggagtcgc gcatcaccat ggggtacctc tagagtcgac caccaagggc accatctctg |
| 721 | cttgggccac cccgttggcc gcagccagct cgctgagagc cgtgaacgac agggcgaacg |
| 781 | ccagcccgcc gacggcgagg gttccgaccg ctgcaactcc cggtgcaacc ttgtcccggt |
| 841 | ctattctctt cactgcacca gctccaatct ggtgtgaatg cccctcgtct gttcgcgcag |
| 901 | gcggggggct ctattcgttt gtcagcatcg aaagtagcca gatcagggat gcgttgcaac |
| 961 | cgcgtatgcc caggtcagaa gagtcgcaca agagttgcag acccctggaa agaaaaatgg |
| 1021 | ccagagggcg aaaacaccct ctgaccagcg gagcgggcga cgggaatcga acccgcgtag |
| 1081 | ctagtttgga agaatgggtg tctgccgacc acatatgggc cggtcaagat aggtttttac |
| 1141 | cccctctcgg ctgcatcctc taagtggaaa gaaattgcag gtcgtagaag cgcgttgaag |
| 1201 | cctgagagtt gcacaggagt tgcaacccgg tagccttgtt cacgacgaga ggagacctag |
| 1261 | ttggcacgtc gcggatgggg atcgctgaag actcagcgca gcgggaggat ccaagcctca |
| 1321 | tacgtcaacc cgcaggacgg tgtgaggtac tacgcgctgc agacctacga caacaagatg |
| 1381 | gacgccgaag cctggctcgc gggcgagaag cggctcatcg agatggagac ctggacccct |
| 1441 | ccacaggacc gggcgaagaa ggcagccgcc agcgccatca cgctggagga gtacacccgg |
| 1501 | aagtggctcg tggagcgcga cctcgcagac ggcaccaggg atctgtacag cgggcacgcg |
| 1561 | gagcgccgca tctacccggt gctaggtgaa gtggcggtca cagagatgac gccagctctg |
| 1621 | gtgcgtgcgt ggtgggccgg gatgggtagg aagcacccga ctgcccgccg gcatgcctac |
| 1681 | aacgtcctcc gggcggtgat gaacacagcg gtcgaggaca agctgatcgc agagaacccg |
| 1741 | tgccggatcg agcagaaggc agccgatgag cgcgacgtag aggcgctgac gcctgaggag |
| 1801 | ctggacatcg tcgccgctga gatcttcgag cactaccgga tcgcggcata catcctggcg |
| 1861 | tggacgagcc tccggttcgg agagctgatc gagcttcgcc gcaaggacat cgtggacgac |
| 1921 | ggcatgacga tgaagctccg ggtgcgccgt ggcgcttccc gcgtggggaa caagatcgtc |
| 1981 | gttggcaacg ccaagaccgt ccggtcgaag cgtcctgtga cggttccgcc tcacgtcgcg |
| 2041 | gagatgatcc gagcgcacat gaaggaccgt acgaagatga acaagggccc cgaggcattc |
| 2101 | ctggtgacca cgacgcaggg caaccggctg tcgaagtccg cgttcaccaa gtcgctgaag |
| 2161 | cgtggctacg ccaagatcgg tcggccggaa ctccgcatcc acgacctccg cgctgtcggc |
| 2221 | gctacgttcg ccgctcaggc aggtgcgacg accaaggagc tgatggcccg tctcggtcac |
| 2281 | acgactccta ggatggcgat gaagtaccag atggcgtctg aggcccgcga cgaggctatc |
| 2341 | gctgaggcga tgtccaagct ggccaagacc tcctgaaacg caaaaagccc ccctcccaag |
| 2401 | gacactgagt cctaaagagg ggggtttctt gtcagtacgc gaagaaccac gcctggccgc |
| 2461 | gagcgccagc accgccgctc tgtgcggaga cctgggcacc agccccgccg ccgccaggag |
| 2521 | cattgccgtt cccgccagaa atctagaggt gaccacaacg acgcgcccgc tttgatcggg |
| 2581 | gacgtctgcg gccgaccatt tacgggtctt gttgtcgttg gcggtcatgg gccgaacata |
| 2641 | ctcacccgga tcggagggcc gaggacaagg tcgaacgagg ggcatgaccc ggtgcggggc |
| 2701 | ttcttgcact cggcataggc gagtgctaag aataacgttg gcactcgcga ccggtgagtc |
| 2761 | gtaggtcggg acggtgaggc caggcccgtc gtcgcagcga gtggcagcga ggacaacttg |
| 2821 | agccgtccgt cgcgggcact gcgcccggcc agcgtaagta gcggggttgc cgtcacccgg |
| 2881 | tgacccccgg tttcatcccc gatccggagg aatcacttcg caatggccaa gacaattgcg |
| 2941 | gatccagctg cagaattcga agcttatcga tgtcgacgta gttaactagc gtacgatcga |
| 3001 | ctgccaggca tcaaataaaa cgaaaggctc agtcgaaaga ctgggccttt cgttttatct |
| 3061 | gttgtttgtc cggccatcat ggccgcggtg atcagctagc caacaaagcg acgttgtgtc |
| 3121 | tcaaaatctc tgatgttaca ttgcacaaga taaaaatata tcatcatgat cgaattcctg |
| 3181 | cagctcacgg taactgatgc cgtatttgca gtaccagcgt acggcccaca gaatgatgtc |
| 3241 | acgctgaaaa tgccggcctt tgaatgggtt catgtgcagc tccatcagca aaaggggatg |
| 3301 | ataagtttat caccaccgac tatttgcaac agtgccgttg atcgtgctat gatcgactga |
| 3361 | tgtcatcagc ggtggagtgc aatgtcgtgc aatacgaatg gcgaaaagcc gagctcatcg |
| 3421 | gtcagcttct caaccttggg gttacccccg gcggtgtgct gctggtccac agctccttcc |
| 3481 | gtagcgtccg gcccctcgaa gatgggccac ttggactgat cgaggccctg cgtgctacgc |
| 3541 | tgggtccggg agggacgctc gtcatgccct cgtggtcagg tctggacgac gagccgttcg |
| 3601 | atcctgccac gtcgcccgtt acaccggacc ttggagttgt ctctgacaca ttctggcgcc |
| 3661 | tgccaaatgt aaagcgcagc gcccatccat ttgcctttgc ggcagcgggg ccacaggcag |
| 3721 | agcagatcat ctctgatcca ttgcccctgc caccttactc gcctgcaagc ccggtcgccc |
| 3781 | gtgtccatga actcgatggg caggtacttc tcctcggcgt gggacacgat gccaacacga |
| 3841 | cgctgcatct tgccgagttg atggcaaagg ttccctatgg ggtgccgaga cactgcacca |
| 3901 | ttcttcagga tggcaagttg gtacgcgtcg attatctcga gaatgaccac tgctgtgagc |
| 3961 | gctttgcctt ggcggacagg tggctcaagg agaagagcct tcagaaggaa ggtccagtcg |
| 4021 | gtcatgcctt tgctcggttg atccgctccc gcgacattgt ggcgacagcc ctgggtcaac |
| 4081 | tgggccgaga tccgttgatc ttcctgcatc cgccagaggc gggatgcgaa gaatgcgatg |
| 4141 | ccgctcgcca gtcgattggc tgagctcatg agcggagaac gagatgacgt tggaggggca |
| 4201 | aggtcgcgct gattgctggg gcaacacggg ggatccacta gtccaccacc agacggccga |
| 4261 | tccccaccgg ccgccggcca cccactgcca ccacgaccag acccagcatc aactgcccgg |
| 4321 | gtgtgaatcc gaacaagcgg accgccgcca ccccgagcag cagccaaatc accaggacaa |
| 4381 | ccgtcgacag catcggggtc gaccaaacac cgaattccac gcccagcaac gccagaccgt |
| 4441 | aggcgatcag ccagtcgatc agcagagccg ccagccggcg ccccatcgga gccagcgaac |
| 4501 | ccggtccggt gtccggcaag cccagcgtct tgccgggata gtcgggcggc gatttcgccg |
| 4561 | tcatcgggca gacccgataa ccaggttccc gttcggcatg ccaccggtta cgatcttgcc |
| 4621 | gaccatggcc ccacaatagg gccggggaga cccggcgtca gtggtgggcg gcacggtcag |
| 4681 | taacgtctgc gcaacacggg gttgactgac gggcaatatc ggctccatag cgtcggccgc |
| 4741 | ggatacagta aaggagcatt ctgtgacgga aaagacgccc gacgacgtct tcaaacttgc |
| 4801 | caaggacgag aaggtcgaat atgtcgacgt ccggttctgt gacctgcctg gcatcatgca |
| 4861 | gcacttcacg attccggctt cggcctttga caagagcgtg tttgacgacg gcttggcctt |
| 4921 | tggctcgtcg attcgcgggt tccagtcgat ccacgaatcc gacatgttgc ttcttcccga |
| 4981 | tcccgagacg gcgcgcatcg acccgttccg cgcggccaag acgctgaata tcaacttctt |
| 5041 | tgtgcacgac ccgttcaccc tggagccgta ctcccgcgac ccgcgcaaca tcgcccgcaa |
| 5101 | ggccgagaac tacctgatca gcactggcat cgccgacacc gcatacttcg gcgccgaggc |
| 5161 | cgagttctac attttcgatt cggtgagctt cgactcgcgc gccaacggct ccttctacga |
| 5221 | ggtggacgcc atctcggggt ggtggaacac cggcgcggcg accgaggccg acggcagtcc |
| 5281 | caaccggggc tacaaggtcc gccacaaggg cgggtatttc ccagtggccc ccaacgacca |
| 5341 | atacgtcgac ctgcgcgaca agatgctgac caacctgatc aactccggct tcatcctgga |
| 5401 | gaagggccac cacgaggtgg gcagcggcgg acaggccgag atcaactacc agttcaattc |
| 5461 | gctgctgcac gccgccgacg acatgcagtt gtacaagtac atcatcaaga acaccgcctg |
| 5521 | gcagaacggc aaaacggtca cgttcatgcc caagccgctg ttcggcgaca acgggtccgg |
| 5581 | catgcactgt catcagtcgc tgtggaagga cggggccccg ctgatgtacg acgagacggg |
| 5641 | ttatgccggt ctgtcggaca cggcccgtca ttacatcggc ggcctgttac accacgcgcc |
| 5701 | gtcgctgctg gccttcacca acccgacggt gaactcctac aagcggctgg ttcccggtta |
| 5761 | cgccccgatc aacctggtct atagccagcg caaccggtcg gcatgcgtgc gcatcccgat |
| 5821 | caccggcagc aacccgaagg ccaagcggct ggagttccga agccccgact cgtcgggcaa |
| 5881 | cccgtatctg gcgttctcgg ccatgctgat ggcaggcctg gacggtatca agaacaagat |
| 5941 | cgagccgcag gcgcccgtcg acaaggatct ctacgagctg ccgccggaag aggccgcgag |
| 6001 | tatcccgcag actccgaccc agctgtcaga tgtgatcgac cgtctcgagg ccgaccacga |
| 6061 | atacctcacc gaaggagggg tgttcacaaa cgacctgatc gagacgtgga tcagtttcaa |
| 6121 | gcgcgaaaac gagatcgagc cggtcaacat ccggccgcat ccctacgaat tcgcgctgta |
| 6181 | ctacgacgtt taaggactct tcgcagtccg ggtgtagagg gagcggcgtg gactagttcc |
| 6241 | actgagcgtc agaccccgta gaaaagatca aaggatcttc ttgagatcct ttttttctgc |
| 6301 | gcgtaatctg ctgcttgcaa acaaaaaaac caccgctacc agcggtggtt tgtttgccgg |
| 6361 | atcaagagct accaactctt tttccgaagg taactggctt cagcagagcg cagataccaa |
| 6421 | atactgtcct tctagtgtag ccgtagttag gccaccactt caagaactct gtagcaccgc |
| 6481 | ctacatacct cgctctgcta atcctgttac cagtggctgc tgccagtggc gataagtcgt |
| 6541 | gtcttaccgg gttggactca agacgatagt taccggataa ggcgcagcgg tcgggctgaa |
| 6601 | cggggggttc gtgcacacag cccagcttgg agcgaacgac ctacaccgaa ctgagatacc |
| 6661 | tacagcgtga gcattgagaa agcgccacgc ttcccgaagg gagaaaggcg gacaggtatc |
| 6721 | cggtaagcgg cagggtcgga acaggagagc gcacgaggga gcttccaggg ggaaacgcct |
| 6781 | ggtatcttta tagtcctgtc gggtttcgcc acctctgact tgagcgtcga tttttgtgat |
| 6841 | gctcgtcagg ggggcggagc ctatggaaaa acgccagcaa cgcggccttt ttacggttcc |
| 6901 | tggccttttg ctggcctttt gctcacatgt tctttcctgc gttatcccct gattctgtgg |
| 6961 | ataaccgtat taccgccttt gagtgagctg ataccgctcg ccgcagccga acgaccgagc |
| 7021 | gcaacgcgtg cggccgctaa actgttacaa cgctcacata tgtggttggc gacgagccca |
| 7081 | aggcagtcgc ctcgctgttc aatctgtgac cggatccgca ggacgtcgat ccgtgggttt |
| 7141 | acctgcggat ttgtcgttac tggcgggtag cttctgaaac ggttcagttt ttgggcgact |
| 7201 | tcgcaaaatt tgcaaaaagt ccgcaggccg ttgccgaaat tcgcaagtga aatgggtgga |
| 7261 | ccagcgttga cacgctgtgc catggtcgag ttagcacacc agtgaagctg cgccgttgac |
| 7321 | accgcctgga cgacggtagg gcgtcagcgt tttcggcaat gaaagaccgt taaggagttg |
| 7381 | tct |

(J) pMP399-mut SodA ΔH28ΔH76, mut glnA1 ΔD54ΔE335 (SEQ ID NO:32)

Full nucleotide sequence of plasmid vector pMP399-mut SodA ΔH28ΔH76, mut glnA1 ΔD54ΔE335 used to simultaneously express the ΔH28ΔH76 mutant sodA and the ΔD54ΔE335 mutant glnA1 in BCG to create GLAD-SAD-BCG ΔH28ΔH76 (chromosome-expressed). It can also be added to 1^{st} and 2^{nd} generation mutants of pro-apoptotic BCG to render, respectively, 3^{rd} and 4^{th} generation pro-apoptotic BCG vaccines.

| | |
|---|---|
| 1 | ggtaccccgt ggccgaatac accttgccag acctggactg ggactacgga |
| 51 | gcactggaac cgcacatctc gggtcagatc aacgagcttc acagcaagca |
| 101 | ccacgccacc tacgtaaagg gcgccaatga cgccgtcgcc aaactcgaag |
| 151 | aggcgcgcgc caaggaagat cactcagcga tcttgctgaa cgaaaagaat |
| 201 | ctagctttca acctcgccgg ccacgttaat accatctggt ggaagaacct |
| 251 | gtcgcctaac ggtggtgaca agcccaccgg cgaactcgcc gcagccatcg |
| 301 | ccgacgcgtt cggttcgttc gacaagttcc gtgcgcagtt ccacgcggcc |
| 351 | gctaccaccg tgcaggggtc gggctgggcg gcactgggct gggacacact |
| 401 | cggcaacaag ctgctgatat tccaggttta cgaccaccag acgaacttcc |
| 451 | cgctaggcat tgttccgctg ctgctgctcg acatgtggga acacgccttc |
| 501 | tacctgcagt acaagaacgt caaagtcgac tttgccaagg cgttttggaa |
| 551 | cgtcgtgaac tgggccgatg tgcagtcacg gtatgcggcc gcgacctcgc |
| 601 | agaccaaggg gttgatattc agctgacccc gctgccgcaa gcgtcgggct |
| 651 | cagtattccg gagtcgcgca tcaccatggg gtacctctag agtcgaccac |
| 701 | caagggcacc atctctgctt gggccacccc gttggccgca gccagctcgc |
| 751 | tgagagccgt gaacgacagg gcgaacgcca gcccgccgac ggcgagggtt |
| 801 | ccgaccgctg caactcccgg tgcaaccttg tcccggtcta ttctcttcac |
| 851 | tgcaccagct ccaatctggt gtgaatgccc ctcgtctgtt cgcgcaggcg |
| 901 | gggggctcta ttcgtttgtc agcatcgaaa gtagccagat cagggatgcg |
| 951 | ttgcaaccgc gtatgcccag gtcagaagag tcgcacaaga gttgcagacc |
| 1001 | cctggaaaga aaaatggcca gagggcgaaa acaccctctg accagcggag |
| 1051 | cgggcgacgg gaatcgaacc cgcgtagcta gtttggaaga atgggtgtct |
| 1101 | gccgaccaca tatgggccgg tcaagatagg tttttacccc ctctcggctg |
| 1151 | catcctctaa gtggaaagaa attgcaggtc gtagaagcgc gttgaagcct |
| 1201 | gagagttgca caggagttgc aacccggtag ccttgttcac gacgagagga |
| 1251 | gacctagttg gcacgtcgcg gatggggatc gctgaagact cagcgcagcg |
| 1301 | ggaggatcca agcctcatac gtcaacccgc aggacggtgt gaggtactac |
| 1351 | gcgctgcaga cctacgacaa caagatggac gccgaagcct ggctcgcggg |
| 1401 | cgagaagcgg ctcatcgaga tggagacctg gacccctcca caggaccggg |
| 1451 | cgaagaaggc agccgccagc gccatcacgc tggaggagta cacccggaag |
| 1501 | tggctcgtgg agcgcgacct cgcagacggc accagggatc tgtacagcgg |
| 1551 | gcacgcggag cgccgcatct acccggtgct aggtgaagtg gcggtcacag |
| 1601 | agatgacgcc agctctggtg cgtgcgtggt gggccgggat gggtaggaag |
| 1651 | cacccgactg cccgccggca tgcctacaac gtcctccggg cggtgatgaa |
| 1701 | cacagcggtc gaggacaagc tgatcgcaga gaacccgtgc cggatcgagc |
| 1751 | agaaggcagc cgatgagcgc gacgtagagg cgctgacgcc tgaggagctg |
| 1801 | gacatcgtcg ccgctgagat cttcgagcac taccggatcg cggcatacat |
| 1851 | cctggcgtgg acgagcctcc ggttcggaga gctgatcgag cttcgccgca |
| 1901 | aggacatcgt ggacgacggc atgacgatga agctccgggt gcgccgtggc |
| 1951 | gcttcccgcg tggggaacaa gatcgtcgtt ggcaacgcca agaccgtccg |
| 2001 | gtcgaagcgt cctgtgacgg ttccgcctca cgtcgcggag atgatccgag |
| 2051 | cgcacatgaa ggaccgtacg aagatgaaca agggccccga ggcattcctg |
| 2101 | gtgaccacga cgcagggcaa ccggctgtcg aagtccgcgt tcaccaagtc |
| 2151 | gctgaagcgt ggctacgcca agatcggtcg gccggaactc cgcatccacg |
| 2201 | acctccgcgc tgtcggcgct acgttcgccg ctcaggcagg tgcgacgacc |
| 2251 | aaggagctga tggcccgtct cggtcacacg actcctagga tggcgatgaa |
| 2301 | gtaccagatg gcgtctgagg cccgcgacga ggctatcgct gaggcgatgt |
| 2351 | ccaagctggc caagacctcc tgaaacgcaa aaagcccccc tcccaaggac |
| 2401 | actgagtcct aaagaggggg gtttcttgtc agtacgcgaa gaaccacgcc |
| 2451 | tggccgcgag cgccagcacc gccgctctgt gcggagacct gggcaccagc |
| 2501 | cccgccgccg ccaggagcat tgccgttccc gccagaaatc tagaggtgac |
| 2551 | cacaacgacg cgcccgcttt gatcggggac gtctgcggcc gaccatttac |
| 2601 | gggtcttgtt gtcgttggcg gtcatgggcc gaacatactc acccggatcg |
| 2651 | gagggccgag gacaaggtcg aacgaggggc atgacccggt gcggggcttc |
| 2701 | ttgcactcgg cataggcgag tgctaagaat aacgttggca ctcgcgaccg |
| 2751 | gtgagtcgta ggtcgggacg gtgaggccag gcccgtcgtc gcagcgagtg |
| 2801 | gcagcgagga caacttgagc cgtccgtcgc gggcactgcg cccggccagc |
| 2851 | gtaagtagcg gggttgccgt cacccggtga cccccggttt catccccgat |
| 2901 | ccggaggaat cacttcgcaa tggccaagac aattgcggat ccagctgcag |
| 2951 | aattcgaagc ttatcgatgt cgacgtagtt aactagcgta cgatcgactg |
| 3001 | ccaggcatca aataaaacga aaggctcagt cgaaagactg ggcctttcgt |
| 3051 | tttatctgtt gtttgtccgg ccatcatggc cgcggtgatc agctagccaa |
| 3101 | caaagcgacg ttgtgtctca aaatctctga tgttacattg cacaagataa |
| 3151 | aaatatatca tcatgatcga attcctgcag ctcacggtaa ctgatgccgt |
| 3201 | atttgcagta ccagcgtacg gcccacagaa tgatgtcacg ctgaaaatgc |
| 3251 | cggcctttga atgggttcat gtgcagctcc atcagcaaaa ggggatgata |
| 3301 | agtttatcac caccgactat ttgcaacagt gccgttgatc gtgctatgat |
| 3351 | cgactgatgt catcagcggt ggagtgcaat gtcgtgcaat acgaatggcg |
| 3401 | aaaagccgag ctcatcggtc agcttctcaa ccttggggtt acccccggcg |
| 3451 | gtgtgctgct ggtccacagc tccttccgta gcgtccggcc cctcgaagat |
| 3501 | gggccacttg gactgatcga ggccctgcgt gctacgctgg gtccgggagg |
| 3551 | gacgctcgtc atgccctcgt ggtcaggtct ggacgacgag ccgttcgatc |
| 3601 | ctgccacgtc gcccgttaca ccggaccttg gagttgtctc tgacacattc |
| 3651 | tggcgcctgc caaatgtaaa gcgcagcgcc catccatttg cctttgcggc |
| 3701 | agcggggcca caggcagagc agatcatctc tgatccattg cccctgccac |
| 3751 | cttactcgcc tgcaagcccg gtcgcccgtg tccatgaact cgatgggcag |
| 3801 | gtacttctcc tcggcgtggg acacgatgcc aacacgacgc tgcatcttgc |
| 3851 | cgagttgatg gcaaaggttc cctatggggt gccgagacac tgcaccattc |
| 3901 | ttcaggatgg caagttggta cgcgtcgatt atctcgagaa tgaccactgc |
| 3951 | tgtgagcgct ttgccttggc ggacaggtgg ctcaaggaga agagccttca |
| 4001 | gaaggaaggt ccagtcggtc atgcctttgc tcggttgatc cgctcccgcg |
| 4051 | acattgtggc gacagccctg ggtcaactgg gccgagatcc gttgatcttc |
| 4101 | ctgcatccgc cagaggcggg atgcgaagaa tgcgatgccg ctcgccagtc |
| 4151 | gattggctga gctcatgagc ggagaacgag atgacgttgg aggggcaagg |
| 4201 | tcgcgctgat tgctggggca acacggggga tccactagtc caccaccaga |
| 4251 | cggccgatcc ccaccggccg ccggccaccc actgccacca cgaccagacc |
| 4301 | cagcatcaac tgcccgggtg tgaatccgaa caagcggacc gccgccaccc |
| 4351 | cgagcagcag ccaaatcacc aggacaaccg tcgacagcat cggggtcgac |
| 4401 | caaacaccga attccacgcc cagcaacgcc agaccgtagg cgatcagcca |
| 4451 | gtcgatcagc agagccgcca gccggcgccc catcggagcc agcgaacccg |
| 4501 | gtccggtgtc cggcaagccc agcgtcttgc cgggatagtc gggcggcgat |
| 4551 | ttcgccgtca tcgggcagac ccgataacca ggttcccgtt cggcatgcca |
| 4601 | ccggttacga tcttgccgac catggcccca caatagggcc ggggagaccc |
| 4651 | ggcgtcagtg gtgggcggca cggtcagtaa cgtctgcgca acacggggtt |
| 4701 | gactgacggg caatatcggc tccatagcgt cggccgcgga tacagtaaag |
| 4751 | gagcattctg tgacggaaaa gacgcccgac gacgtcttca aacttgccaa |
| 4801 | ggacgagaag gtcgaatatg tcgacgtccg gttctgtgac ctgcctggca |
| 4851 | tcatgcagca cttcacgatt ccggcttcgg cctttgacaa gagcgtgttt |
| 4901 | gacgacggct tggcctttgg ctcgtcgatt cgcgggttcc agtcgatcca |
| 4951 | cgaatccgac atgttgcttc ttcccgatcc cgagacggcg cgcatcgacc |
| 5001 | cgttccgcgc ggccaagacg ctgaatatca acttctttgt gcacgacccg |
| 5051 | ttcaccctgg agccgtactc ccgcgacccg cgcaacatcg cccgcaaggc |
| 5101 | cgagaactac ctgatcagca ctggcatcgc cgacaccgca tacttcggcg |
| 5151 | ccgaggccga gttctacatt ttcgattcgg tgagcttcga ctcgcgcgcc |
| 5201 | aacggctcct tctacgaggt ggacgccatc tcggggtggt ggaacaccgg |
| 5251 | cgcggcgacc gaggccgacg gcagtcccaa ccggggctac aaggtccgcc |
| 5301 | acaagggcgg gtatttccca gtggccccca acgaccaata cgtcgacctg |
| 5351 | cgcgacaaga tgctgaccaa cctgatcaac tccggcttca tcctggagaa |
| 5401 | gggccaccac gaggtgggca gcggcggaca ggccgagatc aactaccagt |
| 5451 | tcaattcgct gctgcacgcc gccgacgaca tgcagttgta caagtacatc |
| 5501 | atcaagaaca ccgcctggca gaacggcaaa acggtcacgt tcatgcccaa |
| 5551 | gccgctgttc ggcgacaacg ggtccggcat gcactgtcat cagtcgctgt |
| 5601 | ggaaggacgg ggccccgctg atgtacgacg agacgggtta tgccggtctg |
| 5651 | tcggacacgg cccgtcatta catcggcggc ctgttacacc acgcgccgtc |
| 5701 | gctgctggcc ttcaccaacc cgacggtgaa ctcctacaag cggctggttc |
| 5751 | ccggttacgc cccgatcaac ctggtctata gccagcgcaa ccggtcggca |
| 5801 | tgcgtgcgca tcccgatcac cggcagcaac ccgaaggcca agcggctgga |
| 5851 | gttccgaagc cccgactcgt cgggcaaccc gtatctggcg ttctcggcca |
| 5901 | tgctgatggc aggcctggac ggtatcaaga acaagatcga gccgcaggcg |
| 5951 | cccgtcgaca aggatctcta cgagctgccg ccggaagagg ccgcgagtat |
| 6001 | cccgcagact ccgacccagc tgtcagatgt gatcgaccgt ctcgaggccg |
| 6051 | accacgaata cctcaccgaa ggaggggtgt tcacaaacga cctgatcgag |
| 6101 | acgtggatca gtttcaagcg cgaaaacgag atcgagccgg tcaacatccg |
| 6151 | gccgcatccc tacgaattcg cgctgtacta cgacgtttaa ggactcttcg |
| 6201 | cagtccgggt gtagagggag cggcgtggac tagttccact gagcgtcaga |
| 6251 | ccccgtagaa aagatcaaag gatcttcttg agatcctttt tttctgcgcg |
| 6301 | taatctgctg cttgcaaaca aaaaaaccac cgctaccagc ggtggtttgt |
| 6351 | ttgccggatc aagagctacc aactcttttt ccgaaggtaa ctggcttcag |
| 6401 | cagagcgcag ataccaaata ctgtccttct agtgtagccg tagttaggcc |
| 6451 | accacttcaa gaactctgta gcaccgccta catacctcgc tctgctaatc |
| 6501 | ctgttaccag tggctgctgc cagtggcgat aagtcgtgtc ttaccgggtt |
| 6551 | ggactcaaga cgatagttac cggataaggc gcagcggtcg ggctgaacgg |
| 6601 | ggggttcgtg cacacagccc agcttggagc gaacgaccta caccgaactg |
| 6651 | agatacctac agcgtgagca ttgagaaagc gccacgcttc ccgaagggag |
| 6701 | aaaggcggac aggtatccgg taagcggcag ggtcggaaca ggagagcgca |
| 6751 | cgagggagct tccaggggga aacgcctggt atctttatag tcctgtcggg |
| 6801 | tttcgccacc tctgacttga gcgtcgattt ttgtgatgct cgtcaggggg |
| 6851 | gcggagccta tggaaaaacg ccagcaacgc ggccttttta cggttcctgg |
| 6901 | ccttttgctg gccttttgct cacatgttct ttcctgcgtt atcccctgat |
| 6951 | tctgtggata accgtattac cgcctttgag tgagctgata ccgctcgccg |
| 7001 | cagccgaacg accgagcgca acgcgtgcgg ccgctaaact gttacaacgc |
| 7051 | tcacatatgt ggttggcgac gagcccaagg cagtcgcctc gctgttcaat |
| 7101 | ctgtgaccgg atccgcagga cgtcgatccg tgggtttacc tgcggatttg |
| 7151 | tcgttactgg cgggtagctt ctgaaacggt tcagtttttg ggcgacttcg |
| 7201 | caaaatttgc aaaaagtccg caggccgttg ccgaaattcg caagtgaaat |
| 7251 | gggtggacca gcgttgacac gctgtgccat ggtcgagtta gcacaccagt |
| 7301 | gaagctgcgc cgttgacacc gcctggacga cggtagggcg tcagcgtttt |
| 7351 | cggcaatgaa agaccgttaa ggagttgtct |

(K) pYUB854-sigH (SEQ ID NO:33)

The vector for sigH inactivation by using the phasmid system, added to BCG to construct BCGΔsigH and to BCGΔsecA2 to construct DD-BCG. It can be used to modify 1^{st}, 2^{nd}, and 3^{rd} generation pro-apoptotic BCG vaccines, respectively, into 2^{nd}, 3^{rd}, and 4^{th} generation pro-apoptotic BCG vaccines.

| | |
|---|---|
| 1 | ggatcctgat caggcgcctt aattaagatc cctatagtga gtcgtattat |
| 51 | gcggccgcga attctcatgt ttgaccgctt atcatcgata agctctgctt |
| 101 | tttgttgact tccattgttc attccacgga caaaaacaga gaaaggaaac |
| 151 | gacagaggcc aaaaagctcg ctttcagcac ctgtcgtttc ctttcttttc |
| 201 | agagggtatt ttaaataaaa acattaagtt atgacgaaga agaacggaaa |
| 251 | cgccttaaac cggaaaattt tcataaatag cgaaaacccg cgaggtcgcc |
| 301 | gccccgtaac aaggcggatc gccggaaagg acccgcaaat gataataatt |
| 351 | atcaattcgc gaacttgttt attgcagctt ataatggtta caaataaagc |
| 401 | aatagcatca caaatttcac aaataaagca tttttttcac tgcattctag |
| 451 | ttgtggtttg tccaaactca tcaatgtatc ttatcatgtc tggatctgac |
| 501 | gggtgcgcat gatcgtgctc ctgtcgttga ggacccggct aggctggcgg |
| 551 | ggttgcctta ctggttagca gaatgaatca ccgatacgcg agcgaacgtg |
| 601 | aagcgactgc tgctgcaaaa cgtctgcgac ctgagcaaca acatgaatgg |
| 651 | tcttcggttt ccgtgtttcg taaagtctgg aaacgcggaa gtcagcgctc |
| 701 | ttccgcttcc tcgctcactg actcgctgcg ctcggtcgtt cggctgcggc |
| 751 | gagcggtatc agctcactca aaggcggtaa tacggttatc cacagaatca |
| 801 | ggggataacg caggaaagaa catgtgagca aaaggccagc aaaaggccag |
| 851 | caaaaggcca ggaaccgtaa aaaggccgcg ttgctggcgt ttttccatag |
| 901 | gctccgcccc cctgacgagc atcacaaaaa tcgacgctca agtcagaggt |
| 951 | ggcgaaaccc gacaggacta taaagatacc aggcgtttcc ccctggaagc |
| 1001 | tccctcgtgc gctctcctgt tccgaccctg ccgcttaccg gatacctgtc |
| 1051 | cgcctttctc ccttcgggaa gcgtggcgct ttctcatagc tcacgctgta |
| 1101 | ggtatctcag ttcggtgtag gtcgttcgct ccaagctggg ctgtgtgcac |
| 1151 | gaaccccccg ttcagcccga ccgctgcgcc ttatccggta actatcgtct |
| 1201 | tgagtccaac ccggtaagac acgacttatc gccactggca gcagccactg |
| 1251 | gtaacaggat tagcagagcg aggtatgtag gcggtgctac agagttcttg |
| 1301 | aagtggtggc ctaactacgg ctacactaga aggacagtat ttggtatctg |
| 1351 | cgctctgctg aagccagtta ccttcggaaa aagagttggt agctcttgat |
| 1401 | ccggcaaaca aaccaccgct ggtagcggtg gtttttttgt ttgcaagcag |
| 1451 | cagattacgc gcagaaaaaa aggatctcaa gaagatcctt tgatattttc |
| 1501 | tacggggtct gacgctcagt cgaacgaaaa ctcacgttaa gggattttgg |
| 1551 | tcatggtaat acgactcact agttcacggc ctttcatatg gggaatgcac |
| 1601 | gcttgggata ctgcgtaggt gccacgcacc tggatgccgt tcatcaggtc |
| 1651 | gaaacgcttc attggcacct cggtgatgga ccctaagttg atcgccgagg |
| 1701 | cattgttgac gcagatatcg atgcccccga actgctccac ggtggtggcc |
| 1751 | accgcggacg cgaccgcatc cgggtcgcgg atatccccga cgatcggcag |
| 1801 | tgcctggccg ccggcttcct cgagttcctt ggcggccgtg aacaccgtgc |
| 1851 | ctggcagctt tggatgcggc tcggcggtct tggcgatcaa ggcaatgttg |
| 1901 | gcgccgtcgc gcgcggcccg cttggcgatc gcaaggccga taccgcgact |
| 1951 | ggcgccagag atgaacatgg tcttgccgtt gagtgacatg gcgtcaccct |
| 2001 | aacgccctgc tcgattccac cggactgcgg gacaggccgc atgcgattca |
| 2051 | gcgctggaaa taacccgctg gcgaacacgg ttgattggtt cggttcctgc |
| 2101 | agccacgtgg ctggccagac cggtgcagac agtgtcggtc gcggcctcta |
| 2151 | cattgggacg aggggagtct ggtgtcaacg gcgacgagcc tgttgggtga |
| 2201 | gaagcggttg gctcgcatgc tcgcacgtcc ggtgtccgcg ccggtgctgt |
| 2251 | ccggcgacac cgcaaacgaa gggacccagt taagatcttc gaatgcatcg |
| 2301 | cgcgcaccgt acgtctcgag gaattcctgc aggatatctg gatccacgaa |
| 2351 | gcttcccatg gtgcgcgtgc tagcaaccgt ccgaaatatt ataaattatc |
| 2401 | acacacataa aaacagtgct gttaatgtgt ctattaagtc gattttttgt |
| 2451 | tataacagac actgcttgtc cgatatctga tttaggatac atttntagcc |
| 2501 | acctgggggc gtcaggcgcc gggggcggtg tccggcggcc cccagaggaa |
| 2551 | ctgcgccagt tcctccggat cggtgaagcc ggagagatcc agcggggtct |
| 2601 | cctcgaacac ctcgaagtcg tgcaggaagg tgaaggcgag cagttcgcgg |
| 2651 | gcgaagtcct cggtccgctt ccactgcgcc ccgtcgagca gcgcggccag |
| 2701 | gatctcgcgg tcgccccgga aggcgttgag atgcagttgc accaggctgt |
| 2751 | agcgggagtc tcccgcatag acgtcggtga agtcgacgat cccggtgacc |
| 2801 | tcggtcgcgg ccaggtccac gaagatgttg gtcccgtgca ggtcgccgtg |
| 2851 | gacgaaccgg ggttcgcggc cggccagcag cgtgtccacg tccggcagcc |
| 2901 | agtcctccag gcggtccagc agccggggcg agaggtagcc ccacccgcgg |
| 2951 | tggtcctcga cggtcgccgc gcggcgttcc cgcagcagtt ccgggaagac |
| 3001 | ctcggaatgg ggggtgagca cggtgttccc ggtcagcggc accctgtgca |
| 3051 | gccggccgag cacccggccg agttcgcggg ccagggcgag cagcgcgttc |
| 3101 | cggtcggtcg tgccgtccat cgcggaccgc caggtggtgc cggtcatccg |
| 3151 | gctcatcacc aggtagggcc acggccaggc tccggtgccg ggccgcagct |
| 3201 | cgccgcggcc gaggaggcgg ggcaccggca ccggggcgtc cgccaggacc |
| 3251 | gcgtacgcct ccgactccga cgcgaggctc tccggaccgc accagtgctc |
| 3301 | gccgaacagc ttgatgaccg ggtcgggctc gccgaccagt acggggttgg |
| 3351 | tgctctcgcc gggcacccgc agcaccggcg gcaccggcag cccgagctcc |
| 3401 | tccagggctc ggcgggccag cggctcccag aattcctggt cgttccgcag |
| 3451 | gctcgcgtag gaatcatccg aatcaatacg gtcgagaagt aacagggatt |
| 3501 | cttgtgtcac agcggacctc tattcacagg gtacgggccg gcttaattcc |
| 3551 | gcacggccgg tcgcgacacg gcctgtccgc accgcggtca ggcgttgacg |
| 3601 | atgacgggct ggtcggccac gtcggggacg ttctcggtgg tgctgcggtc |
| 3651 | gggatcgcca atctctacgg gccgaccgag gcgacggtgt acgccaccgc |
| 3701 | ctggttctgc gacggcgagg cgccgccagg ccccgccgat ccccgtcccc |
| 3751 | cgcgtcgtcg agcgcggtgc cgacgacacc gccgcgtggc tcgtcacgga |
| 3801 | ggccgtcccc ggcgtcgcgg cggccgagga gtggcccgag caccagcggt |
| 3851 | tcgccgtggt cgaggcgatg gcggagctgg cccgcgccct ccacgagctg |
| 3901 | cccgtggagg actgcccctc cgaccggcgc ctcgacgcgg cggtcgccga |
| 3951 | ggcccggcgg aacgtcgccg agggcttggt ggacctcgac gacctgcagg |
| 4001 | catgcaagct caggatgtcc acctacaaca aagctctcac caaccgtggc |
| 4051 | tccctcactt tctggctgga tgatggggcg attcangcct ggtatgantc |
| 4101 | agcaacacct tcttcacgan gcagacctca ctagcaaccg tccgaaatat |
| 4151 | tataaattat cgcacacata aaaacagtgc tgttaatgtg tctattaagt |
| 4201 | cgattttttg ttataacaga cactgcttgt ccgatatttg atttaggata |
| 4251 | cacgcgcacc ggttctagac cgagcagatc accgattggc aactggcgtc |
| 4301 | caacgccgag cattcctcga ccgggctgcg ctcggctgaa gtcgaagcgt |
| 4351 | tagaagcgtt gccggacacc gagatcaaag aggcgctgca ggcattgccg |
| 4401 | gaagagttcc ggatggcggt ctactacgcc gatgtcgaag gtttccccta |
| 4451 | caaggagatc gccgagatca tggatactcc gatcggcacc gtgatgtcga |
| 4501 | ggcttcatcg cggccgacgt cagttgcgcg gtcttttagc cgatgtggcc |
| 4551 | agggatcggg ggtttgccag gggcgagcag gcgcacgagg gggtgtcgtc |
| 4601 | atgagcagcc cggtgtcaag ccgccgtttg gcgaacttgg tcaaggagag |
| 4651 | cctgcagggc tcggtgttgg gtggggtcgt aagcgatgcc gtcttgccag |
| 4701 | cggtgtcaga tgacgtaaag ccaggcgcgg gcgaggatgc gtaccgcgtg |
| 4751 | ccggtggtcg tggccgcggg ctcgggtgcg gttgtgcagg tcggcggcct |
| 4801 | agaggttggc tcggcggctg tcgccggcga agtcgcagac accgttgcgg |
| 4851 | agttgtttgt ctgccgccca accgaacccg acgtgggtga ctttgtcgga |
| 4901 | ctagccggtg gagcgggcga cgccggccaa gcaggccagc aattcgggct |
| 4951 | gggcgtcggc gtgcggggcg agtcgttcgg cgctcgtcgg cgcttggccc |
| 5001 | tgtcgacggt cggcgcgtcc ggggcaaccg ccggactccg caaaactcat |
| 5051 | gatggacatc acggctgtca agcttaagtg agtcgtatta cggactggga |
| 5101 | gtcaggcaac tatggatgaa cgaaatagac agatcgctga gataggtgcc |
| 5151 | tcactgatta agcattggta actgtcagac caagtttact catatatact |
| 5201 | ttagattgat ttaccccggt tgataatcag aaaagcccca aaaacaggaa |
| 5251 | gattgtataa gcaaatattt catgacatta acctataaaa ataggcgtat |
| 5301 | cacgaggccc tttcgtcttc aagaattcgc ggccgcaatt aaccctcact |
| 5351 | aaaggatctt aattaa |

(L) pYUB854-trx-trxr (SEQ ID NO:34)

The vector for inactivation of thioredoxin (trxC, also trx) and thioredoxin reductase (trxB2, also trxr) by using the phasmid system. It can be electroporated into BCG to construct BCGΔtrxΔtrxr. It can also be used to modify 1^{st}, 2^{nd}, and 3^{rd} generation pro-apoptotic BCG vaccines, respectively, into 2^{nd}, 3^{rd}, and 4^{th} generation pro-apoptotic BCG vaccines.

| | |
|---|---|
| 1 | ggatcctgat caggcgcctt aattaagatc cctatagtga gtcgtattat gcggccgcga |
| 61 | attctcatgt ttgaccgctt atcatcgata agctctgctt tttgttgact tccattgttc |
| 121 | attccacgga caaaaacaga gaaaggaaac gacagaggcc aaaaagctcg ctttcagcac |
| 181 | ctgtcgtttc ctttcttttc agagggtatt ttaaataaaa acattaagtt atgacgaaga |
| 241 | agaacggaaa cgccttaaac cggaaaattt tcataaatag cgaaaacccg cgaggtcgcc |
| 301 | gccccgtaac aaggcggatc gccggaaagg acccgcaaat gataataatt atcaattcgc |
| 361 | gaacttgttt attgcagctt ataatggtta caaataaagc aatagcatca caaatttcac |
| 421 | aaataaagca tttttttcac tgcattctag ttgtggtttg tccaaactca tcaatgtatc |
| 481 | ttatcatgtc tggatctgac gggtgcgcat gatcgtgctc ctgtcgttga ggacccggct |
| 541 | aggctggcgg ggttgcctta ctggttagca gaatgaatca ccgatacgcg agcgaacgtg |
| 601 | aagcgactgc tgctgcaaaa cgtctgcgac ctgagcaaca acatgaatgg tcttcggttt |
| 661 | ccgtgtttcg taaagtctgg aaacgcggaa gtcagcgctc ttccgcttcc tcgctcactg |
| 721 | actcgctgcg ctcggtcgtt cggctgcggc gagcggtatc agctcactca aaggcggtaa |
| 781 | tacggttatc cacagaatca ggggataacg caggaaagaa catgtgagca aaaggccagc |
| 841 | aaaaggccag caaaaggcca ggaaccgtaa aaaggccgcg ttgctggcgt ttttccatag |
| 901 | gctccgcccc cctgacgagc atcacaaaaa tcgacgctca agtcagaggt ggcgaaaccc |
| 961 | gacaggacta taaagatacc aggcgtttcc ccctggaagc tccctcgtgc gctctcctgt |
| 1021 | tccgaccctg ccgcttaccg gatacctgtc cgcctttctc ccttcgggaa gcgtggcgct |
| 1081 | ttctcatagc tcacgctgta ggtatctcag ttcggtgtag gtcgttcgct ccaagctggg |
| 1141 | ctgtgtgcac gaaccccccg ttcagcccga ccgctgcgcc ttatccggta actatcgtct |
| 1201 | tgagtccaac ccggtaagac acgacttatc gccactggca gcagccactg gtaacaggat |
| 1261 | tagcagagcg aggtatgtag gcggtgctac agagttcttg aagtggtggc ctaactacgg |
| 1321 | ctacactaga aggacagtat ttggtatctg cgctctgctg aagccagtta ccttcggaaa |
| 1381 | aagagttggt agctcttgat ccggcaaaca aaccaccgct ggtagcggtg gtttttttgt |
| 1441 | ttgcaagcag cagattacgc gcagaaaaaa aggatctcaa gaagatcctt tgatattttc |
| 1501 | tacggggtct gacgctcagt cgaacgaaaa ctcacgttaa gggattttgg tcatggtaat |
| 1561 | acgactcact agtagacccg caggctcagc aaatcctgcg cgcgttgaac cgggtacgcc |
| 1621 | gcgatgtcgc cgcgatgggt gccgaccccg cttgggggcc agctgctcgc ccagcggtcg |
| 1681 | tcgacagcat ttcggcggcc ttacggtcgg cgcgcccgaa cagctcaccc ggcgccgctc |
| 1741 | acgccgcccg tccgcacgtc caccccgtcc gaatgatcgc cggcgcggcc ggattgtgcg |
| 1801 | ccgtggccac agcgatcggt gtcggcgccg tggtcgatgc accgccaccc gcaccgagtg |
| 1861 | caccgacaac cgcgcagcac atcacggtgt caaaacctgc cccggtgatt ccgctgtctc |
| 1921 | ggccgcaggt tctcgacctg cttcaccaca ccccggacta tggcccaccc ggaggcccgc |
| 1981 | tgggcgatcc gtcccggcgt acgtcctgcc tgagcggcct cggctatccg gcgtccacgc |
| 2041 | cggtgctggg cgcgcagccg atcgatatcg acgctcggcc cgccgtactg ctggtgatac |
| 2101 | ccgcggacac gcccgacaaa ctggccgttt ttgcggtcgc gccgcactgc agcgccgccg |
| 2161 | ataccgggtt gttggctagc accgtggtcc cccgcgcatg atgggtctgg gtgctgtcgc |
| 2221 | tcgcctgcgg gaacagcagt gcctacgctg gcgttcgttg tctttcgaat gcatcgcgcg |
| 2281 | caccgtacgt ctcgaggaat tcctgcagga tatctggatc cacgaagctt cccatggtgc |
| 2341 | gcgtgctagc aaccgtccga aatattataa attatcacac acataaaaac agtgctgtta |
| 2401 | atgtgtctat taagtcgatt ttttgttata acagacactg cttgtccgat atctgattta |
| 2461 | ggatacattt ntagccacct gggggcgtca ggcgccgggg gcggtgtccg gcggccccca |
| 2521 | gaggaactgc gccagttcct ccggatcggt gaagccggag agatccagcg gggtctcctc |
| 2581 | gaacacctcg aagtcgtgca ggaaggtgaa ggcgagcagt tcgcgggcga agtcctcggt |
| 2641 | ccgcttccac tgcgccccgt cgagcagcgc ggccaggatc tcgcggtcgc cccggaaggc |
| 2701 | gttgagatgc agttgcacca ggctgtagcg ggagtctccc gcatagacgt cggtgaagtc |
| 2761 | gacgatcccg gtgacctcgg tcgcggccag gtccacgaag atgttggtcc cgtgcaggtc |
| 2821 | gccgtggacg aaccggggtt cgcggccggc cagcagcgtg tccacgtccg gcagccagtc |
| 2881 | ctccaggcgg tccagcagcc ggggcgagag gtagccccac ccgcggtggt cctcgacggt |
| 2941 | cgccgcgcgg cgttcccgca gcagttccgg gaagacctcg gaatgggggg tgagcacggt |
| 3001 | gttcccggtc agcggcaccc tgtgcagccg gccgagcacc cggccgagtt cgcgggccag |
| 3061 | ggcgagcagc gcgttccggt cggtcgtgcc gtccatcgcg gaccgccagg tggtgccggt |
| 3121 | catccggctc atcaccaggt agggccacgg ccaggctccg gtgccgggcc gcagctcgcc |
| 3181 | gcggccgagg aggcggggca ccggcaccgg ggcgtccgcc aggaccgcgt acgcctccga |
| 3241 | ctccgacgcg aggctctccg gaccgcacca gtgctcgccg aacagcttga tgaccgggtc |
| 3301 | gggctcgccg accagtacgg ggttggtgct ctcgccgggc acccgcagca ccggcggcac |
| 3361 | cggcagcccg agctcctcca gggctcggcg ggccagcggc tcccagaatt cctggtcgtt |
| 3421 | ccgcaggctc gcgtaggaat catccgaatc aatacggtcg agaagtaaca gggattcttg |
| 3481 | tgtcacagcg gacctctatt cacagggtac gggccggctt aattccgcac ggccggtcgc |
| 3541 | gacacggcct gtccgcaccg cggtcaggcg ttgacgatga cgggctggtc ggccacgtcg |
| 3601 | gggacgttct cggtggtgct gcggtcggga tcgccaatct ctacgggccg accgaggcga |
| 3661 | cggtgtacgc caccgcctgg ttctgcgacg gcgaggcgcc gccaggcccc gccgatcccc |
| 3721 | gtcccccgcg tcgtcgagcg cggtgccgac gacaccgccg cgtggctcgt cacggaggcc |
| 3781 | gtccccggcg tcgcggcggc cgaggagtgg cccgagcacc agcggttcgc cgtggtcgag |
| 3841 | gcgatggcgg agctggcccg cgccctccac gagctgcccg tggaggactg cccctccgac |
| 3901 | cggcgcctcg acgcggcggt cgccgaggcc cggcggaacg tcgccgaggg cttggtggac |
| 3961 | ctcgacgacc tgcaggcatg caagctcagg atgtccacct acaacaaagc tctcaccaac |
| 4021 | cgtggctccc tcactttctg gctggatgat ggggcgattc angcctggta tgantcagca |
| 4081 | acaccttctt cacgangcag acctcactag caaccgtccg aaatattata aattatcgca |
| 4141 | cacataaaaa cagtgctgtt aatgtgtcta ttaagtcgat tttttgttat aacagacact |
| 4201 | gcttgtccga tatttgattt aggatacacg cgcaccggtt ctagaccgaa atcggcaagg |
| 4261 | atctgcgaca ataccggttg gctggtccgc attgtcaacg atgtgagcta atcccggagg |
| 4321 | gcccttggta tgccgagtcc gcgccgcgaa gacggcgatg cgctgcgctg tggcgaccgc |
| 4381 | agtgcggccg tcaccgagat ccgggctgcg ctgaccgcgt tagggatgct ggatcatcag |
| 4441 | gaagaagacc tgacgacggg ccgtaacgtc gcccttgagt tgttcgacgc gcagctcgac |
| 4501 | caggcggtcc gtgccttcca acagcatcgc ggcctgctgg tggacggcat cgtcggtgag |
| 4561 | gccacctacc gcgcgttgaa agaagcctcc taccggctcg gggcccgcac gctgtaccac |
| 4621 | caattcggcg ccccgctcta cggggacgac gtcgctacac tgcaggcccg gctgcaggat |
| 4681 | cttggtttct acaccgggct ggtcgacggt catttcgggt tgcagaccca caatgcgttg |
| 4741 | atgtcctatc agcgtgagta cggacttgcc gcagacggta tctgcggccc agaaacgttg |
| 4801 | cgctccttgt actttctaag ttcgcgagtc agcggtggct cgccacatgc gattcgcgaa |
| 4861 | gaagagctgg tccgcagctc ggggccgaag ctgtctggca aacggatcat cattgatccc |
| 4921 | ggtcgcggcg gcgtggacca cggacttatc gcgcaaggtc cggctgggcc catcagcgaa |
| 4981 | gcagacttga ggccttaagt gagtcgtatt acggactggg agtcaggcaa ctatggatga |
| 5041 | acgaaataga cagatcgctg agataggtgc ctcactgatt aagcattggt aactgtcaga |
| 5101 | ccaagtttac tcatatatac tttagattga tttaccccgg ttgataatca gaaaagcccc |
| 5161 | aaaaacagga agattgtata agcaaatatt tcatgacatt aacctataaa aataggcgta |
| 5221 | tcacgaggcc ctttcgtctt caagaattcg cggccgcaat taaccctcac taaaggatct |
| 5281 | taattaa |

(M) pYUB854-sigE (SEQ ID NO:35)

The vector for inactivation of sigE by using the phasmid system. It can be electroporated into BCG to construct BCGΔsigE. It can also be used to modify pro-apoptotic BCG vaccines to make them more immunogenic.

| | |
|---|---|
| 1 | ggatcctgat caggcgcctt aattaagatc cctatagtga gtcgtattat gcggccgcga |
| 61 | attctcatgt ttgaccgctt atcatcgata agctctgctt tttgttgact tccattgttc |
| 121 | attccacgga caaaaacaga gaaaggaaac gacagaggcc aaaaagctcg ctttcagcac |
| 181 | ctgtcgtttc ctttcttttc agagggtatt ttaaataaaa acattaagtt atgacgaaga |
| 241 | agaacggaaa cgccttaaac cggaaaattt tcataaatag cgaaaacccg cgaggtcgcc |
| 301 | gccccgtaac aaggcggatc gccggaaagg acccgcaaat gataataatt atcaattcgc |
| 361 | gaacttgttt attgcagctt ataatggtta caaataaagc aatagcatca caaatttcac |
| 421 | aaataaagca tttttttcac tgcattctag ttgtggtttg tccaaactca tcaatgtatc |
| 481 | ttatcatgtc tggatctgac gggtgcgcat gatcgtgctc ctgtcgttga ggacccggct |
| 541 | aggctggcgg ggttgcctta ctggttagca gaatgaatca ccgatacgcg agcgaacgtg |
| 601 | aagcgactgc tgctgcaaaa cgtctgcgac ctgagcaaca acatgaatgg tcttcggttt |
| 661 | ccgtgtttcg taaagtctgg aaacgcggaa gtcagcgctc ttccgcttcc tcgctcactg |
| 721 | actcgctgcg ctcggtcgtt cggctgcggc gagcggtatc agctcactca aaggcggtaa |
| 781 | tacggttatc cacagaatca ggggataacg caggaaagaa catgtgagca aaaggccagc |
| 841 | aaaaggccag caaaaggcca ggaaccgtaa aaaggccgcg ttgctggcgt ttttccatag |
| 901 | gctccgcccc cctgacgagc atcacaaaaa tcgacgctca agtcagaggt ggcgaaaccc |
| 961 | gacaggacta taaagatacc aggcgtttcc ccctggaagc tccctcgtgc gctctcctgt |
| 1021 | tccgaccctg ccgcttaccg gatacctgtc cgcctttctc ccttcgggaa gcgtggcgct |
| 1081 | ttctcatagc tcacgctgta ggtatctcag ttcggtgtag gtcgttcgct ccaagctggg |
| 1141 | ctgtgtgcac gaaccccccg ttcagcccga ccgctgcgcc ttatccggta actatcgtct |
| 1201 | tgagtccaac ccggtaagac acgacttatc gccactggca gcagccactg gtaacaggat |
| 1261 | tagcagagcg aggtatgtag gcggtgctac agagttcttg aagtggtggc ctaactacgg |
| 1321 | ctacactaga aggacagtat ttggtatctg cgctctgctg aagccagtta ccttcggaaa |
| 1381 | aagagttggt agctcttgat ccggcaaaca aaccaccgct ggtagcggtg gtttttttgt |
| 1441 | ttgcaagcag cagattacgc gcagaaaaaa aggatctcaa gaagatcctt tgatattttc |
| 1501 | tacggggtct gacgctcagt cgaacgaaaa ctcacgttaa gggattttgg tcatggtaat |
| 1561 | acgactcact agtccgtcgc gcgccccggg atcaccggcc cgaccgccca gcgccgcccg |
| 1621 | gtgcacgacg atgaccccgc cggatcgcag cagccgcacc ccctcggcga cgtaatctgg |
| 1681 | ctggtcgatc gggtcggcgt cgatgaatac caggtcgtag gatgcgtcgg cgagccgggt |
| 1741 | cagcacctct tgggcgcggc cgctgatcag cctggtacgc gacggcccga tgcccgcctc |
| 1801 | ggcaaaggcc tgcctggcaa ggcgtagatg ctcgggctcg atatcgatgg tggtcaagac |
| 1861 | gccgtcgtcg cgcatgcccg acaacagcca caggccgctg acgccggccc cggtacccac |
| 1921 | ttcggccacc gccttgcctc cgctgagctt ggccagcaag cacagcaacg cacccaccgc |
| 1981 | cggtgttacc gccccggccc cgatgtcggt tgcgcgctcg cgggcgccgg ccaggatcac |
| 2041 | gtcttcagat attgacccct cggcgtgcgc ccagagtgat tcgcctcggc tgggggccgg |
| 2101 | ctggccaggc atgtcgtcgt gtccgggggt gccgtccatg cccgcagcgt atgtccaatt |
| 2161 | ggcgacgccg tcgggcaggc gcgcctggtt cgaacgccgg ccgagcaccg agctggacgc |
| 2221 | ttgcggctgt acccgacacg cccggcgtgc cggacgcgac gaaggtcact ttgactcgat |
| 2281 | attccctgga cagcgcaggt aacggtatgg tttctaagcc aaagctcaga ttgctcatat |
| 2341 | atggcccata cgccggtacg cgacggtaat tcccgctcga ggaattcctg caggatatct |
| 2401 | ggatccacga agcttcccat ggtgcgcgtg ctagcaaccg tccgaaatat tataaattat |
| 2461 | cacacacata aaaacagtgc tgttaatgtg tctattaagt cgattttttg ttataacaga |
| 2521 | cactgcttgt ccgatatctg atttaggata catttntagc cacctggggg cgtcaggcgc |
| 2581 | cgggggcggt gtccggcggc ccccagagga actgcgccag ttcctccgga tcggtgaagc |
| 2641 | cggagagatc cagcggggtc tcctcgaaca cctcgaagtc gtgcaggaag gtgaaggcga |
| 2701 | gcagttcgcg ggcgaagtcc tcggtccgct tccactgcgc cccgtcgagc agcgcggcca |
| 2761 | ggatctcgcg gtcgccccgg aaggcgttga gatgcagttg caccaggctg tagcgggagt |
| 2821 | ctcccgcata gacgtcggtg aagtcgacga tcccggtgac ctcggtcgcg gccaggtcca |
| 2881 | cgaagatgtt ggtcccgtgc aggtcgccgt ggacgaaccg gggttcgcgg ccggccagca |
| 2941 | gcgtgtccac gtccggcagc cagtcctcca ggcggtccag cagccggggc gagaggtagc |
| 3001 | cccacccgcg gtggtcctcg acggtcgccg cgcggcgttc ccgcagcagt tccgggaaga |
| 3061 | cctcggaatg gggggtgagc acggtgttcc cggtcagcgg caccctgtgc agccggccga |
| 3121 | gcacccggcc gagttcgcgg gccagggcga gcagcgcgtt ccggtcggtc gtgccgtcca |
| 3181 | tcgcggaccg ccaggtggtg ccggtcatcc ggctcatcac caggtagggc cacggccagg |
| 3241 | ctccggtgcc gggccgcagc tcgccgcggc cgaggaggcg gggcaccggc accggggcgt |
| 3301 | ccgccaggac cgcgtacgcc tccgactccg acgcgaggct ctccggaccg caccagtgct |
| 3361 | cgccgaacag cttgatgacc gggtcgggct cgccgaccag tacggggttg gtgctctcgc |
| 3421 | cgggcacccg cagcaccggc ggcaccggca gcccgagctc ctccagggct cggcgggcca |
| 3481 | gcggctccca gaattcctgg tcgttccgca ggctcgcgta ggaatcatcc gaatcaatac |
| 3541 | ggtcgagaag taacagggat tcttgtgtca cagcggacct ctattcacag ggtacgggcc |
| 3601 | ggcttaattc cgcacggccg gtcgcgacac ggcctgtccg caccgcggtc aggcgttgac |
| 3661 | gatgacgggc tggtcggcca cgtcggggac gttctcggtg gtgctgcggt cgggatcgcc |
| 3721 | aatctctacg ggccgaccga ggcgacggtg tacgccaccg cctggttctg cgacggcgag |
| 3781 | gcgccgccag gccccgccga tccccgtccc ccgcgtcgtc gagcgcggtg ccgacgacac |
| 3841 | cgccgcgtgg ctcgtcacgg aggccgtccc cggcgtcgcg gcggccgagg agtggcccga |
| 3901 | gcaccagcgg ttcgccgtgg tcgaggcgat ggcggagctg gcccgcgccc tccacgagct |
| 3961 | gcccgtggag gactgcccct ccgaccggcg cctcgacgcg gcggtcgccg aggcccggcg |
| 4021 | gaacgtcgcc gagggcttgg tggacctcga cgacctgcag gcatgcaagc tcaggatgtc |
| 4081 | cacctacaac aaagctctca ccaaccgtgg ctccctcact ttctggctgg atgatggggc |
| 4141 | gattcangcc tggtatgant cagcaacacc ttcttcacga ngcagacctc actagcaacc |
| 4201 | gtccgaaata ttataaatta tcgcacacat aaaaacagtg ctgttaatgt gtctattaag |
| 4261 | tcgatttttt gttataacag acactgcttg tccgatattt gatttaggat acacgcgcac |
| 4321 | cggttctaga gcgactactc aacggccgcc gagcgcgtcg gttcggctac cgcatggttg |
| 4381 | ccaatcggtc ccgaatcctg gggttttacc ggctggcgat ggttttccgg caccgcgccg |
| 4441 | cgctacattc gagataccgg tggctcgcta ggtggcggaa ggaggtggtg atggccgacc |
| 4501 | ccggaagcgt gggacatgtg ttccggcgcg cgttttcctg gctcccggcg cagttcgcct |
| 4561 | cccagagtga cgcgccggtc ggcgcgccgc ggcagttccg ttccaccgag cacctgtcaa |
| 4621 | tcgaggccat cgcggctttc gtcgacggcg agctgcggat gaacgcgcac ttgcgggccg |
| 4681 | cgcatcacct ttcgctgtgt gcccaatgcg cggccgaagt ggacgaccaa agtcgtgccc |
| 4741 | gcgccgctct gcgcgattcc cacccgatcc gcatccccag cacgttgctc ggattactgt |
| 4801 | ccgagatccc gcgttgtcca cctgaaggtc catctaaagg ttcgtctgga ggttcatccc |
| 4861 | agggcccgcc cgacggggct gcggcaggct tcggcgaccg cttcgctgac ggcgatggcg |
| 4921 | ggaatcgggg ccggcaatcg cgggtgcgtc gctagccggt gagccacttg tcgcagcgca |
| 4981 | tggcggggtt gctgcgagtt catggcgagt ggtcgcgatc cgtggatact agggtggaca |
| 5041 | cggacaacgc gatgcctgca cgttttagcg cccagattca gaatgaggat gaggtgacct |
| 5101 | ccgaccaagg caacaacggc ggcccgaacg gcgggggtac cctctagtca aggccttaag |
| 5161 | tgagtcgtat tacggactgg gagtcaggca actatggatg aacgaaatag acagatcgct |
| 5221 | gagataggtg cctcactgat taagcattgg taactgtcag accaagttta ctcatatata |
| 5281 | ctttagattg atttaccccg gttgataatc agaaaagccc caaaaacagg aagattgtat |
| 5341 | aagcaaatat ttcatgacat taacctataa aaataggcgt atcacgaggc cctttcgtct |
| 5401 | tcaagaattc gcggccgcaa ttaaccctca ctaaaggatc ttaattaa |

(N) p2NIL/GOAL19-mut trxC-mut trxB2 (SEQ ID NO:36)

The vector for inactivating the active sites of thioredoxin (trxC, also trx) and thioredoxin reductase (trxB2, also trxr) without leaving residual antibiotic resistance. It can be electroporated into BCG to construct BCGΔtrxΔtrxr. It can also be used to modify 1^{st}, 2^{nd}, and 3^{rd} generation pro-apoptotic BCG vaccines, respectively, into 2^{nd}, 3^{rd}, and 4^{th} generation pro-apoptotic BCG vaccines.

| | |
|---|---|
| 1 | taagcggccg cggtacccaa aaaaagcccg ctcattaggc gggctaattc gcctcgaggt |
| 61 | ggcttatcga aattaatacg actcactata gggagaccgg aagcttcacg tggtcgacgg |
| 121 | tatcgataag cttgatatcg aattcctgca gcccggggga tcgaaaaggt taggaatacg |
| 181 | gttagccatt tgcctgcttt tatatagtta tatgggattc acctttatgt tgataagaaa |
| 241 | taaaagaaaa tgccaatagg atnnnnnnnn nnnnnnnnnn nnnnnnnnnn nnnnnnnnnn |
| 301 | nnnnnnnnnn nnnnnnnnnn nnnnnnnnnn nnnnnnnnnn nnnnnnnnnn nnnnnnnnnn |
| 361 | nnnnnnnnnn nnnnnnnnnn nnnnnnnnnn nnnnnnnnnn nnnnnnnnnn nnnnnnnnnn |
| 421 | nnnnnnnnnn nnnnnnnnnn nnnnnnnnnn nnnnnnnnnn nnnnnnnnnn nnnnnnnnnn |
| 481 | nnnnnnnnnn nnnnnnnnnn nnnnnnnnnn nnnnnnnnnn nnnnnnnnnn nnnnnnnnnn |
| 541 | nnnnnnnnnn nnnnnnnnnn nnnnnnnnnn nnnnnnnnnn nnnnnnnnnn nnnnnnnnnn |
| 601 | nnnnnnnnnn nnnnnnnnnn nnnnnnnnnn nnnnnnnnnn nnnnnnnnnn nnnnnnnnnn |
| 661 | nnnnnnnnnn nnnnnnnnnn nnnnnnnnnn nnnnnnnnnn nnnnnnnnnn nnnnnnnnnn |
| 721 | nnnnnnnnnn nnnnnnnnnn nnnnnnnnnn nnnnnnnnnn nnatcggcat tttcttttgc |
| 781 | gtttttattt gttaactgtt aattgtcctt gttcaaggat gctgtctttg acaacagatg |
| 841 | ttttcttgcc tttgatgttc agcaggaagc ttggcgcaaa cgttgattgt ttgtctgcgt |
| 901 | agaatcctct gtttgtcata tagcttgtaa tcacgacatt gtttcctttc gcttgaggta |
| 961 | cagcgaagtg tgagtaagta aaggttacat cgttaggatc aagatccatt tttaacacaa |
| 1021 | ggccagtttt gttcagcggc ttgtatgggc cagttaaaga attagaaaca taaccaagca |
| 1081 | tgtaaatatc gttagacgta atgccgtcaa tcgtcatttt tgatccgcgg gagtcagtga |
| 1141 | acaggtacca tttgccgttc attttaaaga cgttcgcgcg ttcaatttca tctgttactg |
| 1201 | tgttagatgc aatcagcggt ttcatcactt ttttcagtgt gtaatcatcg tttagctcaa |
| 1261 | tcataccgag agcgccgttt gctaactcag ccgtgcgttt tttatcgctt tgcagaagtt |
| 1321 | tttgactttc ttgacggaag aatgatgtgc ttttgccata gtatgctttg ttaaataaag |
| 1381 | attcttcgcc ttggtagcca tcttcagttc cagtgtttgc ttcaaatact aagtatttgt |
| 1441 | ggcctttatc ttctacgtag tgaggatctc tcagcgtatg gttgtcgcct gagctgtagt |
| 1501 | tgccttcatc gatgaactgc tgtacatttt gatacgtttt tccgtcaccg tcaaagattg |
| 1561 | atttataatc ctctacaccg ttgatgttca aagagctgtc tgatgctgat acgttaactt |
| 1621 | gtgcagttgt cagtgtttgt ttgccgtaat gtttaccgga gaaatcagtg tagaataaac |
| 1681 | ggatttttcc gtcagatgta aatgtggctg aacctgacca ttcttgtgtt tggtctttta |
| 1741 | ggatagaatc atttgcatcg aatttgtcgc tgtctttaaa gacgcggcca gcgtttttcc |
| 1801 | agctgtcaat agaagtttcg ccgacttttt gatagaacat gtaaatcgat gtgtcatccg |
| 1861 | catttttagg atctccggct aatgcaaaga cgatgtggta gccgtgatag tttgcgacag |
| 1921 | tgccgtcagc gttttgtaat ggccagctgt cccaaacgtc caggcctttt gcagaagaga |
| 1981 | tatttttaat tgtggacgaa tcgaattcag gaacttgata tttttcattt ttttgctgtt |
| 2041 | cagggatttg cagcatatca tggcgtgtaa tatgggaaat gccgtatgtt tccttatatg |
| 2101 | gcttttggtt cgtttctttc atatgcgcaa acgcttgagt tgcgcctcct gccagcagtg |
| 2161 | cggtagtaaa ggttaatact gttgcttgtt ttgcaaactt tttgatgttc atcgttcatg |
| 2221 | tctccttttt tatgtactgt gttagcggtc tgcttcttcc agccctcctg tttgaagatg |
| 2281 | gcaagttagt tacgcacaat aaaaaaagac ctaaaatatg taaggggtga cgccaaagta |
| 2341 | tcgacctcga gtcaccgggt gacggcaacc ccgctactta cgctggccgg gcgcagtgcc |
| 2401 | cgcgacggac ggctcaagtt gtcctcgctg ccactcgctg cgacgacggg cctggcctca |
| 2461 | ccgtcccgac ctacgactca ccggtcgcga gtgccaacgt tattcttagc actcgcctat |
| 2521 | gccgagtgca agaagccccg caccgggtca tgcccctcgt tcgaccttgt cctcggccct |
| 2581 | ccgatccggg tgagtatgtt cggcccatga ccgccaacga caacaagacc cgtaaatggt |
| 2641 | cggccgcaga cgtccccgat caaagcgggc gcgtcgttgt ggtcactcga ggggggatcc |
| 2701 | cccctgcccg gttattatta tttttgacac cagaccaact ggtaatggta gcgaccggcg |
| 2761 | ctcagctgga attccgccga tactgacggg ctccaggagt cgtcgccacc aatccccata |
| 2821 | tggaaaccgt cgatattcag ccatgtgcct tcttccgcgt gcagcagatg gcgatggctg |
| 2881 | gtttccatca gttgctgttg actgtagcgg ctgatgttga actggaagtc gccgcgccac |
| 2941 | tggtgtgggc cataattcaa ttcgcgcgtc ccgcagcgca gaccgttttc gctcgggaag |
| 3001 | acgtacgggg tatacatgtc tgacaatggc agatcccagc ggtcaaaaca ggcggcagta |
| 3061 | aggcggtcgg gatagttttc ttgcggccct aatccgagcc agtttacccg ctctgctacc |
| 3121 | tgcgccagct ggcagttcag gccaatccgc gccggatgcg gtgtatcgct cgccacttca |
| 3181 | acatcaacgg taatcgccat ttgaccacta ccatcaatcc ggtaggtttt ccggctgata |
| 3241 | aataaggttt tcccctgatg ctgccacgcg tgagcggtcg taatcagcac cgcatcagca |
| 3301 | agtgtatctg ccgtgcactg caacaacgct gcttcggcct ggtaatggcc cgccgccttc |
| 3361 | cagcgttcga cccaggcgtt agggtcaatg cgggtcgctt cacttacgcc aatgtcgtta |
| 3421 | tccagcggtg cacgggtgaa ctgatcgcgc agcggcgtca gcagttgttt tttatcgcca |
| 3481 | atccacatct gtgaaagaaa gcctgactgg cggttaaatt gccaacgctt attacccagc |
| 3541 | tcgatgcaaa aatccatttc gctggtggtc agatgcggga tggcgtggga cgcggcgggg |
| 3601 | agcgtcacac tgaggttttc cgccagacgc cactgctgcc aggcgctgat gtgcccggct |
| 3661 | tctgaccatg cggtcgcgtt cggttgcact acgcgtactg tgagccagag ttgcccggcg |
| 3721 | ctctccggct gcggtagttc aggcagttca atcaactgtt taccttgtgg agcgacatcc |
| 3781 | agaggcactt caccgcttgc cagcggctta ccatccagcg ccaccatcca gtgcaggagc |
| 3841 | tcgttatcgc tatgacggaa caggtattcg ctggtcactt cgatggtttg cccggataaa |
| 3901 | cggaactgga aaaactgctg ctggtgtttt gcttccgtca gcgctggatg cggcgtgcgg |
| 3961 | tcggcaaaga ccagaccgtt catacagaac tggcgatcgt tcggcgtatc gccaaaatca |
| 4021 | ccgccgtaag ccgaccacgg gttgccgttt tcatcatatt taatcagcga ctgatccacc |
| 4081 | cagtcccaga cgaagccgcc ctgtaaacgg ggatactgac gaaacgcctg ccagtattta |
| 4141 | gcgaaaccgc caagactgtt acccatcgcg tgggcgtatt cgcaaaggat cagcgggcgc |
| 4201 | gtctctccag gtagcgaaag ccattttttg atggaccatt tcggcacagc cgggaagggc |
| 4261 | tggtcttcat ccacgcgcgc gtacatcggg caaataatat cggtggccgt ggtgtcggct |
| 4321 | ccgccgcctt catactgcac cgggcgggaa ggatcgacag atttgatcca gcgatacagc |
| 4381 | gcgtcgtgat tagcgccgtg gcctgattca ttccccagcg accagatgat cacactcggg |
| 4441 | tgattacgat cgcgctgcac cattcgcgtt acgcgttcgc tcatcgccgg tagccagcgc |
| 4501 | ggatcatcgg tcagacgatt cattggcacc atgccgtggg tttcaatatt ggcttcatcc |
| 4561 | accacataca ggccgtagcg gtcgcacagc gtgtaccaca gcggatggtt cggataatgc |
| 4621 | gaacagcgca cggcgttaaa gttgttctgc ttcatcagca ggatatcctg caccatcgtc |
| 4681 | tgctcatcca tgacctgacc atgcagagga tgatgctcgt gacggttaac gcctcgaatc |
| 4741 | agcaacggct tgccgttcag cagcagcaga ccattttcaa tccgcacctc gcggaaaccg |
| 4801 | acatcgcagg cttctgcttc aatcagcgtg ccgtcggcgg tgtgcagttc aaccaccgca |
| 4861 | cgatagagat tcgggatttc ggcgctccac agtttcgggt tttcgacgtt cagacgtagt |
| 4921 | gtgacgcgat cggcataacc accacgctca tcgataattt caccgccgaa aggcgcggtg |
| 4981 | ccgctggcga cctgcgtttc accctgccat aaagaaactg ttacccgtag gtagtcacgc |
| 5041 | aactcgccgc acatctgaac ttcagcctcc agtacagcgc ggctgaaatc atcattaaag |
| 5101 | cgagtggcaa catggaaatc gctgatttgt gtagtcggtt tatgcagcaa cgagacgtca |
| 5161 | cggaaaatgc cgctcatccg ccacatatcc tgatcttcca gataactgcc gtcactccaa |
| 5221 | cgcagcacca tcaccgcgag gcggttttct ccggcgcgta aaaatgcgct caggtcaaat |
| 5281 | tcagacggca aacgactgtc ctggccgtaa ccgacccagc gcccgttgca ccacagatga |
| 5341 | aacgccgagt taacgccatc aaaaataatt cgcgtctggc cttcctgtag ccagctttca |
| 5401 | tcaacattaa atgtgagcga gtaacaaccc gtcggattct ccgtgggaac aaacggcgga |
| 5461 | ttgaccgtaa tgggataggt tacgttggtg tagatgggcg catcgtaacc gtgcatctgc |
| 5521 | cagtttgagg ggacgacgac agtatcggcc tcaggaagat cgcactccag ccagctttcc |
| 5581 | ggcaccgctt ctggtgccgg aaaccaggca aagcgccatt cgccattcag gctgcgcaac |
| 5641 | tgttgggaag ggcgatcggt gcgggcctct tcgctattac gccagctggc gaaaggggga |
| 5701 | tgtgctgcaa ggcgattaag ttgggtaacg ccagggtttt cccagtcacg acgttgtaaa |
| 5761 | acgacgggat ccattcttgc ttccctcatc ctcatctcaa cgcatccatg catgtttggg |
| 5821 | cgcatcctga attaggtcag actgcaggcg ctgggcccgg cagtgctcgt gtagtcaacc |
| 5881 | acaacttcgg gcgtccaccc gcatcaagcg caccgccgaa acccttatcc ggcggtcgtt |
| 5941 | cacggccaat tcgggaccga cgcgacggcc tgaaggtggc atttccgcag tgtctgggca |
| 6001 | tgtgtcgtct agagcggccg ccaccgcggt ggagctcagc cagatcctat gtattctata |
| 6061 | gtgtcaccta aatcgtatgt gtatgataca taaggttatg tattaattgt agccgcgttc |
| 6121 | taacgacaat atgtacaagc ctaattgtgt agcatctggc ttactgaagc agaccctatc |
| 6181 | atctctctcg taaactgccg tcagagtcgg tttggttgga cgaaccttct gagtttctgg |
| 6241 | taacgccgtc ccgcacccgg aaatggtcag cgaaccaatc agcagggtca tcgctagaaa |
| 6301 | tcatccttag cgaaagctaa ggattttttt tatctgaatt ggtaccgcgg ccgccggggg |
| 6361 | ccgggggcgg cgccgggcgg cccggggcgt caggcgccgg gggcggtgtc cggcggcccc |
| 6421 | cagaggaact gcgccagttc ctccggatcg gtgaagccgg agagatccag cggggtctcc |
| 6481 | tcgaacacct cgaagtcgtg caggaaggtg aaggcgagca gttcgcgggc gaagtcctcg |
| 6541 | gtccgcttcc actgcgcccc gtcgagcagc gcggccagga tctcgcggtc gccccggaag |
| 6601 | gcgttgagat gcagttgcac caggctgtag cgggagtctc ccgcatagac gtcggtgaag |
| 6661 | tcgacgatcc cggtgacctc ggtcgcggcc aggtccacga agatgttggt cccgtgcagg |
| 6721 | tcgccgtgga cgaaccgggg ttcgcggccg gccagcagcg tgtccacgtc cggcagccag |
| 6781 | tcctccaggc ggtccagcag ccggggcgag aggtagcccc acccgcggtg gtcctcgacg |
| 6841 | gtcgccgcgc ggcgttcccg cagcagttcc gggaagacct cggaatgggg ggtgagcacg |
| 6901 | gtgttcccgg tcagcggcac cctgtgcagc cggccgagca cccggccgag ttcgcgggcc |
| 6961 | agggcgagca gcgcgttccg gtcggtcgtg ccgtccatcg cggaccgcca ggtggtgccg |
| 7021 | gtcatccggc tcatcaccag gtagggccac ggccaggctc cggtgccggg ccgcagctcg |
| 7081 | ccgcggccga ggaggcgggg caccggcacc ggggcgtccg ccaggaccgc gtacgcctcc |
| 7141 | gactccgacg cgaggctctc cggaccgcac cagtgctcgc cgaacagctt gatcaccggg |
| 7201 | tcgggctcgc cgaccagtac ggggttggtg ctctcgccgg gcacccgcag caccggcggc |
| 7261 | accggcagcc cgagctcctc cagggctcgg cgggccagcg gctcccagaa ttcctggtcg |
| 7321 | ttccgcaggc tcgcgtagga atcatccgaa tcaatacggt cgagaagtaa cagggattct |
| 7381 | tgtgtcacag cggacctcta ttcacagggt acgggccggc ttaattccgc acggccggtc |
| 7441 | gcgacacggc ctgtccgcac cgcggtcagg cgttgacgat gacgggctgg tcggccacgt |
| 7501 | cggggacgtt ctcggtggtg ctgcggtcgg gatcgccaat ctctacgggc cgaccgaggc |
| 7561 | gacggtgtac gccaccgcct ggttctgcga cggcgaggcg ccgccaggcc ccgccgatcn |
| 7621 | nnnnnnnnnn nnnnnnnnnn nnnnnnnnnn nnnnnnnnnn nnnnnnnnnn nnnnnnnnnn |
| 7681 | nnnnnnnnnn nnnnnnnnnn nnnnnnnnnn nnnnnnnnnn nnnnnnnnnn nnnnnnnnnn |
| 7741 | nnnnnnnnnn nnnnnnnnnn nnnnnnnnnn nnnnnnnnnn nnnnnnnnnn nnnnnnnnnn |
| 7801 | nnnnnnnnnn nnnnnnnnnn nnnnnnnnnn nnnnnnnnnn nnnnnnnnnn nnnnnnnnnn |
| 7861 | nnnnnnnnnn nnctgcaggc atgcnnnnnn agatccatgg atatctagat ttaaagatct |
| 7921 | ggtaccgcgg ccgcttaatt aagaattccc ctgtaatccg ggcagcgcaa cggaacattc |
| 7981 | atcagtgtaa aaatggaatc aataaagccc tgcgcagcgc gcagggtcag cctgaatacg |
| 8041 | cgtttaatga ccagcacagt cgtgatggca aggtcagaat agcgctgagg tctgcctcgt |
| 8101 | gaagaaggtg ttgctgactc ataccaggat tttgttaaaa ttcgcgttaa atttttgtta |
| 8161 | aatcagctca ttttttaacc aataggccga aatcggcaaa atcccttata aatcaaaaga |
| 8221 | atagaccgag atagggttga gtgttgttcc agtttggaac aagagtccac tattaaagaa |
| 8281 | cgtggactcc aacgtcaaag ggcgaaaaac cgtctatcag ggcgatggcc cactacgtga |
| 8341 | accatcaccc taatcaagtt ttttggggtc gaggtgccgt aaagcactaa atcggaaccc |
| 8401 | taaagggagc ccccgattta gagcttgacg gggaaagccg gcgaacgtgg cgagaaagga |
| 8461 | agggaagaaa gcgaaaggag cgggcgctag ggcgctggca agtgtagcgg tcacgctgcg |
| 8521 | cgtaaccacc acacccgccg cgcttaatgc gccgctacag ggcgcgtact atggttgctt |
| 8581 | tgacgagcac gtataacgtg ctttcctcgt tagaatcaga gcgggagcta aacaggaggc |
| 8641 | cgattaaagg gattttagac aggaacggta cgccagaatc ctgagaagtg tttttataat |
| 8701 | cagtgaggcc accgagcaaa agagtctgtc catcacgcaa attaaccgtt gtcgcaatac |
| 8761 | ttctttgatt agtaataaca tcacttgcct gagtagaaga actcaaacta tcggccttgc |
| 8821 | tggtaatatc cagaacaatc ctgaatcgcc ccatcatcca gccagaaagt gagggagcca |
| 8881 | cggttgatga gagctttgtt gtaggtggac cagttggtga ttttgaactt ttgctttgcc |
| 8941 | acggaacggt ctgcgttgtc gggaagatgc gtgatctgat ccttcaactc agcaaaagtt |
| 9001 | cgatttattc aacaaagccg ccgtcccgtc aagtcagcgt aatgctctgc cagtgttaca |
| 9061 | accaattaac caattctgat tagaaaaact catcgagcat caaatgaaac tgcaatttat |
| 9121 | tcatatcagg attatcaata ccatattttt gaaaaagccg tttctgtaat gaaggagaaa |
| 9181 | actcaccgag gcagttccat aggatggcaa gatcctggta tcggtctgcg attccgactc |
| 9241 | gtccaacatc aatacaacct attaatttcc cctcgtcaaa aataaggtta tcaagtgaga |
| 9301 | aatcaccatg agtgacgact gaatccggtg agaatggcaa aaacttatgc atttctttcc |
| 9361 | agacttgttc aacaggccag ccattacgct cgtcatcaaa atcactcgca tcaaccaaac |
| 9421 | cgttattcat tcgtgattgc gcctgagcga gacgaaatac gcgatcgctg ttaaaaggac |
| 9481 | aattacaaac aggaatcgaa tgcaaccggc gcaggaacac tgccagcgca tcaacaatat |
| 9541 | tttcacctga atcaggatat tcttctaata cctggaatgc tgttttccag gggatcgcag |
| 9601 | tggtgagtaa ccatgcatca tcaggagtac ggataaaatg cttgatggtc ggaagaggca |
| 9661 | taaattccgt cagccagttt agtctgacca tctcatctgt aacatcattg gcaacgctac |
| 9721 | ctttgccatg tttcagaaac aactctggcg catcgggctt cccatacaat cgatagattg |
| 9781 | tcgcacctga ttgcccgaca ttatcgcgag cccatttata cccatataaa tcagcatcca |
| 9841 | tgttggaatt taatcgcggc ctcgagcaag acgtttcccg ttgaatatgg ctcataacac |
| 9901 | cccttgtatt actgtttatg taagcagaca gttttattgt tcatgatgat atatttttat |
| 9961 | cttgtgcaat gtaacatcag agattttgag acacaacgtg gctttgttga ataaatcgaa |
| 10021 | cttttgctga gttgaaggat cagatcacgc atcttcccga caacgcagac cgttccgtgg |
| 10081 | caaagcaaaa gttcaaaatc accaactggt ccacctacaa caaagctctc atcaaccgtg |
| 10141 | gctccctcac tttctggctg gatgatgggg cgattcaggc tgcctcgcgc gtttcggtga |
| 10201 | tgacggtgaa aacctctgac acatgcagct cccggagacg gtcacagctt gtctgtaagc |
| 10261 | ggatgccggg agcagacaag cccgtcaggg cgcgtcagcg ggtgttggcg ggtgtcgggg |
| 10321 | cgcagccatg acccagtcac gtagcgatag cggagtgtat actggcttaa ctatgcggca |
| 10381 | tcagagcaga ttgtactgag agtgcaccat atgcggtgtg aaataccgca cagatgcgta |
| 10441 | aggagaaaat accgcatcag gcgctcttcc gcttcctcgc tcactgactc gctgcgctcg |
| 10501 | gtcgttcggc tgcggcgagc ggtatcagct cactcaaagg cggtaatacg gttatccaca |
| 10561 | gaatcagggg ataacgcagg aaagaacatg tgagcaaaag gccagcaaaa ggccaggaac |
| 10621 | cgtaaaaagg ccgcgttgct ggcgtttttc cataggctcc gcccccctga cgagcatcac |
| 10681 | aaaaatcgac gctcaagtca gaggtggcga aacccgacag gactataaag ataccaggcg |
| 10741 | tttccccctg gaagctccct cgtgcgctct cctgttccga ccctgccgct taccggatac |
| 10801 | ctgtccgcct ttctcccttc gggaagcgtg gcgctttctc aatgctcacg ctgtaggtat |
| 10861 | ctcagttcgg tgtaggtcgt tcgctccaag ctgggctgtg tgcacgaacc ccccgttcag |
| 10921 | cccgaccgct gcgccttatc cggtaactat cgtcttgagt ccaacccggt aagacacgac |
| 10981 | ttatcgccac tggcagcagc cactggtaac aggattagca gagcgaggta tgtaggcggt |
| 11041 | gctacagagt tcttgaagtg gtggcctaac tacggctaca ctagaaggac agtatttggt |
| 11101 | atctgcgctc tgctgaagcc agttaccttc ggaaaaagag ttggtagctc ttgatccggc |
| 11161 | aaacaaacca ccgctggtag cggtggtttt tttgtttgca agcagcagat tacgcgcaga |
| 11221 | aaaaaaggat ctcaagaaga tcctttgatc ttttctacgg ggtctgacgc tcagtggaac |
| 11281 | gaaaactcac gttaagggat tttggtcatg agattatcaa aaaggatctt cacctagatc |
| 11341 | cttttaaatt aaaaatgaag ttttaaatca atctaaagta tatatgagta aacttggtct |
| 11401 | gacagttacc aatgcttaat cagtgaggca cctatctcag cgatctgtct atttcgttca |
| 11461 | tccatagttg cctgactccc cgtcgtgtag ataactacga tacgggaggg cttaccatct |
| 11521 | ggccccagtg ctgcaatgat accgcgagac ccacgctcac cggctccaga tttatcagca |
| 11581 | ataaaccagc cagccggaag ggccgagcgc agaagtggtc ctgcaacttt atccgcctcc |
| 11641 | atccagtcta ttaattgttg ccgggaagct agagtaagta gttcgccagt taatagtttg |
| 11701 | cgcaacgttg ttgccattgc tgcaggcatc gtggtgtcac gctcgtcgtt tggtatggct |
| 11761 | tcattcagct ccggttccca acgatcaagg cgagttacat gatcccccat gttgtgcaaa |
| 11821 | aaagcggtta gctccttcgg tcctccgatc gttgtcagaa gtaagttggc cgcagtgtta |
| 11881 | tcactcatgg ttatggcagc actgcataat tctcttactg tcatgccatc cgtaagatgc |
| 11941 | ttttctgtga ctggtgagta ctcaaccaag tcattctgag aatagtgtat gcggcgaccg |
| 12001 | agttgctctt gcccggcgtc aacacgggat aataccgcgc cacatagcag aactttaaaa |
| 12061 | gtgctcatca ttggaaaacg ttcttcgggg cgaaaactct caaggatctt accgctgttg |
| 12121 | agatccagtt cgatgtaacc cactcgtgca cccaactgat cttcagcatc ttttactttc |
| 12181 | accagcgttt ctgggtgacg cagatcccgc aagaggcccg gcagtaccgg cataaccaag |
| 12241 | cctatgccta cagcatccag ggtgacggtg ccgaggatga cgatgagcgc attgttagat |
| 12301 | ttcatacacg gtgcctgact gcgttagcaa tttaactgtg ataaactacc gcattaaagc |
| 12361 | ttgggctagt tgaggttggg aaccacgtct gagagctcgc gcagcaacgc agccttaccc |
| 12421 | ttggcgccaa cgattcgttt caccggctgg ccgtccttga acaagatcag ggtagggatc |
| 12481 | gagacgacct ggaagttgcg ggcggtctcc gggttggtgt ccacgtcgag cttggcgacg |
| 12541 | gtgaggtctg ttgcgcgctc ggtggcgatt tcctcgagaa cgggcgctac cattgtcgcc |
| 12601 | caaaagtcaa ccagcacagg cttgttgctg gatagcacgt cggtggcaaa ggatgcgtcg |
| 12661 | gtaactttga tggtggcgga cttctcggaa tcggtcatcg ttgtgctcct atcaatgcgt |
| 12721 | cggtactgtc agcttctccg gttgctgcgt gctcggcgag ccagcgctcg gcgtcgatag |
| 12781 | ccgcggcgca gccactgccc gctgcggtaa ccgcctggcg ataggtgcga tccaccaggt |
| 12841 | cgccggcagc gaacacgccc ggcagtgagg tgctggtggt acgcccctgc accaacacgt |
| 12901 | agccgtccgg gtcgacgtcg atggcctcgc gcaccaagcc cgaccgcggc tcgtggccga |
| 12961 | tcgcgacgaa aacaccggtt accggcaggg tggtttcggc accggtgttg gtgtcgcgta |
| 13021 | cccgcaagcc ggtcactgtg gtgtccccgt ccaccgcgac cacggtgtgg ttggtgagga |
| 13081 | accgtatctt gtcgttgttg cgggcgcgat cgagcatgat tttggaagcc cggaactcgt |
| 13141 | cgcggcgatg caccagcgtc acactgcgag cgaatcgggt caggaaggta gcttcctcca |
| 13201 | ttgccgagtc accgccgccg atgacggcga tgtcctgatc gcggaagaag aacgagctca |
| 13261 | ccccgcgccc gagcaattcc tgttcgccgg gcacctgcag atagcgtgcc gctgcgccca |
| 13321 | ttgccaggat cacggctcgg gcccggtggg tctgtccgtc ggcggtgacg accgatttca |
| 13381 | gcggcccgtg aagtgatacc gactcgacgt cttccatacg caggtccgcg ccgaatcgca |
| 13441 | gcgcctgttc ccgcatctca tccatcaact ctggaccggt gatgccgttg cgaaatcccg |
| 13501 | ggtagttctc cacgtcggtg gtggtcatca gcgcgccgcc gaaagacgtg ccctcgaaga |
| 13561 | ccagcggcgc cagctgggca cgggcggcgt agagcgccgc agtgtacccc gcgggaccgg |
| 13621 | agccgataac gatcacgtcg cgaacggggt ggtgtgcgcg gtcatggaca ggcggggcgg |
| 13681 | tcatgcggga tcctatgtat tctatagtgt cacctaaatc gtatgtgtat gatacataag |
| 13741 | gttatgtatt aattgtagcc gcgttctaac gacaatatgt acaagcctaa ttgtgtagca |
| 13801 | tctggcttac tgaagcagac cctatcatct ctctcgtaaa ctgccgtcag agtcggtttg |
| 13861 | gttggacgaa ccttctgagt ttctggtaac gccgtcccgc acccggaaat ggtcagcgaa |
| 13921 | ccaatcagca gggtcatcgc tagaaatcat ccttagcgaa agctaaggat tttttttatc |
| 13981 | tgaattggta ccgcggccgc ttaat |

(O) pMP349-rBLS (SEQ ID NO:37)

pMP349 with recombinant Brucella lumazine synthase behind aceA(icl) promoter used to create DD-BCGrBLS (plasmid-expressed). It can be added to BCG or to 1^{st}, 2^{nd}, 3^{rd} or 4^{th} generation pro-apoptotic BCG vaccines that enhance antigen presentation via apoptosis-associated cross priming pathways.

| | |
|---|---|
| 1 | ctagttccac tgagcgtcag accccgtaga aaagatcaaa ggatcttctt |
| 51 | gagatccttt ttttctgcgc gtaatctgct gcttgcaaac aaaaaaacca |
| 101 | ccgctaccag cggtggtttg tttgccggat caagagctac caactctttt |
| 151 | tccgaaggta actggcttca gcagagcgca gataccaaat actgtccttc |
| 201 | tagtgtagcc gtagttaggc caccacttca agaactctgt agcaccgcct |
| 251 | acatacctcg ctctgctaat cctgttacca gtggctgctg ccagtggcga |
| 301 | taagtcgtgt cttaccgggt tggactcaag acgatagtta ccggataagg |
| 351 | cgcagcggtc gggctgaacg gggggttcgt gcacacagcc cagcttggag |
| 401 | cgaacgacct acaccgaact gagataccta cagcgtgagc attgagaaag |
| 451 | cgccacgctt cccgaaggga gaaaggcgga caggtatccg gtaagcggca |
| 501 | gggtcggaac aggagagcgc acgagggagc ttccaggggg aaacgcctgg |
| 551 | tatctttata gtcctgtcgg gtttcgccac ctctgacttg agcgtcgatt |
| 601 | tttgtgatgc tcgtcagggg ggcggagcct atggaaaaac gccagcaacg |
| 651 | cggccttttt acggttcctg gccttttgct ggccttttgc tcacatgttc |
| 701 | tttcctgcgt tatcccctga ttctgtggat aaccgtatta ccgcctttga |
| 751 | gtgagctgat accgctcgcc gcagccgaac gaccgagcgc aacgcgtgag |
| 801 | cccaccagct ccgtaagttc gggtgctgtg tggctcgtac ccgcgcattc |
| 851 | aggcggcagg gggtctaacg ggtctaaggc ggcgtgtacg gccgccacag |
| 901 | cggctcttag cggcccggaa acgtcctcga aacgacgcat gtgttcctcc |
| 951 | tggttggtac aggtggttgg gggtgctcgg ctgtcgctgg tgtttcatca |
| 1001 | tcagggctcg acgggagagc gggggagtgt gcagttgtgg ggtggcccct |
| 1051 | cagcgaaata tctgacttgg agctcgtgtc ggaccataca ccggtgatta |
| 1101 | atcgtggttt attatcaagc gtgagccacg tcgccgacga atttgagcag |
| 1151 | ctctggctgc cgtactggtc cctggcaagc gacgatctgc tcgaggggat |
| 1201 | ctaccgccaa agccgcgcgt cggccctagg ccgccggtac atcgaggcga |
| 1251 | acccaacagc gctggcaaac ctgctggtcg tggacgtaga ccatccagac |
| 1301 | gcagcgctcc gagcgctcag cgcccggggg tcccatccgc tgcccaacgc |
| 1351 | gatcgtgggc aatcgcgcca acggccacgc acacgcagtg tgggcactca |
| 1401 | acgcccctgt tccacgcacc gaatacgcgc ggcgtaagcc gctcgcatac |
| 1451 | atggcggcgt gcgccgaagg ccttcggcgc gccgtcgatg gcgaccgcag |
| 1501 | ttactcaggc ctcatgacca aaaaccccgg ccacatcgcc tgggaaacgg |
| 1551 | aatggctcca ctcagatctc tacacactca gccacatcga ggccgagctc |
| 1601 | ggcgcgaaca tgccaccgcc gcgctggcgt cagcagacca cgtacaaagc |
| 1651 | ggctccgacg ccgctagggc ggaattgcgc actgttcgat tccgtcaggt |
| 1701 | tgtgggccta tcttcccgcc ctcatgcgga tctacctgcc gacccggaac |
| 1751 | gtggacggac tcggccgcgc gatctatgcc gagtgccacg cgcgaaacgc |
| 1801 | cgaatttccg tgcaacgacg tgtgtcccgg accgctaccg gacagcgagg |
| 1851 | tccgcgccat cgccaacagc atttggcgtt ggatcacaac caagtcgcgc |
| 1901 | atttgggcgg acgggatcgt ggtctacgag gccacactca gtgcgcgcca |
| 1951 | tgcggccatc tcgcggaagg gcgcagcagc gcgcacggcg gcgagcacag |
| 2001 | ttgcgcggcg cgcaaagtcc gcgtcagcca tggaggcatt gctatgagcg |
| 2051 | acggctacag cgacggctac agcgacggct acaactggca gccgactgtc |
| 2101 | cgcaaaaagc ggcgcgtgac cgccgccgaa ggcgctcgaa tcaccggact |
| 2151 | atccgaacgc cacgtcgtcc ggctcgtggc gcaggaacgc agcgagtggt |
| 2201 | tcgccgagca ggctgcacgc cgcgaacgca tccgcgccta tcacgacgac |
| 2251 | gagggccact cttggccgca aacggccaaa catttcgggc tgcatctgga |
| 2301 | caccgttaag cgactcggct atcgggcgag gaaagagcgt gcggcagaac |
| 2351 | aggaagcggc tcaaaaggcc cacaacgaag ccgacaatcc accgctgttc |
| 2401 | taacgcaatt ggggagcggg tgtcgcgggg gttccgtggg gggttccgtt |
| 2451 | gcaacgggtc ggacaggtaa aagtcctggt agacgctagt tttctggttt |
| 2501 | gggccatgcc tgtctcgttg cgtgtttcgt tgcgtccgtt ttgaatacca |
| 2551 | gccagacgag acggggttct acgaatcttg gtcgatacca agccatttcc |
| 2601 | gctgaatatc gtggagctca ccgccagaat cggtggttgt ggtgatgtac |
| 2651 | gtggcgaact ccgttgtagt gcttgtggtg gcatccgtgg cgcggccgcg |
| 2701 | gtaccccgcc attgcgggcg tattatagag ggttagtcag acaagcgcgg |
| 2751 | cgatgcggct gcgctcgctc acgatctgca aggcggcatg ggccgcttcc |
| 2801 | acgcccttca ccttgaaatg agcatggaag aagtcgtgat gctccttgct |
| 2851 | ttcatggaaa tggtgcggcg tcagcacgac gctcagcacc ggcacttccg |
| 2901 | tttcaagctg cacctgcatc atgccgttga taacggccgt cgccacgaaa |
| 2951 | tcatgacgat agatgccgcc gtcgatcacg aaggccgcac cgacgatggc |
| 3001 | tgcatagcgc ccggttctgg ccaatgtctt ggcgtgaagg ggaatttcat |
| 3051 | atgcacccgg cacgtcgaat atctctacct cgacgctgcc acccgtcttt |
| 3101 | gcggccagtt cggcgacaaa gcttttgcgc gcttcgtcaa cgatgtcggc |
| 3151 | gtgccagcgg gcctgaatga atgcgatttt aaaggatgtc ttgttcggac |
| 3201 | agctttggtt catgggtacc ccagacaact ccttaacggt ctttcattgc |
| 3251 | cgaaaacgct gacgccctac cgtcgtccag gcggtgtcaa cggcgcagct |
| 3301 | tcactggtgt gctaactcga ccatggcaca gcgtgtcaac gctggtccac |
| 3351 | ccatttcact tgcgaatttc ggcaacggcc tgcggacttt ttgcaaattt |
| 3401 | tgcgaagtcg cccaaaaact gaaccgtttc agaagctacc cgccagtaac |
| 3451 | gacaaatccg caggtaaacc cacggatcga cgtcctgcgg atccggtcac |
| 3501 | agattgaaca gcgaggcgac tgccttgggc tcgtcgccaa ccacatatgt |
| 3551 | gagcgttgta acatctagag gtgaccacaa cgacgcgccc gctttgatcg |
| 3601 | gggacgtctg cggccgacca tttacgggtc ttgttgtcgt tggcggtcat |
| 3651 | gggccgaaca tactcacccg gatcggaggg ccgaggacaa ggtcgaacga |
| 3701 | ggggcatgac ccggtgcggg gcttcttgca ctcggcatag gcgagtgcta |
| 3751 | agaataacgt tggcactcgc gaccggtgag tcgtaggtcg ggacggtgag |
| 3801 | gccaggcccg tcgtcgcagc gagtggcagc gaggacaact tgagccgtcc |
| 3851 | gtcgcgggca ctgcgcccgg ccagcgtaag tagcggggtt gccgtcaccc |
| 3901 | ggtgaccccc ggtttcatcc ccgatccgga ggaatcactt cgcaatggcc |
| 3951 | aagacaattg cggatccagc tgcagaattc ctgcagctca cggtaactga |
| 4001 | tgccgtattt gcagtaccag cgtacggccc acagaatgat gtcacgctga |
| 4051 | aaatgccggc ctttgaatgg gttcatgtgc agctccatca gcaaaagggg |
| 4101 | atgataagtt tatcaccacc gactatttgc aacagtgccg ttgatcgtgc |
| 4151 | tatgatcgac tgatgtcatc agcggtggag tgcaatgtcg tgcaatacga |
| 4201 | atggcgaaaa gccgagctca tcggtcagct tctcaacctt ggggttaccc |
| 4251 | ccggcggtgt gctgctggtc cacagctcct tccgtagcgt ccggcccctc |
| 4301 | gaagatgggc ccacttggac tgatcgaggc cctgcgtgct acgctgggtc |
| 4351 | cgggagggac gctcgtcatg ccctcgtggt caggtctgga cgacgagccg |
| 4401 | ttcgatcctg ccacgtcgcc cgttacaccg gaccttggag ttgtctctga |
| 4451 | cacattctgg cgcctgccaa atgtaaagcg cagcgcccat ccatttgcct |
| 4501 | ttgcggcagc ggggccacag gcagagcaga tcatctctga tccattgccc |
| 4551 | ctgccacctt actcgcctgc aagcccggtc gcccgtgtcc atgaactcga |
| 4601 | tgggcaggta cttctcctcg gcgtgggaca cgatgccaac acgacgctgc |
| 4651 | atcttgccga gttgatggca aaggttccct atggggtgcc gagacactgc |
| 4701 | accattcttc aggatggcaa gttggtacgc gtcgattatc tcgagaatga |
| 4751 | ccactgctgt gagcgctttg ccttggcggg acaggtggct caaggagaag |
| 4801 | agccttcaga aggaaggtcc agtcggtcat gcctttgctc ggttgatccg |
| 4851 | ctcccgcgac attgtggcga cagccctggg tcaactgggc cgagatccgt |
| 4901 | tgatcttcct gcatccgcca gagggcggga tgcgaagaat gcgatgccgc |
| 4951 | tcgccagtcg attggctgag ctcatgagcg gagaacgaga tgacgttgga |
| 5001 | ggggcaaggt cgcgctgatt gctggggcaa cacgggggat cca |

Throughout this application, various publications are referenced. The disclosures of these publications in their entireties are hereby incorporated by reference into this application in order to more fully describe the state of the art to which this invention pertains.
1. Albert M L, Sauter B, Bhardwaj N. Dendritic cells acquire antigen from apoptotic cells and induce class I-restricted CTLs. Nature 1998; 392:86-9.
2. Aldovini A, Husson R N, Young R A. The uraA locus and homologous recombination in Mycobacterium bovis BCG. J.Bacteriol. 1993; 175:7282-9.
3. Andersen P, Smedegaard B. CD4⁺ T-cell subsets that mediate immunological memory to Mycobacterium tuberculosis infection in mice. Infect.Immun. 2000; 68:621-9.
4. Balcewicz-Sablinska M K, Keane J, Kornfeld H, Remold H G. Pathogenic Mycobacterium tuberculosis evades apoptosis of host macrophages by release of TNF-R2, resulting in inactivation of TNF-alpha. J.Immunol. 1998; 161:2636-41.
5. Bardarov S, Kriakov J, Carriere C et al. Conditionally replicating mycobacteriophages: a system for transposon delivery to Mycobacterum tuberculosis. Proc.Natl.Acad.Sci.U.S.A 1997; 94:10961-6.
6. Behr M A, Small P M. Has BCG attenuated to impotence? Nature 1997; 389:133-4.
7. Berthet F X, Lagraiderie M, Gounon P et al. Attenuation of virulence by disruption of the Mycobacterium tuberculosis erp gene. Science 1998; 282:759-62.
8. Beyer W F, Jr., Fridovich I. Assaying for superoxide dismutase activity: some large consequences of minor changes in conditions. Anal.Biochem. 1987; 161:559-66.
9. Braunstein M, Bardarov S S, Jacobs W R, Jr. Genetic methods for deciphering virulence determinants of Mycobacterium tuberculosis. Methods Enzymol. 2002; 358:67-99.
10. Braunstein M, Espinosa B J, Chan J, Belisle J T, Jacobs W R, Jr. SecA2 functions in the secretion of superoxide dismutase A and in the virulence of Mycobacterium tuberculosis. Mol.Microbiol. 2003; 48:453-64.
11. Cho S, Mehra V, Thoma-Uszynski S et al. Antimicrobial activity of MIC class I-restricted CD8+ T cells in human tuberculosis. Proc.Natl.Acad.Sci.U.S.A 2000; 97:12210-5.
12. Coleman J, Green P J, Inouye M. The use of RNAs complementary to specific mRNAs to regulate the expression of individual bacterial genes. Cell 1984; 37:429-36.
13. Consaul S A, Pavelka M S, Jr. Use of a novel allele of the Escherichia coli aacC4 aminoglycoside resistance gene as a genetic marker in mycobacteria. FEMS Microbiol.Lett. 2004; 234:297-301.
14. Cooksey R C, Crawford J T, Jacobs W R, Jr., Shinnick T M. A rapid method for screening antimicrobial agents for activities against a strain of Mycobacterium tuberculosis expressing firefly luciferase. Antimicrob.Agents Chemother. 1993; 37:1348-52.
15. Cooper J B, McIntyre K, Badasso M O et al. X-ray structure analysis of the iron-dependent superoxide dismutase from Mycobacterium tuberculosis at 2.0 Angstroms resolution reveals novel dimer-dimer interactions. J.Mol.Biol. 1995; 246:531-44.
16. Dussurget O, Stewart G, Neyrolles O, Pescher P, Young D, Marchal G. Role of Mycobacterium tuberculosis copper-zinc superoxide dismutase. Infect.Immun. 2001; 69:529-33.
17. Eddine A N, Kaufmann S H. Improved protection by recombinant BCG. Microbes.Infect. 2005; 7:939-46.
18. Edwards K M, Cynamon M H, Voladri R K et al. Iron-cofactored superoxide dismutase inhibits host responses to Mycobacterium tuberculosis. Am.J.Respir.Crit Care Med. 2001; 164:2213-9.
19. Eisenberg D, Gill H S, Pfluegl G M, Rotstein S H. Structure-function relationships of glutamine synthetases. Biochim.Biophys.Acta 2000; 1477:122-45.
20. Gill H S, Pfluegl G M, Eisenberg D. Preliminary crystallographic studies on glutamine synthetase from Mycobacterium tuberculosis. Acta Crystallogr.D.Biol.Crystallogr. 1999; 55 (Pt 4):865-8.
21. Graham J E, Clark-Curtiss J E. Identification of Mycobacterium tuberculosis RNAs synthesized in response to phagocytosis by human macrophages by selective capture of transcribed sequences (SCOTS). Proc.Natl.Acad.Sci.U.S.A 1999; 96:11554-9.
22. Grode L, Seiler P, Baumann S et al. Increased vaccine efficacy against tuberculosis of recombinant Mycobacterium bovis bacille Calmette-Guerin mutants that secrete listeriolysin. J.Clin.Invest 2005; 115:2472-9.
23. Hanahan D. Studies on transformation of Escherichia coli with plasmids. J.Mol.Biol. 1983; 166:557-80.
24. Hess J, Miko D, Catic A, Lehmensiek V, Russell D G, Kaufinann S H. Mycobacterium bovis Bacille Calmette-Guerin strains secreting listeriolysin of Listeria monocytogenes. Proc.Natl.Acad.Sci.U.S.A 1998; 95:5299-304.
25. Hinds J, Mahenthiralingam E, Kempsell K E et al. Enhanced gene replacement in mycobacteria. Microbiology 1999; 145 (Pt 3):519-27.
26. Ho S N, Hunt H D, Horton R M, Pullen J K, Pease L R. Site-directed mutagenesis by overlap extension using the polymerase chain reaction. Gene 1989; 77:51-9.
27. Hondalus M K, Bardarov S, Russell R, Chan J, Jacobs W R, Jr., Bloom B R. Attenuation of and protection induced by a leucine auxotroph of Mycobacterium tuberculosis. Infect.Immun. 2000; 68:2888-98.
28. Horwitz M A, Harth G, Dillon B J, Maslesa-Galic'S. Recombinant bacillus Calmette-Guerin (BCG) vaccines expressing the Mycobacterium tuberculosis 30-kDa major secretory protein induce greater protective immunity against tuberculosis than conventional BCG vaccines in a highly susceptible animal model. Proc.Natl.Acad.Sci.U.S.A 2000; 97:13853-8.
29. Howard N S, Gomez J E, Ko C, Bishai W R. Color selection with a hygromycin-resistance-based Escherichia coli-mycobacterial shuttle vector. Gene 1995; 166:181-2.
30. Jackson M, Phalen S W, Lagranderie M et al. Persistence and protective efficacy of a Mycobacterium tuberculosis auxotroph vaccine. Infect.Immun. 1999; 67:2867-73.
31. Jacobs W R, Jr., Tuckman M, Bloom B R. Introduction of foreign DNA into mycobacteria using a shuttle phasmid. Nature 1987; 327:532-5.
32. Kaushal D, Schroeder B G, Tyagi S et al. Reduced immunopathology and mortality despite tissue persistence in a Mycobacterium tuberculosis mutant lacking alternative sigma factor, SigH. Proc.Natl.Acad.Sci.U.S.A 2002; 99:8330-5.
33. Keane J, Remold H G, Kornfeld H. Virulent Mycobacterium tuberculosis strains evade apoptosis of infected alveolar macrophages. J.Immunol. 2000; 164:2016-20.
34. Kelley J J, III, Caputo T M, Eaton S F, Laue T M, Bushweller J H. Comparison of backbone dynamics of reduced and oxidized Eschericbia coli glutaredoxin-1 using 15N NMR relaxation measurements. Biochemistry (Mosc). 1997; 36:5029-44.
35. Kernodle, D., Shoen, C., VaiiHook, S., Crozier, I., DeStefano, M., Hager, C., Price, J., Tham, K-T., and Cyanamon, M. Reducing SodA production by Bacillus Calmette-Guerin enhances immunogenicity and protects C57Bl/6 mice against granulomatous lung disease following challenge with Mycobacterium tuberculosis. Abstract #2045, p. 80, in Tuberculosis: Integrating Host and Pathogen Biology, Keystone Symposia, Whistler, British Columbia, Canada, Apr. 2-7, 2005.
36. Kernodle, D. S., Cynamon, M. H., Hager C C, Destefano, M. S., Tham, K., Bochan, M. R., and Edwards, K. M. A tuberculosis vaccine prototype constructed by diminishing an anti-apoptotic microbial factor. Clinical Infectious Diseases 33, 1154. 2001 (Abstract of the 2001 meeting of the Infectious Diseases Society of America).
37. Lakey D L, Voladri R K, Edwards K M et al. Enhanced production of recombinant Mycobacterium tuberculosis antigens in Escherichia coli by replacement of low-usage codons. Infect.Immun. 2000; 68:233-8.
38. Lee M H, Pascopella L, Jacobs W R, Jr., Hatfull G F. Site-specific integration of mycobacteriophage L5: integration-proficient vectors for Mycobacterium smegmatis, Mycobacterium tuberculosis, and bacille Calmette-Guerin. Proc.Natl.Acad.Sci.U.S.A 1991; 88:3111-5.
39. Manganelli R, Fattorini L, Tan D et al. The extra cytoplasmic function sigma factor sigma(E) is essential for Mycobacterium tuberculosis virulence in mice. Infect.Immun. 2004a; 72:3038-41.
40. Manganelli R, Provvedi R, Rodrigue S, Beaucher J, Gaudreau L, Smith I. Sigma factors and global gene regulation in Mycobacterium tuberculosis. J.Bacteriol. 2004b; 186:895-902.
41. Manganelli R, Voskuil M I, Schoolnik G K, Dubnau E, Gomez M, Smith I. Role of the extracytoplasmic-function sigma factor sigma(H) in Mycobacterium tuberculosis global gene expression. Mol.Microbiol. 2002; 45:365-74.
42. Manganelli R, Voskuil MI, Schoolnik G K, Smith I. The Mycobacterium tuberculosis ECF sigma factor sigmae: role in global gene expression and survival in macrophages. Mol.Microbiol. 2001; 41:423-37.
43. McCord J M, Fridovich I. Superoxide dismutase. An enzymic function for erythrocuprein (hemocuprein). J.Biol.Chem. 1969; 244:6049-55.
44. McKinney J D, Honer zu Bentrup K., Munoz-Elias E J et al. Persistence of Mycobacterium tuberculosis in macrophages and mice requires the glyoxylate shunt enzyme isocitrate lyase. Nature 2000; 406:735-8.
45. McMurray D N. Determinants of vaccine-induced resistance in animal models of pulmonary tuberculosis. Scand.J.Infect.Dis. 2001; 33:175-8.
46. Miller B H, Shinnick T M. Evaluation of Mycobacterium tuberculosis genes involved in resistance to killing by human macrophages. Infect.Immun. 2000; 68:387-90.
47. Mostowy S, Tsolaki A G, Small P M, Behr M A. The in vitro evolution of BCG vaccines. Vaccine 2003; 21:4270-4.
48. Murray P J, Aldovini A, Young R A. Manipulation and potentiation of antimycobacterial immunity using recombinant bacille Calmette-Guerin strains that secrete cytokines. Proc.Natl.Acad.Sci.U.S.A 1996; 93:934-9.
49. Otsuki Y, Li Z, Shibata M A. Apoptotic detection methods—from morphology to gene. Prog.Histochem.Cytochem. 2003; 38:275-339.
50. Parish T, Stoker N G. Use of a flexible cassette method to generate a double unmarked Mycobacterium tuberculosis tlyA plcABC mutant by gene replacement. Microbiology 2000; 146:1969-75.
51. Pavelka M S, Jr., Jacobs W R, Jr. Comparison of the construction of unmarked deletion mutations in Mycobacterium smegmatis, Mycobacterium bovis bacillus Calmette-Guerin, and Mycobacterium tuberculosis H37Rv by allelic exchange. J.Bacteriol. 1999; 181:4780-9.
52. Pelicic V, Jackson M, Reyrat J M, Jacobs W R, Jr., Gicquel B, Guilhot C. Efficient allelic exchange and transposon mutagenesis in Mycobacterium tuberculosis. Proc.Natl.Acad.Sci.U.S.A 1997; 94:10955-60.
53. Raman S, Song T, Puyang X, Bardarov S, Jacobs W R, Jr., Husson R N. The alternative sigma factor SigH regulates major components of oxidative and heat stress responses in Mycobacterium tuberculosis. J.Bacteriol. 2001; 183:6119-25.
54. Rehse P H, Kumei M, Tahirov T H. Compact reduced thioredoxin structure from the thermophilic bacteria Thermus thermophilus. Proteins 2005.
55. Repique C J, Li A, Collins F M, Morris S L. DNA immunization in a mouse model of latent tuberculosis: effect of DNA vaccination on reactivation of disease and on reinfection with a secondary challenge. Infect.Immun. 2002; 70:3318-23.
56. Rodriguez A, Regnault A, Kleijmeer M, Ricciardi-Castagnoli P, Amigorena S. Selective transport of internalized antigens to the cytosol for MHC class I presentation in dendritic cells. Nat.Cell Biol. 1999; 1:362-8.
57. Sainbandamurthy V K, Wang X, Chen B et al. A pantothenate auxotroph of Mycobacterium tuberculosis is highly attenuated and protects mice against tuberculosis. Nat.Med. 2002; 8:1171-4.
58. Schaible U E, Winau F, Sieling P A et al. Apoptosis facilitates antigen presentation to T lymphocytes through MHC-I and CD1 in tuberculosis. Nat.Med. 2003; 9:1039-
59. Seder R A, Hill A V. Vaccines against intracellular infections requiring cellular immunity. Nature 2000; 406:793-8.
60. Serbina N V, Flynn J L. Early emergence of CD8⁺ T cells primed for production of type 1 cytokines in the lungs of Mycobacterium tuberculosis-infected mice. Infect.Immun. 1999; 67:3980-8.
61. Serbina N V, Liu C C, Scanga C A, Flynn J L. CD8+ CTL from lungs of Mycobacterium tuberculosis-infected mice express perforin in vivo and lyse infected macrophages. J.Immunol. 2000; 165:353-63.
62. Silva C L, Bonato V L, Lima V M, Faccioli L H, Leao S C. Characterization of the memory/activated T cells that mediate the long-lived host response against tuberculosis after bacillus Calmette-Guerin or DNA vaccination. Immunology 1999; 97:573-81.
63. Smith D A, Parish T, Stoker N G, Bancroft G J. Characterization of auxotrophic mutants of Mycobacterium tuberculosis and their potential as vaccine candidates. Infect.Immun. 2001; 69:1142-50.
64. Steensma D P, Timm M, Witzig T E. Flow cytometric methods for detection and quantification of apoptosis. Methods Mol.Med. 2003; 85:323-32.
65. Tullius M V, Harth G, Horwitz M A. Glutamine syntlietase GlnA1 is essential for growth of Mycobacterium tuberculosis in human THP-1 macrophages and guinea pigs. Infect.Immun. 2003; 71:3927-36.
66. Velikovsky C A, Cassataro J, Giambartolomei G H et al. A DNA vaccine encoding lumazine synthase from Brucella abortus induces protective immunity in BALB/c mice. Infect.Immun. 2002; 70:2507-11.
67. Wang P F, Arscott L D, Gilberger T W, Muller S, Williams C H, Jr. Thioredoxin reductase from Plasmodium falciparum: evidence for interaction between the C-terminal cysteine residues and the active site disulfide-dithiol. Biochemistry (Mosc). 1999; 38:3187-96.
68. Winau F, Hegasy G, Kaufmann S H, Schaible U E. No life without death-apoptosis as prerequisite for T cell activation. Apoptosis. 2005; 10:707-15.
69. Winau F, Kaufmann S H, Schaible U E. Apoptosis paves the detour path for CD8 T cell activation against intracellular bacteria. Cell Microbiol. 2004; 6:599-607.
70. Woolfolk C A, Shapiro B, Stadtman E R. Regulation of glutamine synthetase. I. Purification and properties of glutamine synthetase from Escherichia coli. Arch.Biochem.Biophys. 1966; 116:177-92.
71. Zhang Y, Lathigra R, Garbe T, Catty D, Young D. Genetic analysis of superoxide dismutase, the 23 kilodalton antigen of Mycobacterium tuberculosis. Mol.Microbiol. 1991; 5:381-91.
72. Zhong L, Arner E S, Holmgren A. Structure and mechanism of mammalian thioredoxin reductase: the active site is a redox-active selenolthiol/selenenylsulfide formed from the conserved cysteine-selenocysteine sequence. Proc.Natl.Acad.Sci.U.S.A 2000; 97:5854-9.
73. Govoni G, Vidal S, Gauthier S, Skamene E, Malo D, Gros P. The Bcg/Ity/Lsh locus: genetic transfer of resistance to infections in C57BL/6J mice transgenic for the Nrampl Gly169 allele. Infect.Immun. 1996; 64:2923-9.
74. Blattman J N, Grayson J M, Wherry E J, Kaech S M, Smith K A, Ahmed R. Therapeutic use of IL-2 to enhance antiviral T-cell responses in vivo. Nat.Med. 2003; 9:540-7.
75. Olsen A W, Brandt L, Agger E M, van Pinxteren L A, Andersen P. The influence of remaining live BCG organisms in vaccinated mice on the maintenance of immunity to tuberculosis. Scand.J.Immunol. 2004; 60:273-7.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of modifying a bacterium to enhance the immunogenicity of the bacterium, comprising genetically altering the bacterium to express a dominant-negative mutant of an anti-apoptotic enzyme, whereby the bacterium has enhanced immunogenicity in a subject.

2. A modified bacterium made in accordance with the method of claim 1.

3. An immunogenic composition comprising the modified bacterium of claim 2.

4. (canceled)

5. The method of claim 1, wherein the bacterium is selected from the group consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Yersinia enterolitica, and other Yersinia species.

6. The method of claim 1, wherein the dominant-negative mutant is a dominant-negative mutant of SodA in which a deletion, insertion, and/or substitution of nucleotides in the naturally occurring nucleic acid encodes a molecule that interferes with the SOD activity of the organism.

7. (canceled)

8. The method claim 6, wherein the bacterium is BCG.

9. The method of claim 8, comprising a further pro-apoptotic modification.

10. The method of claim 9, wherein the further pro-apoptotic modification comprises one or more modification selected from the group consisting of inactivation of SigH, inactivation of sigE, inactivation of SecA2, inactivation of thioredoxin, inactivation of thioredoxin reductase and inactivation of glutaredoxin.

11. The method of claim 8, wherein the dominant-negative mutant is a mutant SodA having deletions of histidine at position 28 and histidine at position 76.

12. The method of claim 8, wherein the dominant-negative mutant is a mutant SodA having a deletion of histidine at position 28 or a histidine at position 76.

13. The method of claim 8, wherein the dominant-negative mutant is a mutant SodA having a deletion of glutamic acid at position 54.

14. The method of claim 8, wherein the dominant-negative mutant is a mutant SodA having a deletion of glutamic acid at position 54 and the replacement of histidine with arginine at position 28.

15. The method claim 10, wherein the bacterium comprises a dominant-negative mutant of SodA and an activity reducing mutation of sigH.

16. The method of claim 10, wherein the bacterium comprises a dominant-negative mutant of SodA and an activity reducing mutation of secA2.

17. The method of claim 10, wherein the bacterium comprises a dominant-negative mutant of SodA, an activity reducing mutation of sigH and an activity reducing mutation of secA2.

18-34. (canceled)

35. The modified bacterium of 2, wherein the bacterium is selected from the group consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Yersinia enterolitica, and other Yersinia species.

36. The modified bacterium of claim 2, wherein the dominant-negative mutant is a dominant-negative mutant selected from the group consisting of
a) SodA in which a deletion, insertion, and/or substitution of nucleotides in the naturally occurring nucleic acid encodes a molecule that reduces the SOD activity of the organism; and
b) glutamine synthase in which a deletion, insertion, and/or substitution of nucleotides in the naturally occurring nucleic acid encodes a molecule that reduces the glutamine synthase activity of the organism.

37. The modified bacterium of claims 36, wherein the bacterium is BCG.

38. The modified bacterium of claim 37, comprising a further pro-apoptotic modification.

39. The modified bacterium claim 38, wherein the further pro-apoptotic modification comprises one or more modification selected from the group consisting of inactivation of SigH, inactivation of sigE, inactivation of SecA2, inactivation of thioredoxin, inactivation of thioredoxin reductase and inactivation of glutaredoxin.

40. The modified bacterium claim 37, wherein the dominant-negative mutant is a mutant SodA having deletions of histidine at position 28 and histidine at position 76.

41. The modified bacterium claim 37, wherein the dominant-negative mutant is a mutant SodA having a deletion of histidine at position 28 or a histidine at position 76.

42. The modified bacterium claim 37, wherein the dominant-negative mutant is a mutant SodA having a deletion of glutamic acid at position 54.

43. The modified bacterium claim 37, wherein the dominant-negative mutant is a mutant SodA having a deletion of glutamic acid at position 54 and the replacement of histidine with arginine at position 28.

44. The modified bacterium of claim 39, wherein the bacterium comprises a dominant-negative mutant of SodA and an activity reducing mutation of sigH.

45. The modified bacterium of claim 39, wherein the bacterium comprises a dominant-negative mutant of SodA and an activity reducing mutation of secA2.

46. The modified bacterium of claims 39, wherein the bacterium comprises a dominant-negative mutant of SodA, an activity reducing mutation of sigH and an activity reducing mutation of secA2.

47-60. (canceled)

61. The modified bacterium of claim 2, wherein the bacterium comprises an activity reducing mutation of sigH.

62. The modified bacterium of claim 2, wherein the bacterium comprises an activity reducing mutation of sigH and an activity reducing mutation of secA2.
